(19) United States
(12) Patent Application Publication
(10) Pub. No.: US 2018/0237787 A1
(43) Pub. Date: Aug. 23, 2018

(54) GENE EDITING OF PCSK9

(71) Applicant: President and Fellows of Harvard College, Cambridge, MA (US)

(72) Inventors: Juan Pablo Maianti, Revere, MA (US); David R. Liu, Lexington, MA (US)

(21) Appl. No.: 15/852,526

(22) Filed: Dec. 22, 2017

Related U.S. Application Data
(60) Provisional application No. 62/438,869, filed on Dec. 23, 2016.

(51) Int. Cl. C12N 15/74; C12N 15/113; C12N 15/10; C12N 9/22; C12N 15/85; C12N 15/90

(52) U.S. Cl. CPC C12N 15/74; C12N 15/113; C12N 15/1082; C12N 9/22; C12N 2310/531; C12N 15/907; C12N 15/102; C12N 2310/20; C12N 2310/3519; C12N 15/8509

(57) ABSTRACT

Provided herein are systems, compositions, and methods of introducing loss-of-function mutations in to protein factors involved in the LDL-R-mediated cholesterol clearance pathway, e.g., PCSK9, APOC3, LDL-R, or IDOL. Loss-of-function mutations may be introduced using a CRISPR/Cas9-based nucleobase editor described in. Further provided herein are compositions and methods of treating conditions related to high circulating cholesterol levels.

### RELATED APPLICATIONS

This application claims priority under 35 U.S.C. § 119(e) to U.S. Provisional Application Ser. No. 62/438,869, filed Dec. 23, 2016, which is incorporated herein by reference.

### GOVERNMENT SUPPORT

This invention was made with government support under grant number GM065865, awarded by the National Institutes of Health (NIH). The government has certain rights in the invention.

### BACKGROUND OF THE INVENTION

The liver protein Proprotein Convertase Subtilisin/Kexin Type 9 (PCSK9) is a secreted, globular, auto-activating serine protease that acts as a protein-binding adaptor within endosomal vesicles to bridge a pH-dependent interaction with the low-density lipoprotein receptor (LDL-R) during endocytosis of LDL particles, preventing recycling of the LDL-R to the cell surface and leading to reduction of LDL-cholesterol clearance. Blocking or inhibiting the function of PCSK9 to boost LDL-R-mediated clearance of LDL cholesterol has been of significant interest in the pharmaceutical industry. However, current methods of generating PCSK9 protective variants and loss-of-function mutants in vivo have been ineffective due to the large number of cells that need to be modified to modulate cholesterol levels. Other concerns involve off-target effects, genome instability, or oncogenic modifications that may be caused by genome editing.

### SUMMARY OF THE INVENTION

Provided herein are systems, compositions, kits, and methods for modifying a polynucleotide (e.g., DNA) encoding a PCSK9 protein to produce loss-of-function PCSK9 variants. Also provided herein are systems, compositions, kits, and methods for modifying a polynucleotide (e.g., DNA) encoding a LDLR, IDOL, or APOC3/C5 protein to produce loss-of-function mutants. The methodology for producing the mutatns relies on CRISPR/Cas9-based base-editing technology. The precise targeting methods described herein are superior to previously proposed strategies that create random indels in the PCSK9 genomic locus or other loci described herein using engineered nucleases. The methods also have a more favorable safety profile, due to the low probability of off-target effects. Thus, the base editing methods described herein have low impact on genomic stability, including oncogene activation or tumor suppressor inactivation. In some embodiments, the loss-of-function variants (e.g., PCSK9, LDLR, IDOL, or APOC3/C5 variants) generated using the methods described herein have a cardioprotective function. In some embodiments, the loss-of-function variants (e.g., PCSK9, LDLR, IDOL, or APOC3/C5 variants) generated using the methods described herein reduce LDL levels. In some embodiments, the loss-of-function variants (e.g., PCSK9, LDLR, IDOL, or APOC3/C5 variants) generated using the methods described herein reduce LDL cholesterol levels. In some embodiments, the loss-of-function variants (e.g., PCSK9, LDLR, IDOL, or APOC3/C5 variants) generated using the methods described herein lower overall cholesterol levels. In some embodiments, the loss-of-function variants (e.g., PCSK9, LDLR, IDOL, or APOC3/C5 variants) generated using the methods described herein increase HDL levels.

Some aspects of the present disclosure provide methods of editing a polynucleotide encoding a Proprotein Convertase Subtilisin/Kexin Type 9 (PCSK9) protein, the method comprising contacting the PCSK9-encoding polynucleotide with (i) a fusion protein comprising: (a) a guide nucleotide sequence-programmable DNA binding protein domain; and (b) a cytosine deaminase domain; and (ii) a guide nucleotide sequence targeting the fusion protein of (i) to a target cytosine (C) base in the PCSK9-encoding polynucleotide, wherein the contacting results in deamination of the target C base by the fusion protein, resulting in a cytosine (C) to thymine (T) change in the PCSK9-encoding polynucleotide.

In some embodiments, the guide nucleotide sequence-programmable DNA binding protein domain is selected from the group consisting of nuclease inactive Cas9 (dCas9) domains, nuclease inactive Cpf1 domains, nuclease inactive Argonaute domains, and variants and combinations thereof. In some embodiments, the guide nucleotide sequence-programmable DNA-binding protein domain is a nuclease inactive Cas9 (dCas9) domain. In some embodiments, the amino acid sequence of the dCas9 domain comprises mutations corresponding to a D10A and/or H840A mutation in SEQ ID NO: 1. In some embodiments, a Cas9 nickase is used. In some embodiments, the amino acid sequence of the Cas9 nickase comprises a mutation corresponding to a D10A mutation in SEQ ID NO: 1, and wherein the dCas9 domain comprises a histidine at the position corresponding to amino acid 840 of SEQ ID NO: 1.

In some embodiments, the guide nucleotide sequence-programmable DNA-binding protein domain comprises a nuclease inactive Cpf1 (dCpf1) domain. In some embodiments, the dCpf1 domain is from a species of Acidaminococcus or Lachnospiraceae.

In some embodiments, the guide nucleotide sequence-programmable DNA-binding protein domain comprises a nuclease inactive Argonaute (dAgo) domain. In some embodiments, the dAgo domain is from Natronobacterium gregoryi (dNgAgo).

As a set of non limiting examples, any of the fusion proteins described herein that include a Cas9 domain can use another guide nucleotide sequence-programmable DNA binding protein, such as CasX, CasY, Cpf1, C2c1, C2c2, C2c3, and Argonaute, in place of the Cas9 domain. These may be nuclease inactive variants of the proteins. Guide nucleotide sequence-programmable DNA binding protein include, without limitation, Cas9 (e.g., dCas9 and nCas9), saCas9 (e.g., saCas9d, saCas9n, saKKH Cas9), CasX, CasY, Cpf1, C2c1, C2c2, C2C3, Argonaute, and any of suitable protein described herein. In some embodiments, the fusion protein described herein comprises a Gam protein, a guide nucleotide sequence-programmable DNA binding protein, and a cytidine deaminase domain.

In some embodiments, the cytosine deaminase domain comprises an apolipoprotein B mRNA-editing complex (APOBEC) family deaminase. In some embodiments, the cytosine deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, APOBEC3H deaminase, APOBEC4 deaminase, activation-induced deaminase (AID), and pmCDA1. In some embodiments, the cytosine deaminase comprises the amino acid sequence of any one of SEQ ID NOs: 271-292 and 303.

In some embodiments, the fusion protein of (a) further comprises a uracil glycosylase inhibitor (UGI) domain. In some embodiments, the cytosine deaminase domain is fused to the N-terminus of the guide nucleotide sequence-programmable DNA-binding protein domain. In some embodiments, the UGI domain is fused to the C-terminus of the guide nucleotide sequence-programmable DNA-binding protein domain.

In some embodiments, the cytosine deaminase is fused to the guide nucleotide sequence-programmable DNA-binding protein domain via an optional linker. In some embodiments, the UGI domain is fused to the dCas9 domain via an optional linker. In some embodiments, the fusion protein comprises the structure NH₂-[cytosine deaminase domain]-[optional linker sequence]-[guide nucleotide sequence-programmable DNA-binding protein domain]-[optional linker sequence]-[UGI domain]-COOH.

In some embodiments, the linker comprises (GGGS)ₙ (SEQ ID NO: 1998), (GGGGS)ₙ (SEQ ID NO: 308), (G)ₙ, (EAAAK)ₙ (SEQ ID NO: 309), (GGS)ₙ, SGSETPGTSESATPES (SEQ ID NO: 310), or (XP)ₙ motif, or a combination of any of these, wherein n is independently an integer between 1 and 30, and wherein X is any amino acid. In some embodiments, the linker comprises the amino acid sequence SGSETPGTSESATPES (SEQ ID NO: 310). In some embodiments, the linker is (GGS)ₙ, wherein n is 1, 3, or 7.

In some embodiments, the fusion protein comprises the amino acid sequence of any one of SEQ ID NOs: 10 and 293-302.

In some embodiments, the polynucleotide encoding the PCSK9 protein comprises a coding strand and a complementary strand. In some embodiments, the polynucleotide encoding the PCSK9 protein comprises a coding region and a non-coding region.

In some embodiments, the C to T change occurs in the coding sequence or on the coding strand of the PCSK9-encoding polynucleotide. In some embodiments, the C to T change leads to a mutation in the PCSK9 protein. In some embodiments, the mutation in the PCSK9 protein is a loss-of-function mutation. In some embodiments, the mutation is selected from the mutations listed in Table 3. In some embodiments, the guide nucleotide sequence useful in the present invention is selected from the guide nucleotide sequences listed in Table 3.

In some embodiments, the loss-of-function mutation introduces a premature stop codon in the PCSK9 coding sequence that leads to a truncated or non-functional PCSK9 protein. In some embodiments, the premature stop codon is TAG (Amber), TGA (Opal), or TAA (Ochre).

In some embodiments, the premature stop codon is generated from a CAG to TAG change via the deamination of the first C on the coding strand. In some embodiments, the premature stop codon is generated from a CGA to TGA change via the deamination of the first C on the coding strand. In some embodiments, the premature stop codon is generated from a CAA to TAA change via the deamination of the first C on the coding strand. In some embodiments, the premature stop codon is generated from a TGG to TAG change via the deamination of the second C on the complementary strand. In some embodiments, the premature stop codon is generated from a TGG to TGA change via the deamination of the third C on the complementary strand. In some embodiments, the premature stop codon is generated from a CGG to TAG or CGA to TAA change via the deamination of C on the coding strand and the deamination of C on the complementary strand. In some embodiments, the guide nucleotide sequence is selected from the guide nucleotide sequences listed in Table 6 (SEQ ID NO: 938-1123).

In some embodiments, tandem premature stop codons are introduced. In some embodiments, the mutation is selected from the group consisting of: W10X-W11X, Q99X-Q101X, Q342X-Q344X, and Q554X-Q555X, wherein X is a stop codon. The guide nucleotide sequences for the consecutive mutations may be found in Table 6.

In some embodiments, the premature stop codon is introduced after a structurally destabilizing mutation. In some embodiments, the mutation is selected from the group consisting of: P530S/L-Q531X, P581S/L-R582X, and P618S/L-Q619X, wherein X is a stop codon. In some embodiments, the guide nucleotide sequence used for introducing the premature stop codon is selected from SEQ ID NOs: 938-1123, and wherein the guide nucleotide sequence used for introducing the structurally destabilizing mutation is selected from SEQ ID NOs: 579-937. In some embodiments, the mutation destabilizes PCSK9 protein folding.

In some embodiments, mutation is selected from the mutations listed in Table 4. In some embodiments, the guide nucleotide sequence is selected from the guide nucleotide sequences listed in Table 4 (SEQ ID NOs.: 579-937).

In some embodiments, the C to T change occurs at a splicing site in the non-coding region of the PCSK9-encoding polynucleotide. In some embodiments, the C to T change occurs at an intron-exon junction. In some embodiments, the C to T change occurs at a splicing donor site. In some embodiments, the C to T change occurs at a splicing acceptor site. In some embodiments, the C to T changes occurs at a C base-paired with the G base in a start codon (AUG). In some embodiments, the C to T change prevents PCSK9 mRNA maturation or abrogates PCSK9 expression. In some embodiments, the guide nucleotide sequence is selected from the guide nucleotide sequences listed in Table 8 (SEQ ID NOs: 1124-1309).

In some embodiments, a PAM sequence is located 3′ of the C being changed, e.g., aPAM selected from the group consisting of: NGG, NGAN, NGNG, NGAG, NGCG, NNGRRT, NGRRN, NNNRRT, NGGNG, NNNGATT, NNAGAA, and NAAAC, wherein Y is pyrimidine, R is purine, and N is any nucleobase. In some embodiments a PAM sequence is located 5′ of the C being change, e.g., a PAM selected from the group consisting of: NNT, NNNT, and YNT, wherein Y is pyrimidine, and N is any nucleobase. In some embodiments, no PAM sequence is located at either 5′ or 3′ of the target C base.

In some embodiments, at least 1, 2, 3, 4, 5, 6, 7, 8, 9, or 10 mutations are introduced into the PCSK9-encoding polynucleotide.

In some embodiments, the guide nucleotide sequence is RNA (guide RNA or gRNA). In some embodiments, the guide nucleotide sequence is ssDNA (guide DNA or gDNA).

Other aspects of the present disclosure provide methods of editing a polynucleotide encoding an Apolipoprotein C3 (APOC3) protein, the method comprising contacting the APOC3-encoding polynucleotide with: (i) a fusion protein comprising: (a) a guide nucleotide sequence-programmable DNA binding protein domain; and (b) a cytosine deaminase domain; and (ii) a guide nucleotide sequence targeting the fusion protein of (i) to a target cytosine (C) base in the APOC3-encoding polynucleotide, wherein the contacting results in deamination of the target C base by the fusion protein, resulting in a cytosine (C) to thymine (T) change in the APOC3-encoding polynucleotide. In some embodiments, the guide nucleotide sequence is selected from SEQ ID NOs: 1806-1906.

Other aspects of the present disclosure provide methods of editing a polynucleotide encoding a Low-Density Lipoprotein Receptor (LDL-R) protein, the method comprising contacting the LDL-R-encoding polynucleotide with: (i) a fusion protein comprising: (a) a guide nucleotide sequence-programmable DNA binding protein domain; and (b) a cytosine deaminase domain; and (ii) a guide nucleotide sequence targeting the fusion protein of (i) to a target cytosine (C) base in the LDL-R-encoding polynucleotide, wherein the contacting results in deamination of the target C base by the fusion protein, resulting in a cytosine (C) to thymine (T) change in the LDLR-encoding polynucleotide. In some embodiments, the guide nucleotide sequence is selected from SEQ ID NOs: 1792-1799.

Other aspects of the present disclosure provide methods of editing a polynucleotide encoding an Inducible Degrader of the LDL receptor (IDOL) protein, the method comprising contacting the IDOL-encoding polynucleotide with: (i) a fusion protein comprising: (a) a guide nucleotide sequence-programmable DNA binding protein domain; and (b) a cytosine deaminase domain; and (ii) a guide nucleotide sequence targeting the fusion protein of (i) to a target C base in the IDOL-encoding polynucleotide, wherein the contacting results in deamination of the target C base by the fusion protein, resulting in a cytosine (C) to thymine (T) change in the IDOL-encoding polynucleotide. In some embodiments, the guide nucleotide sequence is selected from SEQ ID NOs: 1788-1791.

In some embodiments, the method is carried out in vitro. In some embodiments, the method is carried out in a cultured cell. In some embodiments, the method is carried out in vivo. In some embodiments, the method is carried out ex vivo.

In some embodiments, the method is carried out in a mammal. In some embodiments, wherein the mammal is a rodent. In some embodiments, the mammal is a primate. In some embodiments, the mammal is human. In some embodiments, the method is carried out in an organ of a subject, e.g., liver.

Other aspects of the present disclosure provide methods of editing a polynucleotide encoding a Proprotein Convertase Subtilisin/Kexin Type 9 (PCSK9) protein, the method comprising contacting the PCSK9-encoding polynucleotide with a fusion protein comprising: (a) a programmable DNA binding protein domain; and (b) a deaminase domain, wherein the contacting results in deamination of the target base by the fusion protein, resulting in base change in the PCSK9-encoding polynucleotide.

In some embodiments, the programmable DNA-binding domain comprises a zinc finger nuclease (ZFN) domain. In some embodiments, the programmable DNA-binding domain comprises a transcription activator-like effector (TALE) domain. In some embodiments, the programmable DNA-binding domain is a guide nucleotide sequence-programmable DNA binding protein domain.

In some embodiments, the programmable DNA-binding domain is selected from the group consisting of: nuclease inactive Cas9 domains (e.g., dCas9 and nCas9), nuclease inactive Cpf1 domains, nuclease inactive Argonaute domains, and variants thereof. In some embodiments, the programmable DNA-binding domain is a CasX, CasY, C2c1, C2c2, or C2c3 domain, or variants thereof. In some embodiments, the programmable DNA-binding domain is a saCas9 (e.g., saCas9d, saCas9n, saKKH Cas9) domain, or variants thereof. In some embodiments, the programmable DNA-binding domain is associated with a guide nucleotide sequence. In some embodiments, the deaminase is a cytosine deaminase. In some embodiments, the target base is a cytosine (C) base and the deamination of the target C base results in a C to deoxyuridine (dU) change, which precedes the introduction of thymine (T) in place of the target C. In some embodiments, the fusion protein described herein comprises a Gam protein, a guide nucleotide sequence-programmable DNA-binding domain, and a cytidine deaminase domain.

Some aspects of the present disclosure provide compositions comprising: (i) a fusion protein comprising: (a) a guide nucleotide sequence-programmable DNA binding protein domain; and (b) a cytosine deaminase domain; and (ii) a guide nucleotide sequence targeting the fusion protein of (i) to a polynucleotide encoding a Proprotein Convertase subtilisin/Kexin Type 9 (PCSK9) protein. In some embodiments, the fusion protein of (i) further comprises a Gam protein.

Other aspects of the present disclosure provide compositions comprising: (i) a fusion protein comprising: (a) a guide nucleotide sequence-programmable DNA binding protein domain; and (b) a cytosine deaminase domain; (ii) a guide nucleotide sequence targeting the fusion protein of (i) to a polynucleotide encoding a Proprotein Convertase subtilisin/Kexin Type 9 (PCSK9) protein; and (ii) a guide nucleotide sequence targeting the fusion protein of (i) to a polynucleotide encoding an Apolipoprotein C3 protein. In some embodiments, the fusion protein of (i) further comprises a Gam protein.

Other aspects of the present disclosure provide compositions comprising: (i) a fusion protein comprising: (a) a guide nucleotide sequence-programmable DNA binding protein domain; and (b) a cytosine deaminase domain; (ii) a guide nucleotide sequence targeting the fusion protein of (i) to a polynucleotide encoding a Proprotein Convertase subtilisin/Kexin Type 9 (PCSK9) protein; (iii) a guide nucleotide sequence targeting the fusion protein of (i) to a polynucleotide encoding an Apolipoprotein C3 protein; and (iv) a guide nucleotide sequence targeting the fusion protein of (i) to a polynucleotide encoding Low-Density Lipoprotein Receptor protein. In some embodiments, the fusion protein of (i) further comprises a Gam protein.

Other aspects of the present disclosure provide compositions comprising: (i) a fusion protein comprising (a) a guide nucleotide sequence-programmable DNA binding protein domain; and (b) a cytosine deaminase domain; a guide nucleotide sequence targeting the fusion protein of (i) to a polynucleotide encoding a Proprotein Convertase subtilisin/Kexin Type 9 (PCSK9) protein; in some embodiments, a guide nucleotide sequence targeting the fusion protein of (i) to a polynucleotide encoding an Apolipoprotein C3 protein; in some embodiments, a guide nucleotide sequence targeting the fusion protein of (i) to a polynucleotide encoding Low-Density Lipoprotein Receptor protein; and in some embodiments, a guide nucleotide sequence targeting the fusion protein of (i) to a polynucleotide encoding Inducible Degrader of the LDL receptor protein. In some embodiments, the fusion protein of (i) further comprises a Gam protein.

In some embodiments, the guide nucleotide sequence of (ii) is selected from SEQ ID NOs: 336-1309. In some embodiments, the guide nucleotide sequence of (iii) is selected from SEQ ID NOs: 1806-1906. In some embodiments, the guide nucleotide sequence of (iv) is selected from SEQ ID NOs: 1792-1799. In some embodiments, the guide nucleotide sequence of (v) is selected from SEQ ID NOs: 1788-1791.

Other aspects of the present disclosure provide compositions comprising a nucleic acid encoding the fusion protein and the guide nucleotide sequence described herein. In some embodiments, the composition further comprising a pharmaceutically acceptable carrier.

Other aspects of the present disclosure provide methods of boosting LDL receptor-mediated clearance of LDL cholesterol, the method comprising administering to a subject in need thereof a therapeutically effective amount of the composition described herein.

Other aspects of the present disclosure provide methods of reducing circulating cholesterol level in a subject, the method comprising administering to a subject in need thereof an therapeutically effective amount of the composition described herein.

Other aspects of the present disclosure provide methods of treating a condition, the method comprising administering to a subject in need thereof an therapeutically effective amount of the composition described herein. In some embodiments, the condition is hypercholesterolemia, elevated total cholesterol levels, elevated low-density lipoprotein (LDL) levels, elevated LDL-cholesterol levels, reduced high-density lipoprotein levels, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, or a combination thereof.

Further provided herein are kits comprising the compositions described herein.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1A depicts a pre-pro-PCSK9 open-reading frame showing naturally-occurring gain-of-function (GOF) variants identified in human populations associated with elevated low-density lipoproteins (LDL) cholesterol, leading to increased LDL receptor (LDL-R) degradation, and other variants that display beneficial loss-of-function (LOF) phenotypes associated with lower LDL cholesterol and cardioprotection. Variants highlighted in red have been mechanistically confirmed. Key catalytic site residues are shown.^{3b}

FIG. 1B is a model of uncleaved pro-Proprotein Convertase Subtilisin/Kexin Type 9 (PCSK9) (based on PDB: 1R6V) showing the position of the catalytic triad residues (Asp186, His226, and Ser386) and selected residues that produce GOF (S127R, F216L, D374Y) or LOF variants (R46L, ΔR97, L253F, A433T) affecting PCSK9 proteolytic auto-activation, protease inactivation, or LDL-R binding affinity (see Tables 1 and 2).

FIG. 1C shows interactions between PCSK9 and the EGF-A domain of LDL-R observed in the X-ray co-structure (PDB: 3BPS).¹⁹

FIG. 2 is a scheme of the basic functions of PCSK9 in hepatocyte cells preventing LDL-R recycling to the cell surface after endocytosis of LDL. Multiple strategies for blocking PCSK9 function are being explored in the pharma sector (Table 12), including two FDA approved anti-PCSK9 antibody therapeutics, other antibodies in phase 2-3, and in pre-clinical phases: adnectin, peptides, small-molecules, antisense oligos, and RNA-interference.

FIG. 3A shows a strategy for preventing PCSK9 mRNA maturation and protein production by altering splicing sites: donor site, branch-point, or acceptor sites.

FIGS. 3B to 3D show consensus sequences of the human spliceosomal intron branch-point, donor and acceptor sites, suggesting that the guanosine of the donor and acceptor sites is an excellent target for base-editing of C→T reactions on the complementary strand.

FIG. 4 shows protein and open-reading frame sequences for PCSK9. Residues highlighted in grey correspond to Table 4 (premature stop codons), or Table 5 (destabilizing variants). The top level nucleotide sequence in this figure depicts SEQ ID NO: 1990. The second level amino acid sequence in this figure depicts SEQ ID NO: 1991.

FIG. 5 is a PCSK9 genomic sequence showing exons (capitalized) and introns (lowercase). Key nucleotides in the exon/intron junctions are underlined. This figure depicts SEQ ID NO: 1994.

FIG. 6 is a graph showing the numbering schemes of the relative location of PAM and the target sequence. This figure depicts SEQ ID NO: 1995.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

“Cholesterol” refers to a lipid molecule biosynthesized by all animal cells. Not wishing to be bound to a specific theory, cholesterol is an essential structural component of all animal cell membranes that is required to maintain both membrane structural integrity and fluidity. Cholesterol enables animal cells to dispense with a cell wall (to protect membrane integrity and cell viability) thus allowing animal cells to change shape and animals to move (unlike bacteria and plant cells which are restricted by their cell walls). In addition to its importance for animal cell structure, cholesterol also serves as a precursor for the biosynthesis of steroid hormones and bile acids. Cholesterol is the principal sterol synthesized by all animals. In vertebrates the hepatic cells typically produce greater amounts than other cells. It is generally absent among prokaryotes (bacteria and archaea).

All animal cells manufacture cholesterol, for both membrane structure and other uses, with relative production rates varying by cell type and organ function. About 20% of total daily cholesterol production occurs in the liver; other sites of higher synthesis rates include the intestines, adrenal glands, and reproductive organs. The liver excretes cholesterol into biliary fluids, which is then stored in the gallbladder. Bile contains bile salts, which solubilize fats in the digestive tract and aid in the intestinal absorption of fat molecules as well as the fat-soluble vitamins, A, D, E, and K. Cholesterol is recycled in the body. Typically, about 50% of the excreted cholesterol by the liver is reabsorbed by the small bowel back into the bloodstream.

As an isolated molecule, cholesterol is only minimally soluble in water; it dissolves into the (water-based) bloodstream only at small concentrations. Instead, cholesterol is transported within lipoproteins, complex discoidal particles with exterior amphiphilic proteins and lipids, whose outward-facing structures are water-soluble and inward-facing surfaces are lipid-soluble; i.e. transport via emulsification. The lipoprotein particles are classified based on their density: low-density lipoproteins (LDL), very low-density lipoproteins (VLDL), high-density lipoproteins (HDL), chylomicrons, etc. Triglycerides and cholesterol esters are carried internally. Phospholipids and cholesterol, being amphipathic, are transported in the monolayer surface of the lipoprotein particle.

Surface LDL receptors are internalized during the process of cholesterol absorption, and its synthesis is regulated by SREBP, the same protein that controls the synthesis of cholesterol de novo, according to its concentration inside the cell. A cell with abundant cholesterol will have its LDL receptor synthesis blocked, to prevent new cholesterol in LDL particles from being taken up. Conversely, LDL receptor synthesis is promoted when a cell is deficient in cholesterol.

Not wishing to be bound to any specific theory, if this physiological process becomes unregulated, excess LDL particles will travel in the blood without the opportunity for uptake by an LDL receptor. These LDL particles are oxidized and taken up by macrophages through scavenger receptors, which then become engorged and form foam cells. These foam cells often become trapped in the walls of blood vessels and contribute to atherosclerotic plaque formation. Differences in cholesterol homeostasis affect the development of early atherosclerosis (carotid intima-media thickness). These plaques are the main causes of heart attacks, strokes, and other serious medical problems, leading to the association of so-called LDL cholesterol (actually a lipoprotein) with “bad” cholesterol.

“Proprotein convertase subtilisin/kexin type 9 (PCSK9)” refers to an enzyme encoded by the PCSK9 gene in humans. PCSK9 binds to the receptor for low-density lipoprotein (LDL) particles. In the liver, the LDL receptor removes LDL particles from the blood through the endocytosis pathway. When PCSK9 binds to the LDL receptor, the receptor is channeled towards the lysosomal pathway and broken down by proteolytic enzymes, limiting the number of times that a given LDL receptor is able to uptake LDL particles from the blood. Thus, blocking PCSK9 activity may lead to more LDL receptors being recycled and present on the surface of the liver cells, and will remove more LDL cholesterol from the blood. Therefore, blocking PCSK9 can lower blood cholesterol levels. PCSK9 orthologs are found across many species. PCSK9 is inactive when first synthesized, a pre-pro enzyme, because a section of the peptide chain blocks its activity; proprotein convertases remove that section to activate the enzyme. Pro-PCSK9 is a secreted, globular, serine protease capable of proteolytic auto-processing of its N-terminal pro-domain into a potent endogenous inhibitor of PCSK9, which blocks its catalytic site. PCSK9's role in cholesterol homeostasis has been exploited medically. Drugs that block PCSK9 can lower the blood level of low-density lipoprotein cholesterol (LDL-C). The first two PCSK9 inhibitors, alirocumab and evolocumab, were approved by the U.S. Food and Drug Administration in 2015 for lowering cholesterol where statins and other drugs were insufficient.

“Low-density lipoprotein (LDL)” refers to one of the five major groups of lipoprotein, from least dense (lower weight-volume ratio particles) to most dense (larger weight-volume ratio particles): chylomicrons, very low-density lipoproteins (VLDL), low-density lipoproteins (LDL), intermediate-density lipoproteins (IDL), and high-density lipoproteins (HDL). Lipoproteins transfer lipids (fats) around the body in the extracellular fluid thereby facilitating fats to be available and taken up by the cells body wide via receptor-mediated endocytosis. Lipoproteins are complex particles composed of multiple proteins, typically 80-100 proteins/particle (organized by a single apolipoprotein B for LDL and the larger particles). A single LDL particle is about 220-275 angstroms in diameter, typically transporting 3,000 to 6,000 fat molecules/particle, varying in size according to the number and mix of fat molecules contained within. The lipids carried include all fat molecules with cholesterol, phospholipids, and triglycerides dominant; amounts of each varying considerably. Lipoproteins can be sampled from blood.

Not wishing to be bound to any specific theory, LDL particles pose a risk for cardiovascular disease when they invade the endothelium and become oxidized, since the oxidized forms are more easily retained by the proteoglycans. A complex set of biochemical reactions regulates the oxidation of LDL particles, mainly stimulated by presence of necrotic cell debris and free radicals in the endothelium. Increasing concentrations of LDL particles are strongly associated with increasing rates of accumulation of atherosclerosis within the walls of arteries over time, eventually resulting in sudden plaque ruptures, decades later, and triggering clots within the artery opening, or a narrowing or closing of the opening, i.e. cardiovascular disease, stroke, and other vascular disease complications.

“Low-Density Lipoprotein (LDL) Receptor” refers to a mosaic protein of 839 amino acids (after removal of 21-amino acid signal peptide) that mediates the endocytosis of cholesterol-rich LDL particles. It is a cell-surface receptor that recognizes the apoprotein B100, which is embedded in the outer phospholipid layer of LDL particles. The receptor also recognizes the apoE protein found in chylomicron remnants and VLDL remnants (IDL). In humans, the LDL receptor protein is encoded by the LDLR gene. LDL receptor complexes are present in clathrin-coated pits (or buds) on the cell surface, which when bound to LDL-cholesterol via adaptin, are pinched off to form clathrin-coated vesicles inside the cell. This allows LDL-cholesterol to be bound and internalized in a process known as endocytosis. This process occurs in all nucleated cells, but mainly in the liver which removes ˜70% of LDL from the circulation.

“Inducible Degrader of the LDL receptor (IDOL)” refers to an ubiquitin ligase that ubiquitinates LDL receptors in endosomes and directs the receptors to the lysosomal compartment for degradation. IDOL is transcriptionally up-regulated by LXR/RXR in response to an increase in intracellular cholesterol. Pharmacologic inhibition of IDOL could reduce plasma LDL cholesterol by increasing plasma LDL receptor density.

“Apolipoprotein C-III (APOC3)” is a protein that in humans is encoded by the APOC3 gene. APOC3 is a component of very low density lipoproteins (VLDL). APOC3 inhibits lipoprotein lipase and hepatic lipase. It is also thought to inhibit hepatic uptake of triglyceride-rich particles. An increase in APOC3 levels induces the development of hypertriglyceridemia. Recent evidence suggests an intracellular role for APOC3 in promoting the assembly and secretion of triglyceride-rich VLDL particles from hepatic cells under lipid-rich conditions. However, two naturally occurring point mutations in human apoC3 coding sequence, A23T and K58E have been shown to abolish the intracellular assembly and secretion of triglyceride-rich VLDL particles from hepatic cells.

The term “Gam protein,” as used herein, refers generally to proteins capable of binding to one or more ends of a double strand break of a double stranded nucleic acid (e.g., double stranded DNA). In some embodiments, the Gam protein prevents or inhibits degradation of one or more strands of a nucleic acid at the site of the double strand break. In some embodiments, a Gam protein is a naturally-occurring Gam protein from bacteriophage Mu, or a non-naturally occurring variant thereof.

The term “loss-of-function mutation” or “inactivating mutation” refers to a mutation that results in the gene product having less or no function (being partially or wholly inactivated). When the allele has a complete loss of function (null allele), it is often called an amorphic mutation in the Muller's morphs schema. Phenotypes associated with such mutations are most often recessive. Exceptions are when the organism is haploid, or when the reduced dosage of a normal gene product is not enough for a normal phenotype (this is called haploinsufficiency).

The term “protective mutation” or “protective variant” refers to a mutation that results in a gene product having an opposing effect or function to the wild type gene. This is often called an antimorphic mutation in the Muller's morphs schema. Phenotypes associated with such mutations are most often dominant. Exceptions are when the organism is haploid, or when the reduced dosage of the antimorphic gene product is not enough to override the wild type phenotype.

The term “gain-of-function mutation” or “activating mutation” refers to a mutation that changes the gene product such that its effect gets stronger (enhanced activation) or even is superseded by a different and abnormal function. A gain of function mutation may also be referred to as a neomorphic mutation. When the new allele is created, a heterozygote containing the newly created allele as well as the original will express the new allele, genetically defining the mutations as dominant phenotypes.

“Hypercholesterolemia,” also called dyslipidemia, is the presence of high levels of cholesterol in the blood. It is a form of high blood lipids and “hyperlipoproteinemia” (elevated levels of lipoproteins in the blood). Elevated levels of non-HDL cholesterol and LDL in the blood may be a consequence of diet, obesity, inherited (genetic) diseases (such as LDL receptor mutations in familial hypercholesterolemia), or the presence of other diseases such as diabetes and an underactive thyroid.

“Hypocholesterolemia” refers to the presence of abnormally low levels of cholesterol in the blood. Although the presence of high total cholesterol (hyper-cholesterolemia) correlates with cardiovascular disease, a defect in the body's production of cholesterol can lead to adverse consequences as well.

The term “genome” refers to the genetic material of a cell or organism. It typically includes DNA (or RNA in the case of RNA viruses). The genome includes both the genes, the coding regions, the noncoding DNA, and the genomes of the mitochondria and chloroplasts. A genome does not typically include genetic material that is artificially introduced into a cell or organism, e.g., a plasmid that is transformed into a bacteria is not a part of the bacterial genome.

A “programmable DNA-binding protein” refers to DNA binding proteins that can be programmed to target to any desired nucleotide sequence within a genome. To program the DNA-binding protein to bind a desired nucleotide sequence, the DNA binding protein may be modified to change its binding specificity, e.g., zinc finger DNA-binding domain, zinc finger nuclease (ZFN), or transcription activator-like effector proteins (TALE). ZFNs are artificial restriction enzymes generated by fusing a zinc finger DNA-binding domain to a DNA-cleavage domain. Zinc finger domains can be engineered to target specific desired DNA sequences and this enables zinc-fingers to bind unique sequences within complex genomes. Transcription activator-like effector nucleases (TALEN) are engineered restriction enzymes that can be engineered to cut specific sequences of DNA. They are made by fusing a TAL effector DNA-binding domain to a nuclease domain (e.g. Fok1). Transcription activator-like effectors (TALEs) can be engineered to bind practically any desired DNA sequence. Methods for programming ZFNs and TALEs are familiar to one skilled in the art. For example, such methods are described in Maeder, et al., Mol. Cell 31 (2): 294-301, 2008; Carroll et al., Genetics Society of America, 188 (4): 773-782, 2011; Miller et al., Nature Biotechnology 25 (7): 778-785, 2007; Christian et al., Genetics 186 (2): 757-61, 2008; Li et al., Nucleic Acids Res. 39 (1): 359-372, 2010; and Moscou et al., Science 326 (5959): 1501, 2009, each of which are incorporated herein by reference.

A “guide nucleotide sequence-programmable DNA-binding protein” refers to a protein, a polypeptide, or a domain that is able to bind DNA, and the binding to its target DNA sequence is mediated by a guide nucleotide sequence. Thus, it is appreciated that the guide nucleotide sequence-programmable DNA-binding protein binds to a guide nucleotide sequence. The “guide nucleotide” may be an RNA or DNA molecule (e.g., a single-stranded DNA or ssDNA molecule) that is complementary to the target sequence and can guide the DNA binding protein to the target sequence. As such, a guide nucleotide sequence-programmable DNA-binding protein may be a RNA-programmable DNA-binding protein (e.g., a Cas9 protein), or an ssDNA-programmable DNA-binding protein (e.g., an Argonaute protein). “Programmable” means the DNA-binding protein may be programmed to bind any DNA sequence that the guide nucleotide targets. Exemplary guide nucleotide sequence-programmable DNA-binding proteins include, but are not limited to, Cas9 (e.g., dCas9 and nCas9), saCas9 (e.g., saCas9d, saCas9d, saKKH Cas9) CasX, CasY, Cpf1, C2c1, C2c2, C2c3, Argonaute, and any other suitable protein described herein, or variants thereof.

In some embodiments, the guide nucleotide sequence exists as a single nucleotide molecule and comprises comprise two domains: (1) a domain that shares homology to a target nucleic acid (e.g., and directs binding of a guide nucleotide sequence-programmable DNA-binding protein to the target); and (2) a domain that binds a guide nucleotide sequence-programmable DNA-binding protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), which is incorporated herein by reference. Other examples of gRNAs (e.g., those including domain 2) can be found in U.S. Patent Application Publication US20160208288 and U.S. Patent Application Publication US20160200779 each of which is herein incorporated by reference.

Because the guide nucleotide sequence hybridizes to a target DNA sequence, the guide nucleotide sequence-programmable DNA-binding proteins are able to specifically bind, in principle, to any sequence complementary to the guide nucleotide sequence. Methods of using guide nucleotide sequence-programmable DNA-binding protein, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); each of which are incorporated herein by reference).

As used herein, the term “Cas9” or “Cas9 nuclease” refers to an RNA-guided nuclease comprising a Cas9 protein, a fragment, or a variant thereof. A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), which is incorporated herein by reference.

Cas9 nuclease sequences and structures are well known to those of skill in the art (see, e.g., Ferretti et al., Proc. Natl. Acad. Sci. 98:4658-4663(2001); Deltcheva E. et al., Nature 471:602-607(2011); and Jinek et al., Science 337:816-821(2012), each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski et al., (2013) RNA Biology 10:5, 726-737; which are incorporated herein by reference. In some embodiments, wild type Cas9 corresponds to Cas9 from Streptococcus pyogenes (NCBI Reference Sequence: NC_002737.2, SEQ ID NO: 5 (nucleotide); and Uniport Reference Sequence: Q99ZW2, SEQ ID NO: 1 (amino acid).

Streptococcus pyogenes Cas9 (wild-type) nucleotide sequence
(SEQ ID NO: 5)
ATGGATAAGAAATACTCAATAGGCTTAGATATCGGCACAAATAGCGTCGGATGGGC
GGTGATCACTGATGAATATAAGGTTCCGTCTAAAAAGTTCAAGGTTCTGGGAAATAC
AGACCGCCACAGTATCAAAAAAAATCTTATAGGGGCTCTTTTATTTGACAGTGGAGA
GACAGCGGAAGCGACTCGTCTCAAACGGACAGCTCGTAGAAGGTATACACGTCGGA
AGAATCGTATTTGTTATCTACAGGAGATTTTTTCAAATGAGATGGCGAAAGTAGATG
ATAGTTTCTTTCATCGACTTGAAGAGTCTTTTTTGGTGGAAGAAGACAAGAAGCATG
AACGTCATCCTATTTTTGGAAATATAGTAGATGAAGTTGCTTATCATGAGAAATATC
CAACTATCTATCATCTGCGAAAAAAATTGGTAGATTCTACTGATAAAGCGGATTTGC
GCTTAATCTATTTGGCCTTAGCGCATATGATTAAGTTTCGTGGTCATTTTTTGATTGA
GGGAGATTTAAATCCTGATAATAGTGATGTGGACAAACTATTTATCCAGTTGGTACA
AACCTACAATCAATTATTTGAAGAAAACCCTATTAACGCAAGTGGAGTAGATGCTA
AAGCGATTCTTTCTGCACGATTGAGTAAATCAAGACGATTAGAAAATCTCATTGCTC
AGCTCCCCGGTGAGAAGAAAAATGGCTTATTTGGGAATCTCATTGCTTTGTCATTGG
GTTTGACCCCTAATTTTAAATCAAATTTTGATTTGGCAGAAGATGCTAAATTACAGC
TTTCAAAAGATACTTACGATGATGATTTAGATAATTTATTGGCGCAAATTGGAGATC
AATATGCTGATTTGTTTTTGGCAGCTAAGAATTTATCAGATGCTATTTTACTTTCAGA
TATCCTAAGAGTAAATACTGAAATAACTAAGGCTCCCCTATCAGCTTCAATGATTAA
ACGCTACGATGAACATCATCAAGACTTGACTCTTTTAAAAGCTTTAGTTCGACAACA
ACTTCCAGAAAAGTATAAAGAAATCTTTTTTGATCAATCAAAAAACGGATATGCAG
GTTATATTGATGGGGGAGCTAGCCAAGAAGAATTTTATAAATTTATCAAACCAATTT
TAGAAAAAATGGATGGTACTGAGGAATTATTGGTGAAACTAAATCGTGAAGATTTG
CTGCGCAAGCAACGGACCTTTGACAACGGCTCTATTCCCCATCAAATTCACTTGGGT
GAGCTGCATGCTATTTTGAGAAGACAAGAAGACTTTTATCCATTTTTAAAAGACAAT
CGTGAGAAGATTGAAAAAATCTTGACTTTTCGAATTCCTTATTATGTTGGTCCATTGG
CGCGTGGCAATAGTCGTTTTGCATGGATGACTCGGAAGTCTGAAGAAACAATTACCC
CATGGAATTTTGAAGAAGTTGTCGATAAAGGTGCTTCAGCTCAATCATTTATTGAAC
GCATGACAAACTTTGATAAAAATCTTCCAAATGAAAAAGTACTACCAAAACATAGT
TTGCTTTATGAGTATTTTACGGTTTATAACGAATTGACAAAGGTCAAATATGTTACTG
AAGGAATGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGATTATTTC
AAAAAAATAGAATGTTTTGATAGTGTTGAAATTTCAGGAGTTGAAGATAGATTTAAT
GCTTCATTAGGTACCTACCATGATTTGCTAAAAATTATTAAAGATAAAGATTTTTTG
GATAATGAAGAAAATGAAGATATCTTAGAGGATATTGTTTTAACATTGACCTTATTT
GAAGATAGGGAGATGATTGAGGAAAGACTTAAAACATATGCTCACCTCTTTGATGA
TAAGGTGATGAAACAGCTTAAACGTCGCCGTTATACTGGTTGGGGACGTTTGTCTCG
AAAATTGATTAATGGTATTAGGGATAAGCAATCTGGCAAAACAATATTAGATTTTTT
GAAATCAGATGGTTTTGCCAATCGCAATTTTATGCAGCTGATCCATGATGATAGTTT
GACATTTAAAGAAGACATTCAAAAAGCACAAGTGTCTGGACAAGGCGATAGTTTAC
ATGAACATATTGCAAATTTAGCTGGTAGCCCTGCTATTAAAAAAGGTATTTTACAGA
CTGTAAAAGTTGTTGATGAATTGGTCAAAGTAATGGGGCGGCATAAGCCAGAAAAT
ATCGTTATTGAAATGGCACGTGAAAATCAGACAACTCAAAAGGGCCAGAAAAATTC
GCGAGAGCGTATGAAACGAATCGAAGAAGGTATCAAAGAATTAGGAAGTCAGATTC
TTAAAGAGCATCCTGTTGAAAATACTCAATTGCAAAATGAAAAGCTCTATCTCTATT
ATCTCCAAAATGGAAGAGACATGTATGTGGACCAAGAATTAGATATTAATCGTTTAA
GTGATTATGATGTCGATCACATTGTTCCACAAAGTTTCCTTAAAGACGATTCAATAG
ACAATAAGGTCTTAACGCGTTCTGATAAAAATCGTGGTAAATCGGATAACGTTCCAA
GTGAAGAAGTAGTCAAAAAGATGAAAAACTATTGGAGACAACTTCTAAACGCCAAG
TTAATCACTCAACGTAAGTTTGATAATTTAACGAAAGCTGAACGTGGAGGTTTGAGT
GAACTTGATAAAGCTGGTTTTATCAAACGCCAATTGGTTGAAACTCGCCAAATCACT
AAGCATGTGGCACAAATTTTGGATAGTCGCATGAATACTAAATACGATGAAAATGA
TAAACTTATTCGAGAGGTTAAAGTGATTACCTTAAAATCTAAATTAGTTTCTGACTTC
CGAAAAGATTTCCAATTCTATAAAGTACGTGAGATTAACAATTACCATCATGCCCAT
GATGCGTATCTAAATGCCGTCGTTGGAACTGCTTTGATTAAGAAATATCCAAAACTT
GAATCGGAGTTTGTCTATGGTGATTATAAAGTTTATGATGTTCGTAAAATGATTGCT
AAGTCTGAGCAAGAAATAGGCAAAGCAACCGCAAAATATTTCTTTTACTCTAATATC
ATGAACTTCTTCAAAACAGAAATTACACTTGCAAATGGAGAGATTCGCAAACGCCCT
CTAATCGAAACTAATGGGGAAACTGGAGAAATTGTCTGGGATAAAGGGCGAGATTT
TGCCACAGTGCGCAAAGTATTGTCCATGCCCCAAGTCAATATTGTCAAGAAAACAG
AAGTACAGACAGGCGGATTCTCCAAGGAGTCAATTTTACCAAAAAGAAATTCGGAC
AAGCTTATTGCTCGTAAAAAAGACTGGGATCCAAAAAAATATGGTGGTTTTGATAGT
CCAACGGTAGCTTATTCAGTCCTAGTGGTTGCTAAGGTGGAAAAAGGGAAATCGAA
GAAGTTAAAATCCGTTAAAGAGTTACTAGGGATCACAATTATGGAAAGAAGTTCCTT
TGAAAAAAATCCGATTGACTTTTTAGAAGCTAAAGGATATAAGGAAGTTAAAAAAG
ACTTAATCATTAAACTACCTAAATATAGTCTTTTTGAGTTAGAAAACGGTCGTAAAC
GGATGCTGGCTAGTGCCGGAGAATTACAAAAAGGAAATGAGCTGGCTCTGCCAAGC
AAATATGTGAATTTTTTATATTTAGCTAGTCATTATGAAAAGTTGAAGGGTAGTCCA
GAAGATAACGAACAAAAACAATTGTTTGTGGAGCAGCATAAGCATTATTTAGATGA
GATTATTGAGCAAATCAGTGAATTTTCTAAGCGTGTTATTTTAGCAGATGCCAATTT
AGATAAAGTTCTTAGTGCATATAACAAACATAGAGACAAACCAATACGTGAACAAG
CAGAAAATATTATTCATTTATTTACGTTGACGAATCTTGGAGCTCCCGCTGCTTTTAA
ATATTTTGATACAACAATTGATCGTAAACGATATACGTCTACAAAAGAAGTTTTAGA
TGCCACTCTTATCCATCAATCCATCACTGGTCTTTATGAAACACGCATTGATTTGAGT
CAGCTAGGAGGTGACTGA
Streptococcus pyogenes Cas9 (wild-type) protein sequence
(SEQ ID NO: 1)
MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(single underline: HNH domain; double underline: RuvC domain)

In some embodiments, wild-type Cas9 corresponds to Cas9 from Streptococcus pyogenes (NCBI Reference Sequence: NC_017053.1, SEQ ID NO 2003 (nucleotide); SEQ ID NO: 2004 (amino acid)):

(SEQ ID NO: 2003)
ATGGATAAGAAATACTCAATAGGCTTAGATATCGGCACAAATAGCGTCGGATGGGC
GGTGATCACTGATGATTATAAGGTTCCGTCTAAAAAGTTCAAGGTTCTGGGAAATAC
AGACCGCCACAGTATCAAAAAAAATCTTATAGGGGCTCTTTTATTTGGCAGTGGAGA
GACAGCGGAAGCGACTCGTCTCAAACGGACAGCTCGTAGAAGGTATACACGTCGGA
AGAATCGTATTTGTTATCTACAGGAGATTTTTTCAAATGAGATGGCGAAAGTAGATG
ATAGTTTCTTTCATCGACTTGAAGAGTCTTTTTTGGTGGAAGAAGACAAGAAGCATG
AACGTCATCCTATTTTTGGAAATATAGTAGATGAAGTTGCTTATCATGAGAAATATC
CAACTATCTATCATCTGCGAAAAAAATTGGCAGATTCTACTGATAAAGCGGATTTGC
GCTTAATCTATTTGGCCTTAGCGCATATGATTAAGTTTCGTGGTCATTTTTTGATTGA
GGGAGATTTAAATCCTGATAATAGTGATGTGGACAAACTATTTATCCAGTTGGTACA
AATCTACAATCAATTATTTGAAGAAAACCCTATTAACGCAAGTAGAGTAGATGCTAA
AGCGATTCTTTCTGCACGATTGAGTAAATCAAGACGATTAGAAAATCTCATTGCTCA
GCTCCCCGGTGAGAAGAGAAATGGCTTGTTTGGGAATCTCATTGCTTTGTCATTGGG
ATTGACCCCTAATTTTAAATCAAATTTTGATTTGGCAGAAGATGCTAAATTACAGCT
TTCAAAAGATACTTACGATGATGATTTAGATAATTTATTGGCGCAAATTGGAGATCA
ATATGCTGATTTGTTTTTGGCAGCTAAGAATTTATCAGATGCTATTTTACTTTCAGAT
ATCCTAAGAGTAAATAGTGAAATAACTAAGGCTCCCCTATCAGCTTCAATGATTAAG
CGCTACGATGAACATCATCAAGACTTGACTCTTTTAAAAGCTTTAGTTCGACAACAA
CTTCCAGAAAAGTATAAAGAAATCTTTTTTGATCAATCAAAAAACGGATATGCAGGT
TATATTGATGGGGGAGCTAGCCAAGAAGAATTTTATAAATTTATCAAACCAATTTTA
GAAAAAATGGATGGTACTGAGGAATTATTGGTGAAACTAAATCGTGAAGATTTGCT
GCGCAAGCAACGGACCTTTGACAACGGCTCTATTCCCCATCAAATTCACTTGGGTGA
GCTGCATGCTATTTTGAGAAGACAAGAAGACTTTTATCCATTTTTAAAAGACAATCG
TGAGAAGATTGAAAAAATCTTGACTTTTCGAATTCCTTATTATGTTGGTCCATTGGCG
CGTGGCAATAGTCGTTTTGCATGGATGACTCGGAAGTCTGAAGAAACAATTACCCCA
TGGAATTTTGAAGAAGTTGTCGATAAAGGTGCTTCAGCTCAATCATTTATTGAACGC
ATGACAAACTTTGATAAAAATCTTCCAAATGAAAAAGTACTACCAAAACATAGTTTG
CTTTATGAGTATTTTACGGTTTATAACGAATTGACAAAGGTCAAATATGTTACTGAG
GGAATGCGAAAACCAGCATTTCTTTCAGGTGAACAGAAGAAAGCCATTGTTGATTTA
CTCTTCAAAACAAATCGAAAAGTAACCGTTAAGCAATTAAAAGAAGATTATTTCAA
AAAAATAGAATGTTTTGATAGTGTTGAAATTTCAGGAGTTGAAGATAGATTTAATGC
TTCATTAGGCGCCTACCATGATTTGCTAAAAATTATTAAAGATAAAGATTTTTTGGA
TAATGAAGAAAATGAAGATATCTTAGAGGATATTGTTTTAACATTGACCTTATTTGA
AGATAGGGGGATGATTGAGGAAAGACTTAAAACATATGCTCACCTCTTTGATGATA
AGGTGATGAAACAGCTTAAACGTCGCCGTTATACTGGTTGGGGACGTTTGTCTCGAA
AATTGATTAATGGTATTAGGGATAAGCAATCTGGCAAAACAATATTAGATTTTTTGA
AATCAGATGGTTTTGCCAATCGCAATTTTATGCAGCTGATCCATGATGATAGTTTGA
CATTTAAAGAAGATATTCAAAAAGCACAGGTGTCTGGACAAGGCCATAGTTTACAT
GAACAGATTGCTAACTTAGCTGGCAGTCCTGCTATTAAAAAAGGTATTTTACAGACT
GTAAAAATTGTTGATGAACTGGTCAAAGTAATGGGGCATAAGCCAGAAAATATCGT
TATTGAAATGGCACGTGAAAATCAGACAACTCAAAAGGGCCAGAAAAATTCGCGAG
AGCGTATGAAACGAATCGAAGAAGGTATCAAAGAATTAGGAAGTCAGATTCTTAAA
GAGCATCCTGTTGAAAATACTCAATTGCAAAATGAAAAGCTCTATCTCTATTATCTA
CAAAATGGAAGAGACATGTATGTGGACCAAGAATTAGATATTAATCGTTTAAGTGA
TTATGATGTCGATCACATTGTTCCACAAAGTTTCATTAAAGACGATTCAATAGACAA
TAAGGTACTAACGCGTTCTGATAAAAATCGTGGTAAATCGGATAACGTTCCAAGTGA
AGAAGTAGTCAAAAAGATGAAAAACTATTGGAGACAACTTCTAAACGCCAAGTTAA
TCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGC
ATGTGGCACAAATTTTGGATAGTCGCATGAATACTAAATACGATGAAAATGATAAA
CTTATTCGAGAGGTTAAAGTGATTACCTTAAAATCTAAATTAGTTTCTGACTTCCGA
AAAGATTTCCAATTCTATAAAGTACGTGAGATTAACAATTACCATCATGCCCATGAT
GCGTATCTAAATGCCGTCGTTGGAACTGCTTTGATTAAGAAATATCCAAAACTTGAA
TCGGAGTTTGTCTATGGTGATTATAAAGTTTATGATGTTCGTAAAATGATTGCTAAGT
CTGAGCAAGAAATAGGCAAAGCAACCGCAAAATATTTCTTTTACTCTAATATCATGA
ACTTCTTCAAAACAGAAATTACACTTGCAAATGGAGAGATTCGCAAACGCCCTCTAA
TCGAAACTAATGGGGAAACTGGAGAAATTGTCTGGGATAAAGGGCGAGATTTTGCC
ACAGTGCGCAAAGTATTGTCCATGCCCCAAGTCAATATTGTCAAGAAAACAGAAGT
ACAGACAGGCGGATTCTCCAAGGAGTCAATTTTACCAAAAAGAAATTCGGACAAGC
TTATTGCTCGTAAAAAAGACTGGGATCCAAAAAAATATGGTGGTTTTGATAGTCCAA
CGGTAGCTTATTCAGTCCTAGTGGTTGCTAAGGTGGAAAAAGGGAAATCGAAGAAG
TTAAAATCCGTTAAAGAGTTACTAGGGATCACAATTATGGAAAGAAGTTCCTTTGAA
AAAAATCCGATTGACTTTTTAGAAGCTAAAGGATATAAGGAAGTTAAAAAAGACTT
AATCATTAAACTACCTAAATATAGTCTTTTTGAGTTAGAAAACGGTCGTAAACGGAT
GCTGGCTAGTGCCGGAGAATTACAAAAAGGAAATGAGCTGGCTCTGCCAAGCAAAT
ATGTGAATTTTTTATATTTAGCTAGTCATTATGAAAAGTTGAAGGGTAGTCCAGAAG
ATAACGAACAAAAACAATTGTTTGTGGAGCAGCATAAGCATTATTTAGATGAGATT
ATTGAGCAAATCAGTGAATTTTCTAAGCGTGTTATTTTAGCAGATGCCAATTTAGAT
AAAGTTCTTAGTGCATATAACAAACATAGAGACAAACCAATACGTGAACAAGCAGA
AAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCC
ACTCTTATCCATCAATCCATCACTGGTCTTTATGAAACACGCATTGATTTGAGTCAGC
TAGGAGGTGACTGA
(SEQ ID NO: 2004)
MDKKYSIGLDIGTNSVGWAVITDDYKVPSKKFKVLGNTDRHSIKKNLIGALLFGSGETA
EATRLKRTARRRYTRRKNRICYLQEIFSNEMAKVDDSFFHRLEESFLVEEDKKHERHPIF
GNIVDEVAYHEKYPTIYHLRKKLADSTDKADLRLIYLALAHMIKFRGHFLIEGDLNPDNS
DVDKLFIQLVQIYNQLFEENPINASRVDAKAILSARLSKSRRLENLIAQLPGEKRNGLFGN
LIALSLGLTPNFKSNFDLAEDAKLQLSKDTYDDDLDNLLAQIGDQYADLFLAAKNLSDA
ILLSDILRVNSEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNASLGAYHDLL
KIIKDKDFLDNEENEDILEDIVLTLTLFEDRGMIEERLKTYAHLFDDKVMKQLKRRRYTG
WGRLSRKLINGIRDKQSGKTILDFLKSDGFANRNFMQLIHDDSLTFKEDIQKAQVSGQG
HSLHEQIANLAGSPAIKKGILQTVKIVDELVKVMGHKPENIVIEMARENQTTQKGQKNS
RERMKRIEEGIKELGSQILKEHPVENTQLQNEKLYLYYLQNGRDMYVDQELDINRLSDY
DVDHIVPQSFIKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGD
(single underline: HNH domain; double underline: RuvC domain)

In some embodiments, wild type Cas9 corresponds to, or comprises, Cas9 from Streptococcus pyogenes (SEQ ID NO: 2005 (nucleotide) and/or SEQ ID NO: 2006 (amino acid)):

(SEQ ID NO: 2005)
ATGGATAAAAAGTATTCTATTGGTTTAGACATCGGCACTAATTCCGTTGGATGGGCT
GTCATAACCGATGAATACAAAGTACCTTCAAAGAAATTTAAGGTGTTGGGGAACAC
AGACCGTCATTCGATTAAAAAGAATCTTATCGGTGCCCTCCTATTCGATAGTGGCGA
AACGGCAGAGGCGACTCGCCTGAAACGAACCGCTCGGAGAAGGTATACACGTCGCA
AGAACCGAATATGTTACTTACAAGAAATTTTTAGCAATGAGATGGCCAAAGTTGAC
GATTCTTTCTTTCACCGTTTGGAAGAGTCCTTCCTTGTCGAAGAGGACAAGAAACAT
GAACGGCACCCCATCTTTGGAAACATAGTAGATGAGGTGGCATATCATGAAAAGTA
CCCAACGATTTATCACCTCAGAAAAAAGCTAGTTGACTCAACTGATAAAGCGGACCT
GAGGTTAATCTACTTGGCTCTTGCCCATATGATAAAGTTCCGTGGGCACTTTCTCATT
GAGGGTGATCTAAATCCGGACAACTCGGATGTCGACAAACTGTTCATCCAGTTAGTA
CAAACCTATAATCAGTTGTTTGAAGAGAACCCTATAAATGCAAGTGGCGTGGATGC
GAAGGCTATTCTTAGCGCCCGCCTCTCTAAATCCCGACGGCTAGAAAACCTGATCGC
ACAATTACCCGGAGAGAAGAAAAATGGGTTGTTCGGTAACCTTATAGCGCTCTCACT
AGGCCTGACACCAAATTTTAAGTCGAACTTCGACTTAGCTGAAGATGCCAAATTGCA
GCTTAGTAAGGACACGTACGATGACGATCTCGACAATCTACTGGCACAAATTGGAG
ATCAGTATGCGGACTTATTTTTGGCTGCCAAAAACCTTAGCGATGCAATCCTCCTAT
CTGACATACTGAGAGTTAATACTGAGATTACCAAGGCGCCGTTATCCGCTTCAATGA
TCAAAAGGTACGATGAACATCACCAAGACTTGACACTTCTCAAGGCCCTAGTCCGTC
AGCAACTGCCTGAGAAATATAAGGAAATATTCTTTGATCAGTCGAAAAACGGGTAC
GCAGGTTATATTGACGGCGGAGCGAGTCAAGAGGAATTCTACAAGTTTATCAAACC
CATATTAGAGAAGATGGATGGGACGGAAGAGTTGCTTGTAAAACTCAATCGCGAAG
ATCTACTGCGAAAGCAGCGGACTTTCGACAACGGTAGCATTCCACATCAAATCCACT
TAGGCGAATTGCATGCTATACTTAGAAGGCAGGAGGATTTTTATCCGTTCCTCAAAG
ACAATCGTGAAAAGATTGAGAAAATCCTAACCTTTCGCATACCTTACTATGTGGGAC
CCCTGGCCCGAGGGAACTCTCGGTTCGCATGGATGACAAGAAAGTCCGAAGAAACG
ATTACTCCATGGAATTTTGAGGAAGTTGTCGATAAAGGTGCGTCAGCTCAATCGTTC
ATCGAGAGGATGACCAACTTTGACAAGAATTTACCGAACGAAAAAGTATTGCCTAA
GCACAGTTTACTTTACGAGTATTTCACAGTGTACAATGAACTCACGAAAGTTAAGTA
TGTCACTGAGGGCATGCGTAAACCCGCCTTTCTAAGCGGAGAACAGAAGAAAGCAA
TAGTAGATCTGTTATTCAAGACCAACCGCAAAGTGACAGTTAAGCAATTGAAAGAG
GACTACTTTAAGAAAATTGAATGCTTCGATTCTGTCGAGATCTCCGGGGTAGAAGAT
CGATTTAATGCGTCACTTGGTACGTATCATGACCTCCTAAAGATAATTAAAGATAAG
GACTTCCTGGATAACGAAGAGAATGAAGATATCTTAGAAGATATAGTGTTGACTCTT
ACCCTCTTTGAAGATCGGGAAATGATTGAGGAAAGACTAAAAACATACGCTCACCT
GTTCGACGATAAGGTTATGAAACAGTTAAAGAGGCGTCGCTATACGGGCTGGGGAC
GATTGTCGCGGAAACTTATCAACGGGATAAGAGACAAGCAAAGTGGTAAAACTATT
CTCGATTTTCTAAAGAGCGACGGCTTCGCCAATAGGAACTTTATGCAGCTGATCCAT
GATGACTCTTTAACCTTCAAAGAGGATATACAAAAGGCACAGGTTTCCGGACAAGG
GGACTCATTGCACGAACATATTGCGAATCTTGCTGGTTCGCCAGCCATCAAAAAGGG
CATACTCCAGACAGTCAAAGTAGTGGATGAGCTAGTTAAGGTCATGGGACGTCACA
AACCGGAAAACATTGTAATCGAGATGGCACGCGAAAATCAAACGACTCAGAAGGG
GCAAAAAAACAGTCGAGAGCGGATGAAGAGAATAGAAGAGGGTATTAAAGAACTG
GGCAGCCAGATCTTAAAGGAGCATCCTGTGGAAAATACCCAATTGCAGAACGAGAA
ACTTTACCTCTATTACCTACAAAATGGAAGGGACATGTATGTTGATCAGGAACTGGA
CATAAACCGTTTATCTGATTACGACGTCGATCACATTGTACCCCAATCCTTTTTGAAG
GACGATTCAATCGACAATAAAGTGCTTACACGCTCGGATAAGAACCGAGGGAAAAG
TGACAATGTTCCAAGCGAGGAAGTCGTAAAGAAAATGAAGAACTATTGGCGGCAGC
TCCTAAATGCGAAACTGATAACGCAAAGAAAGTTCGATAACTTAACTAAAGCTGAG
AGGGGTGGCTTGTCTGAACTTGACAAGGCCGGATTTATTAAACGTCAGCTCGTGGAA
ACCCGCCAAATCACAAAGCATGTTGCACAGATACTAGATTCCCGAATGAATACGAA
ATACGACGAGAACGATAAGCTGATTCGGGAAGTCAAAGTAATCACTTTAAAGTCAA
AATTGGTGTCGGACTTCAGAAAGGATTTTCAATTCTATAAAGTTAGGGAGATAAATA
ACTACCACCATGCGCACGACGCTTATCTTAATGCCGTCGTAGGGACCGCACTCATTA
AGAAATACCCGAAGCTAGAAAGTGAGTTTGTGTATGGTGATTACAAAGTTTATGAC
GTCCGTAAGATGATCGCGAAAAGCGAACAGGAGATAGGCAAGGCTACAGCCAAAT
ACTTCTTTTATTCTAACATTATGAATTTCTTTAAGACGGAAATCACTCTGGCAAACGG
AGAGATACGCAAACGACCTTTAATTGAAACCAATGGGGAGACAGGTGAAATCGTAT
GGGATAAGGGCCGGGACTTCGCGACGGTGAGAAAAGTTTTGTCCATGCCCCAAGTC
AACATAGTAAAGAAAACTGAGGTGCAGACCGGAGGGTTTTCAAAGGAATCGATTCT
TCCAAAAAGGAATAGTGATAAGCTCATCGCTCGTAAAAAGGACTGGGACCCGAAAA
AGTACGGTGGCTTCGATAGCCCTACAGTTGCCTATTCTGTCCTAGTAGTGGCAAAAG
TTGAGAAGGGAAAATCCAAGAAACTGAAGTCAGTCAAAGAATTATTGGGGATAACG
ATTATGGAGCGCTCGTCTTTTGAAAAGAACCCCATCGACTTCCTTGAGGCGAAAGGT
TACAAGGAAGTAAAAAAGGATCTCATAATTAAACTACCAAAGTATAGTCTGTTTGA
GTTAGAAAATGGCCGAAAACGGATGTTGGCTAGCGCCGGAGAGCTTCAAAAGGGGA
ACGAACTCGCACTACCGTCTAAATACGTGAATTTCCTGTATTTAGCGTCCCATTACG
AGAAGTTGAAAGGTTCACCTGAAGATAACGAACAGAAGCAACTTTTTGTTGAGCAG
CACAAACATTATCTCGACGAAATCATAGAGCAAATTTCGGAATTCAGTAAGAGAGT
CATCCTAGCTGATGCCAATCTGGACAAAGTATTAAGCGCATACAACAAGCACAGGG
ATAAACCCATACGTGAGCAGGCGGAAAATATTATCCATTTGTTTACTCTTACCAACC
TCGGCGCTCCAGCCGCATTCAAGTATTTTGACACAACGATAGATCGCAAACGATACA
CTTCTACCAAGGAGGTGCTAGACGCGACACTGATTCACCAATCCATCACGGGATTAT
ATGAAACTCGGATAGATTTGTCACAGCTTGGGGGTGACGGATCCCCCAAGAAGAAG
AGGAAAGTCTCGAGCGACTACAAAGACCATGACGGTGATTATAAAGATCATGACAT
CGATTACAAGGATGACGATGACAAGGCTGCAGGA
(SEQ ID NO: 2006)
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(single underline: HNH domain; double underline: RuvC domain)

In some embodiments, wild type Cas9 corresponds to Cas9 from Streptococcus Aureus. S. aureus Cas9 wild type (SEQ ID NO: 6)

(SEQ ID NO: 6)

MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG

In some embodiments, wild type Cas9 corresponds to Cas9 from Streptococcus thermophilus.

Streptococcus thermophilus wild type CRISPR3
Cas9 (St3Cas9)

(SEQ ID NO: 7)

MTKPYSIGLDIGTNSVGWAVITDNYKVPSKKMKVLGNTSKKYIKKNLLGV
LLFDSGITAEGRRLKRTARRRYTRRRNRILYLQEIFSTEMATLDDAFFQR
LDDSFLVPDDKRDSKYPIFGNLVEEKVYHDEFPTIYHLRKYLADSTKKAD
LRLVYLALAHMIKYRGHFLIEGEFNSKNNDIQKNFQDFLDTYNAIFESDL
SLENSKQLEEIVKDKISKLEKKDRILKLFPGEKNSGIFSEFLKLIVGNQA
DFRKCFNLDEKASLHFSKESYDEDLETLLGYIGDDYSDVFLKAKKLYDAI
LLSGFLTVTDNETEAPLSSAMIKRYNEHKEDLALLKEYIRNISLKTYNEV
FKDDTKNGYAGYIDGKTNQEDFYVYLKNLLAEFEGADYFLEKIDREDFLR
KQRTFDNGSIPYQIHLQEMRAILDKQAKFYPFLAKNKERIEKILTFRIPY
YVGPLARGNSDFAWSIRKRNEKITPWNFEDVIDKESSAEAFINRMTSFDL
YLPEEKVLPKHSLLYETFNVYNELTKVRFIAESMRDYQFLDSKQKKDIVR
LYFKDKRKVTDKDIIEYLHAIYGYDGIELKGIEKQFNSSLSTYHDLLNII
NDKEFLDDSSNEAIIEEIIHTLTIFEDREMIKQRLSKFENIFDKSVLKKL
SRRHYTGWGKLSAKLINGIRDEKSGNTILDYLIDDGISNRNFMQLIHDDA
LSFKKKIQKAQIIGDEDKGNIKEVVKSLPGSPAIKKGILQSIKIVDELVK
VMGGRKPESIVVEMARENQYTNQGKSNSQQRLKRLEKSLKELGSKILKEN
IPAKLSKIDNNALQNDRLYLYYLQNGKDMYTGDDLDIDRLSNYDIDHIIP
QAFLKDNSIDNKVLVSSASNRGKSDDFPSLEVVKKRKTFWYQLLKSKLIS
QRKFDNLTKAERGGLLPEDKAGFIQRQLVETRQITKHVARLLDEKFNNKK
DENNRAVRTVKIITLKSTLVSQFRKDFELYKVREINDFHHAHDAYLNAVI
ASALLKKYPKLEPEFVYGDYPKYNSFRERKSATEKVYFYSNIMNIFKKSI
SLADGRVIERPLIEVNEETGESVWNKESDLATVRRVLSYPQVNVVKKVEE
QNHGLDRGKPKGLFNANLSSKPKPNSNENLVGAKEYLDPKKYGGYAGISN
SFAVLVKGTIEKGAKKKITNVLEFQGISILDRINYRKDKLNFLLEKGYKD
IELIIELPKYSLFELSDGSRRMLASILSTNNKRGEIHKGNQIFLSQKFVK
LLYHAKRISNTINENHRKYVENHKKEFEELFYYILEFNENYVGAKKNGKL
LNSAFQSWQNHSIDELCSSFIGPTGSERKGLFELTSRGSAADFEFLGVKI
PRYRDYTPSSLLKDATLIHQSVTGLYETRIDLAKLGEG
Streptococcus thermophilus CRISPR1 Cas9 wild
type (St1Cas9)

(SEQ ID NO: 8)

MSDLVLGLDIGIGSVGVGILNKVTGEIIHKNSRIFPAAQAENNLVRRTNR
QGRRLTRRKKHRRVRLNRLFEESGLITDFTKISINLNPYQLRVKGLTDEL
SNEELFIALKNMVKHRGISYLDDASDDGNSSIGDYAQIVKENSKQLETKT
PGQIQLERYQTYGQLRGDFTVEKDGKKHRLINVFPTSAYRSEALRILQTQ
QEFNPQITDEFINRYLEILTGKRKYYHGPGNEKSRTDYGRYRTSGETLDN
IFGILIGKCTFYPDEFRAAKASYTAQEFNLLNDLNNLTVPTETKKLSKEQ
KNQIINYVKNEKAMGPAKLFKYIAKLLSCDVADIKGYRIDKSGKAEIHTF
EAYRKMKTLETLDIEQMDRETLDKLAYVLTLNTEREGIQEALEHEFADGS
FSQKQVDELVQFRKANSSIFGKGWHNFSVKLMMELIPELYETSEEQMTIL
TRLGKQKTTSSSNKTKYIDEKLLTEEIYNPVVAKSVRQAIKIVNAAIKEY
GDFDNIVIEMARETNEDDEKKAIQKIQKANKDEKDAAMLKAANQYNGKAE
LPHSVFHGHKQLATKIRLWHQQGERCLYTGKTISIHDLINNSNQFEVDHI
LPLSITFDDSLANKVLVYATANQEKGQRTPYQALDSMDDAWSFRELKAFV
RESKTLSNKKKEYLLTEEDISKFDVRKKFIERNLVDTRYASRVVLNALQE
HFRAHKIDTKVSVVRGQFTSQLRRHWGIEKTRDTYHHHAVDALIIAASSQ
LNLWKKQKNTLVSYSEDQLLDIETGELISDDEYKESVFKAPYQHFVDTLK
SKEFEDSILFSYQVDSKFNRKISDATIYATRQAKVGKDKADETYVLGKIK
DIYTQDGYDAFMKIYKKDKSKFLMYRHDPQTFEKVIEPILENYPNKQINE
KGKEVPCNPFLKYKEEHGYIRKYSKKGNGPEIKSLKYYDSKLGNHIDITP
KDSNNKVVLQSVSPWRADVYFNKTTGKYEILGLKYADLQFEKGTGTYKIS
QEKYNDIKKKEGVDSDSEFKFTLYKNDLLLVKDTETKEQQLFRFLSRTMP
KQKHYVELKPYDKQKFEGGEALIKVLGNVANSGQCKKGLGKSNISIYKVR
TDVLGNQHIIKNEGDKPKLDF

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis I (NCBI Ref: NC_018721.1); Listeria innocua (NCBI Ref: NP_472073.1), Campylobacter jejuni (NCBI Ref: YP_002344900.1) or Neisseria. meningitidis (NCBI Ref: YP_002342100.1) or to a Cas9 from any of the organisms listed in Example 1 (SEQ ID NOs: 11-260).

In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9. In some embodiments, the fragment is at least 100 amino acids in length. In some embodiments, the fragment is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length.

To be used as in the fusion protein of the present disclosure as the guide nucleotide sequence-programmable DNA binding protein domain, a Cas9 protein needs to be nuclease inactive. A nuclease-inactive Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., (2013) Cell. 28; 152(5):1173-83, each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)).

dCas9 (D10A and H840A)

(SEQ ID NO: 2)

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
(single underline: HNH domain;
double underline: RuvC domain).

The dCas9 of the present disclosure encompasses completely inactive Cas9 or partially inactive Cas9. For example, the dCas9 may have one of the two nuclease domain inactivated, while the other nuclease domain remains active. Such a partially active Cas9 may also be referred to as a “Cas9 nickase”, due to its ability to cleave one strand of the targeted DNA sequence. The Cas9 nickase suitable for use in accordance with the present disclosure has an active HNH domain and an inactive RuvC domain and is able to cleave only the strand of the target DNA that is bound by the sgRNA (which is the opposite strand of the strand that is being edited via cytidine deamination). The Cas9 nickase of the present disclosure may comprise mutations that inactivate the RuvC domain, e.g., a D10A mutation. It is to be understood that any mutation that inactivates the RuvC domain may be included in a Cas9 nickase, e.g., insertion, deletion, or single or multiple amino acid substitution in the RuvC domain. In a Cas9 nickase described herein, while the RuvC domain is inactivated, the HNH domain remains activate. Thus, while the Cas9 nickase may comprise mutations other than those that inactivate the RuvC domain (e.g., D10A), those mutations do not affect the activity of the HNH domain. In a non-limiting Cas9 nickase example, the histidine at position 840 remains unchanged. The sequence of an exemplary Cas9 nickase suitable for the present disclosure is provided below.

S. pyogenes Cas9 Nickase (D10A)

(SEQ ID NO: 3)

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
(single underline: HNH domain;
double underline: RuvC domain)
S. aureus Cas9 Nickase (D10A)

(SEQ ID NO: 4)

MKRNYILGLAIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG

It is appreciated that when the term “dCas9” or “nuclease-inactive Cas9” is used herein, it refers to Cas9 variants that are inactive in both HNH and RuvC domains as well as Cas9 nickases. For example, the dCas9 used in the present disclosure may include the amino acid sequence set forth in SEQ ID NO: 2 or SEQ ID NO: 3. In some embodiments, the dCas9 may comprise other mutations that inactivate RuvC or HNH domain. Additional suitable mutations that inactivate Cas9 will be apparent to those of skill in the art based on this disclosure and knowledge in the field, and are within the scope of this disclosure. Such additional exemplary suitable nuclease-inactive Cas9 domains include, but are not limited to, D839A and/or N863A (See, e.g., Prashant et al., Nature Biotechnology. 2013; 31(9): 833-838, which are incorporated herein by reference), or), or K603R (See, e.g., Chavez et al., Nature Methods 12, 326-328, 2015, which is incorporated herein by reference). The term Cas9, dCas9, or Cas9 variant also encompasses Cas9, dCas9, or Cas9 variants from any organism. Also appreciated is that dCas9, Cas9 nickase, or other appropriate Cas9 variants from any organisms may be used in accordance with the present disclosure.

A “deaminase” refers to an enzyme that catalyzes the removal of an amine group from a molecule, or deamination, for example through hydrolysis. In some embodiments, the deaminase is a cytidine deaminase, catalyzing the deamination of cytidine (C) to uridine (U), deoxycytidine (dC) to deoxyuridine (dU), or 5-methyl-cytidine to thymidine (T, 5-methyl-U), respectively. Subsequent DNA repair mechanisms ensure that a dU is replaced by T, as described in Komor et al (Nature, Programmable editing of a target base in genomic DNA without double-stranded DNA cleavage, 533, 420-424 (2016), which is incorporated herein by reference). In some embodiments, the deaminase is a cytosine deaminase, catalyzing and promoting the conversion of cytosine to uracil (e.g., in RNA) or thymine (e.g., in DNA). In some embodiments, the deaminase is a naturally-occurring deaminase from an organism, such as a human, chimpanzee, gorilla, monkey, cow, dog, rat, or mouse. In some embodiments, the deaminase is a variant of a naturally-occurring deaminase from an organism, and the variants do not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

A “cytosine deaminase” refers to an enzyme that catalyzes the chemical reaction “cytosine+H₂O↔uracil+NH₃” or “5-methyl-cytosine+H₂O↔thymine+NH₃.” As it may be apparent from the reaction formula, such chemical reactions result in a C to U/T nucleobase change. In the context of a gene, such nucleotide change, or mutation, may in turn lead to an amino acid change in the protein, which may affect the protein's function, e.g., loss-of-function or gain-of-function. Subsequent DNA repair mechanisms ensure that uracil bases in DNA are replaced by T, as described in Komor et al. (Nature, Programmable editing of a target base in genomic DNA without double-stranded DNA cleavage, 533, 420-424 (2016), which is incorporated herein by reference).

One exemplary suitable class of cytosine deaminases is the apolipoprotein B mRNA-editing complex (APOBEC) family of cytosine deaminases encompassing eleven proteins that serve to initiate mutagenesis in a controlled and beneficial manner. The apolipoprotein B editing complex 3 (APOBEC3) enzyme provides protection to human cells against a certain HIV-1 strain via the deamination of cytosines in reverse-transcribed viral ssDNA. These cytosine deaminases all require a Zn²⁺-coordinating motif (His-X-Glu-X₂₃₋₂₆-Pro-Cys-X₂₋₄-Cys; SEQ ID NO: 1996) and bound water molecule for catalytic activity. The glutamic acid residue acts to activate the water molecule to a zinc hydroxide for nucleophilic attack in the deamination reaction. Each family member preferentially deaminates at its own particular “hotspot,” for example, WRC (W is A or T, R is A or G) for hAID, or TTC for hAPOBEC3F. A recent crystal structure of the catalytic domain of APOBEC3G revealed a secondary structure comprising a five-stranded β-sheet core flanked by six α-helices, which is believed to be conserved across the entire family. The active center loops have been shown to be responsible for both ssDNA binding and in determining “hotspot” identity. Overexpression of these enzymes has been linked to genomic instability and cancer, thus highlighting the importance of sequence-specific targeting. Another suitable cytosine deaminase is the activation-induced cytidine deaminase (AID), which is responsible for the maturation of antibodies by converting cytosines in ssDNA to uracils in a transcription-dependent, strand-biased fashion.

The term “base editors” or “nucleobase editors,” as used herein, broadly refer to any of the fusion proteins described herein. In some embodiments, the nucleobase editors are capable of precisely deaminating a target base to convert it to a different base, e.g., the base editor may target C bases in a nucleic acid sequence and convert the C to T base. In some embodiments, the base editor comprises a Cas9 (e.g., dCas9 and nCas9), CasX, CasY, Cpf1, C2c1, C2c2, C2c3, or Argonaute protein fused to a cytidine deaminase. For example, in some embodiments, the base editor may be a cytosine deaminase-dCas9 fusion protein. In some embodiments, the base editor may be a cytosine deaminase-Cas9 nickase fusion protein. In some embodiments, the base editor may be a deaminase-dCas9-UGI fusion protein. In some embodiments, the base editor may be an UGI-deaminase-dCas9 fusion protein. In some embodiments, the base editor may be an UGI-deaminase-Cas9 nickase fusion protein. In some embodiments, the base editor may be an APOBEC1-dCas9-UGI fusion protein. In some embodiments, the base editor may be an APOBEC1-Cas9 nickase-UGI fusion protein. In some embodiments, the base editor may be an APOBEC1-dCpf1-UGI fusion protein. In some embodiments, the base editor may be an APOBEC1-dNgAgo-UGI fusion protein. In some embodiments, the base editor comprises a CasX protein fused to a cytidine deaminase. In some embodiments, the base editor comprises a CasY protein fused to a cytidine deaminase. In some embodiments, the base editor comprises a Cpf1 protein fused to a cytidine deaminase. In some embodiments, the base editor comprises a C2c1 protein fused to a cytidine deaminase. In some embodiments, the base editor comprises a C2c2 protein fused to a cytidine deaminase. In some embodiments, the base editor comprises a C2c3 protein fused to a cytidine deaminase. In some embodiments, the base editor comprises an Argonaute protein fused to a cytidine deaminase. In some embodiments, the fusion protein described herein comprises a Gam protein, a guide nucleotide sequence-programmable DNA binding protein, and a cytidine deaminase domain. In some embodiments, the base editor comprises a Gam protein, fused to a CasX protein, which is fused to a cytidine deaminase. In some embodiments, the base editor comprises a Gam protein, fused to a CasY protein, which is fused to a cytidine deaminase. In some embodiments, the base editor comprises a Gam protein, fused to a Cpf1 protein, which is fused to a cytidine deaminase. In some embodiments, the base editor comprises a Gam protein, fused to a C2c1 protein, which is fused to a cytidine deaminase. In some embodiments, the base editor comprises a Gam protein, fused to a C2c2 protein, which is fused to a cytidine deaminase. In some embodiments, the base editor comprises a Gam protein, fused to a C2c3 protein, which is fused to a cytidine deaminase. In some embodiments, the base editor comprises a Gam protein, fused to an Argonaute protein, which is fused to a cytidine deaminase. In some embodiments, the base editor comprises a Gam protein, fused to a saCas9 protein, which is fused to a cytidine deaminase. Non-limiting exemplary sequences of the nucleobase editors described herein are provided in Example 1, SEQ ID NOs: 293-302. Such nucleobase editors and methods of using them for genome editing have been described in the art, e.g., in U.S. Pat. No. 9,068,179, US Patent Application Publications US 20150166980, US20150166981, US20150166982, US20150166984, and US20150165054, and U.S. Provisional Application Ser. Nos. 62/245,828, 62/279,346, 62/311,763, 62/322,178, 62/357,352, 62/370,700, and 62/398,490, and in Komor et al., Nature, Programmable editing of a target base in genomic DNA without double-stranded DNA cleavage, 533, 420-424 (2016), each of which is incorporated herein by reference.

The term “target site” or “target sequence” refers to a sequence within a nucleic acid molecule (e.g., a DNA molecule) that is deaminated by the fusion protein provided herein. In some embodiments, the target sequence is a polynucleotide (e.g., a DNA), wherein the polynucleotide comprises a coding strand and a complementary strand. The meaning of a “coding strand” and “complementary strand,” as used herein, is the same as the common meaning of the terms in the art. In some embodiments, the target sequence is a sequence in the genome of a mammal. In some embodiments, the target sequence is a sequence in the genome of a human. In some embodiments, the target sequence is a sequence in the genome of a non-human animal The term “target codon” refers to the amino acid codon that is edited by the base editor and converted to a different codon via deamination. The term “target base” refers to the nucleotide base that is edited by the base editor and converted to a different base via deamination. In some embodiments, the target codon in the coding strand is edited (e.g., deaminated). In some embodiments, the target codon in the complimentary strand is edited (e.g., deaminated).

The term “uracil glycosylase inhibitor” or “UGI,” as used herein, refers to a protein that is capable of inhibiting a uracil-DNA glycosylase base-excision repair enzyme.

The term “linker,” as used herein, refers to a chemical group or a molecule linking two molecules or moieties, e.g., two domains of a fusion protein, such as, for example, a nuclease-inactive Cas9 domain and a nucleic acid editing domain (e.g., a deaminase domain). In some embodiments, a linker joins a gRNA binding domain of an RNA-programmable nuclease, including a Cas9 nuclease domain, and a catalytic domain of a nucleic-acid editing domain (e.g., a deaminase domain). In some embodiments, a linker joins a gRNA binding domain of an RNA-programmable nuclease (e.g., Cas9) and a Gam protein. In some embodiments, a linker joins a gRNA binding domain of an RNA-programmable nuclease (e.g., Cas9) and a UGI domain. In some embodiments, a linker joins a UGI domain and a Gam protein. In some embodiments, a linker joins a catalytic domain of a nucleic-acid editing domain (e.g., a deaminase domain) and a UGI domain. In some embodiments, a linker joins a catalytic domain of a nucleic-acid editing domain (e.g., a deaminase domain) and a Gam protein. Typically, the linker is positioned between, or flanked by, two groups, molecules, domains, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer polymer (e.g. a non-natural polymer, non-peptidic polymer), or chemical moiety. In some embodiments, the linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid,” and “polynucleotide,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), which are incorporated herein by reference.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent (e.g., mouse, rat). In some embodiments, the subject is a domesticated animal. In some embodiments, the subject is a sheep, a goat, a cattle, a cat, or a dog. In some embodiments, the subject is a research animal. In some embodiments, the subject is genetically engineered, e.g., a genetically engineered non-human subject. The subject may be of either sex and at any stage of development.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence. The fusion proteins (e.g., base editors) described herein are made recombinantly. Recombinant technology is familiar to those skilled in the art.

An “intron” refers to any nucleotide sequence within a gene that is removed by RNA splicing during maturation of the final RNA product. The term intron refers to both the DNA sequence within a gene and the corresponding sequence in RNA transcripts. Sequences that are joined together in the final mature RNA after RNA splicing are exons. Introns are found in the genes of most organisms and many viruses, and can be located in a wide range of genes, including those that generate proteins, ribosomal RNA (rRNA), and transfer RNA (tRNA). When proteins are generated from intron-containing genes, RNA splicing takes place as part of the RNA processing pathway that follows transcription and precedes translation.

An “exon” refers to any part of a gene that will become a part of the final mature RNA produced by that gene after introns have been removed by RNA splicing. The term exon refers to both the DNA sequence within a gene and to the corresponding sequence in RNA transcripts. In RNA splicing, introns are removed and exons are covalently joined to one another as part of generating the mature messenger RNA.

“Splicing” refers to the processing of a newly synthesized messenger RNA transcript (also referred to as a primary mRNA transcript). After splicing, introns are removed and exons are joined together (ligated) for form mature mRNA molecule containing a complete open reading frame that is decoded and translated into a protein. For nuclear-encoded genes, splicing takes place within the nucleus either co-transcriptionally or immediately after transcription. The molecular mechanism of RNA splicing has been extensively described, e.g., in Pagani et al., Nature Reviews Genetics 5, 389-396, 2004; Clancy et al., Nature Education 1 (1): 31, 2011; Cheng et al., Molecular Genetics and Genomics 286 (5-6): 395-410, 2014; Taggart et al., Nature Structural & Molecular Biology 19 (7): 719-2, 2012, the contents of each of which are incorporated herein by reference. One skilled in the art is familiar with the mechanism of RNA splicing.

“Alternative splicing” refers to a regulated process during gene expression that results in a single gene coding for multiple proteins. In this process, particular exons of a gene may be included within or excluded from the final, processed messenger RNA (mRNA) produced from that gene. Consequently, the proteins translated from alternatively spliced mRNAs will contain differences in their amino acid sequence and, often, in their biological functions. Notably, alternative splicing allows the human genome to direct the synthesis of many more proteins than would be expected from its 20,000 protein-coding genes. Alternative splicing is sometimes also termed differential splicing. Alternative splicing occurs as a normal phenomenon in eukaryotes, where it greatly increases the biodiversity of proteins that can be encoded by the genome; in humans, ˜95% of multi-exonic genes are alternatively spliced. There are numerous modes of alternative splicing observed, of which the most common is exon skipping. In this mode, a particular exon may be included in mRNAs under some conditions or in particular tissues, and omitted from the mRNA in others. Abnormal variations in splicing are also implicated in disease; a large proportion of human genetic disorders result from splicing variants. Abnormal splicing variants are also thought to contribute to the development of cancer, and splicing factor genes are frequently mutated in different types of cancer. The regulation of alternative splicing is also described in the art, e.g., in Douglas et al., Annual Review of Biochemistry 72 (1): 291-336, 2003; Pan et al., Nature Genetics 40 (12): 1413-1415, 2008; Martin et al., Nature Reviews 6 (5): 386-398, 2005; Skotheim et al., The International Journal of Biochemistry & Cell Biology 39 (7-8): 1432-49, 2007, each of which is incorporated herein by reference.

A “coding frame” or “open reading frame” refers to a stretch of codons that encodes a polypeptide. Since DNA is interpreted in groups of three nucleotides (codons), a DNA strand has three distinct reading frames. The double helix of a DNA molecule has two anti-parallel strands so, with the two strands having three reading frames each, there are six possible frame translations. A functional protein may be produced when translation proceeds in the correct coding frame. An insertion or a deletion of one or two bases in the open reading frame causes a shift in the coding frame that is also referred to as a “frameshift mutation.” A frameshift mutation typical results in premature translation termination and/or truncated or non-functional protein.

These and other exemplary substituents are described in more detail in the Detailed Description, Examples, and Claims. The invention is not intended to be limited in any manner by the above exemplary listing of substituents.

### DETAILED DESCRIPTION OF CERTAIN EMBODIMENTS

Disclosed herein are novel genome/base-editing systems, methods, and compositions for generating engineered and naturally-occurring protective variants of the liver protein Proprotein Convertase Subtilisin/Kexin Type 9 (PCSK9) to boost LDL receptor-mediated clearance of LDL cholesterol, alone and in combination with other protective gene variants that could synergistically improve circulating cholesterol and triglyceride levels.

Proprotein convertase subtilisin-kexin type 9 (PCSK9), also known as neural apoptosis-regulated convertase 1 (“NARC-I”), is a proteinase K-like subtilase identified as the 9th member of the secretory subtilase family. The gene for PCSK9 localizes to human chromosome Ip33-p34.3. PCSK9 is expressed in cells capable of proliferation and differentiation including, for example, hepatocytes, kidney mesenchymal cells, intestinal ileum, and colon epithelia as well as embryonic brain telencephalon neurons. See, e.g., Seidah et al., 2003 PNAS 100:928-933, which is incorporated herein by reference.

Original synthesis of PCSK9 is in the form of an inactive enzyme precursor, or zymogen, of 72-kDa, which undergoes autocatalytic, intramolecular processing in the endoplasmic reticulum (“ER”) to activate its functionality. This internal processing event has been reported to occur at the SSVFAQ↓SIP motif, and has been reported as a requirement of exit from the ER. “↓” indicates cleavage site. See, Benjannet et al., 2004 J. Biol. Chem. 279:48865-48875, and Seidah et al., 2003 PNAS 100:928-933, each of which are incorporated herein by reference. The cleaved protein is then secreted. The cleaved peptide remains associated with the activated and secreted enzyme. The gene sequence for human PCSK9, which is ˜22-kb long with 12 exons encoding a 692 amino acid protein, can be found, for example, at Deposit No. NP_777596.2. Human, mouse and rat PCSK9 nucleic acid sequences have been deposited; see, e.g., GenBank Accession Nos.: AX127530 (also AX207686), AX207688, and AX207690, respectively. The translated protein contains a signal peptide in the NH2-terminus, and in cells and tissues an about 74 kDa zymogen (precursor) form of the full-length protein is found in the endoplasmic reticulum. During initial processing in the cell, the about 14 kDa prodomain peptide is autocatalytically cleaved to yield a mature about 60 kDa protein containing the catalytic domain and a C-terminal domain often referred to as the cysteine-histidine rich domain (CHRD). This about 60 kDa form of PCSK9 is secreted from liver cells. The secreted form of PCSK9 appears to be the physiologically active species, although an intracellular functional role of the about 60 kDa form has not been ruled out.

Wild Type PCSK9 Gene (>gi|299523249|ref|NM_174936.3|Homo sapiens proprotein convertase subtilisin/kexin type 9 (PCSK9), transcript variant 1, SEQ ID NO: 1990)

GTCCGATGGGGCTCTGGTGGCGTGATCTGCGCGCCCCAGGCGTCAAGCACCCACAC
CCTAGAAGGTTTCCGCAGCGACGTCGAGGCGCTCATGGTTGCAGGCGGGCGCCGCC
GTTCAGTTCAGGGTCTGAGCCTGGAGGAGTGAGCCAGGCAGTGAGACTGGCTCGGG
CGGGCCGGGACGCGTCGTTGCAGCAGCGGCTCCCAGCTCCCAGCCAGGATTCCGCG
CGCCCCTTCACGCGCCCTGCTCCTGAACTTCAGCTCCTGCACAGTCCTCCCCACCGC
AAGGCTCAAGGCGCCGCCGGCGTGGACCGCGCACGGCCTCTAGGTCTCCTCGCCAG
GACAGCAACCTCTCCCCTGGCCCTCATGGGCACCGTCAGCTCCAGGCGGTCCTGGTG
GCCGCTGCCACTGCTGCTGCTGCTGCTGCTGCTCCTGGGTCCCGCGGGCGCCCGTGC
GCAGGAGGACGAGGACGGCGACTACGAGGAGCTGGTGCTAGCCTTGCGTTCCGAGG
AGGACGGCCTGGCCGAAGCACCCGAGCACGGAACCACAGCCACCTTCCACCGCTGC
GCCAAGGATCCGTGGAGGTTGCCTGGCACCTACGTGGTGGTGCTGAAGGAGGAGAC
CCACCTCTCGCAGTCAGAGCGCACTGCCCGCCGCCTGCAGGCCCAGGCTGCCCGCCG
GGGATACCTCACCAAGATCCTGCATGTCTTCCATGGCCTTCTTCCTGGCTTCCTGGTG
AAGATGAGTGGCGACCTGCTGGAGCTGGCCTTGAAGTTGCCCCATGTCGACTACATC
GAGGAGGACTCCTCTGTCTTTGCCCAGAGCATCCCGTGGAACCTGGAGCGGATTACC
CCTCCACGGTACCGGGCGGATGAATACCAGCCCCCCGACGGAGGCAGCCTGGTGGA
GGTGTATCTCCTAGACACCAGCATACAGAGTGACCACCGGGAAATCGAGGGCAGGG
TCATGGTCACCGACTTCGAGAATGTGCCCGAGGAGGACGGGACCCGCTTCCACAGA
CAGGCCAGCAAGTGTGACAGTCATGGCACCCACCTGGCAGGGGTGGTCAGCGGCCG
GGATGCCGGCGTGGCCAAGGGTGCCAGCATGCGCAGCCTGCGCGTGCTCAACTGCC
AAGGGAAGGGCACGGTTAGCGGCACCCTCATAGGCCTGGAGTTTATTCGGAAAAGC
CAGCTGGTCCAGCCTGTGGGGCCACTGGTGGTGCTGCTGCCCCTGGCGGGTGGGTAC
AGCCGCGTCCTCAACGCCGCCTGCCAGCGCCTGGCGAGGGCTGGGGTCGTGCTGGT
CACCGCTGCCGGCAACTTCCGGGACGATGCCTGCCTCTACTCCCCAGCCTCAGCTCC
CGAGGTCATCACAGTTGGGGCCACCAATGCCCAAGACCAGCCGGTGACCCTGGGGA
CTTTGGGGACCAACTTTGGCCGCTGTGTGGACCTCTTTGCCCCAGGGGAGGACATCA
TTGGTGCCTCCAGCGACTGCAGCACCTGCTTTGTGTCACAGAGTGGGACATCACAGG
CTGCTGCCCACGTGGCTGGCATTGCAGCCATGATGCTGTCTGCCGAGCCGGAGCTCA
CCCTGGCCGAGTTGAGGCAGAGACTGATCCACTTCTCTGCCAAAGATGTCATCAATG
AGGCCTGGTTCCCTGAGGACCAGCGGGTACTGACCCCCAACCTGGTGGCCGCCCTGC
CCCCCAGCACCCATGGGGCAGGTTGGCAGCTGTTTTGCAGGACTGTATGGTCAGCAC
ACTCGGGGCCTACACGGATGGCCACAGCCGTCGCCCGCTGCGCCCCAGATGAGGAG
CTGCTGAGCTGCTCCAGTTTCTCCAGGAGTGGGAAGCGGCGGGGCGAGCGCATGGA
GGCCCAAGGGGGCAAGCTGGTCTGCCGGGCCCACAACGCTTTTGGGGGTGAGGGTG
TCTACGCCATTGCCAGGTGCTGCCTGCTACCCCAGGCCAACTGCAGCGTCCACACAG
CTCCACCAGCTGAGGCCAGCATGGGGACCCGTGTCCACTGCCACCAACAGGGCCAC
GTCCTCACAGGCTGCAGCTCCCACTGGGAGGTGGAGGACCTTGGCACCCACAAGCC
GCCTGTGCTGAGGCCACGAGGTCAGCCCAACCAGTGCGTGGGCCACAGGGAGGCCA
GCATCCACGCTTCCTGCTGCCATGCCCCAGGTCTGGAATGCAAAGTCAAGGAGCATG
GAATCCCGGCCCCTCAGGAGCAGGTGACCGTGGCCTGCGAGGAGGGCTGGACCCTG
ACTGGCTGCAGTGCCCTCCCTGGGACCTCCCACGTCCTGGGGGCCTACGCCGTAGAC
AACACGTGTGTAGTCAGGAGCCGGGACGTCAGCACTACAGGCAGCACCAGCGAAGG
GGCCGTGACAGCCGTTGCCATCTGCTGCCGGAGCCGGCACCTGGCGCAGGCCTCCC
AGGAGCTCCAGTGACAGCCCCATCCCAGGATGGGTGTCTGGGGAGGGTCAAGGGCT
GGGGCTGAGCTTTAAAATGGTTCCGACTTGTCCCTCTCTCAGCCCTCCATGGCCTGG
CACGAGGGGATGGGGATGCTTCCGCCTTTCCGGGGCTGCTGGCCTGGCCCTTGAGTG
GGGCAGCCTCCTTGCCTGGAACTCACTCACTCTGGGTGCCTCCTCCCCAGGTGGAGG
TGCCAGGAAGCTCCCTCCCTCACTGTGGGGCATTTCACCATTCAAACAGGTCGAGCT
GTGCTCGGGTGCTGCCAGCTGCTCCCAATGTGCCGATGTCCGTGGGCAGAATGACTT
TTATTGAGCTCTTGTTCCGTGCCAGGCATTCAATCCTCAGGTCTCCACCAAGGAGGC
AGGATTCTTCCCATGGATAGGGGAGGGGGCGGTAGGGGCTGCAGGGACAAACATCG
TTGGGGGGTGAGTGTGAAAGGTGCTGATGGCCCTCATCTCCAGCTAACTGTGGAGA
AGCCCCTGGGGGCTCCCTGATTAATGGAGGCTTAGCTTTCTGGATGGCATCTAGCCA
GAGGCTGGAGACAGGTGCGCCCCTGGTGGTCACAGGCTGTGCCTTGGTTTCCTGAGC
CACCTTTACTCTGCTCTATGCCAGGCTGTGCTAGCAACACCCAAAGGTGGCCTGCGG
GGAGCCATCACCTAGGACTGACTCGGCAGTGTGCAGTGGTGCATGCACTGTCTCAGC
CAACCCGCTCCACTACCCGGCAGGGTACACATTCGCACCCCTACTTCACAGAGGAA
GAAACCTGGAACCAGAGGGGGCGTGCCTGCCAAGCTCACACAGCAGGAACTGAGCC
AGAAACGCAGATTGGGCTGGCTCTGAAGCCAAGCCTCTTCTTACTTCACCCGGCTGG
GCTCCTCATTTTTACGGGTAACAGTGAGGCTGGGAAGGGGAACACAGACCAGGAAG
CTCGGTGAGTGATGGCAGAACGATGCCTGCAGGCATGGAACTTTTTCCGTTATCACC
CAGGCCTGATTCACTGGCCTGGCGGAGATGCTTCTAAGGCATGGTCGGGGGAGAGG
GCCAACAACTGTCCCTCCTTGAGCACCAGCCCCACCCAAGCAAGCAGACATTTATCT
TTTGGGTCTGTCCTCTCTGTTGCCTTTTTACAGCCAACTTTTCTAGACCTGTTTTGCTT
TTGTAACTTGAAGATATTTATTCTGGGTTTTGTAGCATTTTTATTAATATGGTGACTT
TTTAAAATAAAAACAAACAAACGTTGTCCTAACAAAAAAAAAAAAAAAAAAAAA
Human PCSK9 Amino Acid Sequence
(SEQ ID NO: 1991)
MGTVSSRRSWWPLPLLLLLLLLLGPAGARAQEDEDGDYEELVLALRSEEDGLAEAPEH
GTTATFHRCAKDPWRLPGTYVVVLKEETHLSQSERTARRLQAQAARRGYLTKILHVFH
GLLPGFLVKMSGDLLELALKLPHVDYIEEDSSVFAQSIPWNLERITPPRYRADEYQPPDG
GSLVEVYLLDTSIQSDHREIEGRVMVTDFENVPEEDGTRFHRQASKCDSHGTHLAGVVS
GRDAGVAKGASMRSLRVLNCQGKGTVSGTLIGLEFIRKSQLVQPVGPLVVLLPLAGGYS
RVLNAACQRLARAGVVLVTAAGNFRDDACLYSPASAPEVITVGATNAQDQPVTLGTLG
TNFGRCVDLFAPGEDIIGASSDCSTCFVSQSGTSQAAAHVAGIAAMMLSAEPELTLAELR
QRLIHFSAKDVINEAWFPEDQRVLTPNLVAALPPSTHGAGWQLFCRTVWSAHSGPTRM
ATAVARCAPDEELLSCSSFSRSGKRRGERMEAQGGKLVCRAHNAFGGEGVYAIARCCL
LPQANCSVHTAPPAEASMGTRVHCHQQGHVLTGCSSHWEVEDLGTHKPPVLRPRGQPN
QCVGHREASIHASCCHAPGLECKVKEHGIPAPQEQVTVACEEGWTLTGCSALPGTSHVL
GAYAVDNTCVVRSRDVSTTGSTSEGAVTAVAICCRSRHLAQASQELQ
Mouse PCSK 9 Amino Acid Sequence
(SEQ ID NO: 1992)
MGTHCSAWLRWPLLPLLPPLLLLLLLLCPTGAGAQDEDGDYEELMLALPSQEDGLADE
AAHVATATFRRCSKEAWRLPGTYIVVLMEETQRLQIEQTAHRLQTRAARRGYVIKVLHI
FYDLFPGFLVKMSSDLLGLALKLPHVEYIEEDSFVFAQSIPWNLERIIPAWHQTEEDRSPD
GSSQVEVYLLDTSIQGAHREIEGRVTITDFNSVPEEDGTRFHRQASKCDSHGTHLAGVVS
GRDAGVAKGTSLHSLRVLNCQGKGTVSGTLIGLEFIRKSQLIQPSGPLVVLLPLAGGYSR
ILNAACRHLARTGVVLVAAAGNFRDDACLYSPASAPEVITVGATNAQDQPVTLGTLGT
NFGRCVDLFAPGKDIIGASSDCSTCFMSQSGTSQAAAHVAGIVARMLSREPTLTLAELRQ
RLIHFSTKDVINMAWFPEDQQVLTPNLVATLPPSTHETGGQLLCRTVWSAHSGPTRTAT
ATARCAPEEELLSCSSFSRSGRRRGDWIEAIGGQQVCKALNAFGGEGVYAVARCCLVPR
ANCSIHNTPAARAGLETHVHCHQKDHVLTGCSFHWEVEDLSVRRQPALRSRRQPGQCV
GHQAASVYASCCHAPGLECKIKEHGISGPSEQVTVACEAGWTLTGCNVLPGASLTLGAY
SVDNLCVARVHDTARADRTSGEATVAAAICCRSRPSAKASWVQ
Rat PCSK9 Amino Acid Sequence
(SEQ ID NO: 1993)
MGIRCSTWLRWPLSPQLLLLLLLCPTGSRAQDEDGDYEELMLALPSQEDSLVDEASHVA
TATFRRCSKEAWRLPGTYVVVLMEETQRLQVEQTAHRLQTWAARRGYVIKVLHVFYD
LFPGFLVKMSSDLLGLALKLPHVEYIEEDSLVFAQSIPWNLERIIPAWQQTEEDSSPDGSS
QVEVYLLDTSIQSGHREIEGRVTITDFNSVPEEDGTRFHRQASKCDSHGTHLAGVVSGRD
AGVAKGTSLHSLRVLNCQGKGTVSGTLIGLEFIRKSQLIQPSGPLVVLLPLAGGYSRILNT
ACQRLARTGVVLVAAAGNFRDDACLYSPASAPEVITVGATNAQDQPVTLGTLGTNFGR
CVDLFAPGKDIIGASSDCSTCYMSQSGTSQAAAHVAGIVAMMLNRDPALTLAELRQRLI
LFSTKDVINMAWFPEDQRVLTPNRVATLPPSTQETGGQLLCRTVWSAHSGPTRTATATA
RCAPEEELLSCSSFSRSGRRRGDRIEAIGGQQVCKALNAFGGEGVYAVARCCLLPRVNC
SIHNTPAARAGPQTPVHCHQKDHVLTGCSFHWEVENLRAQQQPLLRSRHQPGQCVGHQ
EASVHASCCHAPGLECKIKEHGIAGPAEQVTVACEAGWTLTGCNVLPGASLPLGAYSVD
NVCVARIRDAGRADRTSEEATVAAAICCRSRPSAKASWVHQ

PCSK9 has been ascribed a role in the differentiation of hepatic and neuronal cells, is highly expressed in embryonic liver, and has been strongly implicated in cholesterol homeostasis. Recent studies suggest a specific role in cholesterol biosynthesis or uptake for PCSK9. In a study of cholesterol-fed rats, Maxwell et al. found that PCSK9 was downregulated in a similar manner as three other genes involved in cholesterol biosynthesis, Maxwell et al., 2003 J Lipid Res. 44:2109-2119, which are incorporated herein by reference. Interestingly, as well, the expression of PCSK9 was regulated by sterol regulatory element-binding proteins (“SREBP”), as seen with other genes involved in cholesterol metabolism. These findings were later supported by a study of PCSK9 transcriptional regulation which demonstrated that such regulation was quite typical of other genes implicated in lipoprotein metabolism; Dubuc et al., 2004 Arterioscler. Thromb. Vase. Biol 24:1454-1459, which is incorporated herein by reference. PCSK9 expression was upregulated by statins in a manner attributed to the cholesterol-lowering effects of the drugs. Further, the PCSK9 promoters possessed two conserved sites involved in cholesterol regulation, a sterol regulatory element and a SpI site. Adenoviral expression of PCSK9 has been shown to lead to a notable time-dependent increase in circulating LDL (Benjannet et al., 2004 J Biol Chem. 279:48865-48875, which is incorporated herein by reference). More, mice deleted of the PCSK9 gene have increased levels of hepatic LDL receptors and more rapidly clear LDL from the plasma; Rashid et al., 2005 Proc. Natl Acad. Sci. USA 102:5374-5379, which is incorporated herein by reference.

Recently it was reported that medium from HepG2 cells transiently transfected with PCSK9 reduced the amount of cell surface LDLR and internalization of LDL when transferred to untransfected HepG2 cells; see Cameron et al., 2006 Human Mol Genet. 15:1551-1558, which is incorporated herein by reference. It was concluded that either PCSK9 or a factor acted upon by PCSK9 is secreted and is capable of degrading LDLR both in transfected and untransfected cells. More recently, it was demonstrated that purified PCSK9 added to the medium of HepG2 cells had the effect of reducing the number of cell-surface LDLRs in a dose- and time-dependent manner; Lagace et al., 2006 J Clin. Invest. 116:2995-3005, which are incorporated herein by reference.

Numerous PCSK9 variants are disclosed and/or claimed in several patent publications including, but not limited to the following: PCT Publication Nos. WO2001031007, WO2001057081, WO2002014358, WO2001098468, WO2002102993, WO2002102994, WO2002046383, WO2002090526, WO2001077137, and WO2001034768; US Publication Nos. US 2004/0009553 and US 2003/0119038, and European Publication Nos. EP 1 440 981, EP 1 067 182, and EP 1 471 152, each of which are incorporated herein by reference.

Several mutant forms of PCSK9 are well characterized, including S127R, N157K, F216L, R218S, and D374Y, with S127R, F216L, and D374Y being linked to autosomal dominant hypercholesterolemia (ADH). Benjannet et al. (J. Biol. Chem., 279(47):48865-48875 (2004)) demonstrated that the S127R and D374Y mutations result in a significant decrease in the level of pro-PCSK9 processed in the ER to form the active secreted zymogen. As a consequence it is believed that wild-type PCSK9 increases the turnover rate of the LDL receptor causing inhibition of LDL clearance (Maxwell et al., PNAS, 102(6):2069-2074 (2005); Benjannet et al., and Lalanne et al), while PCSK9 autosomal dominant mutations result in increased levels of LDLR, increased clearance of circulating LDL, and a corresponding decrease in plasma cholesterol levels. See, Rashid et al., PNAS, 102(15):5374-5379 (2005); Abifadel et al., 2003 Nature Genetics 34:154-156; Timms et al., 2004 Hum. Genet. 114:349-353; and Leren, 2004 Clin. Genet. 65:419-422, each of which are incorporated herein by reference.

A later-published study on the S127R mutation of Abifadel et al., reported that patients carrying such a mutation exhibited higher total cholesterol and apoB100 in the plasma attributed to (1) an overproduction of apoB100-containing lipoproteins, such as low density lipoprotein (“LDL”), very low density lipoprotein (“VLDL”) and intermediate density lipoprotein (“IDL”), and (2) an associated reduction in clearance or conversion of said lipoproteins. Together, the studies referenced above evidence the fact that PCSK9 plays a role in the regulation of LDL production. Expression or upregulation of PCSK9 is associated with increased plasma levels of LDL cholesterol, and inhibition or the lack of expression of PCSK9 is associated with low LDL cholesterol plasma levels. Significantly, lower levels of LDL cholesterol associated with sequence variations in PCSK9 have conferred protection against coronary heart disease; Cohen et al., 2006 N. Engl. J. Med. 354:1264-1272.

Lalanne et al. demonstrated that LDL catabolism was impaired and apolipoprotein B-containing lipoprotein synthesis was enhanced in two patients harboring S127R mutations in PCSK9 (J. Lipid Research, 46:1312-1319 (2005)). Sun et al. also provided evidence that mutant forms of PCSK9 are also the cause of unusually severe dominant hypercholesterolaemia as a consequence of its effect of increasing apolipoprotein B secretion (Sun et al., Hum. Mol. Genet., 14(9):1161-1169 (2005)). These results were consistent with earlier results which demonstrated adenovirus-mediated overexpression of PCSK9 in mice results in severe hypercholesteromia due to drastic decreases in the amount of LDL receptor Dubuc et al., Thromb. Vasc. Biol., 24:1454-1459 (2004), in addition to results demonstrating mutant forms of PCSK9 also reduce the level of LDL receptor (Park et al., J. Biol. Chem., 279:50630-50638 (2004). The overexpression of PCSK9 in cell lines, including liver-derived cells, and in livers of mice in vivo, results in a pronounced reduction in LDLR protein levels and LDLR functional activity without changes in LDLR mRNA level (Maxwell et al., Proc. Nat. Amer. Sci., 101:7100-7105 (2004); Benjannet S. et al., J. Bio. Chem. 279: 48865-48875 (2004)).

Various therapeutic approaches to the inhibition of PSCK9 have been proposed, including: inhibition of PSCK9 synthesis by gene silencing agents, e.g., RNAi; inhibition of PCSK9 binding to LDLR by monoclonal antibodies, small peptides or adnectins; and inhibition of PCSK9 autocatalytic processing by small molecule inhibitors. These strategies have been described in Hedrick et al., Curr Opin Investig Drugs 2009; 10:938-46; Hooper et al., Expert Opin Biol Ther, 2013; 13:429-35; Rhainds et al., Clin Lipid, 2012; 7:621-40; Seidah et al; Expert Opin Ther Targets 2009; 13:19-28; and Seidah et al., Nat Rev Drug Discov 2012; 11:367-83, each of which are incorporated herein by reference.

### Strategies for Generating PCSK9 Mutants

Some aspects of the present disclosure provide systems, compositions, and methods of editing polynucleotides encoding the PCSK9 protein to introducing mutations into the PCSK9 gene. The gene editing methods described herein, rely on nucleobase editors as described in U.S. Pat. No. 9,068,179, US Patent Application Publications US20150166980, US20150166981, US20150166982, US20150166984, and US20150165054, and U.S. Provisional Applications 62/245,828, 62/279,346, 62/311,763, 62/322,178, 62/357,352, 62/370,700, and 62/398,490, and in Komor et al., Nature, Programmable editing of a target base in genomic DNA without double-stranded DNA cleavage, 533, 420-424 (2016), each of which are incorporated herein by reference.

The nucleobase editors highly efficient at precisely editing a target base in the PCSK9 gene and a DNA double stand break is not necessary for the gene editing, thus reducing genome instability and preventing possible oncogenic modifications that may be caused by other genome editing methods. The nucleobase editors described herein may be programmed to target and modify a single base. In some embodiments, the target base is a cytosine (C) base and may be converted to a thymine (T) base via deamination by the nucleobase editor.

To edit the polynucleotide encoding the PCSK9 protein, the polynucleotide is contacted with a nucleobase editors described herein. In some embodiments, the PCSK9-encoding polynucleotide is contacted with a nucleobase editor and a guide nucleotide sequence, wherein the guide nucleotide sequence targets the nucleobase editor the target base (e.g., a C base) in the PCSK9-encoding polynucleotide.

In some embodiments, the PCSK9-encoding polynucleotide is the PCSK9 gene locus in the genomic DNA of a cell. In some embodiments, the cell is a cultured cell. In some embodiments, the cell is in vivo. In some embodiments, the cell is in vitro. In some embodiments, the cell is ex vivo. In some embodiments, the cell is from a mammal. In some embodiments, the mammal is a human. In some embodiments, the mammal is a rodent. In some embodiments, the rodent is a mouse. In some embodiments, the rodent is a rat.

As would be understood be those skilled in the art, the PCSK9-encoding polynucleotide may be a DNA molecule comprising a coding strand and a complementary strand, e.g., the PCSK9 gene locus in a genome. As such, the PCSK9-encoding polynucleotide may also include coding regions (e.g., exons) and non-coding regions (e.g., introns of splicing sites). In some embodiments, the target base (e.g., a C base) is located in the coding region (e.g., an exon) of the PCSK9-encoding polynucleotide (e.g., the PCSK9 gene locus). As such, the conversion of a base in the coding region may result in an amino acid change in the PCSK9 protein sequence, i.e., a mutation. In some embodiments, the mutation is a loss of function mutation. In some embodiments, the loss-of-function mutation is a naturally occurring loss-of-function mutation, e.g., G106R, L253F, A443T, R93C, etc. In some embodiments, the loss-of-function mutation is engineered (i.e., not naturally occurring), e.g., G24D, S47F, R46H, S153N, H193Y, etc.

In some embodiments, the target base is located in a non-coding region of the PCSK9 gene, e.g., in an intron or a splicing site. In some embodiments, a target base is located in a splicing site and the editing of such target base causes alternative splicing of the PSCK9 mRNA. In some embodiments, the alternative splicing leads to leading to loss-of-function PCSK9 mutants. In some embodiments, the alternative splicing leads to the introduction of a premature stop codon in a PSCK9 mRNA, resulting in truncated and unstable PCSK9 proteins. In some embodiments, PCSK9 mutants that are defective in folding are produced.

PCSK9 variants that are particularly useful in creating using the present disclosure are loss-of-function variants that may boost LDL receptor-mediated clearance of LDL cholesterol, alone or in combination with other genes involved in the pathway, e.g., APOC3, LDL-R, or Idol. In some embodiments, the PCKS9 loss-of-function variants produced using the methods of the present disclosure express efficiently in a cell. In some embodiments, the PCKS9 loss-of-function variants produced using the methods of the present disclosure is activated and exported to engage the clathrin-coated pits from unmodified cells in a paracrine mechanism, thus competing with the wild-type PCSK9 protein. In some embodiments, the PCSK9 loss-of-function variant comprises mutations in residues in the LDL-R bonding region that make direct contact with the LDL-R protein. In some embodiments, the residues in the LDL-R bonding region that make direct contact with the LDL-R protein are selected from the group consisting of R194, R237, F379, 5372, D374, D375, D378, R46, R237, and A443.

As described herein, a loss-of-function PCSK9 variant, may have reduced activity compared to a wild type PCSK9 protein. PCSK9 activity refers to any known biological activity of the PCSK9 protein in the art. For example, in some embodiments, PCSK9 activity refers to its protease activity. In some embodiments, PCSK9 activity refers to its ability to be secreted through the cellular secretory pathway. In some embodiments, PCSK9 activity refers to its ability to act as a protein-binding adaptor in clathrin-coated vesicles. In some embodiments, PCSK9 activity refers to its ability to interact with LDL receptor. In some embodiments, PCSK9 activity refers to its ability to prevent LDL receptor recycling. These examples are not meant to be limiting.

In some embodiments, the activity of a loss-of-function PCSK9 variant may be reduced by at lead 20%, at least 30%, at least 40%, at least 50%, at least 60%, at least 70%, at least 80%, at least 90%, at least 99%, or more. In some embodiments, the loss-of-function PCSK9 variant has no more than 50%, no more than 40%, no more than 30%, no more than 20%, no more than 10%, no more than 5%, no more than 1% or less activity compared to a wild type PCSK9 protein. Non-limiting, exemplary assays for determining PCSK9 activity have been described in the art, e.g., in US Patent Application Publication US20120082680, which are incorporated herein by reference.

To edit the PCSK9 gene, the PCSK9 gene (a polynucleotide molecule) may contact the nucleobase editor, wherein the nucleobase editor binds to its target sequence and edits the desired base. For example, the nucleobase editor may be expressed in a cell where PCSK9 gene editing is desired (e.g., a liver cell), to thereby allowing contact of the PCSK9 gene with the nucleobase editor. In some embodiments, the binding of the nucleobase editor to its target sequence in the PCSK9 is mediated by a guide nucleotide sequence, e.g., a nucleotide molecule comprising a nucleotide sequence that is complementary to one of the strands of the target sequence in the PCSK9 gene. Thus, by designing the guide nucleotide sequence, the nucleobase editor may be programmed to edit any target base in the PCSK9 gene. In some embodiments, the guide nucleotide sequence is co-expressed with the nucleobase editor in a cell where editing is desired.

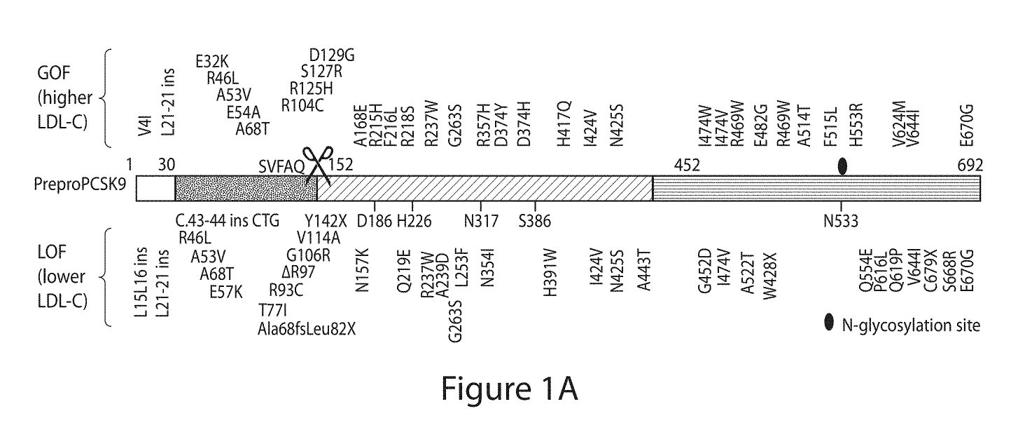

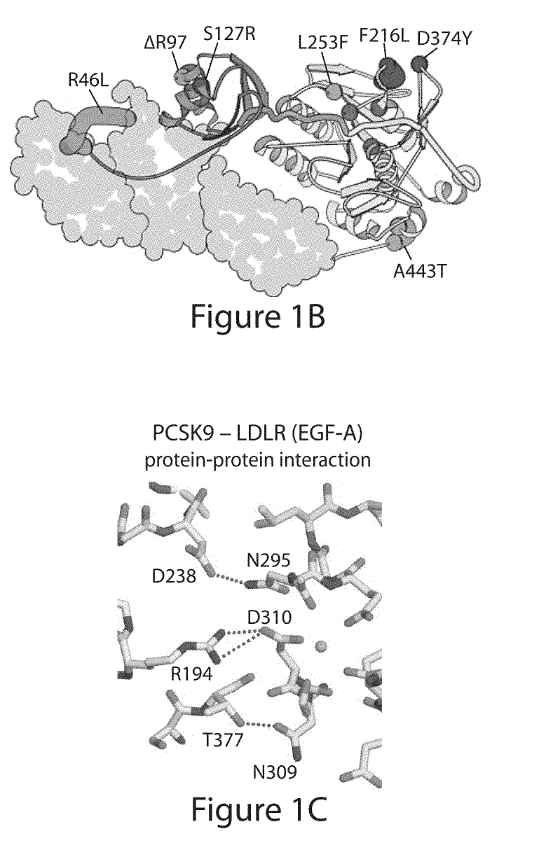

Provided herein are non-limiting, exemplary PCSK9 loss-of-function variants that may be produced via base editing (Table 1 and FIG. 1) and strategies for making them.

TABLE 1
Exemplary Loss-of-Function PCSK9 Mutations

| | | Effect on PCSK9 |
|---|---|---|
| Natural variants | Engineered variants | function/structure |
| G106R, L253F, N354I, Q152H | D186N, H226Y, S386L, | prevent autoactivation |
| | A290V/T, S153N | |
| R46L, R237W | R46C, R46H, R237Q | loss-of-function, but normal |
| | | expression |
| A443T, Q219E | A220V/T | faster protease inactivation |
| R46L, R237W | R46C/H, H193Y, R194Q/W, | diminished affinity |
| | N295A, S372F, S373N, D374N, | for LDL-R |
| | S376N, C375Y, T377I, C378Y, | |
| | F379 | |
| G236S, G106R, G670E | C375Y, C378Y, C679Y, other C | destabilized protein |
| | to Y, P to S/L, | folding |
| | G to R, E to K, etc. identifiable | |
| | by screening | |
| A53V, L15insL, | E49K, S47F, P12S/L, P14S/L, | modify ER entry leader |
| R46L | G24D, G27D, R29C | peptide |
| cytosine (C) 161 to thymine | guanine (G) to adenosine (A) in | modification or destabilization |
| (T) | intron-exon junctions, modify | of mRNA |
| | ATG | |
| | (Methionine) start codon to ATA | |
| | (Isoleucine) | |
| Y142X, C679X, | Q to Amber, R to Opal, W to | premature stop codons |
| A68frame shift, R97del (X is | Opal/Amber | |
| a stop codon) | (preferably in tandem, or in | |
| | flexible loops) | |
| R46L, A53V | N533A, S688F | post-translational |
| | | modification sites |

### Codon Change

Using the nucleobase editors described herein, several amino acid codons may be converted to a different codon via deamination of a target base within the codon. For example, in some embodiments, a cytosine (C) base is converted to a thymine (T) base via deamination by a nucleobase editor comprising a cytosine deaminase domain (e.g., APOBEC1 or AID). It is worth noting that during a C to T change via deamination (e.g., by a cytosine deaminase such as APOBEC1 or AID), the cytosine is first converted to a uridine (U), leading to a G:U mismatch. The G:U mismatch is then converted by DNA repair and replication pathways to T:A pair, thus introducing the thymine at the position of the original cytosine. As it is familiar to one skilled in the art, conversion of a base in an amino acid codon may lead to a change of the amino acid the codon encodes. Cytosine deaminases are capable of converting a cytosine (C) base to a thymine (T) base via deamination. Thus, it is envisioned that, for amino acid codons containing a C base, the C base may be directly converted to T. For example, leucine codon (CTC) may be changed to a TTC (phenylalanine) codon via the deamination of the first C on the coding strand. For amino acid codons that contain a guanine (G) base, a C base is present on the complementary strand; and the G base may be converted to an adenosine (A) via the deamination of the C on the complementary strand. For example, an ATG (Met/M) codon may be converted to a ATA (Ile/I) codon via the deamination of the third C on the complementary strand. In some embodiments, two C to T changes are required to convert a codon to a different codon. Non-limiting examples of possible mutations that may be made in the PCSK9-encoding polynucleotide by the nucleobase editors of the present disclosure are summarized in Table 2.

TABLE 2
Exemplary Codon Changes in PCSK9 Gene via Base Editing

| Target codon | Base-editing reaction (s) | Edited codon |
|---|---|---|
| CTT (Leu/L) | 1st base C to T on coding strand | TTT (Phe/F) |
| CTC (Leu/L) | 1st base C to T on coding strand | TTC (Phe/F) |
| ATG (Met/M) | 3rd base C to T on complementary | ATA (Ile/I) |
| | strand | |
| GTT (Val/V) | 1st base C to T on complementary stand | ATT (Ile/I) |
| GTA (Val/V) | 1st base C to T on complementary stand | ATA (Ile/I) |
| GTC (Val/V) | 1st base C to T on complementary | ATC (Ile/I) |
| | strand | |
| GTG (Val/V) | 1st base C to T on complementary | ATG (Met/M) |
| | strand | |
| TCT (Ser/S) | 2nd base C to T on coding strand | TTT (Phe/F) |
| TCC (Ser/S) | 2nd base C to T on coding strand | TTC (Phe/F) |
| TCA (Ser/S) | 2nd base C to T on coding strand | TTA (Leu/L) |
| TCG (Ser/S) | 2nd base C to T on coding strand | TTG (Leu/L) |
| AGT (Ser/S) | 2nd base C to T on complementary | AAT (Asp/N) |
| | strand | |
| AGC (Ser/S) | 2nd base C to T on complementary | AAC (Aps/N) |
| | strand | |
| CCT (Pro/P) | 1st base C to T on coding strand | TCT (Ser/S) |
| CCC (Pro/P) | 1st base C to T on coding strand | TCC (Ser/S) |
| CCA (Pro/P) | 1st base C to T on coding strand | TCA (Ser/S) |
| CCG (Pro/P) | 1st base C to T on coding strand | TCG (Ser/S) |
| CCT (Pro/P) | 2nd base C to T on coding strand | CTT (Leu/L) |
| CCC (Pro/P) | 2nd base C to T on coding strand | CTC (Leu/L) |
| CCA (Pro/P) | 2nd base C to T on coding strand | CTA (Leu/L) |
| CCG (Pro/P) | 2nd base C to T on coding strand | CTG (Leu/L) |
| ACT (Thr/T) | 2nd base C to T on coding strand | ATT (Leu/L) |
| ACC (Thr/T) | 2nd base C to T on coding strand | ATC (Leu/L) |
| ACA (Thr/T) | 2nd base C to T on coding strand | ATA (Leu/L) |
| ACG (Thr/T) | 2nd base C to T on coding strand | ATG (Met/M) |
| GCT (Ala/A) | 2nd base C to T on coding strand | GTT (Val/V) |
| GCC (Ala/A) | 2nd base C to T on coding strand | GTC (Val/V) |
| GCA (Ala/A) | 2nd base C to T on coding strand | GTA (Val/V) |
| GCG (Ala/A) | 2nd base C to T on coding strand | GTG (Val/V) |
| GCT (Ala/A) | 1st base C to T on complementary stand | ACT (Thr/T) |
| GCC (Ala/A) | 1st base C to T on complementary stand | ACC (Thr/T) |
| GCA (Ala/A) | 1st base C to T on complementary stand | ACA (Thr/T) |
| GCG (Ala/A) | 1st base C to T on complementary stand | ACG (Thr/T) |
| CAT (His/H) | 1st base C to T on complementary stand | TAT (Tyr/Y) |
| CAC (His/H) | 1st base C to T on complementary stand | TAC (Tyr/Y) |
| GAT (Asp/D) | 1st base C to T on complementary stand | AAT (Asp/N) |
| GAC (Asp/D) | 1st base C to T on complementary stand | AAC (Asp/N) |
| GAA (Glu/E) | 1st base C to T on complementary stand | AAA (Lys/K) |
| GAG (Glu/E) | 1st base C to T on complementary stand | AAG (Lys/K) |
| TGT (Cys/C) | 2nd base C to T on complementary | TAT (Tyr/Y) |
| | stand | |
| TGC (Cys/C) | 2nd base C to T on complementary | TAC (Tyr/Y) |
| | stand | |
| CGT (Arg/R) | 1st base C to T on coding strand | TGT (Cys/C) |
| CGC (Arg/R) | 1st base C to T on coding strand | TGC (Cys/C) |
| AGA (Arg/R) | 2nd base C to T on complementary | AAA (Lys/K) |
| | stand | |
| AGG (Arg/R) | 2nd base C to T on complementary | AAG (Lys/K) |
| | stand | |
| CGG (Arg/R) | 2nd base C to T on complementary | CAG (Gln/Q) |
| | stand | |
| CGG (Arg/R) | 1st base C to T on coding strand | TGG (Trp/W) |
| GGT (Gly/G) | 2nd base C to T on complementary | GAT (Asp/D) |
| | stand | |
| GGC (Gly/G) | 2nd base C to T on complementary | GAC (Asp/D) |
| | stand | |
| GGA (Gly/G) | 2nd base C to T on complementary | GAA (Glu/E) |
| | stand | |
| GGG (Gly/G) | 2nd base C to T on complementary | GAG (Glu/E) |
| | stand | |
| GGT (Gly/G) | 1st base C to T on complementary stand | AGT (Ser/S) |
| GGC (Gly/G) | 1st base C to T on complementary stand | AGC (Ser/S) |
| GGA (Gly/G) | 1st base C to T on complementary stand | AGA (Arg/R) |
| GGG (Gly/G) | 1st base C to T on complementary stand | AGG (Arg/R) |

In some embodiments, to bind to its target sequence and edit the desired base, the nucleobase editors depend on its guide nucleotide sequence (e.g., a guide RNA In some embodiments, the guide nucleotide sequence is a gRNA sequence. An gRNA typically comprises a tracrRNA framework allowing for Cas9 binding, and a guide sequence, which confers sequence specificity to fusion proteins disclosed herein. In some embodiments, the guide RNA comprises a structure 5′-[guide sequence]-guuuuagagcuagaaauagcaaguuaaaauaaaggcuaguccguuaucaacuugaaaaaguggcaccgagucggugcuuuuu-3′ (SEQ ID NO: 1997), wherein the guide sequence comprises a sequence that is complementary to the target sequence. The guide sequence is typically about 20 nucleotides long. For example, the guide sequence may be 15-25 nucleotides long. In some embodiments, the guide sequence is 15, 16, 17, 18, 19, 20, 21, 22, 23, 24, or 25 nucleotides long. Such suitable guide RNA sequences typically comprise guide sequences that are complementary to a nucleic sequence within 50 nucleotides upstream or downstream of the target nucleotide to be edited.

Guide sequences that may be used to target the nucleobase editor to its target sequence to induce specific mutations are provided in Table 3. It is to be understood that the mutations and guide sequences presented herein are for illustration purpose only and are not meant to be limiting.

TABLE 3
Exemplary PCSK9 Loss-of-Function Mutations via Codon Change

| | | Location | | | | | |
|---|---|---|---|---|---|---|---|
| Residue | Codon | of | | | gRNA size | | SEQ ID |
| Change | Change | mutation | guide sequence | (PAM) | (C edited) | BE type^{a} | NOs |
| R46C | CGT to | Pro- | GCCUUGCGUUCCGAGGAGGA | (CGG) | 20 (C7) | SpBE3 | 336-342 |
| | TGT | domain | GUGCUAGCCUUGCGUUCCGA | (GGAG) | 20 (C13) | EQR-SpBE3 | |
| | | | UGCUAGCCUUGCGUUCCGAG | (GAG) | 20 (C12) | SpBE3 | |
| | | | GCUAGCCUUGCGUUCCGAGG | (AGG) | 20 (C11) | SpBE3 | |
| | | | CUAGCCUUGCGUUCCGAGGA | (GGAC) | 20 (C10) | VQR-SpBE3 | |
| | | | GCCUUGCGUUCCGAGGAGGA | (CGG) | 20 (C7) | SpBE3 | |
| | | | GCGUUCCGAGGAGGACGGCC | (TGG) | 20 (C2) | SpBE3 | |
| G106R | GGA to | Pro- | GUAUCCCCGGCGGGCAGCCU | (GGG) | 20 (C6) | SpBE3 | 343, |
| | AGA | domain | GGUAUCCCCGGCGGGCAGCC | (TGG) | 20 (C7) | SpBE3 | 344 |
| | | loop, | | | | | |
| | | affects | | | | | |
| | | folding | | | | | |
| L253F | CTC to | Catalytic | CCUGCGCGUGCUCAACUGCC | (AAG) | 20 (C11) | SpBE3 | 345-352 |
| | TTC | domain, | CUGCGCGUGCUCAACUGCCA | (AGG) | 20 (C10) | SpBE3 | |
| | | affects | UGCGCGUGCUCAACUGCCAA | (GGG) | 20 (C9) | SpBE3 | |
| | | self- | GCGCGUGCUCAACUGCCAAG | (GGAA) | 20 (C8) | EQR-SpBE3 | |
| | | cleavage | GCGUGCUCAACUGCCAAGGG | (AAG) | 20 (C6) | SpBE3 | |
| | | | CGUGCUCAACUGCCAAGGGA | (AGG) | 20 (C5) | SpBE3 | |
| | | | GUGCUCAACUGCCAAGGGAA | (GGG) | 20 (C3) | SpBE3 | |
| | | | CUCAACUGCCAAGGGAAGGG | (CACGGT) | 20 (C1) | KKH-SaBE3 | |
| A443T | GCC to | Catalytic | GCGGCCACCAGGUUGGGGGU | (CAG) | 20 (C2) | SpBE3 | 353-363 |
| | ACC | domain, | CAGGGCGGCCACCAGGUUGG | (GGG) | 20 (C6) | SpBE3 | |
| | | enhanced | GCAGGGCGGCCACCAGGUUG | (GGG) | 20 (C7) | SpBE3 | |
| | | furin | GGCAGGGCGGCCACCAGGUU | (GGG) | 20 (C8) | SpBE3 | |
| | | cleavage | GGGCAGGGCGGCCACCAGGU | (TGG) | 20 (C9) | SpBE3 | |
| | | | UGGGGGGCAGGGCGGCCACC | (AGG) | 20 (C12) | SpBE3 | |
| | | | CUGGGGGGCAGGGCGGCCAC | (CAG) | 20 (C13) | SpBE3 | |
| | | | GGGCGGCCACCAGGUUGGGG | (GTCAGT) | 20 (C4) | KKH-SaBE3 | |
| | | | GGCAGGGCGGCCACCAGGUU | (GGGGGT) | 20 (C7) | SaBE3 | |
| | | | GGCAGGGCGGCCACCAGGUU | (GGGGG) | 20 (C8) | St3BE3 | |
| | | | GGGCAGGGCGGCCACCAGGU | (TGGGG) | 20 (C9) | St3BE3 | |
| R93C | CGC to | Pro- | AGCGCACUGCCCGCCGCCUG | (CAG) | 20 (C3) | SpBE3 | 364, |
| | TGC | domain | GCGCACUGCCCGCCGCCUGC | (AGG) | 20 (C2) | SpBE3 | 365 |
| A53V | GCC to | Pro- | GACGGCCUGGCCGAAGCACC | (CGAG) | 20 (C11) | EQR-SpBE3 | 366-369 |
| | GTC | domain | ACGGCCUGGCCGAAGCACCC | (GAG) | 20 (C10) | SpBE3 | |
| | | | CUGGCCGAAGCACCCGAGCA | (CGG) | 20 (C5) | SpBE3 | |
| | | | UGGCCGAAGCACCCGAGCAC | (GGAA) | 20 (C4) | EQR-SpBE3 | |
| A68T | GCC to | Pro- | GCGCAGCGGUGGAAGGUGGC | (TGTG) | 20 (C2) | VQR-SpBE3 | 370-379 |
| | ACC | domain | CUUGGCGCAGCGGUGGAAGG | (TGG) | 20 (C6) | SpBE3 | |
| | | | ACCUUGGCGCAGCGGUGGAA | (GGTG) | 20 (C8) | VQR-SpBE3 | |
| | | | CACCUUGGCGCAGCGGUGGA | (AGG) | 20 (C9) | SpBE3 | |
| | | | GCACCUUGGCGCAGCGGUGG | (AAG) | 20 (C10) | SpBE3 | |
| | | | CCGCACCUUGGCGCAGCGGU | (GGAA) | 20 (C12) | VQR-SpBE3 | |
| | | | CCCGCACCUUGGCGCAGCGG | (TGG) | 20 (C13) | SpBE3 | |
| | | | GCGCAGCGGUGGAAGGUGGC | (TGTGGT) | 20 (C2) | KKH-SaBE3 | |
| | | | CGCACCUUGGCGCAGCGGUG | (GAAGGT) | 20 (C11) | KKH-SaBE3 | |
| | | | CACCUUGGCGCAGCGGUGGA | (AGGTG) | 20 (C9) | St3BE3 | |
| E57K | GAG to | Pro- | CGUGCUCGGGUGCUUCGGCC | (AGG) | 20 (C7) | SpBE3 | 380-382 |
| | AAG | domain | CCGUGCUCGGGUGCUUCGGC | (CAG) | 20 (C8) | SpBE3 | |
| | | | GGUUCCGUGCUCGGGUGCUU | (CGG) | 20 (C12) | SpBE3 | |
| G263S | GGC to | Catalytic | CGCUAACCGUGCCCUUCCCU | (TGG) | 20 (C1) | SpBE3 | 383-385 |
| | AGC | domain | CCUAUGAGGGUGCCGCUAAC | (CGTG) | 20 (C14) | VQR-SpBE3 | |
| | | | CGCUAACCGUGCCCUUCCCUU | (GGCAGT) | 21 (C−1) | KKH-SaBE3 | |
| H391Y | CAC to | Catalytic | CUGCUGCCCACGUGGCUGGU | (AAG) | 20 (C9) | SpBE3 | 386, |
| | TAC | domain | GGCUGCUGCCCACGUGGCUG | (GTAAGT) | 20 (C11) | KKH-SaBE3 | 387 |
| G452D | GGT to | V-domain | CAACCUGCAAAAAGGGCCUG | (GGAT) | 20 (C4) | VQR-SpBE3 | 388-394 |
| | GAT | start | CCAACCUGCAAAAAGGGCCU | (GGG) | 20 (C5) | SpBE3 | |
| | | residue | GCCAACCUGCAAAAAGGGCC | (TGG) | 20 (C6) | SpBE3 | |
| | | | CAGCUGCCAACCUGCAAAAA | (GGG) | 20 (C11) | SpBE3 | |
| | | | ACAGCUGCCAACCUGCAAAA | (AGG) | 20 (C12) | SpBE3 | |
| | | | AACAGCUGCCAACCUGCAAA | (AAG) | 20 (C13) | SpBE3 | |
| | | | GCCAACCUGCAAAAAGGGCC | (TGGGAT) | 20 (C6) | SaBE3 | |
| A522T | GCT to | C- | CGUAGACACCCUCACCCCCAA | (AAG) | 21 (C−1) | SpBE3 | 395 |
| | ACT | terminal | | | | | |
| | | domain | | | | | |
| P616L | CCC to | C- | AGCAUGGAAUCCCGGCCCCU | (CAG) | 20 (C11/12) | SpBE3 | 396-406 |
| | CTC | terminal | GCAUGGAAUCCCGGCCCCUC | (AGG) | 20 (C10/11) | SpBE3 | |
| | | domain | CAUGGAAUCCCGGCCCCUCA | (GGAG) | 20 (C9/10) | EQR-SpBE3 | |
| | | | AUGGAAUCCCGGCCCCUCAG | (GAG) | 20 (C8/9) | SpBE3 | |
| | | | GAAUCCCGGCCCCUCAGGAG | (CAG) | 20 (C5/6) | SpBE3 | |
| | | | AAUCCCGGCCCCUCAGGAGC | (AGG) | 20 (C4/5) | SpBE3 | |
| | | | AUCCCGGCCCCUCAGGAGCA | (GGTG) | 20 (C3/4) | VQR-SpBE3 | |
| | | | CCCGGCCCCUCAGGAGCAGG | (TGAA) | 20 (C1/2) | EQR-SpBE3 | |
| | | | GGAAUCCCGGCCCCUCAGGA | (GCAGGT) | 20 (C6/7) | KKH-SaBE3 | |
| | | | GCAUGGAAUCCCGGCCCCUC | (AGGAG) | 20 (C11/12) | St3BE3 | |
| | | | AAUCCCGGCCCCUCAGGAGC | (AGGTG) | 20 (C4/5) | St3BE3 | |
| T771I | ACC to | Pro- | GCAGCACCUGCUUUGUGUCA | (CAG) | 20 (C7) | SpBE3 | 407-413 |
| | ATC | domain | CAGCACCUGCUUUGUGUCAC | (AGAG) | 20 (C6) | EQR-SpBE3 | |
| | | | AGCACCUGCUUUGUGUCACA | (GAG) | 20 (C5) | SpBE3 | |
| | | | GCACCUGCUUUGUGUCACAG | (AGTG) | 20 (C4) | VQR-SpBE3 | |
| | | | ACCUGCUUUGUGUCACAGAG | (TGG) | 20 (C2) | SpBE3 | |
| | | | CCUGCUUUGUGUCACAGAGU | (GGG) | 20 (C1) | SpBE3 | |
| | | | GCAGCACCUGCUUUGUGUCA | (CAGAGT) | 20 (C7) | SaBE3 | |
| M1I | ATG to | Translation | GCCCAUGAGGGCCAGGGGAG | (AGG) | 20 (C4) | SpBE3 | 414-426 |
| | ATA | start | UGCCCAUGAGGGCCAGGGGA | (GAG) | 20 (C5) | SpBE3 | |
| | | site, no | GUGCCCAUGAGGGCCAGGGG | (AGAG) | 20 (C6) | EQR-SpBE3 | |
| | | alternative | GGUGCCCAUGAGGGCCAGGG | (GAG) | 20 (C7) | SpBE3 | |
| | | nearby | CGGUGCCCAUGAGGGCCAGG | (GGAG) | 20 (C8) | EQR-SpBE3 | |
| | | | ACGGUGCCCAUGAGGGCCAG | (GGG) | 20 (C9) | SpBE3 | |
| | | | GACGGUGCCCAUGAGGGCCA | (GGG) | 20 (C10) | SpBE3 | |
| | | | UGACGGUGCCCAUGAGGGCC | (AGGG) | 20 (C11) | SpBE3 | |
| | | | UGACGGUGCCCAUGAGGGCC | (AGG) | 20 (C11) | SpBE3 | |
| | | | CUGACGGUGCCCAUGAGGGC | (CAG) | 20 (C12) | SpBE3 | |
| | | | GUGCCCAUGAGGGCCAGGGG | (AGAGGT) | 20 (C6) | KKH-SaBE3 | |
| | | | ACGGUGCCCAUGAGGGCCAG | (GGGAG) | 20 (C9) | St3BE3 | |
| | | | UGACGGUGCCCAUGAGGGCC | (AGGGG) | 20 (C10) | St3BE3 | |
| G24D | GGT to | Leader | CCCAGGAGCAGCAGCAGCAG | (CAG) | 20 (C1) | SpBE3 | 427-432 |
| | GAT | peptide | GGACCCAGGAGCAGCAGCAG | (CAG) | 20 (C4) | SpBE3 | |
| | | | GCGGGACCCAGGAGCAGCAG | (CAG) | 20 (C7) | SpBE3 | |
| | | | CCCGCGGGACCCAGGAGCAG | (CAG) | 20 (C1/10) | SpBE3 | |
| | | | GCGCCCGCGGGACCCAGGAG | (CAG) | 20 (C13) | SpBE3 | |
| | | | GGCGCAGGCCUCCCAGGAGC | (TCCAGT) | 20 (C12) | KKH-SaBE3 | |
| G27D | GGC to | Leader | GCGCCCGCGGGACCCAGGAG | (CAG) | 20 (C4) | SpBE3 | 433-438 |
| | GAC | peptide | CGGGCGCCCGCGGGACCCAG | (GAG) | 20 (C7) | SpBE3 | |
| | | | ACGGGCGCCCGCGGGACCCA | (GGAG) | 20 (C8) | EQR-SpBE3 | |
| | | | CACGGGCGCCCGCGGGACCC | (AGG) | 20 (C9) | SpBE3 | |
| | | | GCACGGGCGCCCGCGGGACC | (GAG) | 20 (C10) | SpBE3 | |
| | | | CACGGGCGCCCGCGGGACCC | (AGGAG) | 20 (C9) | St3BE3 | |
| R29C | CGT to | Leader | CCCGCGGGCGCCCGUGCGCA | (GGAG) | 20 (C13) | EQR-SpBE3 | 439-449 |
| | TGT | peptide | CCGCGGGCGCCCGUGCGCAG | (GAG) | 20 (C12) | SpBE3 | |
| | | | CGCGGGCGCCCGUGCGCAGG | (AGG) | 20 (C11) | SpBE3 | |
| | | | GCGGGCGCCCGUGCGCAGGA | (GGAC) | 20 (C10) | VQR-SpBE3 | |
| | | | GGCGCCCGUGCGCAGGAGGA | (CGAG) | 20 (C7) | EQR-SpBE3 | |
| | | | GCGCCCGUGCGCAGGAGGAC | (GAG) | 20 (C6) | SpBE3 | |
| | | | CGCCCGUGCGCAGGAGGACG | (AGG) | 20 (C5) | SpBE3 | |
| | | | GCCCGUGCGCAGGAGGACGA | (GGAC) | 20 (C4) | VQR-SpBE3 | |
| | | | CGUGCGCAGGAGGACGAGGA | (CGG) | 20 (C1) | SpBE3 | |
| | | | CGUGCGCAGGAGGACGAGGAC | (GGCG) | 21 (C−1) | VRER-SpBE3 | |
| | | | CGUGCGCAGGAGGACGAGGA | (CGGCG) | 20 (C1) | St3BE3 | |
| S47F | TCC to | Leader | GCCUUGCGUUCCGAGGAGGA | (CGG) | 20 (C6) | SpBE3 | 450-425 |
| | TTC | peptide | GCGUUCCGAGGAGGACGGCC | (TGG) | 20 (C5) | SpBE3 | |
| | | | UCCGAGGAGGACGGCCUGGC | (CGAA) | 20 (C2) | VQR-SpBE3 | |
| P12S | CCA to | Leader | CCACCAGGACCGCCUGGAGC | (TGAC) | 20 (C1) | VQR-SpBE3 | 453-458 |
| | UCA | peptide | GCGGCCACCAGGACCGCCUG | (GAG) | 20 (C5) | SpBE3 | |
| | | | AGCGGCCACCAGGACCGCCU | (GGAG) | 20 (C6) | EQR-SpBE3 | |
| | | | CAGCGGCCACCAGGACCGCC | (TGG) | 20 (C8) | SpBE3 | |
| | | | CACCAGGACCGCCUGGAGCU | (GACGGT) | 20 (C−1) | KKH-SaBE3 | |
| | | | CAGCGGCCACCAGGACCGCC | (TGGAG) | 20 (C8/1) | St3BE3 | |
| P14S | CCA to | Leader | CAGCGGCCACCAGGACCGCC | (TGG) | 20 (C1) | SpBE3 | 459-462 |
| | UCA | peptide | AGCAGUGGCAGCGGCCACCA | (GGAC) | 20 (C9) | VQR-SpBE3 | |
| | | | CAGCAGUGGCAGCGGCCACC | (AGG) | 20 (C10) | SpBE3 | |
| | | | GCAGCAGUGGCAGCGGCCAC | (GAG) | 20 (C11) | SpBE3 | |
| R46H | CGT to | similar to | UCGGAACGCAAGGCUAGCAC | (CAG) | 20 (C7) | SpBE3 | 463, |
| | CAT | R46L | GGCAAGGCUAGCACCAGCUCCU | (CGTAGT) | 22 (C−2) | KKH-SaBE3 | 464 |
| E49K | GAG to | Affects | UCCUCCUCGGAACGCAAGGC | (TAG) | 20 (C5) | SpBE3 | 465-467 |
| | AAG | leader | GCCGUCCUCCUCGGAACGCA | (AGG) | 20 (C9) | SpBE3 | |
| | | peptide | GGCCGUCCUCCUCGGAACGC | (AAG) | 20 (C10) | SpBE3 | |
| | | cleavage | | | | | |
| R237Q | CGG to | LDLR | GUGGUCAGCGGCCGGGAUGC | (CGG) | 20 (C13) | SpBE3 | 468-478 |
| | CAG | binding | UGGUCAGCGGCCGGGAUGCC | (GGCG) | 20 (C12) | VRER-SpBE3 | |
| | | | GUCAGCGGCCGGGAUGCCGG | (CGTG) | 20 (C10) | VQR-SpBE3 | |
| | | | CAGCGGCCGGGAUGCCGGCG | (TGG) | 20 (C8) | SpBE3 | |
| | | | GCCGGGAUGCCGGCGUGGCC | (AAG) | 20 (C3) | SpBE3 | |
| | | | CCGGGAUGCCGGCGUGGCCA | (AGG) | 20 (C2) | SpBE3 | |
| | | | CGGGAUGCCGGCGUGGCCAA | (GGG) | 20 (C1) | SpBE3 | |
| | | | CGGGAUGCCGGCGUGGCCAAG | (GGTG) | 21 (C−1) | VQR-SpBE3 | |
| | | | GCCGGGAUGCCGGCGUGGCC | (AAGGGT) | 20 (C3) | SaBE3 | |
| | | | GUGGUCAGCGGCCGGGAUGC | (CGGCG) | 20 (C13) | St3BE3 | |
| | | | CGGGAUGCCGGCGUGGCCAA | (GGGTG) | 20 (C1) | St3BE3 | |
| S153N | AGC to | LDLR | CUUUGCCCAGAGCAUCCCGU | (GGAA) | 20 (C13) | VQR-SpBE3 | 479-486 |
| | AAC | binding, | CCAGAGCAUCCCGUGGAACC | (TGG) | 20 (C7) | SpBE3 | |
| | | autocatalytic | CAGAGCAUCCCGUGGAACCU | (GGAG) | 20 (C6) | EQR-SpBE3 | |
| | | processing | AGAGCAUCCCGUGGAACCUG | (GAG) | 20 (C5) | SpBE3 | |
| | | | GAGCAUCCCGUGGAACCUGG | (AGCG) | 20 (C4) | VRER-SpBE3 | |
| | | | GCAUCCCGUGGAACCUGGAG | (CGG) | 20 (C2) | SpBE3 | |
| | | | AGCAUCCCGUGGAACCUGGA | (GCGGAT) | 20 (C3) | SaBE3 | |
| | | | CCAGAGCAUCCCGUGGAACC | (TGGAG) | 20 (C7) | St3BE3 | |
| R194Q | CGG to | LDLR | CGGUGGUCACUCUGUAUGCU | (GGTG) | 20 (C1) | VQR-SpBE3 | 487-490 |
| | CAG | binding | CCGGUGGUCACUCUGUAUGC | (TGG) | 20 (C2) | SpBE3 | |
| | | | UCCCGGUGGUCACUCUGUAU | (GCTGGT) | 20 (C4) | KKH-SaBE3 | |
| | | | CCGGUGGUCACUCUGUAUGC | (TGGTG) | 20 (C2) | St3BE3 | |
| R194W | CGG to | LDLR | CAGAGUGACCACCGGGAAAU | (CGAG) | 20 (C13) | EQR-SpBE3 | 491-499 |
| | TGG | binding | AGAGUGACCACCGGGAAAUC | (GAG) | 20 (C12) | SpBE3 | |
| | | | GAGUGACCACCGGGAAAUCG | (AGG) | 20 (C11) | SpBE3 | |
| | | | AGUGACCACCGGGAAAUCGA | (GGG) | 20 (C10) | SpBE3 | |
| | | | GACCACCGGGAAAUCGAGGG | (CAG) | 20 (C7) | SpBE3 | |
| | | | ACCACCGGGAAAUCGAGGGC | (AGG) | 20 (C6) | SpBE3 | |
| | | | CCACCGGGAAAUCGAGGGCA | (GGG) | 20 (C5) | SpBE3 | |
| | | | GACCACCGGGAAAUCGAGGG | (CAGGGT) | 20 (C7) | SaBE3 | |
| | | | CGGGAAAUCGAGGGCAGGGU | (CATGGT) | 20 (C1) | KKH-SaBE3 | |
| A220V | GCC to | Furing | UCGUCGAGCAGGCCAGCAAG | (TGTG) | 20 (C13) | VQR-SpBE3 | 500-504 |
| | GTC | cleavage | GUCGAGCAGGCCAGCAAGUG | (TGAC) | 20 (C11) | VQR-SpBE3 | |
| | | region | GAGCAGGCCAGCAAGUGUGA | (CAG) | 20 (C8) | SpBE3 | |
| | | | GCCAGCAAGUGUGACAGUCA | (TGG) | 20 (C2) | SpBE3 | |
| | | | UCGAGCAGGCCAGCAAGUGU | (GACAGT) | 20 (C10) | KKH-SaBE3 | |
| A220T | GCC to | Furing | GGCCUGCUCGACGAACACAA | (GGAC) | 20 (C3) | VQR-SpBE3 | 505-508 |
| | ACC | cleavage | UGGCCUGCUCGACGAACACA | (AGG) | 20 (C4) | SpBE3 | |
| | | region | CUGGCCUGCUCGACGAACAC | (AAG) | 20 (C5) | SpBE3 | |
| | | | ACACUUGCUGGCCUGCUCGA | (CGAA) | 20 (C12) | VQR-SpBE3 | |
| A290V | GCG to | S1 pocket | CUGCCCCUGGCGGGUGGGUA | (CAG) | 20 (C11) | SpBE3 | 509, |
| | GTG | | CCCUGGCGGGUGGGUACAGC | (CGCG) | 20 (C7) | VRER-SpBE3 | 510 |
| A290T | GCC to | S1 pocket | CCAGGGGCAGCAGCACCACC | (AGTG) | 20 (C1) | VQR-SpBE3 | 511-514 |
| | ACC | | GCCAGGGGCAGCAGCACCAC | (GAG) | 20 (C2) | SpBE3 | |
| | | | UACCCACCCGCCAGGGGCAG | (CAG) | 20 (C11) | SpBE3 | |
| | | | CCGCCAGGGGCAGCAGCACC | (ACCAGT) | 20 (C4) | KKH-SaBE3 | |
| D374N | GAC to | LDLR | GCAGUCGCUGGAGGCACCAA | (TGAT) | 20 (C6) | VQR-SpBE3 | 515-517 |
| | AAC | binding | CUGCAGUCGCUGGAGGCACC | (AATGAT) | 20 (C7) | KKH-SaBE3 | |
| | | | GUGCUGCAGUCGCUGGAGGC | (ACCAAT) | 20 (C10) | KKH-SaBE3 | |
| T377I | ACC to | LDLR | GCAGCACCUGCUUUGUGUCA | (CAG) | 20 (C7) | SpBE3 | 518-525 |
| | ATC | binding | CAGCACCUGCUUUGUGUCAC | (AGAG) | 20 (C6) | EQR-SpBE3 | |
| | | | AGCACCUGCUUUGUGUCACA | (GAG) | 20 (C5) | SpBE3 | |
| | | | GCACCUGCUUUGUGUCACAG | (AGTG) | 20 (C4) | VQR-SpBE3 | |
| | | | ACCUGCUUUGUGUCACAGAG | (TGG) | 20 (C2) | SpBE3 | |
| | | | CCUGCUUUGUGUCACAGAGU | (GGG) | 20 (C1) | SpBE3 | |
| | | | CCUGCUUUGUGUCACAGAGUG | (GGAC) | 21 (C−1) | VQR-SpBE3 | |
| | | | GCAGCACCUGCUUUGUGUCA | (CAGAGT) | 20 (C7) | SaBE3 | |
| C378Y | TGC to | LDLR | GCAGGUGCUGCAGUCGCUGG | (AGG) | 20 (C2) | SpBE3 | 526-531 |
| | TAC | binding | AGCAGGUGCUGCAGUCGCUG | (GAG) | 20 (C3) | SpBE3 | |
| | | | AAGCAGGUGCUGCAGUCGCU | (GGAG) | 20 (C4) | EQR-SpBE3 | |
| | | | AAAGCAGGUGCUGCAGUCGC | (TGG) | 20 (C5) | SpBE3 | |
| | | | GUGACACAAAGCAGGUGCUG | (CAG) | 20 (C12) | SpBE3 | |
| | | | AAAGCAGGUGCUGCAGUCGC | (TGGAG) | 20 (C5) | St3BE3 | |
| S386L | TCA to | Catalytic | ACAUCACAGGCUGCUGCCCA | (CGTG) | 20 (C5) | VQR-SpBE3 | 532-534 |
| | TTA | triad | AUCACAGGCUGCUGCCCACG | (TGG) | 20 (C3) | SpBE3 | |
| | | | CACAGGCUGCUGCCCACGUG | (GCTGGT) | 20 (C1) | KKH-SaBE3 | |
| S688F | TCC to | Phosphorylation | CGCAGGCCUCCCAGGAGCUC | (CAG) | 20 (C10) | SpBE3 | 535-539 |
| | TTC | site | GCAGGCCUCCCAGGAGCUCC | (AGTG) | 20 (C9) | VQR-SpBE3 | |
| | | | AGGCCUCCCAGGAGCUCCAG | (TGAC) | 20 (C7) | VQR-SpBE3 | |
| | | | CCUCCCAGGAGCUCCAGUGA | (CAG) | 20 (C4) | SpBE3 | |
| | | | GGCGCAGGCCUCCCAGGAGC | (TCCAGT) | 20 (C12) | KKH-SaBE3 | |
| D186N | GAC to | Catalytic | CUAGGAGAUACACCUCCACC | (AGG) | 20 (C1) | SpBE3 | 540, |
| | AAC | triad | UCUAGGAGAUACACCUCCAC | (CAG) | 20 (C2) | SpBE3 | 541 |
| H226Y | CAT to | Catalytic | UGACAGUCAUGGCACCCACC | (TGG) | 20 (C8) | SpBE3 | 542-551 |
| | TAT | triad | CAGUCAUGGCACCCACCUGG | (CAG) | 20 (C5) | SpBE3 | |
| | | | AGUCAUGGCACCCACCUGGC | (AGG) | 20 (C4) | SpBE3 | |
| | | | GUCAUGGCACCCACCUGGCA | (GGG) | 20 (C3) | SpBE3 | |
| | | | UCAUGGCACCCACCUGGCAG | (GGG) | 20 (C2) | SpBE3 | |
| | | | CAUGGCACCCACCUGGCAGG | (GGTG) | 20 (C1) | VQR-SpBE3 | |
| | | | AGUCAUGGCACCCACCUGGC | (AGGGGT) | 20 (C4) | SaBE3 | |
| | | | CAUGGCACCCACCUGGCAGG | (GGTGGT) | 20 (C1) | KKH-SaBE3 | |
| | | | AGUCAUGGCACCCACCUGGC | (AGGGG) | 20 (C4) | St3BE3 | |
| | | | UCAUGGCACCCACCUGGCAG | (GGGTG) | 20 (C2) | St3BE3 | |
| H193Y | CAC to | Folds | CAGAGUGACCACCGGGAAAU | (CGAG) | 20 (C10) | EQR-SpBE3 | 552-559 |
| | TAC | region | AGAGUGACCACCGGGAAAUC | (GAG) | 20 (C9) | SpBE3 | |
| | | that binds | GAGUGACCACCGGGAAAUCG | (AGG) | 20 (C8) | SpBE3 | |
| | | LDLR | AGUGACCACCGGGAAAUCGA | (GGG) | 20 (C7) | SpBE3 | |
| | | | GACCACCGGGAAAUCGAGGG | (CAG) | 20 (C4) | SpBE3 | |
| | | | ACCACCGGGAAAUCGAGGGC | (AGG) | 20 (C3) | SpBE3 | |
| | | | CCACCGGGAAAUCGAGGGCA | (GGG) | 20 (C2) | SpBE3 | |
| | | | GACCACCGGGAAAUCGAGGG | (CAGGGT) | 20 (C4) | SaBE3 | |
| S372F | TCC to | LDLR | AUUGGUGCCUCCAGCGACUG | (CAG) | 20 (C11) | SpBE3 | 560 |
| | TTC | binding | | | | | |
| S373N | AGC to | LDLR | GCAGUCGCUGGAGGCACCAA | (TGAT) | 20 (C6) | VQR-SpBE3 | 561-563 |
| | AAC | binding | CUGCAGUCGCUGGAGGCACC | (AATGAT) | 20 (C8/4) | KKH-SaBE3 | |
| | | | GUGCUGCAGUCGCUGGAGGC | (ACCAAT) | 20 (C11/7) | KKH-SaBE3 | |
| C375Y | TGC to | LDLR | GCAGUCGCUGGAGGCACCAA | (TGAT) | 20 (C2) | VQR-SpBE3 | 564-565 |
| | TAC | binding, | GCAGGUGCUGCAGUCGCUGG | (AGG) | 20 (C10) | SpBE3 | |
| | | disrupting | AGCAGGUGCUGCAGUCGCUG | (GAG) | 20 (C11) | SpBE3 | |
| | | formation | AAGCAGGUGCUGCAGUCGCU | (GGAG) | 20 (C12) | EQR-SpBE3 | |
| | | of key | CUGCAGUCGCUGGAGGCACC | (AATGAT) | 20 (C8,4,1) | KKH-SaBE3 | |
| | | disulfide | GUGCUGCAGUCGCUGGAGGC | (ACCAAT) | 20 | KKH-SaBE3 | |
| | | bond | | | (C11,7,4) | | |
| S376N | AGC to | LDLR | GCAGGUGCUGCAGUCGCUGG | (AGG) | 20 (C8) | SpBE3 | 570-576 |
| | AAC | binding | AGCAGGUGCUGCAGUCGCUG | (GAG) | 20 (C9) | SpBE3 | |
| | | | AAGCAGGUGCUGCAGUCGCU | (GGAG) | 20 (C10) | EQR-SpBE3 | |
| | | | AAAGCAGGUGCUGCAGUCGC | (TGG) | 20 (C11) | SpBE3 | |
| | | | CUGCAGUCGCUGGAGGCACC | (AATGAT) | 20 (C1) | KKH-SaBE3 | |
| | | | GUGCUGCAGUCGCUGGAGGC | (ACCAAT) | 20 (C4) | KKH-SaBE3 | |
| | | | AAAGCAGGUGCUGCAGUCGC | (TGGAG) | 20 (C13) | St3BE3 | |
| T384I | ACA to | Near | CAUCACAGGCUGCUGCCCACG | (TGG) | 21 (C−1) | SpBE3 | 577, |
| | ATA | oxyanion | ACAUCACAGGCUGCUGCCCA | (CGTG) | 20 (C2) | VQR-SpBE3 | 578 |
| | | hole | | | | | |

*Single underline indicate C to T change on the coding strand
Double underline indicate C to T change on the complementary strand
Guide sequences (the portion of the guide RNA that targets the nucleobase editor to the target sequence) are provided, which may be used with any tracrRNA framework sequences provided herein to generate the full guide RNA sequence
^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.

In some embodiments, the loss-of-function PCSK9 variant produced using the method described herein comprises a R46C mutation (CGT to TGT), mimicking the natural protective variant R46L. The PCSK9 R46L variant has been characterized to possess cholesterol-lowering effect and to reduce the risk of early-onset myocardial infraction. See, e.g., in Strom et al., Clinica Chimica Acta, Volume 411, Issues 3-4, 2, Pages 229-233, 2010; Saavedra et al., Arterioscler Thromb Vasc Biol., 34(12):2700-5, 2014; Cameron et al., Hum. Mol. Genet., 15 (9): 1551-1558, 2006; and Bonnefond et al., Diabetologia, Volume 58, Issue 9, pp 2051-2055, 2015, each of which is incorporated herein by reference.

In some embodiments, the loss-of-function PCSK9 variant produced using the method described herein comprises a L253F mutation (CTC to TTC). PCSK9 L253F variant has been shown to reduce plasma LDL-Cholesterol levels. See, e.g., in Kotowski et al., Am J Hum Genet., 78(3): 410-422, 2006; Zhao et al., Am J Hum Genet., 79(3): 514-523, 2006; Huang et al., Circ Cardiovasc Genet., 2(4): 354-361, 2009; and Hampton et al., PNAS, vol 104, No. 37, 14604-14609, 2007, each of which are incorporated herein by reference.

In some embodiments, the loss-of-function PCSK9 variant produced using the method described herein comprises a A443T mutation (GCC to ACC). PCSK9 A443T mutant has been shown to be associated with reduced plasma LCL-Chlesterol levels. See, e.g., in Mayne et al., Lipids in Health and Disease, 2013-12:70, 2013; Allard et al., Hum Mutat., 26(5):497, 2005; Huang et al., Circ Cardiovasc Genet., 2(4): 354-361, 2009; and Benjannet et al., Journal of Biological Chemistry, Vol. 281, No. 41, 2006, each of which are incorporated herein by reference.

In some embodiments, the loss-of-function PCSK9 variant produced using the method described herein comprises a R93C mutation (CGC to TGC). PCSK9 R93C variant has been shown to be associated with reduced plasma LCL-Chlesterol levels. See, e.g., in Mayne et al., Lipids in Health and Disease, 2013-12:70, 2013; Miyake et al., Atherosclerosis, 196(1):29-36, 2008; and Tang et al., Nature Communications, 6, Article number: 10206, 2015, each of which are incorporated herein by reference.

In some embodiments, cellular PCSK9 activity may be reduced by reducing the level of properly folded and active PCSK9 protein. Introducing destabilizing mutations into the wild type PCSK9 protein may cause misfolding or deactivation of the protein. A PCSK9 variant comprising one or more destabilizing mutations described herein may have reduced activity compared to the wild type PCSK9 protein. For example, the activity of a PCSK9 variant comprising one or more destabilizing mutations described herein may be reduced by at least about 20%, at least about 30%, at least about 40%, at least about 50%, at least about 60%, at least about 70%, at least about 80%, at least about 90%, at least about 95%, at least about 99%, or more.

Further, the present disclosure also contemplates the use of destabilizing mutations to counteract the effect of gain-of-function PCSK9 variant. Gain-of-function PCSK9 variants (e.g., the gain-of-function variants described in FIG. 1A have been described in the art and are found to be associated with hypercholesterolemia (e.g., in Peterson et al., J Lipid Res. 2008 June; 49(6): 1152-1156; Benjannet et al., J Biol Chem. 2012 Sep. 28; 287(40):33745-55; Abifadel et al., Atherosclerosis. 2012 August; 223(2):394-400; and Cameron et al., Hum. Mol. Genet. (1 May 2006) 15(9): 1551-1558, each of which is incorporated herein by reference). Introducing destabilizing mutations into these gain-of-function PCSK9 variants may cause misfolding and deactivation of these gain-of-function variants, thereby counteracting the hyper-activity caused by the gain-of-function mutation. Further, gain-of-function mutations in several other key factors in the LDL-R mediated cholesterol clearance pathway, e.g., LDL-R, APOB, or APOC, have also been described in the art. Thus, making destabilizing mutations in these factors to counteract the deleterious effect of the gain-of-function mutation using the compositions and methods described herein, is also within the scope of the present disclosure.

As such, the present disclosure further provides mutations that cause misfolding of PCSK9 protein or structurally destabilization of PCSK9 protein. Non-limiting, exemplary destabilizing PCSK9 mutations that may be made using the methods described herein are shown in Table 4.

TABLE 4
Exemplary PCSK9 Variants to Destabilize Protein Folding

| | | | | | | SEG |
|---|---|---|---|---|---|---|
| Residue | | | | gRNA size | | ID |
| change | Codon change | Guide sequence | (PAM) | (C edited) | BE type^{a} | NOs |
| P25S/L | CCC to CTC or | UCCUGGGUCCCGCGGGCGCC | (CGTG) | 20 (C9/10) | VQR-SpBE3 | 579-585 |
| | CCC to TCC | CUGGGUCCCGCGGGCGCCCG | (TGCG) | 20 (C7/8) | VRER-SpBE3 | |
| | | GUCCCGCGGGCGCCCGUGCG | (CAG) | 20 (C3/4) | SpBE3 | |
| | | UCCCGCGGGCGCCCGUGCGC | (AGG) | 20 (C2/3) | SpBE3 | |
| | | CCCGCGGGCGCCCGUGCGCA | (GGAG) | 20 (C1/2) | EQR-SpBE3 | |
| | | CCGCGGGCGCCCGUGCGCAG | (GAG) | 20 (C1/−1) | SpBE3 | |
| | | UCCCGCGGGCGCCCGUGCGC | (AGGAG) | 20 (C2) | St3BE3 | |
| P56S/L | CCC to CTC or | CUGGCCGAAGCACCCGAGCA | (CGG) | 20 (C13) | SpBE3 | 586-888 |
| | CCC to TCC | UGGCCGAAGCACCCGAGCAC | (GGAA) | 20 (C12/13) | VQR-SpBE3 | |
| | | AGCACCCGAGCACGGAACCA | (CAG) | 20 (C5/6) | SpBE3 | |
| C67Y | TGC to TAC | GCAGCGGUGGAAGGUGGCUG | (TGG) | 20 (C2) | SpBE3 | 589-595 |
| | | GCGCAGCGGUGGAAGGUGGC | (TGTG) | 20 (C4) | VQR-SpBE3 | |
| | | CUUGGCGCAGCGGUGGAAGG | (TGG) | 20 (C8) | SpBE3 | |
| | | ACCUUGGCGCAGCGGUGGAA | (GGTG) | 20 (C10) | VQR-SpBE3 | |
| | | CACCUUGGCGCAGCGGUGGA | (AGG) | 20 (C11) | SpBE3 | |
| | | GCGCAGCGGUGGAAGGUGGC | (TGTGGT) | 20 (C4) | KKH-SaBE3 | |
| | | CACCUUGGCGCAGCGGUGGA | (AGGTG) | 20 (C11) | St3BE3 | |
| P71S/L | CCG to TCG or | CAGGAUCCGUGGAGGUUGCC | (TGG) | 20 (C7/8) | SpBE3 | 596 |
| | CCG to CTG | | | | | |
| P75S/L | CCT to TCT | UGGAGGUUGCCUGGCACCUA | (CGTG) | 20 (C10/11) | VQR-SpBE3 | 597-605 |
| | or | GAGGUUGCCUGGCACCUACG | (TGG) | 20 (C8/9) | SpBE3 | |
| | CCT to CTT | AGGUUGCCUGGCACCUACGU | (GGTG) | 20 (C7/8) | VQR-SpBE3 | |
| | | GUUGCCUGGCACCUACGUGG | (TGG) | 20 (C5/6) | SpBE3 | |
| | | UUGCCUGGCACCUACGUGGU | (GGTG) | 20 (C4/5) | VQR-SpBE3 | |
| | | UGGAGGUUGCCUGGCACCUA | (CGTGGT) | 20 (C10/11) | KKH-SaBE3 | |
| | | AGGUUGCCUGGCACCUACGU | (GGTGGT) | 20 (C7/8) | KKH-SaBE3 | |
| | | GAGGUUGCCUGGCACCUACG | (TGGTG) | 20 (C8/9) | St3BE3 | |
| | | GUUGCCUGGCACCUACGUGG | (TGGTG) | 20 (C5/6) | St3BE3 | |
| P120S/L | CCT to TCT | GUCUUCCAUGGCCUUCUUCC | (TGG) | 20 (C12/13) | SpBE3 | 606-612 |
| | or | GGCCUUCUUCCUGGCUUCCU | (GGTG) | 20 (C3/4) | VQR-SpBE3 | |
| | CCT to CTT | UGGCCUUCUUCCUGGCUUCC | (TGG) | 20 (C4/5) | SpBE3 | |
| | | CCUUCUUCCUGGCUUCCUGG | (TGAA) | 20 (C1/2) | VQR-SpBE3 | |
| | | CAUGGCCUUCUUCCUGGCUU | (CCTGGT) | 20 (C7/8) | KKH-SaBE3 | |
| | | CUUCUUCCUGGCUUCCUGGU | (GAAGAT) | 20 (C1/2) | KKH-SaBE3 | |
| | | UGGCCUUCUUCCUGGCUUCC | (TGGTG) | 20 (C4/5) | St3BE3 | |
| P138S/L | CCC to CTC or | GCCUUGAAGUUGCCCCAUGU | (CGAC) | 20 (C13) | VQR-SpBE3 | 613-619 |
| | CCC to TCC | UUGCCCCAUGUCGACUACAU | (CGAG) | 20 (C4/5) | EQR-SpBE3 | |
| | | UGCCCCAUGUCGACUACAUC | (GAG) | 20 (C3/4) | SpBE3 | |
| | | GCCCCAUGUCGACUACAUCG | (AGG) | 20 (C2/3) | SpBE3 | |
| | | GCCCAUGUCGACUAGAUCGA | (GGAG) | 20 (C1/2) | EQR-SpBE3 | |
| | | CCCAUGUCGACUACAUCGAG | (GAG) | 20 (C1/−1) | SpBE3 | |
| | | GCCCCAUGUCGACUACAUCG | (AGGAG) | 20 (C2/3) | St3BE3 | |
| P155S/L | CCG to TCG or | CCAGAGCAUCCCGUGGAACC | (TGG) | 20 (C10/11) | SpBE3 | 620-627 |
| | CCG to CTG | CAGAGCAUCCCGUGGAACCU | (GGAG) | 20 (C9/10) | EQR-SpBE3 | |
| | | AGAGCAUCCCGUGGAACCUG | (GAG) | 20 (C8/9) | SpBE3 | |
| | | GAGCAUCCCGUGGAACCUGG | (AGCG) | 20 (C7/8) | VRER-SpBE3 | |
| | | GCAUCCCGUGGAACCUGGAG | (CGG) | 20 (C5/6) | SpBE3 | |
| | | CAUCCCGUGGAACCUGGAGC | (GGAT) | 20 (C4/5) | VQR-SpBE3 | |
| | | AGCAUCCCGUGGAACCUGGA | (GCGGAT) | 20 (C6/7) | SaBE3 | |
| | | CCAGAGCAUCCCGUGGAACC | (TGGAG) | 20 (C10) | St3BE3 | |
| P163S/L | CCT to TCT | GGAUUACCCCUCCACGGUAC | (CGG) | 20 (C9,10,12,13) | SpBE3 | 628-636 |
| and | or | GAUUACCCCUCCACGGUACC | (GGG) | 20 (C8,9,11,12) | SpBE3 | |
| P164S/L | CCT to CTT | AUUACCCCUCCACGGUACCG | (GGCG) | 20 (C7,8,10,11) | VRER-SpBE3 | |
| | and/or | UACCCCUCCACGGUACCGGG | (CGG) | 20 (C5,6,8,9) | SpBE3 | |
| | CCA to TCA or | ACCCCUCCACGGUACCGGGC | (GGAT) | 20 (C4,5,7,8) | VQR-SpBE3 | |
| | CCA to CTA | CCUCCACGGUACCGGGCGGA | (TGAA) | 20 (C1,2,4,5) | VQR-SpBE3 | |
| | | UUACCCCUCCACGGUACCGG | (GCGGAT) | 20 (C6,7,9,10) | SaBE3 | |
| | | CCCUCCACGGUACCGGGCGG | (ATGAAT) | 20 (C2,3,5,6) | SaBE3 | |
| | | GAUUACCCCUCCACGGUACC | (GGGCG) | 20 (C8,9,11,12) | St3BE3 | |
| P173S/L | | UGAAUACCAGCCGCCCGGUA | (AGAC) | 20 (C11/12) | VQR-SpBE3 | 637, 638 |
| and | | CCCCCCGGUAAGACCCCCAUC | (TGTG) | 21 (C1,−1,3,4) | VQR-SpBE3 | |
| P164S/L | | | | | | |
| G176R/E | GGA to AGA | CUGCCUCCGUCUUUCCAAGG | (CGAC) | 20 (C7/8) | VQR-SpBE3 | 639-642 |
| | or | GGCUGCCUCCGUCUUUCCAA | (GGCG) | 20 (C9/10) | VRER-SpBE3 | |
| | GGA to GAA | AGGCUGCCUCCGUCUUUCCA | (AGG) | 20 (C12/13) | SpBE3 | |
| | | AGGCUGCCUCCGUCUUUCCA | (AGGCG) | 20 (C9/10) | St3BE3 | |
| P209S/L | CCC to CTC or | UUCGAGAAUGUGCCCGAGGA | (GGAC) | 20 (C13/14) | VQR-SpBE3 | 643-646 |
| | CCC to TCC | GAGAAUGUGCCCGAGGAGGA | (CGG) | 20 (C10/11) | SpBE3 | |
| | | AGAAUGUGCCCGAGGAGGAC | (GGG) | 20 (C9/10) | SpBE3 | |
| | | GAAUGUGCCCGAGGAGGACG | (GGAC) | 20 (C8/9) | VQR-SpBE3 | |
| G213R/E | GGG to AGG or | GAAGCGGGUCCCGUCCUCCU | (CGGG) | 20 (C10/11) | VQR-SpBE3 | 647-649 |
| | GGG to GAG | AAGCGGGUCCCGUCCUCCUC | (GGG) | 20 (C9/10) | SpBE3 | |
| | | GAAGCGGGUCCCGUCCUCCU | (CGG) | 20 (C10/11) | SpBE3 | |
| C223Y | TGT to TAT | ACACUUGCUGGCCUGCUCGA | (CGAA) | 20 (C2) | VQR-SpBE3 | 650, 651 |
| | | GUCACACUUGCUGGCCUGCU | (CGAC) | 20 (C5) | VQR-SpBE3 | |
| G232R/E | GGG to AGG or | CCCCUGCCAGGUGGGUGCCA | (TGAC) | 20 (C2/3) | VQR-SpBE3 | 652-659 |
| | GGG to GAG | CUGACCACCCCUGCCAGGUG | (GGTG) | 20 (C8/9) | VQR-SpBE3 | |
| | | CGCUGACCACCCCUGCCAGG | (TGGG) | 20 (C10/11) | VQR-SpBE3 | |
| | | GCUGACCACCCCUGCCAGGU | (GGG) | 20 (C9/10) | VQR-SpBE3 | |
| | | CGCUGACCACCCCUGCCAGG | (TGG) | 20 (C10/11) | SpBE3 | |
| | | GCCGCUGACCACCCCUGCCA | (GGTG) | 20 (C12/13) | VQR-SpBE3 | |
| | | CCGCUGACCACCCCUGCCAG | (GTGGGT) | 20 (C11/12) | SaBE3 | |
| | | GCUGACCACCCCUGCCAGGU | (GGGTG) | 20 (C9/10) | St3BE3 | |
| C255Y | TGC to TAC | GCAGUUGAGCACGCGCAGGC | (TGCG) | 20 (C2) | VRER-SpBE3 | 660-663 |
| | | CUUGGCAGUUGAGCACGCGC | (AGG) | 20 (C6) | SpBE3 | |
| | | CCUUGGCAGUUGAGCACGCG | (CAG) | 20 (C7) | SpBE3 | |
| | | CUUCCCUUGGCAGUUGAGCA | (CGCG) | 20 (C11) | VRER-SpBE3 | |
| G257R | GGG to AGG | CCUUGGCAGUUGAGCACGCG | (GAG) | 20 (C1/2) | SpBE3 | 664-666 |
| | | CUUCCCUUGGCAGUUGAGCA | (CGCG) | 20 (C5/6) | VRER-SpBE3 | |
| | | GUGCCCUUCCCUUGGCAGUU | (GAG) | 20 (C10/11) | SpBE3 | |
| P279S/L | CCT to TCT | GGUCCAGCCUGUGGGGCCAC | (TGG) | 20 (C8/9) | SpBE3 | 667-674 |
| | or | GUCCAGCCUGUGGGGCCACU | (GGTG) | 20 (C7/8) | VQR-SpBE3 | |
| | CCT to CTT | CCAGCCUGUGGGGCCACUGG | (TGG) | 20 (C5/6) | SpBE3 | |
| | | CAGCCUGUGGGGCCACUGGU | (GGTG) | 20 (C4/5) | VQR-SpBE3 | |
| | | GUCCAGCCUGUGGGGCCACU | (GGTGGT) | 20 (C7/8) | KKH-SaBE3 | |
| | | CUGGUCCAGCCUGUGGGGCC | (ACTGGT) | 20 (C10/11) | KKH-SaBE3 | |
| | | GGUCCAGCCUGUGGGGCCAC | (TGGTG) | 20 (C8/9) | St3BE3 | |
| | | CCAGCCUGUGGGGCCACUGG | (TGGTG) | 20 (C5/6) | St3BE3 | |
| G281R | GGG to AGG | GCCCCACAGGCUGGACCAGC | (TGG) | 20 (C4/5) | SpBE3 | 675-677 |
| | | AGUGGCCCCACAGGCUGGAC | (CAG) | 20 (C8/9) | SpBE3 | |
| | | CACCAGUGGCCCCACAGGCU | (GGAC) | 20 (C12/13) | VQR-SpBE3 | |
| P282S/L | CCA to TCA or | CCACUGGUGGUGCUGCUGCCCC | (TGG) | 22 (C−1/−2) | SpBE3 | 678 |
| | CCA to CTA | | | | | |
| P288S/L | CCC to CTC or | UGGUGCUGCUGCCCCUGGCG | (GGTG) | 20 (C12/13) | VQR-SpBE3 | 679-685 |
| | CCC to TCC | GUGCUGCUGCCCCUGGCGGG | (TGG) | 20 (C10/11) | SpBE3 | |
| | | UGCUGCUGCCCCUGGCGGGU | (GGG) | 20 (C9/10) | SpBE3 | |
| | | CUGCCCCUGGCGGGUGGGUA | (CAG) | 20 (C4/5) | SpBE3 | |
| | | CCCCUGGCGGGUGGGUACAGC | (CGCG) | 21 (C1/−1) | VRER-SpBE3 | |
| | | GGUGCUGCUGCCCCUGGCGG | (GTGGGT) | 20 (C11/12) | SaBE3 | |
| | | GUGGUGCUGCUGCCCCUGGC | (GGGTG) | 20 (C13/14) | St3BE3 | |
| G292R/E | GGG to AGG | UACCCACCCGCCAGGGGCAG | (CAG) | 20 (C4/5) | SpBE3 | 686-693 |
| | or | CUGUACCCACCCGCCAGGGG | (CAG) | 20 (C7/8) | SpBE3 | |
| | GGG to GAG | GCGGCUGUACCCACCCGCCA | (GGGG) | 20 (C11/12) | VQR-SpBE3 | |
| | | CGGCUGUACCCACCCGCCAG | (GGG) | 20 (C10/11) | SpBE3 | |
| | | CGCGGCUGUACCCACCCGCC | (AGGG) | 20 (C12/13) | VQR-SpBE3 | |
| | | GCGGCUGUACCCACCCGCCA | (GGG) | 20 (C11/12) | SpBE3 | |
| | | CGCGGCUGUACCCACCCGCC | (AGG) | 20 (C12/13) | SpBE3 | |
| | | CGCGGCUGUACCCACCCGCC | (AGGGG) | 20 (C12/13) | St3BE3 | |
| C301Y | TGC to TAC | GGCGCUGGCAGGCGGCGUUG | (AGG) | 20 (C9) | SpBE3 | 694-699 |
| | | GGCAGGCGGCGUUGAGGACG | (CGG) | 20 (C3) | SpBE3 | |
| | | GUGGCAGGCGGCGUUGAGGA | (CGCG) | 20 (C5) | VRER-SpBE3 | |
| | | GCGCUGGCAGGCGGCGUUGA | (GGAC) | 20 (C8) | VQR-SpBE3 | |
| | | AGGCGCUGGCAGGCGGCGUU | (GAG) | 20 (C10) | SpBE3 | |
| | | CAGGCGCUGGCAGGCGGCGU | (TGAG) | 20 (C11) | EQR-SpBE3 | |
| C323Y | TGC to TAC | GGCAUCGUCCCGGAAGUUGC | (CGG) | 20 (C3) | SpBE3 | 700-704 |
| | | AGAGGCAGGCAUCGUCCCGG | (AAG) | 20 (C10) | SpBE3 | |
| | | GUAGAGGCAGGCAUCGUCCC | (GGAA) | 20 (C12) | VQR-SpBE3 | |
| | | AGUAGAGGCAGGCAUCGUCC | (CGG) | 20 (C13) | SpBE3 | |
| | | GUAGAGGCAGGCAUCGUCCC | (GGAAGT) | 20 (C12) | KKH-SaBE3 | |
| P327S/L | CCA to TCA or | UAGUCCCCAGCCUGAGCUCC | (CGAG) | 20 (C7/8) | EQR-SpBE3 | 705-713 |
| | CCA to CTA | ACUCCCCAGCCUCAGCUCCC | (GAG) | 20 (C6/7) | SpBE3 | |
| | | CUCCCCAGCCUCAGCUCCCG | (AGG) | 20 (C5/6) | SpBE3 | |
| | | CCCAGCCUCAGCUCCCGAGG | (TAG) | 20 (C3/4) | SpBE3 | |
| | | CCAGCCUCAGCUCCCGAGGU | (AGG) | 20 (C2/3) | SpBE3 | |
| | | CCAGCCUCAGCUCCCGAGGUA | (GGTG) | 21 (C1/−1) | VQR-SpBE3 | |
| | | UACUCCCCAGCCUCAGCUCC | (CGAGGT) | 20 (C7/8) | KKH-SaBE3 | |
| | | CCCCAGCCUCAGCUCCCGAG | (GTAGGT) | 20 (C3/4) | KKH-SaBE3 | |
| | | CCAGCCUCAGCUCCCGAGGU | (AGGTG) | 20 (C1/2) | St3BE3 | |
| P331S/L | CCC to CTC or | CAGCCUCAGCUCCCGAGGUA | (GGTG) | 20 (C12/13) | VQR-SpBE3 | 714-718 |
| | CCC to TCC | UCAGCUCCCGAGGUAGGUGC | (TGG) | 20 (C7/8) | SpBE3 | |
| | | CAGCUCCCGAGGUAGGUGCU | (GGG) | 20 (C6/7) | SpBE3 | |
| | | AGCUCCCGAGGUAGGUGCUG | (GGG) | 20 (C5/6) | SpBE3 | |
| | | UCAGCUCCCGAGGUAGGUGC | (TGGGG) | 20 (C7/8) | St3BE3 | |
| G337R | GGG to AGG | CCAACUGUGAUGACCUGGAA | (AGG) | 20 (C1/2) | SpBE3 | 719-726 |
| | | CCAACUGUGAUGACCUGGAAA | (GGTG) | 21 (C1/−1) | VQR-SpBE3 | |
| | | CCCAACUGUGAUGACCUGGA | (AAG) | 20 (C2/3) | SpBE3 | |
| | | GGCCCCAACUGUGAUGACCU | (GGAA) | 20 (C5/6) | VQR-SpBE3 | |
| | | UGGCCCCAACUGUGAUGACC | (TGG) | 20 (C6/7) | SpBE3 | |
| | | AUUGGUGGCCCCAACUGUGA | (TGAC) | 20 (C11/12) | VQR-SpBE3 | |
| | | CCCCAACUGUGAUGACCUGG | (AAAGGT) | 20 (C3/4) | KKH-SaBE3 | |
| | | CCAACUGUGAUGACCUGGAA | (AGGTG) | 20 (C1/2) | St3BE3 | |
| P345S/L | CCG to TCG or | CCAAGACCAGCCGGUGACCC | (TGG) | 20 (C11/12) | SpBE3 | 727-734 |
| | CCG to CTG | CAAGACCAGCCGGUGACCCU | (GGG) | 20 (C10/11) | SpBE3 | |
| | | AAGACCAGCCGGUGACCCUG | (GGG) | 20 (C9/10) | SpBE3 | |
| | | AGACCAGCCGGUGACCCUGG | (GGAC) | 20 (C8/9) | VQR-SpBE3 | |
| | | GCCGGUGACCCUGGGGACUU | (TGG) | 20 (C2/3) | SpBE3 | |
| | | CCGGUGACCCUGGGGACUUU | (GGG) | 20 (C1/2) | SpBE3 | |
| | | CGGUGACCCUGGGGACUUUG | (GGG) | 20 (C1/−1) | SpBE3 | |
| | | CCAAGACCAGCCGGUGACCC | (TGGGG) | 20 (C11/12) | St3BE3 | |
| | | GCCGGUGACCCUGGGGACUU | (TGGGG) | 20 (C2/3) | St3BE3 | |
| C358Y | TGT to TAT | GUCCACACAGCGGCCAAAGU | (TGG) | 20 (C8) | SpBE3 | 735-738 |
| | | AGAGGUCCACACAGCGGCCA | (AAG) | 20 (C12) | SpBE3 | |
| | | CAGCGGCCAAAGUUGGUCCC | (CAAAGT) | 20 (C1) | KKH-SaBE3 | |
| | | AGGUCCACACAGCGGCCAAA | (GTTGGT) | 20 (C10) | KKH-SaBE3 | |
| P364S/L | CCA to TCA or | GACCUCUUUGCCCCAGGGGA | (GGAC) | 20 (C13/14) | VQR-SpBE3 | 739-743 |
| | CCA to CTA | GCCCCAGGGGAGGACAUCAU | (TGG) | 20 (C4/5) | SpBE3 | |
| | | CCCCAGGGGAGGACAUCAUU | (GGTG) | 20 (C3/4) | VQR-SpBE3 | |
| | | UUGCCCCAGGGGAGGACAUC | (ATTGGT) | 20 (C6/7) | KKH-SaBE3 | |
| | | GCCCCAGGGGAGGACAUCAU | (TGGTG) | 20 (C4/5) | St3BE3 | |
| G365R/E | GGG to AGG | CCUGGGGCAAAGAGGUCCAC | (ACAG) | 20 (C1/−1) | VQR-SpBE3 | 744-748 |
| | or | UGUCCUCCCCUGGGGCAAAG | (AGG) | 20 (C9/10) | SpBE3 | |
| | GGG to GAG | AUGUCCUCCCCUGGGGCAAA | (GAG) | 20 (C10/11) | SpBE3 | |
| | | GAUGUCCUCCCCUGGGGCAA | (AGAG) | 20 (C11/12) | EQR-SpBE3 | |
| | | GAUGUCCUCCCCUGGGGCAA | (AGAGGT) | 20 (C11/12) | KKH-SaBE3 | |
| G384R/E | GGG to AGG | CCACUCUGUGACACAAAGCA | (GGTG) | 20 (C1/2) | VQR-SpBE3 | 749-754 |
| | or | CCCACUCUGUGACACAAAGC | (AGG) | 20 (C2/3) | SpBE3 | |
| | GGG to GAG | UCCCACUCUGUGACACAAAG | (CAG) | 20 (C3/4) | SpBE3 | |
| | | AUGUCCCACUCUGUGACACA | (AAG) | 20 (C6/7) | SpBE3 | |
| | | GCCUGUGAUGUCCCACUCUG | (TGAC) | 20 (C13/14) | VQR-SpBE3 | |
| | | CCCACUCUGUGACACAAAGC | (AGGTG) | 20 (C2/3) | St3BE3 | |
| P404S/L | CCG to TCG or | UGCCGAGCCGGAGCUCACCC | (TGG) | 20 (C8/9) | SpBE3 | 755-758 |
| | CCG to CTG | GAGCCGGAGCUCACCCUGGC | (CGAG) | 20 (C4/5) | EQR-SpBE3 | |
| | | AGCCGGAGCUCACCCUGGCC | (GAG) | 20 (C3/4) | SpBE3 | |
| | | CGAGCCGGAGCUCACCCUGG | (CCGAGT) | 20 (C5/6) | SaBE3 | |
| P430S/L | CCT to TCT | AGGCCUGGUUCCCUGAGGAC | (CAG) | 20 (C12/13) | SpBE3 | 759-764 |
| | or | GGCCUGGUUCCCUGAGGACC | (AGCG) | 20 (C11/12) | VRER-SpBE3 | |
| | CCT to CTT | CCUGGUUCCCUGAGGACCAG | (CGG) | 20 (C9/10) | SpBE3 | |
| | | CUGGUUCCCUGAGGACCAGC | (GGG) | 20 (C8/9) | SpBE3 | |
| | | CCCUGAGGACCAGCGGGUAC | (TGAC) | 20 (C2/3) | VQR-SpBE3 | |
| | | GCCUGGUUCCCUGAGGACCA | (GCGGGT) | 20 (C10/11) | SaBE3 | |
| P438S/L | CCC to CTC | CCUGCCCCCCAGCACCCAUG | (GGG) | 20 (C10/11) | SpBE3 | 765-768 |
| | | CCCUGCCCCCCAGCACCCAU | (GGG) | 20 (C11/12) | SpBE3 | |
| | | GCGGGUACUGACCCCCAACC | (TGG) | 20 (C12/13) | SpBE3 | |
| | | CGGGUACUGACCCCCAACCU | (GGTG) | 20 (C13/14) | VQR-SpBE3 | |
| P445S/L | CCC to CTC or | CCUGCCCCCCAGCACCCAUG | (GGG) | 20 (C5,6,8,9) | SpBE3 | 769-775 |
| and | CCC to TCC | CCCUGCCCCCCAGCACCCAU | (GGG) | 20 (C6,7,9,10) | SpBE3 | |
| P446S/L | | GCCCUGCCCCCCAGCACCCA | (TGG) | 20 (C7,8,10,11) | SpBE3 | |
| | | GCCCCCCAGCACCCAUGGGG | (CAG) | 20 (C2,3,5,6) | SpBE3 | |
| | | CCCCCCAGCACCCAUGGGGC | (AGG) | 20 (C1,2,4,5,) | SpBE3 | |
| | | UGCCCCCCAGCACCCAUGGG | (GCAGGT) | 20 (C3,4,6,7) | KKH-SaBE3 | |
| | | GCCCUGCCCCCCAGCACCCA | (TGGGG) | 20 (C7,8,10,11) | St3BE3 | |
| P446S/L | CCC to CTC or | CCCAGCACCCAUGGGGCAGGU | (AAG) | 21 (C1/−1) | SpBE3 | 776 |
| | CCC to TCC | | | | | |
| G450R/E | GGG to AGG | CCAUGGGUGCUGGGGGGCAG | (GGCG) | 20 (C1/2) | VRER-SpBE3 | 777-794 |
| | or | CCCCAUGGGUGCUGGGGGGC | (AGGG) | 20 (C3/4) | VQR-SpBE3 | |
| | GGG to GAG | CCCAUGGGUGCUGGGGGGCA | (GGG) | 20 (C2/3) | SpBE3 | |
| | | CCCCAUGGGUGCUGGGGGGC | (AGG) | 20 (C3/4) | SpBE3 | |
| | | GCCCCAUGGGUGCUGGGGGG | (CAG) | 20 (C4/5) | SpBE3 | |
| | | ACCUGCCCCAUGGGUGCUGG | (GGGG) | 20 (C8/9) | VQR-SpBE3 | |
| | | CCUGCCCCAUGGGUGCUGGG | (GGG) | 20 (C7/8) | SpBE3 | |
| | | UACCUGCCCCAUGGGUGCUG | (GGGG) | 20 (C9/10) | VQR-SpBE3 | |
| | | ACCUGCCCCAUGGGUGCUGG | (GGG) | 20 (C8/9) | SpBE3 | |
| | | UUACCUGCCCCAUGGGUGCU | (GGGG) | 20 (C10/11) | VQR-SpBE3 | |
| | | UACCUGCCCCAUGGGUGCUG | (GGG) | 20 (C9/10) | SpBE3 | |
| | | UUACCUGCCCCAUGGGUGCU | (GGG) | 20 (C10/11) | SpBE3 | |
| | | CUUACCUGCCCCAUGGGUGC | (TGGG) | 20 (C11/12) | SpBE3 | |
| | | CUUACCUGCCCCAUGGGUGC | (TGG) | 20 (C11/12) | SpBE3 | |
| | | CCCAUGGGUGCUGGGGGGCA | (GGGCG) | 20 (C2/3) | St3BE3 | |
| | | UACCUGCCCCAUGGGUGCUG | (GGGGG) | 20 (C9/10) | St3BE3 | |
| | | UUACCUGCCCCAUGGGUGCU | (GGGGG) | 20 (C10/11) | St3BE3 | |
| | | CUUACCUGCCCCAUGGGUGC | (TGGGG) | 20 (C11/12) | St3BE3 | |
| C457Y | | CAAAACAGCUGCCAACCUGCAAA | (AAG) | 23 (C−3) | SpBE3 | 795 |
| P467S/L | CCT to TCT or | GGGGCCUACACGGAUGGCCA | (CAG) | 20 (C5/6) | SpBE3 | 796-797 |
| | CCT to CTT | ACACUCGGGGCCUACACGGA | (TGG) | 20 (C11/12) | SpBE3 | |
| C477Y | TGC to TAC | GGCGCAGCGGGCGACGGCUG | (TGG) | 20 (C5) | SpBE3 | 798-800 |
| | | GGGGCGCAGCGGGCGACGGC | (TGTG) | 20 (C7) | VQR-SpBE3 | |
| | | AUCUGGGGCGCAGCGGGCGA | (CGG) | 20 (C11) | SpBE3 | |
| P478S/L | CCA to TCA or | GCCCCAGAUGAGGAGCUGCU | (GAG) | 20 (C4/5) | SpBE3 | 801-804 |
| | CCA to CTA | GCCCGCUGCGCCCCAGAUGA | (GGAG) | 20 (C13) | EQR-SpBE3 | |
| | | CCCGCUGCGCCCCAGAUGAG | (GAG) | 20 (C12/13) | SpBE3 | |
| | | CGCCCCAGAUGAGGAGCUGC | (TGAG) | 20 (C5/6) | EQR-SpBE3 | |
| C486Y | TGC to TAC | CAGCUCAGCAGCUCCUCAUC | (TGG) | 20 (C1) | SpBE3 | 805-809 |
| | | CAGCUCAGCAGCUCCUCAUC | (TGGG) | 20 (C1) | VQR-SpBE3 | |
| | | CAGCUCAGCAGCUCCUCAUCU | (GGG) | 21 (C−1) | SpBE3 | |
| | | GAGAAACUGGAGCAGCUCAG | (CAG) | 20 (C13) | SpBE3 | |
| | | CAGCUCAGCAGCUCCUCAUC | (TGGGG) | 20 (C1) | St3BE3 | |
| G493R/E | GGG to AGG | CUUCCCACUCCUGGAGAAAC | (TGG) | 20 (C5/6) | SpBE3 | 810-816 |
| | or | UCCCACUCCUGGAGAAACUG | (GAG) | 20 (C3/4) | SpBE3 | |
| | GGG to GAG | UUCCCACUCCUGGAGAAACU | (GGAG) | 20 (C4/5) | EQR-SpBE3 | |
| | | CCGCCGCUUCCCACUCCUGG | (AGAA) | 20 (C11/12) | SpBE3 | |
| | | CCCGCCGCUUCCCACUCCUG | (GAG) | 20 (C12/13) | SpBE3 | |
| | | CUUCCCACUCCUGGAGAAAC | (TGGAG) | 20 (C5/6) | St3BE3 | |
| | | CCCCGCCGCUUCCCACUCCU | (GGAGAAA) | 20 (C13/14) | St1BE3 | |
| G504R/E | GGG to AGG | CCCUUGGGCCUUAGAGUCAA | (AGAC) | 20 (C2/3) | VQR-SpBE3 | 817-822 |
| | or | CCCCUUGGGCCUUAGAGUCA | (AAG) | 20 (C3/4) | SpBE3 | |
| | GGG to GAG | GCUUGCCCCCUUGGGCCUUA | (GAG) | 20 (C9/10) | SpBE3 | |
| | | AGCUUGCCCCCUUGGGCCUU | (AGAG) | 20 (C10/11) | EQR-SpBE3 | |
| | | CAGCUUGCCCCCUUGGGCCU | (TAG) | 20 (C12/13) | SpBE3 | |
| | | CAGCUUGCCCCCUUGGGCCU | (TAGAGT) | 20 (C11/12) | SaBE3 | |
| C509Y | TGC to TAC | GGCAGACCAGCUUGCCCCCU | (TGG) | 20 (C3) | SpBE3 | 823-825 |
| | | GGCAGACCAGCUUGCCCCCU | (TGGG) | 20 (C3) | VQR-SpBE3 | |
| | | GCAGACCAGCUUGCCCCCUU | (GGG) | 20 (C2) | SpBE3 | |
| G516R/E | GGG to AGG | CCCCAAAAGCGUUGUGGGCC | (CGG) | 20 (C3/4) | SpBE3 | 826-830 |
| | or | CUCACCCCCAAAAGCGUUGU | (GGG) | 20 (C8/9) | SpBE3 | |
| | GGG to GAG | CCUCACCCCCAAAAGCGUUG | (TGGG) | 20 (C9/10) | VQR-SpBE3 | |
| | | CCUCACCCCCAAAAGCGUUG | (TGG) | 20 (C9/10) | SpBE3 | |
| | | ACCCUCACCCCCAAAAGCGU | (TGTG) | 20 (C10/11) | VQR-SpBE3 | |
| C526Y | TGC to TAC | GGCAGCACCUGGCAAUGGCG | (TAG) | 20 (C6/3) | SpBE3 | 831-836 |
| and | | GCAGCACCUGGCAAUGGCGU | (AGAC) | 20 (C5/2) | VQR-SpBE3 | |
| C527Y | | AGCAGGCAGCACCUGGCAAU | (GGCG) | 20 (C10/7) | VRER-SpBE3 | |
| | | UAGCAGGCAGCACCUGGCAA | (TGG) | 20 (C11/8) | SpBE3 | |
| | | CAUGGCACCCACCUGGCAGG | (GGTGGT) | 20 (C12/9) | KKH-SaBE3 | |
| | | UAGCAGGCAGCACCUGGCAA | (TGGCG) | 20 (C8/5) | St3BE3 | |
| P530S/L | CCC to CTC or | CUGCUACCCCAGGCCAACUG | (CAG) | 20 (C7/8) | SpBE3 | 837, 838 |
| | CCC to TCC | UGCUACCCCAGGCCAACUGC | (AGCG) | 20 (C6/7) | VRER-SpBE3 | |
| C534Y | TGC to TAC | ACGCUGCAGUUGGCCUGGGG | (TAG) | 20 (C7) | SpBE3 | 839-848 |
| | | UGCAGUUGGCCUGGGGUAGC | (AGG) | 20 (C3) | SpBE3 | |
| | | CUGCAGUUGGCCUGGGGUAG | (GAG) | 20 (C4) | SpBE3 | |
| | | GUGGACGCUGCAGUUGGCCU | (GGGG) | 20 (C11) | VQR-SpBE3 | |
| | | UGGACGCUGCAGUUGGCCUG | (GGG) | 20 (C10) | VQR-SpBE3 | |
| | | UGUGGACGCUGCAGUUGGCC | (TGGG) | 20 (C12) | VQR-SpBE3 | |
| | | GUGGACGCUGCAGUUGGCCU | (GGG) | 20 (C11) | VQR-SpBE3 | |
| | | UGUGGACGCUGCAGUUGGCC | (TGG) | 20 (C12) | SpBE3 | |
| | | UGUGGACGCUGCAGUUGGCC | (TGGGGT) | 20 (C12) | SaBE3 | |
| | | UGUGGACGCUGCAGUUGGCC | (TGGGG) | 20 (C12) | St3BE3 | |
| P540S/L | CCA to TCA or | GUCCACACAGCUCCACCAGC | (TGAG) | 20 (C13) | EQR-SpBE3 | 849-856 |
| and | CCA to CTA | UCCACACAGCUCCACCAGCU | (GAG) | 20 (C12/13) | SpBE3 | |
| P541S/L | | CCACACAGCUCCACCAGCUG | (AGG) | 20 (C11/12) | SpBE3 | |
| | | ACAGCUCCACCAGCUGAGGC | (CAG) | 20 (C7,8,10,11) | SpBE3 | |
| | | UCCACCAGCUGAGGCCAGCA | (TGG) | 20 (C2,3,5,6) | SpBE3 | |
| | | CCACCAGCUGAGGCCAGCAU | (GGG) | 20 (C1,2,4,5) | SpBE3 | |
| | | CCACCAGCUGAGGCCAGCAUG | (GGG) | 21 (C1,−1,3,4) | SpBE3 | |
| | | UCCACCAGCUGAGGCCAGCA | (TGGGG) | 20 (C2,3,5,6) | St3BE3 | |
| P541S/L | CCA to TCA or | ACCAGCUGAGGCCAGCAUGG | (GGAC) | 20 (C2/3) | VQR-SpBE3 | 857 |
| | CCA to CTA | | | | | |
| C552Y | TGC to TAC | CUGUUGGUGGCAGUGGACAC | (GGG) | 20 (C11) | SpBE3 | 858-860 |
| | | CCUGUUGGUGGCAGUGGACA | (CGGG) | 20 (C12) | VQR-SpBE3 | |
| | | CCUGUUGGUGGCAGUGGACA | (CGG) | 20 (C12) | VQR-SpBE3 | |
| P576S/L | CCG to TCG or | GCCGCCUGUGCUGAGGCCAC | (GAG) | 20 (C2,3,5,6) | SpBE3 | 861-867 |
| and/or | CCG to CTG | CCCACAAGCCGCCUGUGCUG | (AGG) | 20 (C9,10,12,13) | SpBE3 | |
| P557S/L | and/or | CCGCCUGUGCUGAGGCCACG | (AGG) | 20 (C1,2,4,5) | SpBE3 | |
| | CCT to TCT | AGCCGCCUGUGCUGAGGCCA | (CGAG) | 20 (C3,4,6,7) | EQR-SpBE3 | |
| | or | ACCCACAAGCCGCCUGUGCU | (GAG) | 20 (C10/11) | SpBE3 | |
| | CCT to CTT | CACCCACAAGCCGCCUGUGC | (TGAG) | 20 (C11/12) | EQR-SpBE3 | |
| | | AGCCGCCUGUGCUGAGGCCA | (CGAGGT) | 20 (C4,5,6,7) | KKH-SaBE3 | |
| P577S/L | CCT to TCT | CCUGUGCUGAGGCCACGAGGU | (CAG) | 21 (C1/−1) | SpBE3 | 868 |
| | or | | | | | |
| | CCT to CTT | | | | | |
| P581S/L | CCA to TCA or | GGCCACGAGGUCAGCCCAAC | (CAG) | 20 (C3/4) | SpBE3 | 869-872 |
| | CCA to CTA | GCCACGAGGUCAGCCCAACC | (AGTG) | 20 (C2/3) | VQR-SpBE3 | |
| | | CCACGAGGUCAGCCCAACCAG | (TGCG) | 21 (C1/−1) | VRER-SpBE3 | |
| | | GAGGCCACGAGGUCAGCCCA | (ACCAGT) | 20 (C5/6) | KKH-SaBE3 | |
| P585S/L | CCC to CTC or | CACGAGGUCAGCCCAACCAG | (TGCG) | 20 (C12/13) | VRER-SpBE3 | 873-877 |
| | CCC to TCC | CGAGGUCAGCCCAACCAGUG | (CGTG) | 20 (C10/11) | VQR-SpBE3 | |
| | | GGUCAGCCCAACCAGUGCGU | (GGG) | 20 (C4,7,8) | SpBE3 | |
| | | AGGUCAGCCCAACCAGUGCG | (TGG) | 20 (C5,8,9) | SpBE3 | |
| | | CCCAACCAGUGCGUGGGCCA | (CAG) | 20 (C1/2) | SpBE3 | |
| C588Y | TGC to TAC | CACUGGUUGGGCUGACCUCG | (TGG) | 20 (C1) | SpBE3 | 878-880 |
| | | CGCACUGGUUGGGCUGACCU | (CGTG) | 20 (C3) | VQR-SpBE3 | |
| | | GGCCCACGCACUGGUUGGGC | (TGAC) | 20 (C9) | VQR-SpBE3 | |
| C600Y | TGC to TAC | GCAGCAGGAAGCGUGGAUGC | (TGG) | 20 (C5/2) | SpBE3 | 881-883 |
| and | | GGCAUGGCAGCAGGAAGCGU | (GGAT) | 20 (C11/8) | VQR-SpBE3 | |
| C601Y | | GGGGCAUGGCAGCAGGAAGC | (GTGGAT) | 20 (C13/10) | VRER-SpBE3 | |
| C601Y | TGC to TAC | GGGCAUGGCAGCAGGAAGCG | (TGG) | 20 (C9) | SpBE3 | 884-886 |
| | | UGGGGCAUGGCAGCAGGAAG | (CGTG) | 20 (C10) | VQR-SpBE3 | |
| | | CCUGGGGCAUGGCAGCAGGA | (AGCG) | 20 (C12) | VRER-SpBE3 | |
| P604S/L | CCA to TCA or | UGCCCCAGGUCUGGAAUGCA | (AAG) | 20 (C5/6) | SpBE3 | 887-889 |
| | CCA to CTA | UGCUGCCAUGCCCCAGGUCU | (GGAA) | 20 (C13) | VQR-SpBE3 | |
| | | CAUGCCCCAGGUCUGGAAUG | (CAAAGT) | 20 (C7/8) | KKH-SaBE3 | |
| C608Y | TGC to TAC | GACUUUGCAUUCCAGACCUG | (GGG) | 20 (C8) | SpBE3 | 890-896 |
| | | UGCAUUCCAGACCUGGGGCA | (TGG) | 20 (C3) | SpBE3 | |
| | | UGACUUUGCAUUCCAGACCU | (GGGG) | 20 (C9) | VQR-SpBE3 | |
| | | UGACUUUGCAUUCCAGACCU | (GGG) | 20 (C9) | SpBE3 | |
| | | UUGACUUUGCAUUCCAGACC | (TGGG) | 20 (C10) | VQR-SpBE3 | |
| | | UUGACUUUGCAUUCCAGACC | (TGG) | 20 (C10) | SpBE3 | |
| | | UUGACUUUGCAUUCCAGACC | (TGGGG) | 20 (C10) | St3BE3 | |
| P616S/L | CCG to TCG or | GCAUGGAAUCCCGGCCCCUC | (AGG) | 20 (C11/12) | SpBE3 | 897-907 |
| and/or | CCG to CTG | CAUGGAAUCCCGGCCCCUCA | (GGAG) | 20 (C10/11) | EQR-SpBE3 | |
| P618S/L | and/or | AUGGAAUCCCGGCCCCUCAG | (GAG) | 20 (C9/10) | SpBE3 | |
| | CCT to TCT | GAAUCCCGGCCCCUCAGGAG | (CAG) | 20 (C6/7) | SpBE3 | |
| | or | AAUCCCGGCCCCUCAGGAGC | (AGG) | 20 (C5,6,11,12) | SpBE3 | |
| | CCT to CTT | AUCCCGGCCCCUCAGGAGCA | (GGTG) | 20 (C4,5,10,11) | VQR-SpBE3 | |
| | | CCCGGCCCCUCAGGAGCAGG | (TGAA) | 20 (C2,3,8,9) | VQR-SpBE3 | |
| | | CCGGCCCCUCAGGAGCAGGUG | (AAG) | 21 (C1,−1,6,7) | SpBE3 | |
| | | GGAAUCCCGGCCCCUCAGGA | (GCAGGT) | 20 (C7/8) | KKH-SaBE3 | |
| | | GCAUGGAAUCCCGGCCCCUC | (AGGAG) | 20 (C10/11) | St3BE3 | |
| | | AAUCCCGGCCCCUCAGGAGC | (AGGTG) | 20 (C5,6,11,12) | St3BE3 | |
| P618S/L | CCT to TCT | GGCCCCUCAGGAGCAGGUGA | (AGAG) | 20 (C5/6) | EQR-SpBE3 | 908-911 |
| | or | GCCCCUCAGGAGCAGGUGAA | (GAG) | 20 (C4/5) | SpBE3 | |
| | CCT to CTT | CCCCUCAGGAGCAGGUGAAG | (AGG) | 20 (C3/4) | SpBE3 | |
| | | GGAAUCCCGGCCCCUCAGGA | (GCAGGT) | 20 (C12/13) | KKH-SaBE3 | |
| C626Y | TGC to TAC | CGCAGGCCACGGUCACCUGC | (GAG) | 20 (C3) | SpBE3 | 912-914 |
| | | CAGGCCACGGUCACCUGCCA | (GAG) | 20 (C1) | SpBE3 | |
| | | GCAGGCCACGGUCACCUGCC | (AGAG) | 20 (C2) | EQR-SpBE3 | |
| C635Y | TGC to TAC | CACUGCAGCCAGUCAGGGUC | (CAG) | 20 (C6) | SpBE3 | 915-918 |
| | | GGAGGGCACUGCAGCCAGUC | (AGGG) | 20 (C12) | VQR-SpBE3 | |
| | | GAGGGCACUGCAGCCAGUCA | (GGG) | 20 (C11) | VQR-SpBE3 | |
| | | GGAGGGCACUGCAGCCAGUC | (AGG) | 20 (C13) | SpBE3 | |
| P639S/L | CCT to TCT | CCCUGGGACCUCCCACGUCC | (TGG) | 20 (C2/3) | SpBE3 | 919-922 |
| | or | CCUGGGACCUCCCACGUCCU | (GGG) | 20 (C1/2) | SpBE3 | |
| | CCT to CTT | CCCUGGGACCUCCCACGUCC | (TGGGG) | 20 (C2/3) | St3BE3 | |
| | | CCUGGGACCUCCCACGUCCU | (GGGGG) | 20 (C1/2) | St3BE3 | |
| G640R/E | GGG to AGG or | CCCAGGGAGGGCACUGCAGC | (CAG) | 20 (C2/3) | SpBE3 | 923-925 |
| | GGG to GAG | AGGUCCCAGGGAGGGCACUG | (CAG) | 20 (C6/7) | VQR-SpBE3 | |
| | | GUCCCAGGGAGGGCACUGCA | (GCCAGT) | 20 (C4/6) | KKH-SaBE3 | |
| C654Y | TGT to TAT | GACUACACACGUGUUGUCUA | (CGG) | 20 (C8) | SpBE3 | 926-930 |
| | | ACACGUGUUGUCUACGGCGU | (AGG) | 20 (C2) | SpBE3 | |
| | | CACACGUGUUGUCUACGGCG | (TAG) | 20 (C3) | SpBE3 | |
| | | ACUACACACGUGUUGUCUAC | (GGCG) | 20 (C7) | VRER-SpBE3 | |
| | | GACUACACACGUGUUGUCUA | (CGGCG) | 20 (08) | St3BE3 | |
| G670R/E | GGG to AGG | CCCUUCGCUGGUGCUGCCUG | (TAG) | 20 (C2/3) | SpBE3 | 931-935 |
| | | CCUUCGCUGGUGCUGCCUGU | (AGTG) | 20 (C1/2) | VQR-SpBE3 | |
| | | GCUGUCACGGCCCCUUCGCU | (GGTG) | 20 (C13/14) | VQR-SpBE3 | |
| | | GGCUGUCACGGCCCCUUCGC | (TGG) | 20 (C12/13) | SpBE3 | |
| | | GCCCCUUCGCUGGUGCUGCC | (TGTAGT) | 20 (C4/5) | KKH-SaBE3 | |
| C678Y | TGC to TAC | GCAGAUGGCAACGGCUGUCA | (CGG) | 20 (C2) | SpBE3 | 936, 937 |
| and | | GCUCCGGCAGCAGAUGGCAA | (CGG) | 20 (C11/8) | SpBE3 | |
| C679Y | | | | | | |

*Guide sequences (the portion of the guide RNA that targets the nucleobase editor to the target sequence) are provided, which may be used with any tracrRNA framework sequences provided herein to generate the full guide RNA sequence
^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.

In some embodiments, PCSK9 variants comprising more than one mutations described herein are contemplated. For example, a PCSK9 variant may be produced using the methods described herein that includes 1, 2, 3, 4, 5, 6, 7, 8, 9, 10, or more mutations selected from Tables 3 and 4. To make multiple mutations in the PCSK9 gene, a plurality of guide nucleotide sequences may be used, each guide nucleotide sequence targeting one target base. The nucleobase editor is capable of editing each and every base dictated by the guide nucleotide sequence. For example, 1, 2, 3, 4, 5, 6, 7, 8, 9, 10, or more guide nucleotide sequences may be used in a gene editing reaction. In some embodiments, the guide nucleotide sequences are RNAs (e.g., gRNA). In some embodiments, the guide nucleotide sequences are single stranded DNA molecules.

### Premature Stop Codons

Some aspects of the present disclosure provide strategies of editing PCSK9 gene to reduce the amount of full-length, functional PCSK9 protein being produced. In some embodiments, stop codons may be introduced into the coding sequence of PCSK9 gene upstream of the normal stop codon (referred to as a “premature stop codon”). Premature stop codons cause premature translation termination, in turn resulting in truncated and nonfunctional proteins and induces rapid degradation of the mRNA via the non-sense mediated mRNA decay pathway. See, e.g., Baker et al., Current Opinion in Cell Biology 16 (3): 293-299, 2004; Chang et al., Annual Review of Biochemistry 76: 51-74, 2007; and Behm-Ansmant et al., Genes & Development 20 (4): 391-398, 2006, each of which is incorporated herein by reference.

The nucleobase editors described herein may be used to convert several amino acid codons to a stop codon (e.g., TAA, TAG, or TGA). For example, nucleobase editors including a cytosine deaminase domain are capable of converting a cytosine (C) base to a thymine (T) base via deamination. Thus, it is envisioned that, for amino acid codons containing a C base, the C base may be converted to T. For example, a CAG (Gln/Q) codon may be changed to a TAG (amber) codon via the deamination of the first C on the coding strand. For sense codons that contain a guanine (G) base, a C base is present on the complementary strand; and the G base may be converted to an adenosine (A) via the deamination of the C on the complementary strand. For example, a TGG (Trp/W) codon may be converted to a TAG (amber) codon via the deamination of the second C on the complementary strand. In some embodiments, two C to T changes are required to convert a codon to a nonsense codon. For example, a CGG (R) codon is converted to a TAG (amber) codon via the deamination of the first C on the coding strand and the deamination of the second C on the complementary strand. Non-limiting examples of codons that may be changed to stop codons via base editing are provided in Table 5.

TABLE 5
Conversion to Stop Codon

| Target codon | Base-editing process | Edited codon |
|---|---|---|
| CAG (Gln/Q) | 1^{st} base C to T on coding strand | TAG (amber) |
| TGG (Trp/W) | 2^{nd} base C to T on complementary | TAG (amber) |
| | strand | |
| CGA (Arg/R) | 1^{st} base C to T on coding strand | TGA (opal) |
| CAA (Gln/Q) | 1^{st} base C to T on coding strand | TAA (ochre) |
| TGG (Trp/W) | 3^{rd} base C to T on complementary | TGA (opal) |
| | strand | |
| CGG (Arg/R) | 1^{st} base C to T on coding strand and | TAG (amber) |
| | 2^{nd} base C to T on complementary | |
| | strand | |
| CGA (Arg/R) | 1^{st} base C to T on coding strand and | TAA (orchre) |
| | 2^{nd} base C to T on complementary | |
| | strand | |

*single underline: changes on the coding strand double underline: changes on the complementary strand

Accordingly, the present disclosure provides non-limiting examples of amino acid codons that may be converted to premature stop codons in PCSK9 gene. In some embodiments, the introduction of stop codons may be efficacious in generating truncations when the target residue is located in a flexible loop. In some embodiments, two codons adjacent to each other may both be converted to stop codons, resulting in two stop codons adjacent to each other (also referred to as “tandem stop codons”). “Adjacent” means there are no more than 5 amino acids between the two stop codons. For example, the two stop codons may be immediately adjacent to each other (0 amino acids in between) or have 1, 2, 3, 4, or 5 amino acids in between. The introduction of tandem stop codons may be especially efficacious in generating truncation and nonfunctional PCSK9 mutations. Non-limiting examples of tandem stop codons that may be introduced include: W10X-W11X, Q99X-Q101X, Q342X-Q344X, and Q554X-Q555X, wherein X indicates the stop codon. In some embodiments, a stop codon may be introduced after a structurally destabilizing mutation (e.g., the structurally destabilizing mutations listed in Table 2) to effectively produce truncation PCSK9 proteins. Non-limiting examples of a structurally destabilizing mutation followed by a stop codon include: P530S/L-Q531X, P581S/L-R582X, and P618S/L-Q619X, wherein X indicates the stop codon.

Exemplary codons that may be changed to stop codons by the nucleobase editors described herein and the guide nucleotide sequence that may be used are listed in Table 6. The examples are for illustration purpose only and are not meant to be limiting.

TABLE 6
Introducing Premature Stop Codon into PCSK9 Gene via Base Editing

| Target | Stop | Predicted | | | gRNA size | | SEQ |
|---|---|---|---|---|---|---|---|
| codon | codon | truncation* | guide sequence | (PAM) | (C edited) | BE type^{a} | ID NO |
| W10 | TAG | ++ | CCAGGACCGCCUGGAGCUGAC | (GGTG) | 21 (C−1) | VQR-SpBE3 | 938-946 |
| (TGG) | or | | CCAGGACCGCCUGGAGCUGA | (CGG) | 20 (C1) | SpBE3 | |
| and/or | TGA | | CCACCAGGACCGCCUGGAGC | (TGAC) | 20 (C4,5,1,2) | VQR-SpBE3 | |
| W11 | | | GCGGCCACCAGGACCGCCUG | (GAG) | 20 (C8,9,5,6) | SpBE3 | |
| (TGG) | | | AGCGGCCACCAGGACCGCCU | (GGAG) | 20 (C9,10,6,7) | EQR-SpBE3 | |
| | | | CAGCGGCCACCAGGACCGCC | (TGG) | 20 (C10,11,7,8) | SpBE3 | |
| | | | CACCAGGACCGCCUGGAGCU | (GACGGT) | 20 (C3,4,1) | KKH-SaBE3 | |
| | | | CCAGGACCGCCUGGAGCUGA | (CGGTG) | 20 (C−1) | St3BE3 | |
| | | | CAGCGGCCACCAGGACCGCC | (TGGAG) | 20 (C10,11,7,8) | St3BE3 | |
| Q31 | TAG | + | GGCGCCCGUGCGCAGGAGGA | (CGAG) | 20 (C13) | EQR-SpBE3 | 947-954 |
| (CAG) | | | GCGCCCGUGCGCAGGAGGAC | (GAG) | 20 (C12) | SpBE3 | |
| | | | CGCCCGUGCGCAGGAGGACG | (AGG) | 20 (C11) | SpBE3 | |
| | | | GCCCGUGCGCAGGAGGACGA | (GGAC) | 20 (C10) | VQR-SpBE3 | |
| | | | CGUGCGCAGGAGGACGAGGA | (CGG) | 20 (C7) | SpBE3 | |
| | | | GUGCGCAGGAGGACGAGGAC | (GGCG) | 20 (C6) | VRER-SpBE3 | |
| | | | GCGCAGGAGGACGAGGACGG | (CGAC) | 20 (C4) | VQR-SpBE3 | |
| | | | CGUGCGCAGGAGGACGAGGA | (CGGCG) | 20 (C7) | St3BE3 | |
| W77 | TAG | + | CAGGCAACCUCCACGGAUCC | (TGG) | 20 (C11/12) | SpBE3 | 955 |
| (TGG) | or | | | | | | |
| | TGA | | | | | | |
| Q90 | TAG | + | GACCCACCUCUCGCAGUCAG | (AGCG) | 20 (C14*) | VRER-SpBE3 | 956 |
| (CAG) | | | | | | | |
| Q99 | TAG | ++ with | UGCAGGCCCAGGCUGCCCGC | (CGG) | 20 (C3/9) | SpBE3 | 957-961 |
| (CAG) | | Q101X | GCAGGCCCAGGCUGCCCGCC | (GGG) | 20 (C2/8) | SpBE3 | |
| and/or | | | CAGGCCCAGGCUGCCCGCCG | (GGG) | 20 (C1/7) | SpBE3 | |
| Q101 | | | GCAGGCCCAGGCUGCCCGCC | (GGGGAT) | 20 (C2/8) | SaBE3 | |
| (CAG) | | | UGCAGGCCCAGGCUGCCCGC | (CGGGG) | 20 (C3/9) | St3BE3 | |
| Q101 | TAG | ++ with | AGGCCCAGGCUGCCCGCCGG | (GGAT) | 20 (C6) | EQR-SpBE3 | 962 |
| (CAG) | | Q99X | | | | | |
| Q152 | TAG | ++ | UGUCUUUGCCCAGAGCAUCC | (CGTG) | 20 (C10) | VQR-SpBE3 | 963-967 |
| (CAG) | | | UCUUUGCCCAGAGCAUCCCG | (TGG) | 20 (C9) | SpBE3 | |
| | | | CUUUGCCCAGAGCAUCCCGU | (GGAA) | 20 (C7) | VQR-SpBE3 | |
| | | | CCAGAGCAUCCCGUGGAACC | (TGG) | 20 (C1) | SpBE3 | |
| | | | CCAGAGCAUCCCGUGGAACC | (TGGAG) | 20 (C1) | St3BE3 | |
| W156 | TAG | + | CCACGGGAUGCUCUGGGCAA | (AGAC) | 20 (C1/2) | VQR-SpBE3 | 968-972 |
| (TGG) | or | | UCCACGGGAUGCUCUGGGCA | (AAG) | 20 (C2/3) | SpBE3 | |
| | TGA | | CCAGGUUCCACGGGAUGCUC | (TGGG) | 20 (C8/9) | VQR-SpBE3 | |
| | | | CAGGUUCCACGGGAUGCUCU | (GGG) | 20 (C7/8) | SpBE3 | |
| | | | CCAGGUUCCACGGGAUGCUC | (TGG) | 20 (C8/9) | SpBE3 | |
| Q172 | TAG | ++ | GCGGAUGAAUACCAGCCCCC | (CGG) | 20 (C13) | SpBE3 | 973-975 |
| (CAG) | | | AUGAAUACCAGCCCCCCGGU | (AAG) | 20 (C9) | SpBE3 | |
| | | | UGAAUACCAGCCCCCCGGUA | (AGAC) | 20 (C8) | VQR-SpBE3 | |
| Q190 | TAG | ++ | CCAGCAUACAGAGUGACCAC | (CGG) | 20 (C9) | SpBE3 | 976-981 |
| (CAG) | | | CAGCAUACAGAGUGACCACC | (GGG) | 20 (C8) | SpBE3 | |
| | | | CCAGCAUACAGAGUGACCAC | (CGGG) | 20 (C7) | VQR-SpBE3 | |
| | | | AGCAUACAGAGUGACCACCG | (GGAA) | 20 (C7) | VQR-SpBE3 | |
| | | | CAGAGUGACCACCGGGAAAU | (CGAG) | 20 (C1) | EQR-SpBE3 | |
| | | | AGCAUACAGAGUGACCACCG | (GGAAAT) | 20 (C7) | KKH-SaBE3 | |
| Q219 | TAG | ++ | CUUCCACAGACAGGUAAGCA | (CGG) | 20 (C11) | SpBE3 | 982-984 |
| (CAG) | | | GACAGGUAAGCACGGCCGUC | (TGAT) | 20 (C3) | VQR-SpBE3 | |
| | | | CAGACAGGUAAGCACGGCCG | (TCTGAT) | 20 (C5) | KKH-SaBE3 | |
| Q256 | TAA | − | CGUGCUCAACUGCCAAGGGA | (AGG) | 20 (C14) | SpBE3 | 985-992 |
| (CAA) | | | GUGCUCAACUGCCAAGGGAA | (GGG) | 20 (C13) | SpBE3 | |
| | | | CGUGCUCAACUGCCAAGGGA | (AGGG) | 20 (C13) | VQR-SpBE3 | |
| | | | CAACUGCCAAGGGAAGGGCA | (CGG) | 20 (C8) | SpBE3 | |
| | | | UGCCAAGGGAAGGGCACGGU | (TAG) | 20 (C4) | SpBE3 | |
| | | | GCCAAGGGAAGGGCACGGUU | (AGCG) | 20 (C3) | VRER-SpBE3 | |
| | | | CAAGGGAAGGGCACGGUUAG | (CGG) | 20 (C1) | SpBE3 | |
| | | | CUCAACUGCCAAGGGAAGGG | (CACGGT) | 20 (C10) | KKH-SaBE3 | |
| Q275 | TAG | − | UUCGGAAAAGCCAGCUGGUC | (CAG) | 20 (C12) | SpBE3 | 993-996 |
| (CAG) | | | AAAAGCCAGCUGGUCCAGCC | (TGTG) | 20 (C7) | VQR-SpBE3 | |
| | | | AAGCCAGCUGGUCCAGCCUG | (TGG) | 20 (C5) | SpBE3 | |
| | | | AAGCCAGCUGGUCCAGCCUG | (TGGGG) | 20 (C5) | St3BE3 | |
| Q278 | TAG | + | AAGCCAGCUGGUCCAGCCUG | (TGG) | 20 (C14) | SpBE3 | 997-1008 |
| (CAG) | | | AGCCAGCUGGUCCAGCCUGU | (GGG) | 20 (C13/4) | SpBE3 | |
| and/or | | | GCCAGCUGGUCCAGCCUGUG | (GGG) | 20 (C12/3) | SpBE3 | |
| Q275 | | | AGCCAGCUGGUCCAGCCUGU | (GGGG) | 20 (C13/4) | SpBE3 | |
| (CAG) | | | GGUCCAGCCUGUGGGGCCAC | (TGG) | 20 (C5) | SpBE3 | |
| | | | GUCCAGCCUGUGGGGCCACU | (GGTG) | 20 (C4) | VQR-SpBE3 | |
| | | | CCAGCCUGUGGGGCCACUGG | (TGG) | 20 (C2) | SpBE3 | |
| | | | CAGCCUGUGGGGCCACUGGU | (GGTG) | 20 (C1) | VQR-SpBE3 | |
| | | | CUGGUCCAGCCUGUGGGGCC | (ACTGGT) | 20 (C7) | KKH-SaBE3 | |
| | | | GUCCAGCCUGUGGGGCCACU | (GGTGGT) | 20 (C4) | KKH-SaBE3 | |
| | | | GGUCCAGCCUGUGGGGCCAC | (TGGTG) | 20 (C5) | St3BE3 | |
| | | | CCAGCCUGUGGGGCCACUGG | (TGGTG) | 20 (C2) | St3BE3 | |
| Q302 | TAG | − | CAACGCCGCCUGCCAGCGCC | (TGG) | 20 (C14) | SpBE3 | 1009-1019 |
| (CAG) | | | AACGCCGCCUGCCAGCGCCU | (GGCG) | 20 (C13) | VRER-SpBE3 | |
| | | | CGCCGCCUGCCAGCGCCUGG | (CGAG) | 20 (C11) | EQR-SpBE3 | |
| | | | GCCGCCUGCCAGCGCCUGGC | (GAG) | 20 (C10) | SpBE3 | |
| | | | CCGCCUGCCAGCGCCUGGCG | (AGG) | 20 (C9) | SpBE3 | |
| | | | CGCCUGCCAGCGCCUGGCGA | (GGG) | 20 (C8) | SpBE3 | |
| | | | UGCCAGCGCCUGGCGAGGGC | (TGG) | 20 (C4) | SpBE3 | |
| | | | GCCAGCGCCUGGCGAGGGCU | (GGG) | 20 (C3) | SpBE3 | |
| | | | CCAGCGCCUGGCGAGGGCUG | (GGG) | 20 (C2) | SpBE3 | |
| | | | UGCCAGCGCCUGGCGAGGGC | (TGGGGT) | 20 (C4) | SaBE3 | |
| | | | UGCCAGCGCCUGGCGAGGGC | (TGGGG) | 20 (C4) | St3BE3 | |
| Q342 | TAA | ++ with | CACCAAUGCCCAAGACCAGC | (CGG) | 20 (C11) | SpBE3 | 1020-1028 |
| (CAA) | and/or | Q344X | ACCAAUGCCCAAGACCAGCC | (GGTG) | 20 (C10) | VQR-SpBE3 | |
| and/or | TAG | | CAAUGCCCAAGACCAGCCGG | (TGAC) | 20 (C8) | VQR-SpBE3 | |
| Q344 | | | CCAAGACCAGCCGGUGACCC | (TGG) | 20 (C2/8) | SpBE3 | |
| (CAG) | | | CAAGACCAGCCGGUGACCCU | (GGG) | 20 (C1/7) | SpBE3 | |
| | | | CAAGACCAGCCGGUGACCCUG | (GGG) | 21 (C−1/6) | SpBE3 | |
| | | | GCCACCAAUGCCCAAGACCA | (GCCGGT) | 20 (C13) | KKH-SaBE3 | |
| | | | CACCAAUGCCCAAGACCAGC | (CGGTG) | 20 (C11) | St3BE3 | |
| | | | CCAAGACCAGCCGGUGACCC | (TGGGG) | 20 (C2/8) | St3BE3 | |
| Q344 | TAG | ++ with | AGACCAGCCGGUGACCCUGG | (GGAC) | 20 (C5) | VQR-SpBE3 | 1029 |
| (CAG) | | Q342X | | | | | |
| Q382 | TAG | − | CUGCUUUGUGUCACAGAGUG | (GGAC) | 20 (C14) | VQR-SpBE3 | 1030-1032 |
| (CAG) | | | UGUCACAGAGUGGGACAUCA | (CAG) | 20 (C6) | SpBE3 | |
| | | | GUCACAGAGUGGGACAUCAC | (AGG) | 20 (C5) | SpBE3 | |
| Q387 | TAG | − | ACAUCACAGGCUGCUGCCCA | (CGTG) | 20 (C7) | VQR-SpBE3 | 1033-1036 |
| (CAG) | | | AUCACAGGCUGCUGCCCACG | (TGG) | 20 (C5) | SpBE3 | |
| | | | CAGGCUGCUGCCCACGUGGC | (TGG) | 20 (C1) | SpBE3 | |
| | | | CACAGGCUGCUGCCCACGUG | (GCTGGT) | 20 (C3) | KKH-SaBE3 | |
| Q413 | TAG | | GGCCGAGUUGAGGCAGAGAC | (TGAT) | 20 (C14) | VQR-SpBE3 | 1037 |
| (CAG) | | | | | | | |
| W428 | TAG | | AGGGAACCAGGCCUCAUUGA | (TGAC) | 20 (C7/8) | VQR-SpBE3 | 1038-1040 |
| (TGG) | or | | CUCAGGGAACCAGGCCUCAU | (TGAT) | 20 (C10/11) | VQR-SpBE3 | |
| | TGA | | UCCUCAGGGAACCAGGCCUC | (ATTGAT) | 20 (C11/12) | KKH-SaBE3 | |
| Q433 | TAG | | CCCUGAGGACCAGCGGGUAC | (TGAC) | 20 (C11) | VQR-SpBE3 | 1041-1042 |
| (CAG) | | | CAGCGGGUACUGACCCCCAA | (CCTGGT) | 20 (C1) | KKH-SaBE3 | |
| W453 | TAG | ++ | CAGCUGCCAACCUGCAAAAA | (GGG) | 20 (C8/9) | SpBE3 | 1043-1049 |
| (TGG) | or | | GCCAACCUGCAAAAAGGGCC | (TGGG) | 20 (C2/3) | VQR-SpBE3 | |
| | TGA | | GCCAACCUGCAAAAAGGGCC | (TGG) | 20 (C2/3) | SpBE3 | |
| | | | ACAGCUGCCAACCUGCAAAA | (AGGG) | 20 (C8/9) | VQR-SpBE3 | |
| | | | ACAGCUGCCAACCUGCAAAA | (AGG) | 20 (C8/9) | SpBE3 | |
| | | | AACAGCUGCCAACCUGCAAA | (AAG) | 20 (C9/10) | SpBE3 | |
| | | | GCCAACCUGCAAAAAGGGCC | (TGGGAT) | 20 (C2/3) | SaBE3 | |
| Q454 | TAG | ++ | GCAGGUUGGCAGCUGUUUUG | (CAG) | 20 (C10) | SpBE3 | 1050-1053 |
| (CAG) | | | CAGGUUGGCAGCUGUUUUGC | (AGG) | 20 (C9) | SpBE3 | |
| | | | AGGUUGGCAGCUGUUUUGCA | (GGAC) | 20 (C8) | VQR-SpBE3 | |
| | | | GCAGCUGUUUUGCAGGACUG | (TATGGT) | 20 (C2) | KKH-SaBE3 | |
| W461 | TAG | − | GACCAUACAGUCCUGCAAAA | (CAG) | 20 (C3/4) | SpBE3 | 1054 |
| (TGG) | or | | | | | | |
| | TGA | | | | | | |
| Q503 | TAG | + | UAAGGCCCAAGGGGGCAAGC | (TGG) | 20 (C8) | SpBE3 | 1055-1057 |
| (CAA) | | | ACUCUAAGGCCCAAGGGGGC | (AAG) | 20 (C12) | SpBE3 | |
| | | | UCUAAGGCCCAAGGGGGCAA | (GCTGGT) | 20 (C10) | KKH-SaBE3 | |
| Q531 | TAG | ++ with | CUGCUACCCCAGGCCAACUG | (CAG) | 20 (C10) | SpBE3 | 1058-1060 |
| (CAG) | | P530S | UGCUACCCCAGGCCAACUGC | (AGCG) | 20 (C9) | VQR-SpBE3 | |
| | | | CAGGCCAACUGCAGCGUCCAC | (CAG) | 22 (C−2) | SpBE3 | |
| | | | A | | | | |
| Q554 | TAG | ++ with | CCAACAGGGCCACGUCCUCA | (CAG) | 20 (C2/5) | SpBE3 | 1061-1065 |
| (CAA) | and/or | Q555X | CAACAGGGCCACGUCCUCAC | (AGG) | 20 (C1/4) | SpBE3 | |
| and/or | TAA | | CAGGGCCACGUCCUCACAGG | (TAG) | 20 (C1) | SpBE3 | |
| Q555 | | | CAGGGCCACGUCCUCACAGG | (AGG) | 21 (C−1) | SpBE3 | |
| (CAG) | | | U | | | | |
| | | | ACCAACAGGGCCACGUCCUC | (ACAGGT) | 20 (C3/6) | KKH-SaBE3 | |
| W566 | TAG | ++ | CCCAGUGGGAGCUGCAGCCU | (GGGG) | 20 (C2/3) | VQR-SpBE3 | 1066-1072 |
| (TGG) | or | | CCAGUGGGAGCUGCAGCCUG | (GGG) | 20 (C1/2) | SpBE3 | |
| | TGA | | UCCCAGUGGGAGCUGCAGCC | (TGGG) | 20 (C3/4) | VQR-SpBE3 | |
| | | | CCCAGUGGGAGCUGCAGCCU | (GGG) | 20 (C2/3) | SpBE3 | |
| | | | UCCCAGUGGGAGCUGCAGCC | (TGG) | 20 (C3/4) | SpBE3 | |
| | | | CCACCUCCCAGUGGGAGCUG | (CAG) | 20 (C7/8) | SpBE3 | |
| | | | UCCCAGUGGGAGCUGCAGCC | (TGGGG) | 20 (C4/5) | St3BE3 | |
| R582 | TGA | ++ with | GGCCACGAGGUCAGCCCAAC | (CAG) | 20 (C12/6) | SpBE3 | 1073-1077 |
| (CGA) | and/or | P581S/L | GCCACGAGGUCAGCCCAACC | (AGTG) | 20 (C11/5) | VQR-SpBE3 | |
| and/or | TAG | | CACGAGGUCAGCCCAACCAG | (TGCG) | 20 (C9/3) | VRER-SpBE3 | |
| Q584 | | | CGAGGUCAGCCCAACCAGUG | (CGTG) | 20 (C6/1) | VQR-SpBE3 | |
| (CAG) | | | GAGGCCACGAGGUCAGCCCA | (ACCAGT) | 20 (C8) | KKH-SaBE3 | |
| Q584 | TAG | − | GGUCAGCCCAACCAGUGCGU | (GGG) | 20 (C4) | SpBE3 | 1078-1085 |
| (CAG) | | | AGGUCAGCCCAACCAGUGCG | (TGG) | 20 (C5) | SpBE3 | |
| | | | GGCCACGAGGUCAGCCCAAC | (CAG) | 20 (C12) | SpBE3 | |
| | | | GCCACGAGGUCAGCCCAACC | (AGTG) | 20 (C11) | VQR-SpBE3 | |
| | | | CACGAGGUCAGCCCAACCAG | (TGCG) | 20 (C9) | VRER-SpBE3 | |
| | | | CGAGGUCAGCCCAACCAGUG | (CGTG) | 20 (C7) | VQR-SpBE3 | |
| | | | AGGUCAGCCCAACCAGUGCG | (TGG) | 20 (C5) | SpBE3 | |
| | | | GGUCAGCCCAACCAGUGCGU | (GGG) | 20 (C4/13) | SpBE3 | |
| Q587 | TAG | − | CCCAACCAGUGCGUGGGCCA | (CAG) | 20 (C7) | SpBE3 | 1086-1092 |
| (CAG) | | | CCAGUGCGUGGGCCACAGGG | (AGG) | 20 (C2) | SpBE3 | |
| | | | ACCAGUGCGUGGGCCACAGG | (GAG) | 20 (C3) | SpBE3 | |
| | | | AACCAGUGCGUGGGCCACAG | (GGAG) | 20 (C4) | EQR-SpBE3 | |
| | | | CAACCAGUGCGUGGGCCACA | (GGG) | 20 (C5) | SpBE3 | |
| | | | CCAACCAGUGCGUGGGCCAC | (AGG) | 20 (C6) | SpBE3 | |
| | | | CAACCAGUGCGUGGGCCACA | (GGGAG) | 20 (C5) | St3BE3 | |
| Q619 | TAG | ++ with | CAGGAGCAGGUGAAGAGGCC | (CGTG) | 20 (C1) | VQR-SpBE3 | 1093-1098 |
| (CAG) | | P168S | CCCCUCAGGAGCAGGUGAAG | (AGG) | 20 (C6) | SpBE3 | |
| | | | GCCCCUCAGGAGCAGGUGAA | (GAG) | 20 (C7) | SpBE3 | |
| | | | GGCCCCUCAGGAGCAGGUGA | (AGAG) | 20 (C8) | EQR-SpBE3 | |
| | | | CGGCCCCUCAGGAGCAGGUG | (AAG) | 20 (C9) | SpBE3 | |
| | | | CCCGGCCCCUCAGGAGCAGG | (TGAA) | 20 (C11) | VQR-SpBE3 | |
| Q621 | TAG | ++ | GGCCCCUCAGGAGCAGGUGA | (AGAG) | 20 (C14) | EQR-SpBE3 | 1099-1106 |
| (CAG) | | | GCCCCUCAGGAGCAGGUGAA | (GAG) | 20 (C13) | SpBE3 | |
| | | | CCCCUCAGGAGCAGGUGAAG | (AGG) | 20 (C12) | SpBE3 | |
| | | | CAGGAGCAGGUGAAGAGGCC | (CGTG) | 20 (C7) | VQR-SpBE3 | |
| | | | GGAGCAGGUGAAGAGGCCCG | (TGAG) | 20 (C5) | EQR-SpBE3 | |
| | | | GAGCAGGUGAAGAGGCCCGU | (GAG) | 20 (C4) | SpBE3 | |
| | | | AGCAGGUGAAGAGGCCCGUG | (AGG) | 20 (C3) | SpBE3 | |
| | | | CAGGUGAAGAGGCCCGUGAG | (CCGGGT) | 21 (C−1) | SaBE3 | |
| | | | G | | | | |
| W630 | TGA | + | CCAGCCCUCCUCGCAGGCCA | (CGG) | 20 (C1/2) | SpBE3 | 1107-1110 |
| (TGG) | | | CAGGGUCCAGCCCUCCUCGC | (AGG) | 20 (C7/8) | SpBE3 | |
| | | | UCAGGGUCCAGCCCUCCUCG | (CAG) | 20 (C8/9) | SpBE3 | |
| | | | GUCCAGCCCUCCUCGCAGGC | (CACGGT) | 20 (C3/4) | KKH-SaBE3 | |
| Q686 | TAG | − | GGCACCUGGCGCAGGCCUCC | (CAG) | 20 (C12) | SpBE3 | 1111-1119 |
| (CAG) | | | GCACCUGGCGCAGGCCUCCC | (AGG) | 20 (C11) | SpBE3 | |
| | | | CACCUGGCGCAGGCCUCCCA | (GGAG) | 20 (C10) | EQR-SpBE3 | |
| | | | ACCUGGCGCAGGCCUCCCAG | (GAG) | 20 (C9) | SpBE3 | |
| | | | CGCAGGCCUCCCAGGAGCUC | (CAG) | 20 (C3) | SpBE3 | |
| | | | GCAGGCCUCCCAGGAGCUCC | (AGTG) | 20 (C2) | VQR-SpBE3 | |
| | | | CAGGCCUCCCAGGAGCUCCAG | (TGAC) | 21 (C−1) | VQR-SpBE3 | |
| | | | GGCGCAGGCCUCCCAGGAGC | (TCCAGT) | 20 (C5) | SaBE3 | |
| | | | GCACCUGGCGCAGGCCUCC | (CAGGAG) | 19 (C11) | St3BE3 | |
| Q689 | TAG | − | CCUCCCAGGAGCUCCAGUGA | (CAG) | 20 (C6) | SpBE3 | 1120-1123 |
| (CAG) | | | AGGCCUCCCAGGAGCUCCAG | (TGAC) | 20 (C9) | VQR-SpBE3 | |
| | | | GCAGGCCUCCCAGGAGCUCC | (AGTG) | 20 (C11) | VQR-SpBE3 | |
| | | | CGCAGGCCUCCCAGGAGCUC | (CAG) | 20 (C12) | SpBE3 | |

*Residues found in loop/linker regions are labeled + or ++ Guide sequences (the portion of the guide RNA that targets the nucleobase editor to the target sequence) are provided, which may be used with any tracrRNA framework sequences provided herein to generate the full guide RNA sequence
^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.

### Target Base in Non-Coding Region of PCSK9 Gene-Splicing Variants

Some aspects of the present disclosure provide strategies of reducing cellular PCSK9 activity via preventing PCSK9 mRNA maturation and production. In some embodiments, such strategies involve alterations of splicing sites in the PCSK9 gene. Altered splicing site may lead to altered splicing and maturation of the PCSK9 mRNA. For example, in some embodiments, an altered splicing site may lead to the skipping of an exon, in turn leading to a truncated protein product or an altered reading frame. In some embodiments, an altered splicing site may lead to translation of an intron sequence and premature translation termination when an in frame stop codon is encountered by the translating ribosome in the intron. In some embodiments, a start codon is edited and protein translation initiates at the next ATG codon, which may not be in the correct coding frame.

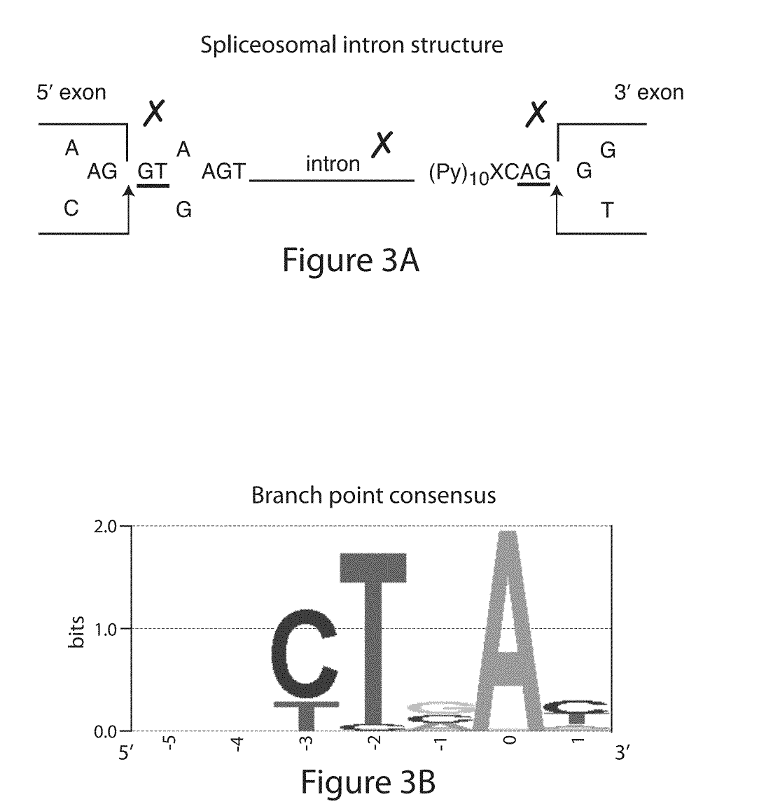

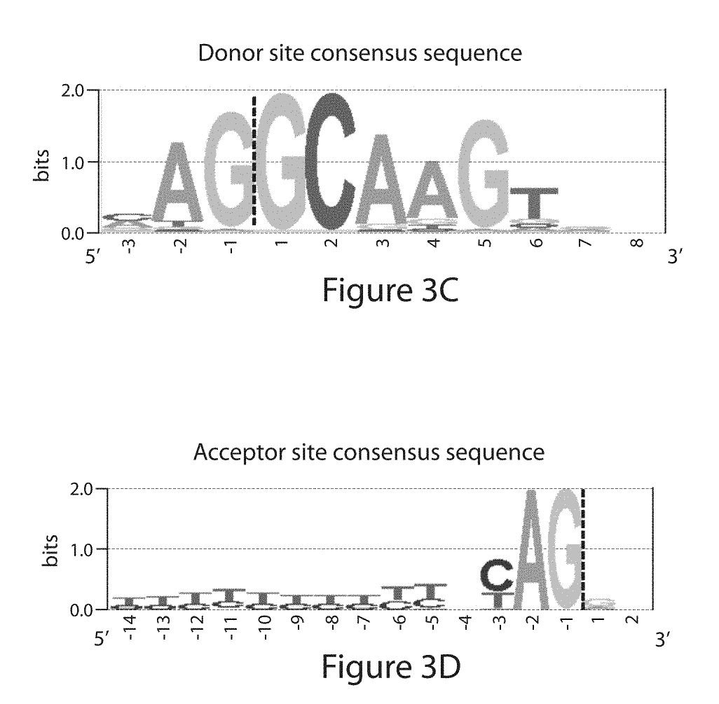

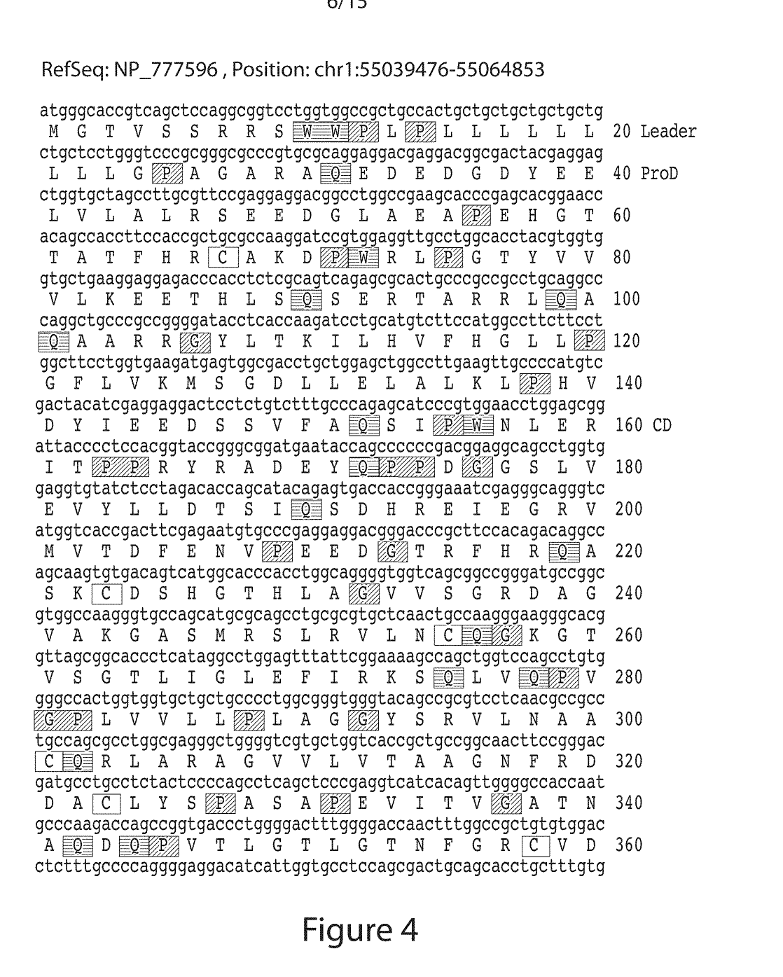

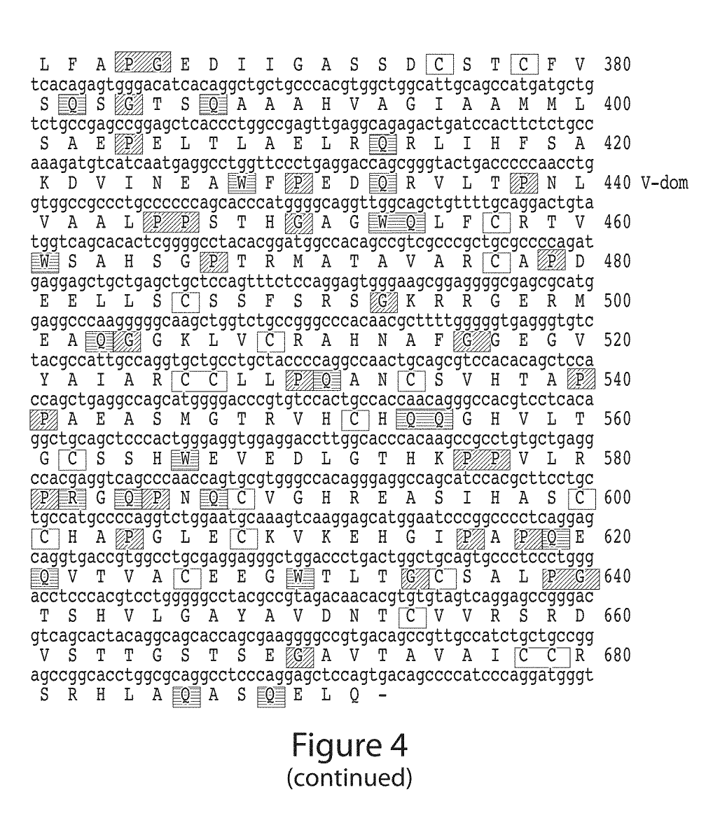

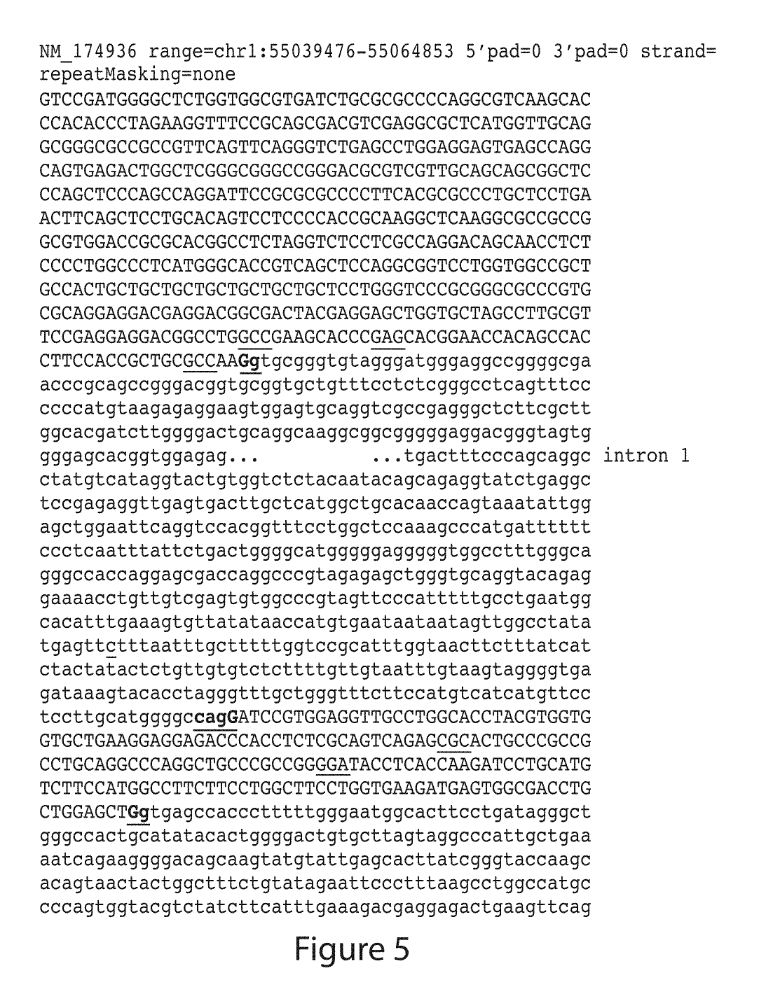

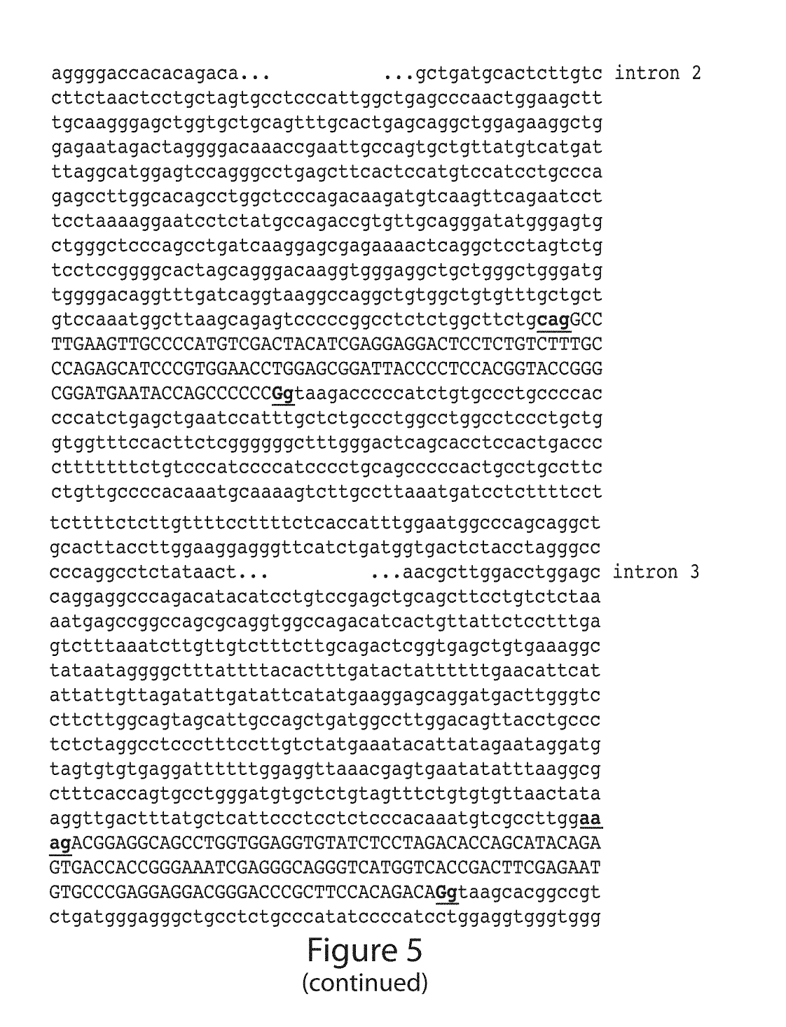

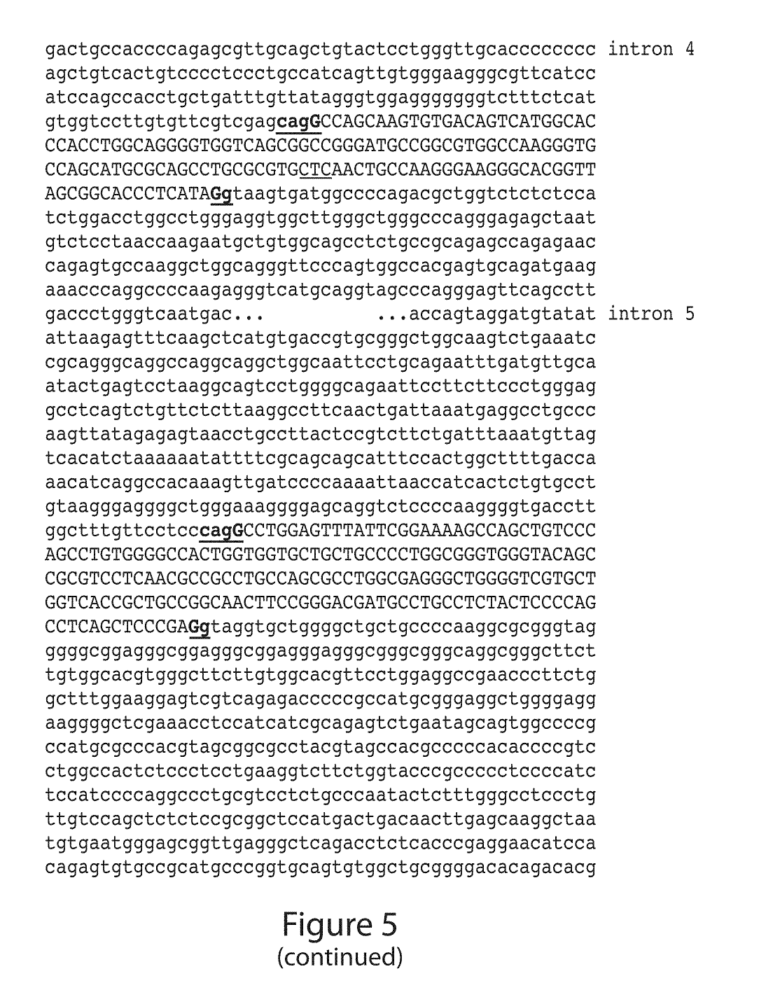

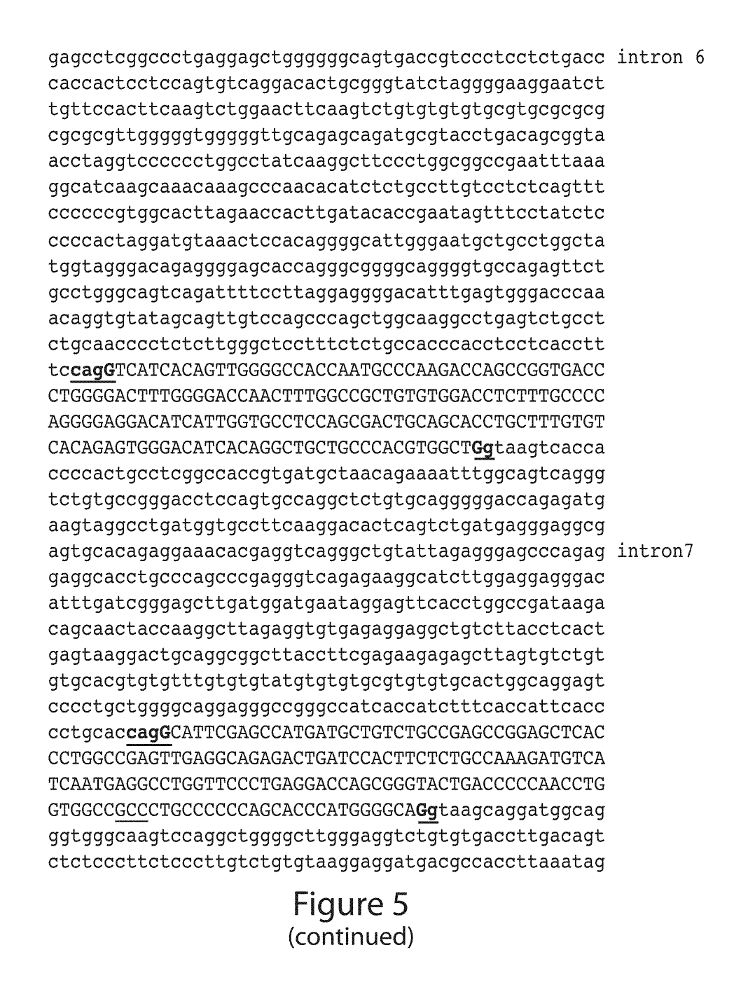

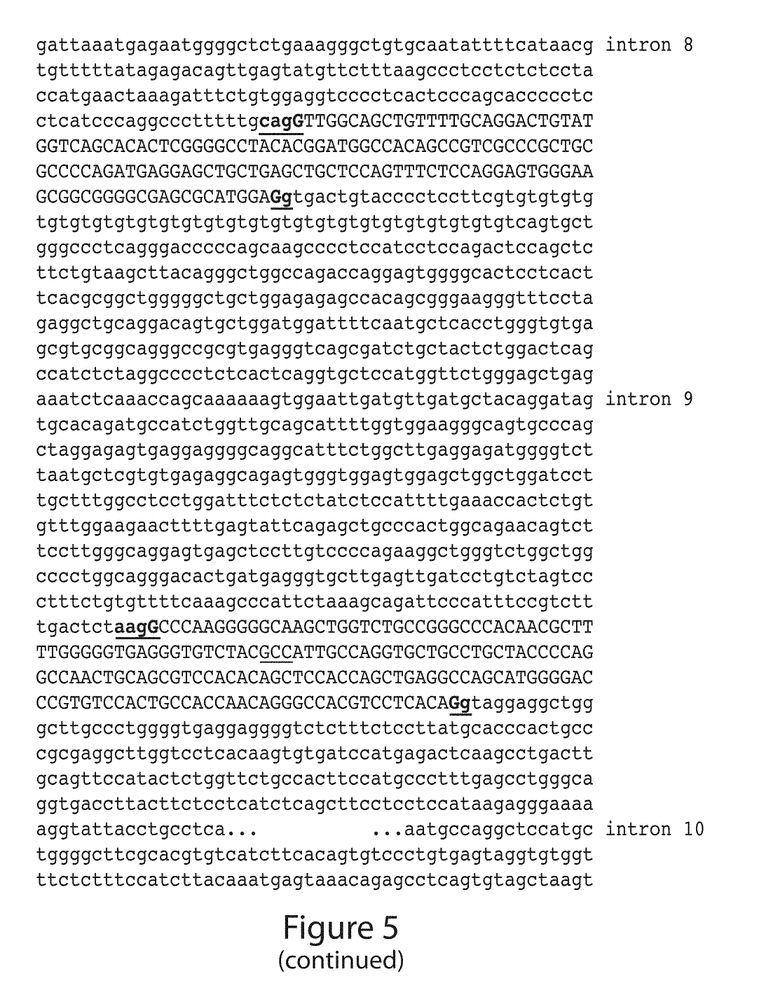

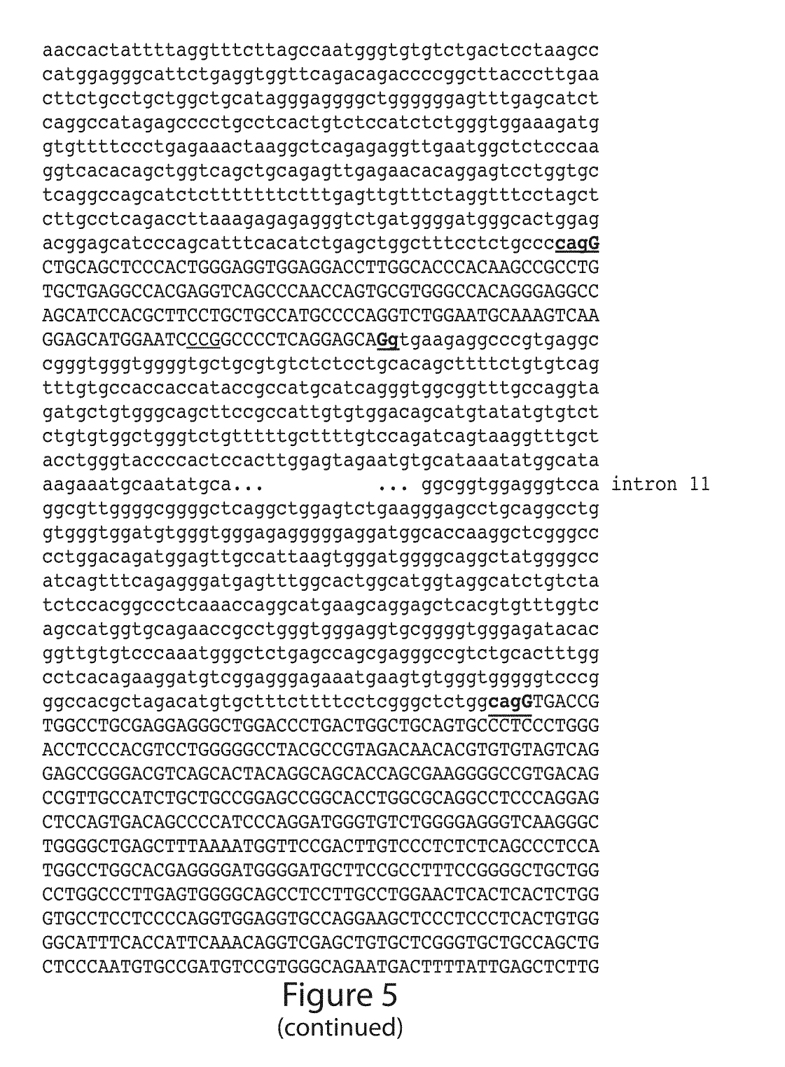

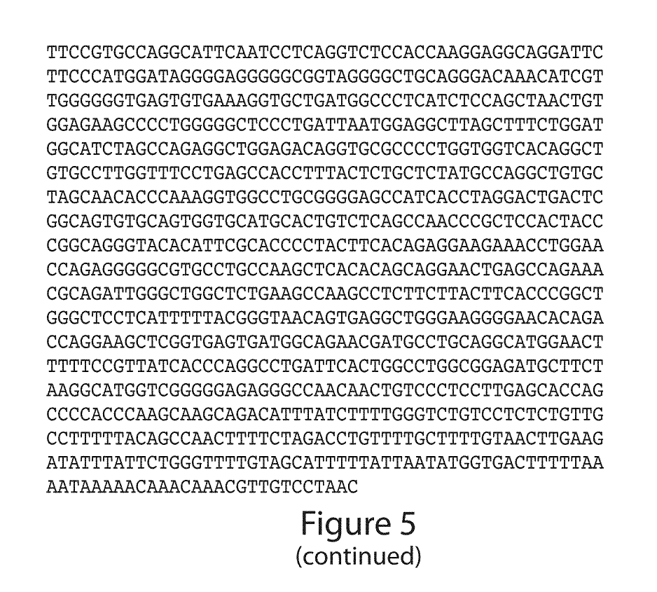

The splicing sites typically comprises an intron donor site, a Lariat branch point, and an intron acceptor site. The mechanism of splicing are familiar to those skilled in the art. As illustrated in FIG. 3, the intron donor site has a consensus sequence of GGGTRAGT, and the C bases paired with the G bases in the intron donor site consensus sequence may be targeted by a nucleobase editors described herein, thereby altering the intron donor site. The Lariat branch point also has consensus sequences, e.g., YTRAC, wherein Y is a pyrimidine and R is a purine. The C base in the Lariat branch point consensus sequence may be targeted by the nucleobase editors described herein, leading to the skipping of the following exon. The intron acceptor site has a consensus sequence of YNCAGG, wherein Y is a pyrimidine and N is any nucleotide. The C base of the consensus sequence of the intron acceptor site, and the C base paired with the G bases in the consensus sequence of the intron acceptor site may be targeted by the nucleobase editors described herein, thereby altering the intron acceptor site, in turn leading the skipping of an exon. General strategies of altering the splicing sites of the PCSK9 gene are described in Table 7.

TABLE 7
Exemplary Alteration of Intron-Exon Junction via Base Editing

| Target | Consensus | Base-editing | Edited | |
|---|---|---|---|---|
| site | Sequence | reaction (s) | sequence | Outcome |
| Intron | GGGTRAGT | 2^{nd} or 3^{rd} base | GAGTRAGT | Intron sequence is translated |
| donor | (example) | C to T on | (example) | as exon, in frame premature |
| | | complementary | | STOP codon |
| | | strand | | |
| Lariat | YTRAC | 5^{th} base C to T | YTRAT | The following exon is |
| branch | (example) | on coding | (example) | skipped from the mature |
| point | | strand | | mRNA, which may affect the |
| | | | | coding frame |
| Intron | Y(rich)NCAGG | 2^{nd} to last base | Y(rich)NCAAG | The exon is skipped from the |
| acceptor | (example) | C to T on | (example) | mature mRNA, which may |
| | | complementary | | affect the coding frame |
| | | strand | | |
| Start | ATG (Met/M) | 3^{rd} base C to T | ATA (Ile/I) | The next ATG is used as |
| codon | | on | | start codon, which may |
| | | complementary | | affect the coding frame |
| | | strand | | |

As described herein, gene sequence for human PCSK9 (SEQ ID NO: 1990) is ˜22-kb long and contains 12 exons and 11 introns. Each of the exon-intron junction may be altered to disrupt the processing and maturation of the PCSK9 mRNA. Thus, provided in Table 8 are non-limiting examples of alterations that may be made in the PCSK9 gene using the nucleobase editors described herein, and the guide sequences that may be used for each alteration.

TABLE 8
Alteration of Intron/Exon Junctions in PCSK9 Gene via Base Editing

| Target | Stop | Predicted | | | gRNA size | | SEQ |
|---|---|---|---|---|---|---|---|
| codon | codon | truncation* | guide sequence | (PAM) | (C edited) | BE type^{a} | ID NO |
| W10 | TAG or | ++ | CCAGGACCGCCUGGAGCUGAC | (GGTG) | 21 (C−1) | VQR-SpBE3 | 1124-1132 |
| (TGG) | TGA | | CCAGGACCGCCUGGAGCUGA | (CGG) | 20 (C1) | SpBE3 | |
| and/or | | | CCACCAGGACCGCCUGGAGC | (TGAC) | 20 (C4,5,1,2) | VQR-SpBE3 | |
| W11 | | | GCGGCCACCAGGACCGCCUG | (GAG) | 20 (C8,9,5,6) | SpBE3 | |
| (TGG) | | | AGCGGCCACCAGGACCGCCU | (GGAG) | 20 (C9,10,6,7) | EQR-SpBE3 | |
| | | | CAGCGGCCACCAGGACCGCC | (TGG) | 20 (C10,11,7,8) | SpBE3 | |
| | | | CACCAGGACCGCCUGGAGCU | (GACGGT) | 20 (C3,4,1) | KKH-SaBE3 | |
| | | | CCAGGACCGCCUGGAGCUGA | (CGGTG) | 20 (C−1) | St3BE3 | |
| | | | CAGCGGCCACCAGGACCGCC | (TGGAG) | 20 (C10,11,7,8) | St3BE3 | |
| Q31 | TAG | + | GGCGCCCGUGCGCAGGAGGA | (CGAG) | 20 (C13) | EQR-SpBE3 | 1133-1140 |
| (CAG) | | | GCGCCCGUGCGCAGGAGGAC | (GAG) | 20 (C12) | SpBE3 | |
| | | | CGCCCGUGCGCAGGAGGACG | (AGG) | 20 (C11) | SpBE3 | |
| | | | GCCCGUGCGCAGGAGGACGA | (GGAC) | 20 (C10) | VQR-SpBE3 | |
| | | | CGUGCGCAGGAGGACGAGGA | (CGG) | 20 (C7) | SpBE3 | |
| | | | GUGCGCAGGAGGACGAGGAC | (GGCG) | 20 (C6) | VRER-SpBE3 | |
| | | | GCGCAGGAGGACGAGGACGG | (CGAC) | 20 (C4) | VQR-SpBE3 | |
| | | | CGUGCGCAGGAGGACGAGGA | (CGGCG) | 20 (C7) | St3BE3 | |
| W77 | TAG or | + | CAGGCAACCUCCACGGAUCC | (TGG) | 20 (C11/12) | SpBE3 | 1141 |
| (TGG) | TGA | | | | | | |
| Q90 | TAG | + | GACCCACCUCUCGCAGUCAG | (AGCG) | 20 (C14*) | VRER-SpBE3 | 1142 |
| (CAG) | | | | | | | |
| Q99 | TAG | ++ with | UGCAGGCCCAGGCUGCCCGC | (CGG) | 20 (C3/9) | SpBE3 | 1143-1147 |
| (CAG) | | Q101X | GCAGGCCCAGGCUGCCCGCC | (GGG) | 20 (C2/8) | SpBE3 | |
| and/or | | | CAGGCCCAGGCUGCCCGCCG | (GGG) | 20 (C1/7) | SpBE3 | |
| Q101 | | | GCAGGCCCAGGCUGCCCGCC | (GGGGAT) | 20 (C2/8) | SaBE3 | |
| (CAG) | | | UGCAGGCCCAGGCUGCCCGC | (CGGGG) | 20 (C3/9) | St3BE3 | |
| Q101 | TAG | ++ with | AGGCCCAGGCUGCCCGCCGG | (GGAT) | 20 (C6) | EQR-SpBE3 | 1148 |
| (CAG) | | Q99X | | | | | |
| Q152 | TAG | ++ | UGUCUUUGCCCAGAGCAUCC | (CGTG) | 20 (C10) | VQR-SpBE3 | 1149-1153 |
| (CAG) | | | UCUUUGCCCAGAGCAUCCCG | (TGG) | 20 (C9) | SpBE3 | |
| | | | CUUUGCCCAGAGCAUCCCGU | (GGAA) | 20 (C7) | VQR-SpBE3 | |
| | | | CCAGAGCAUCCCGUGGAACC | (TGG) | 20 (C1) | SpBE3 | |
| | | | CCAGAGCAUCCCGUGGAACC | (TGGAG) | 20 (C1) | St3BE3 | |
| W156 | TAG or | + | CCACGGGAUGCUCUGGGCAA | (AGAC) | 20 (C1/2) | VQR-SpBE3 | 1154-1158 |
| (TGG) | TGA | | UCCACGGGAUGCUCUGGGCA | (AAG) | 20 (C2/3) | SpBE3 | |
| | | | CCAGGUUCCACGGGAUGCUC | (TGGG) | 20 (C8/9) | VQR-SpBE3 | |
| | | | CAGGUUCCACGGGAUGCUCU | (GGG) | 20 (C7/8) | SpBE3 | |
| | | | CCAGGUUCCACGGGAUGCUC | (TGG) | 20 (C8/9) | SpBE3 | |
| Q172 | TAG | ++ | GCGGAUGAAUACCAGCCCCC | (CGG) | 20 (C13) | SpBE3 | 1159-1161 |
| (CAG) | | | AUGAAUACCAGCCCCCCGGU | (AAG) | 20 (C9) | SpBE3 | |
| | | | UGAAUACCAGCCCCCCGGUA | (AGAC) | 20 (C8) | VQR-SpBE3 | |
| Q190 | TAG | ++ | CCAGCAUACAGAGUGACCAC | (CGG) | 20 (C9) | SpBE3 | 1162-1167 |
| (CAG) | | | CAGCAUACAGAGUGACCACC | (GGG) | 20 (C8) | SpBE3 | |
| | | | CCAGCAUACAGAGUGACCAC | (CGGG) | 20 (C7) | VQR-SpBE3 | |
| | | | AGCAUACAGAGUGACCACCG | (GGAA) | 20 (C7) | VQR-SpBE3 | |
| | | | CAGAGUGACCACCGGGAAAU | (CGAG) | 20 (C1) | EQR-SpBE3 | |
| | | | AGCAUACAGAGUGACCACCG | (GGAAAT) | 20 (C7) | KKH-SaBE3 | |
| Q219 | TAG | ++ | CUUCCACAGACAGGUAAGCA | (CGG) | 20 (C11) | SpBE3 | 1168-1170 |
| (CAG) | | | GACAGGUAAGCACGGCCGUC | (TGAT) | 20 (C3) | VQR-SpBE3 | |
| | | | CAGACAGGUAAGCACGGCCG | (TCTGAT) | 20 (C5) | KKH-SaBE3 | |
| Q256 | TAA | − | CGUGCUCAACUGCCAAGGGA | (AGG) | 20 (C14) | SpBE3 | 1171-1178 |
| (CAA) | | | GUGCUCAACUGCCAAGGGAA | (GGG) | 20 (C13) | SpBE3 | |
| | | | CGUGCUCAACUGCCAAGGGA | (AGGG) | 20 (C13) | VQR-SpBE3 | |
| | | | CAACUGCCAAGGGAAGGGCA | (CGG) | 20 (C8) | SpBE3 | |
| | | | UGCCAAGGGAAGGGCACGGU | (TAG) | 20 (C4) | SpBE3 | |
| | | | GCCAAGGGAAGGGCACGGUU | (AGCG) | 20 (C3) | VRER-SpBE3 | |
| | | | CAAGGGAAGGGCACGGUUAG | (CGG) | 20 (C1) | SpBE3 | |
| | | | CUCAACUGCCAAGGGAAGGG | (CACGGT) | 20 (C10) | KKH-SaBE3 | |
| Q275 | TAG | − | UUCGGAAAAGCCAGCUGGUC | (CAG) | 20 (C12) | SpBE3 | 1179-1182 |
| (CAG) | | | AAAAGCCAGCUGGUCCAGCC | (TGTG) | 20 (C7) | VQR-SpBE3 | |
| | | | AAGCCAGCUGGUCCAGCCUG | (TGG) | 20 (C5) | SpBE3 | |
| | | | AAGCCAGCUGGUCCAGCCUG | (TGGGG) | 20 (C5) | St3BE3 | |
| Q278 | TAG | + | AAGCCAGCUGGUCCAGCCUG | (TGG) | 20 (C14) | SpBE3 | 1183-1194 |
| (CAG) | | | AGCCAGCUGGUCCAGCCUGU | (GGG) | 20 (C13/4) | SpBE3 | |
| and/or | | | GCCAGCUGGUCCAGCCUGUG | (GGG) | 20 (C12/3) | SpBE3 | |
| Q275 | | | AGCCAGCUGGUCCAGCCUGU | (GGGG) | 20 (C13/4) | SpBE3 | |
| (CAG) | | | GGUCCAGCCUGUGGGGCCAC | (TGG) | 20 (C5) | SpBE3 | |
| | | | GUCCAGCCUGUGGGGCCACU | (GGTG) | 20 (C4) | VQR-SpBE3 | |
| | | | CCAGCCUGUGGGGCCACUGG | (TGG) | 20 (C2) | SpBE3 | |
| | | | CAGCCUGUGGGGCCACUGGU | (GGTG) | 20 (C1) | VQR-SpBE3 | |
| | | | CUGGUCCAGCCUGUGGGGCC | (ACTGGT) | 20 (C7) | KKH-SaBE3 | |
| | | | GUCCAGCCUGUGGGGCCACU | (GGTGGT) | 20 (C4) | KKH-SaBE3 | |
| | | | GGUCCAGCCUGUGGGGCCAC | (TGGTG) | 20 (C5) | St3BE3 | |
| | | | CCAGCCUGUGGGGCCACUGG | (TGGTG) | 20 (C2) | St3BE3 | |
| Q302 | TAG | − | CAACGCCGCCUGCCAGCGCC | (TGG) | 20 (C14) | SpBE3 | 1195-1205 |
| (CAG) | | | AACGCCGCCUGCCAGCGCCU | (GGCG) | 20 (C13) | VRER-SpBE3 | |
| | | | CGCCGCCUGCCAGCGCCUGG | (CGAG) | 20 (C11) | EQR-SpBE3 | |
| | | | GCCGCCUGCCAGCGCCUGGC | (GAG) | 20 (C10) | SpBE3 | |
| | | | CCGCCUGCCAGCGCCUGGCG | (AGG) | 20 (C9) | SpBE3 | |
| | | | CGCCUGCCAGCGCCUGGCGA | (GGG) | 20 (C8) | SpBE3 | |
| | | | UGCCAGCGCCUGGCGAGGGC | (TGG) | 20 (C4) | SpBE3 | |
| | | | GCCAGCGCCUGGCGAGGGCU | (GGG) | 20 (C3) | SpBE3 | |
| | | | CCAGCGCCUGGCGAGGGCUG | (GGG) | 20 (C2) | SpBE3 | |
| | | | UGCCAGCGCCUGGCGAGGGC | (TGGGGT) | 20 (C4) | SaBE3 | |
| | | | UGCCAGCGCCUGGCGAGGGC | (TGGGG) | 20 (C4) | St3BE3 | |
| Q342 | TAA | ++ with | CACCAAUGCCCAAGACCAGC | (CGG) | 20 (C11) | SpBE3 | 1206-1214 |
| (CAA) | and/or | Q344X | ACCAAUGCCCAAGACCAGCC | (GGTG) | 20 (C10) | VQR-SpBE3 | |
| and/or | TAG | | CAAUGCCCAAGACCAGCCGG | (TGAC) | 20 (C8) | VQR-SpBE3 | |
| Q344 | | | CCAAGACCAGCCGGUGACCC | (TGG) | 20 (C2/8) | SpBE3 | |
| (CAG) | | | CAAGACCAGCCGGUGACCCU | (GGG) | 20 (C1/7) | SpBE3 | |
| | | | CAAGACCAGCCGGUGACCCUG | (GGG) | 21 (C−1/6) | SpBE3 | |
| | | | GCCACCAAUGCCCAAGACCA | (GCCGGT) | 20 (C13) | KKH-SaBE3 | |
| | | | CACCAAUGCCCAAGACCAGC | (CGGTG) | 20 (C11) | St3BE3 | |
| | | | CCAAGACCAGCCGGUGACCC | (TGGGG) | 20 (C2/8) | St3BE3 | |
| Q344 | TAG | ++ with | AGACCAGCCGGUGACCCUGG | (GGAC) | 20 (C5) | VQR-SpBE3 | 1215 |
| (CAG) | | Q342X | | | | | |
| Q382 | TAG | − | CUGCUUUGUGUCACAGAGUG | (GGAC) | 20 (C14) | VQR-SpBE3 | 1216-1218 |
| (CAG) | | | UGUCACAGAGUGGGACAUCA | (CAG) | 20 (C6) | SpBE3 | |
| | | | GUCACAGAGUGGGACAUCAC | (AGG) | 20 (C5) | SpBE3 | |
| Q387 | TAG | − | ACAUCACAGGCUGCUGCCCA | (CGTG) | 20 (C7) | VQR-SpBE3 | 1219-1222 |
| (CAG) | | | AUCACAGGCUGCUGCCCACG | (TGG) | 20 (C5) | SpBE3 | |
| | | | CAGGCUGCUGCCCACGUGGC | (TGG) | 20 (C1) | SpBE3 | |
| | | | CACAGGCUGCUGCCCACGUG | (GCTGGT) | 20 (C3) | KKH-SaBE3 | |
| Q413 | TAG | | GGCCGAGUUGAGGCAGAGAC | (TGAT) | 20 (C14) | VQR-SpBE3 | 1223 |
| (CAG) | | | | | | | |
| W428 | TAG or | | AGGGAACCAGGCCUCAUUGA | (TGAC) | 20 (C7/8) | VQR-SpBE3 | 1224-1226 |
| (TGG) | TGA | | CUCAGGGAACCAGGCCUCAU | (TGAT) | 20 (C10/11) | VQR-SpBE3 | |
| | | | UCCUCAGGGAACCAGGCCUC | (ATTGAT) | 20 (C11/12) | KKH-SaBE3 | |
| Q433 | TAG | | CCCUGAGGACCAGCGGGUAC | (TGAC) | 20 (C11) | VQR-SpBE3 | 1227, 1228 |
| (CAG) | | | CAGCGGGUACUGACCCCCAA | (CCTGGT) | 20 (C1) | KKH-SaBE3 | |
| W453 | TAG or | ++ | CAGCUGCCAACCUGCAAAAA | (GGG) | 20 (C8/9) | SpBE3 | 1229-1235 |
| (TGG) | TGA | | GCCAACCUGCAAAAAGGGCC | (TGGG) | 20 (C2/3) | VQR-SpBE3 | |
| | | | GCCAACCUGCAAAAAGGGCC | (TGG) | 20 (C2/3) | SpBE3 | |
| | | | ACAGCUGCCAACCUGCAAAA | (AGGG) | 20 (C8/9) | VQR-SpBE3 | |
| | | | ACAGCUGCCAACCUGCAAAA | (AGG) | 20 (C8/9) | SpBE3 | |
| | | | AACAGCUGCCAACCUGCAAA | (AAG) | 20 (C9/10) | SpBE3 | |
| | | | GCCAACCUGCAAAAAGGGCC | (TGGGAT) | 20 (C2/3) | SaBE3 | |
| Q454 | TAG | ++ | GCAGGUUGGCAGCUGUUUUG | (CAG) | 20 (C10) | SpBE3 | 1236-1239 |
| (CAG) | | | CAGGUUGGCAGCUGUUUUGC | (AGG) | 20 (C9) | SpBE3 | |
| | | | AGGUUGGCAGCUGUUUUGCA | (GGAC) | 20 (C8) | VQR-SpBE3 | |
| | | | GCAGCUGUUUUGCAGGACUG | (TATGGT) | 20 (C2) | KKH-SaBE3 | |
| W461 | TAG or | − | GACCAUACAGUCCUGCAAAA | (CAG) | 20 (C3/4) | SpBE3 | 1240 |
| (TGG) | TGA | | | | | | |
| Q503 | TAG | + | UAAGGCCCAAGGGGGCAAGC | (TGG) | 20 (C8) | SpBE3 | 1241-1243 |
| (CAA) | | | ACUCUAAGGCCCAAGGGGGC | (AAG) | 20 (C12) | SpBE3 | |
| | | | UCUAAGGCCCAAGGGGGCAA | (GCTGGT) | 20 (C10) | KKH-SaBE3 | |
| Q531 | TAG | ++ with | CUGCUACCCCAGGCCAACUG | (CAG) | 20 (C10) | SpBE3 | 1244-1246 |
| (CAG) | | P530S | UGCUACCCCAGGCCAACUGC | (AGCG) | 20 (C9) | VQR-SpBE3 | |
| | | | CAGGCCAACUGCAGCGUCCACA | (CAG) | 22 (C−2) | SpBE3 | |
| Q554 | TAG | ++ with | CCAACAGGGCCACGUCCUCA | (CAG) | 20 (C2/5) | SpBE3 | 1247-1251 |
| (CAA) | and/or | Q555X | CAACAGGGCCACGUCCUCAC | (AGG) | 20 (C1/4) | SpBE3 | |
| and/or | TAA | | CAGGGCCACGUCCUCACAGG | (TAG) | 20 (C1) | SpBE3 | |
| Q555 | | | CAGGGCCACGUCCUCACAGGU | (AGG) | 21 (C−1) | SpBE3 | |
| (CAG) | | | ACCAACAGGGCCACGUCCUC | (ACAGGT) | 20 (C3/6) | KKH-SaBE3 | |
| W566 | TAG or | ++ | CCCAGUGGGAGCUGCAGCCU | (GGGG) | 20 (C2/3) | VQR-SpBE3 | 1252-1258 |
| (TGG) | TGA | | CCAGUGGGAGCUGCAGCCUG | (GGG) | 20 (C1/2) | SpBE3 | |
| | | | UCCCAGUGGGAGCUGCAGCC | (TGGG) | 20 (C3/4) | VQR-SpBE3 | |
| | | | CCCAGUGGGAGCUGCAGCCU | (GGG) | 20 (C2/3) | SpBE3 | |
| | | | UCCCAGUGGGAGCUGCAGCC | (TGG) | 20 (C3/4) | SpBE3 | |
| | | | CCACCUCCCAGUGGGAGCUG | (CAG) | 20 (C7/8) | SpBE3 | |
| | | | UCCCAGUGGGAGCUGCAGCC | (TGGGG) | 20 (C4/5) | St3BE3 | |
| R582 | TGA | ++ with | GGCCACGAGGUCAGCCCAAC | (CAG) | 20 (C12/6) | SpBE3 | 1259-1263 |
| (CGA) | and/or | P581S/L | GCCACGAGGUCAGCCCAACC | (AGTG) | 20 (C11/5) | VQR-SpBE3 | |
| and/or | TAG | | CACGAGGUCAGCCCAACCAG | (TGCG) | 20 (C9/3) | VRER-SpBE3 | |
| Q584 | | | CGAGGUCAGCCCAACCAGUG | (CGTG) | 20 (C6/1) | VQR-SpBE3 | |
| (CAG) | | | GAGGCCACGAGGUCAGCCCA | (ACCAGT) | 20 (C8) | KKH-SaBE3 | |
| Q584 | TAG | − | GGUCAGCCCAACCAGUGCGU | (GGG) | 20 (C4) | SpBE3 | 1264-1271 |
| (CAG) | | | AGGUCAGCCCAACCAGUGCG | (TGG) | 20 (C5) | SpBE3 | |
| | | | GGCCACGAGGUCAGCCCAAC | (CAG) | 20 (C12) | SpBE3 | |
| | | | GCCACGAGGUCAGCCCAACC | (AGTG) | 20 (C11) | VQR-SpBE3 | |
| | | | CACGAGGUCAGCCCAACCAG | (TGCG) | 20 (C9) | VRER-SpBE3 | |
| | | | CGAGGUCAGCCCAACCAGUG | (CGTG) | 20 (C7) | VQR-SpBE3 | |
| | | | AGGUCAGCCCAACCAGUGCG | (TGG) | 20 (C5) | SpBE3 | |
| | | | GGUCAGCCCAACCAGUGCGU | (GGG) | 20 (C4/13) | SpBE3 | |
| Q587 | TAG | − | CCCAACCAGUGCGUGGGCCA | (CAG) | 20 (C7) | SpBE3 | 1272-1278 |
| (CAG) | | | CCAGUGCGUGGGCCACAGGG | (AGG) | 20 (C2) | SpBE3 | |
| | | | ACCAGUGCGUGGGCCACAGG | (GAG) | 20 (C3) | SpBE3 | |
| | | | AACCAGUGCGUGGGCCACAG | (GGAG) | 20 (C4) | EQR-SpBE3 | |
| | | | CAACCAGUGCGUGGGCCACA | (GGG) | 20 (C5) | SpBE3 | |
| | | | CCAACCAGUGCGUGGGCCAC | (AGG) | 20 (C6) | SpBE3 | |
| | | | CAACCAGUGCGUGGGCCACA | (GGGAG) | 20 (C5) | St3BE3 | |
| Q619 | TAG | ++ with | CAGGAGCAGGUGAAGAGGCC | (CGTG) | 20 (C1) | VQR-SpBE3 | 1279-1284 |
| (CAG) | | P618S | CCCCUCAGGAGCAGGUGAAG | (AGG) | 20 (C6) | SpBE3 | |
| | | | GCCCCUCAGGAGCAGGUGAA | (GAG) | 20 (C7) | SpBE3 | |
| | | | GGCCCCUCAGGAGCAGGUGA | (AGAG) | 20 (C8) | EQR-SpBE3 | |
| | | | CGGCCCCUCAGGAGCAGGUG | (AAG) | 20 (C9) | SpBE3 | |
| | | | CCCGGCCCCUCAGGAGCAGG | (TGAA) | 20 (C11) | VQR-SpBE3 | |
| Q621 | TAG | ++ | GGCCCCUCAGGAGCAGGUGA | (AGAG) | 20 (C14) | EQR-SpBE3 | 1285-1292 |
| (CAG) | | | GCCCCUCAGGAGCAGGUGAA | (GAG) | 20 (C13) | SpBE3 | |
| | | | CCCCUCAGGAGCAGGUGAAG | (AGG) | 20 (C12) | SpBE3 | |
| | | | CAGGAGCAGGUGAAGAGGCC | (CGTG) | 20 (C7) | VQR-SpBE3 | |
| | | | GGAGCAGGUGAAGAGGCCCG | (TGAG) | 20 (C5) | EQR-SpBE3 | |
| | | | GAGCAGGUGAAGAGGCCCGU | (GAG) | 20 (C4) | SpBE3 | |
| | | | AGCAGGUGAAGAGGCCCGUG | (AGG) | 20 (C3) | SpBE3 | |
| | | | CAGGUGAAGAGGCCCGUGAGG | (CCGGGT) | 21 (C−1) | SaBE3 | |
| W630 | TGA | + | CCAGCCCUCCUCGCAGGCCA | (CGG) | 20 (C1/2) | SpBE3 | 1293-1296 |
| (TGG) | | | CAGGGUCCAGCCCUCCUCGC | (AGG) | 20 (C7/8) | SpBE3 | |
| | | | UCAGGGUCCAGCCCUCCUCG | (CAG) | 20 (C8/9) | SpBE3 | |
| | | | GUCCAGCCCUCCUCGCAGGC | (CACGGT) | 20 (C3/4) | KKH-SaBE3 | |
| Q686 | TAG | − | GGCACCUGGCGCAGGCCUCC | (CAG) | 20 (C12) | SpBE3 | 1297-1305 |
| (CAG) | | | GCACCUGGCGCAGGCCUCCC | (AGG) | 20 (C11) | SpBE3 | |
| | | | CACCUGGCGCAGGCCUCCCA | (GGAG) | 20 (C10) | EQR-SpBE3 | |
| | | | ACCUGGCGCAGGCCUCCCAG | (GAG) | 20 (C9) | SpBE3 | |
| | | | CGCAGGCCUCCCAGGAGCUC | (CAG) | 20 (C3) | SpBE3 | |
| | | | GCAGGCCUCCCAGGAGCUCC | (AGTG) | 20 (C2) | VQR-SpBE3 | |
| | | | CAGGCCUCCCAGGAGCUCCAG | (TGAC) | 21 (C−1) | VQR-SpBE3 | |
| | | | GGCGCAGGCCUCCCAGGAGC | (TCCAGT) | 20 (C5) | SaBE3 | |
| | | | GCACCUGGCGCAGGCCUCC | (CAGGAG) | 19 (C11) | St3BE3 | |
| Q689 | TAG | − | CCUCCCAGGAGCUCCAGUGA | (CAG) | 20 (C6) | SpBE3 | 1306-1309 |
| (CAG) | | | AGGCCUCCCAGGAGCUCCAG | (TGAC) | 20 (C9) | VQR-SpBE3 | |
| | | | GCAGGCCUCCCAGGAGCUCC | (AGTG) | 20 (C11) | VQR-SpBE3 | |
| | | | CGCAGGCCUCCCAGGAGCUC | (CAG) | 20 (C12) | SpBE3 | |

*Guide sequences (the portion of the guide RNA that targets the nucleobase editor to the target sequence) are provided, which may be used with any tracrRNA framework sequences provided herein to generate the full guide RNA sequence
^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.

Scoring of Guide RNA Sequences for Efficient Base Editing with High Specificity and Low Off-Target Binding

To achieve efficient and specific genome modifications using base editing requires judicious selection of a genomic sequence containing a target C, for which a specific complementary guide RNA sequence can be generated, and if required, a nearby PAM that matches the DNA-binding domain that is fused to the cytidine deaminase (e.g. Cas9, dCas9, Cas9n, Cpf1, NgAgo, etc.), as described in Komor et al., Nature, 533, 420-424 (2016), which is incorporated herein by reference. The guide RNA sequence and PAM preference define the genomic target sequence(s) of programable DNA-binding domains (e.g. Cas9, dCas9, Cas9n, Cpf1, NgAgo, etc.). Because of the repetitive nature of some genomic sequences as well as the stochastic frequency of representation of short sequences throughout the genome it is necessary to identify guide RNAs for programming base editors that have the lowest number of potential off target sites, taking into consideration 1, 2, 3, 4 or more mismatches against all other sequences in the genome as described in Hsu et al (Nature biotechnology, 2013, 31(9):827-832), Fusi et al (bioRxiv 021568; doi: http://dx.doi.org/10.1101/021568), Chari et al (Nature Methods, 2015, 12(9):823-6), Doench et al (Nature Biotechnology, 2014, 32(12):1262-7), Wang et al (Science, 2014, 343(6166): 80-4), Moreno-Mateos et al (Nature Methods, 2015, 12(10):982-8), Housden et al (Science Signaling, 2015, 8(393):rs9), Haeussler et al, (Genome Biol. 2016; 17: 148), each of which is incorporated herein by reference, The potential for the formation of bulges between the guide RNA and the target DNA may also be considered as described in Bae et al (Bioinformatics, 2014, 30, 1473-5), which is incorporated herein by reference. Non-limiting examples of calculated specificity scores for selected guide RNAs from Tables 3-8 are shown in Tables 9-13. Other calculated parameters that may influence DNA-binding domains programming efficiency are shown, as described in Housden et al (Science Signaling, 2015, 8(393):rs9), Farboud et al (Genetics, 2015, 199(4):959-71), each of which is incorporated herein by reference.

TABLE 9
Efficiency and Specificity Scores for gRNAs for PCSK9 Protective Loss-of-Function Mutations
via Codon Change. Guide sequences correspond to SEQ ID NOs: 1310-1437 from top to bottom.

| | | | | gRNA | | | | | | | | | | |
|---|---|---|---|---|---|---|---|---|---|---|---|---|---|---|
| Target | | | | size | | | | | | | | | | |
| vari- | BE | guide | | (C | | | | | | | | | Prox/ | Off- |
| ants | type^{a} | sequence | PAM | edited) | Eff.^{b} | Hsu^{c} | Fusi | Chari | Doench | Wang | M.-M. | Housden | GC | targets^{d} |
| R194W | SaBE3 | GACCACCGGGA | (CAGG | 20 (C7) | 7.0 | 99 | — | 98 | 11 | 86 | 60 | 7 | +GG | 0-0-0- |
| | | AAUCGAGGG | GT) | | | | | | | | | | | 1-10 |
| H193Y | SaBE3 | GACCACCGGGA | (CAGG | 20 (C4) | 7.0 | 99 | — | 98 | 11 | 86 | 60 | 7 | +GG | 0-0-0- |
| | | AAUCGAGGG | GT) | | | | | | | | | | | 1-10 |
| R237R | VQR- | GUCAGCGGCCG | (CGTG) | 20 (C10) | 7.4 | 98 | — | 95 | 3 | 83 | 75 | 7 | +GG | 0-0-0- |
| | SpBE3 | GGAUGCCGG | | | | | | | | | | | | 1-18 |
| R194W | SpBE3 | GACCACCGGGA | (CAG) | 20 (C7) | 7.0 | 93 | 59 | 98 | 14 | 86 | 60 | 7 | +GG | 0-0-1- |
| | | AAUCGAGGG | | | | | | | | | | | | 4-41 |
| L253F | EQR- | GCGCGUGCUCA | (GGAA) | 20 (C8) | 9.1 | 90 | — | 97 | 83 | 77 | 74 | 9 | + | 0-0-0- |
| | SpBE3 | ACUGCCAAG | | | | | | | | | | | | 4-36 |
| A220V | VQR- | UCGUCGAGCA | (TGTG) | 20 (C13) | 4.5 | 100 | — | 87 | 16 | 67 | 54 | 4 | − | 0-0-0- |
| | SpBE3 | GGCCAGCAAG | | | | | | | | | | | | 0-2 |
| R46L | SpBE3 | GCUAGCCUUG | (AGG) | 20 (C11) | 6.4 | 90 | 63 | 94 | 21 | 81 | 80 | 6 | +GG | 0-0-2- |
| | | CGUUCCGAGG | | | | | | | | | | | | 0-35 |
| A68T | KKH- | CGCACCUUGGC | (GAAG | 20 (C11) | 5.1 | 98 | — | 85 | 2 | 48 | 53 | 5 | + | 0-0-0- |
| | SaBE3 | GCAGCGGUG | GT) | | | | | | | | | | | 0-10 |
| P616L | KKH- | GGAAUCCCGGC | (GCAG | 20 | 4.0 | 94 | — | 86 | 23 | 87 | 53 | 4 | − | 0-0-0- |
| | SaBE3 | CCCUCAGGA | GT) | (C6/7) | | | | | | | | | | 1-26 |
| R194W | SpBE3 | AGUGACCACCG | (GGG) | 20 (C10) | 7.3 | 92 | 65 | 88 | 66 | 80 | 54 | 7 | − | 0-0-0- |
| | | GGAAAUCGA | | | | | | | | | | | | 2-45 |
| H193Y | SpBE3 | AGUGACCACCG | (GGG) | 20 (C7) | 7.3 | 92 | 65 | 88 | 66 | 80 | 54 | 7 | − | 0-0-0- |
| | | GGAAAUCGA | | | | | | | | | | | | 2-45 |
| H193Y | SpBE3 | ACCACCGGGAA | (AGG) | 20 (C3) | 5.9 | 92 | 65 | 88 | 66 | 80 | 54 | 7 | − | 0-0-0- |
| | | AUCGAGGGC | | | | | | | | | | | | 2-45 |
| A443T | KKH- | GGGCGGCCACC | (GTCA | 20 (C4) | 6.4 | 90 | — | 88 | 14 | 90 | 77 | 6 | +GG | 0-0-0- |
| | SaBE3 | AGGUUGGGG | GT) | | | | | | | | | | | 4-36 |
| G263S | KKH- | CGCUAACCGUG | (GGCA | 21 (C−1) | 5.9 | 94 | 47 | 86 | 47 | 57 | 59 | 5 | − | 0-0-0- |
| | SaBE3 | CCCUUCCCUU | GT) | | | | | | | | | | | 2-20 |
| M1I | St3BE3 | ACGGUGCCCAU | (GGGA | 20 (C9) | 5.1 | 87 | 59 | 81 | 10 | 77 | 92 | 5 | + | 0-0-2- |
| | | GAGGGCCAG | G) | | | | | | | | | | | 3-29 |
| A220T | VQR- | GGCCUGCUCGA | (GGAC) | 20 (C3) | 4.5 | 90 | — | 86 | 88 | 79 | 57 | 4 | − | 0-0-0- |
| | SpBE3 | CGAACACAA | | | | | | | | | | | | 3-43 |
| R46L | SpBE3 | UGCUAGCCUU | (GAG) | 20 (C12) | 6.6 | 97 | 64 | 81 | 56 | 63 | 44 | 6 | + | 0-0-0- |
| | | GCGUUCCGAG | | | | | | | | | | | | 2-26 |
| A68T | VQR- | CCGCACCUUGG | (GGAA) | 20 (C12) | 5.2 | 93 | — | 39 | 4 | 45 | 85 | 5 | + | 0-0-0- |
| | SpBE3 | CGCAGCGGU | | | | | | | | | | | | 5-28 |
| A68T | St3BE3 | CACCUUGGCGC | (AGGT | 20 (C9) | 4.9 | 95 | 46 | 83 | 2 | 33 | 57 | 4 | + | 0-0-0- |
| | | AGCGGUGGA | G) | | | | | | | | | | | 2-33 |
| H226 | St3BE3 | UCAUGGCACCC | (GGGT | 20 (C2) | 6.0 | 84 | 58 | 93 | 38 | 80 | 61 | 6 | + | 0-0-0- |
| | | ACCUGGCAG | G) | | | | | | | | | | | 6-60 |
| R237R | St3BE3 | CGGGAUGCCGG | (GGGT | 20 (C1) | 7.6 | 91 | 41 | 60 | 10 | 62 | 85 | 7 | + | 0-0-0- |
| | | CGUGGCCAA | G) | | | | | | | | | | | 3-15 |
| R237Q | St3BE3 | CGGGAUGCCGG | (GGGT | 20 (C1) | 7.6 | 91 | 41 | 60 | 10 | 62 | 85 | 7 | + | 0-0-0- |
| | | CGUGGCCAA | G) | | | | | | | | | | | 3-15 |
| S386 | KKH- | CACAGGCUGCU | (GCTG | 20 (C1) | 7.7 | 95 | — | 81 | 4 | 56 | 73 | 7 | + | 0-0-0- |
| | SaBE3 | GCCCACGUG | GT) | | | | | | | | | | | 3-23 |
| H226 | SaBE3 | AGUCAUGGCA | (AGGG | 20 (C4) | 4.9 | 91 | 49 | 85 | 4 | 49 | 50 | 4 | + | 0-0-0- |
| | | CCCACCUGGC | GT) | | | | | | | | | | | 0-31 |
| A220T | VQR- | ACACUUGCUG | (CGAA) | 20 (C12) | 5.8 | 91 | — | 84 | 40 | 69 | 56 | 5 | + | 0-0-0- |
| | SpBE3 | GCCUGCUCGA | | | | | | | | | | | | 0-85 |
| R46L | EQR- | GUGCUAGCCU | (GGAG) | 20 (C13) | 3.6 | 98 | — | 33 | 35 | 76 | 58 | 3 | − | 0-0-0- |
| | SpBE3 | UGCGUUCCGA | | | | | | | | | | | | 1-23 |
| H391W | KKH- | GGCUGCUGCCC | (GTAA | 20 (C11) | 5.9 | 91 | — | 82 | 17 | 70 | 48 | 5 | + | 0-0-0- |
| (Y) | SaBE3 | ACGUGGCUG | GT) | | | | | | | | | | | 8-36 |
| A68T | SpBE3 | CCCGCACCUUG | (TGG) | 20 (C13) | 4.3 | 89 | 50 | 70 | 16 | 83 | 64 | 4 | +GG | 0-0-0- |
| | | GCGCAGCGG | | | | | | | | | | | | 4-76 |
| R194W | SpBE3 | GAGUGACCACC | (AGG) | 20 (C11) | 6.2 | 93 | 62 | 76 | 14 | 79 | 36 | 6 | − | 0-0-0- |
| | | GGGAAAUCG | | | | | | | | | | | | 3-38 |
| H193Y | SpBE3 | GAGUGACCACC | (AGG) | 20 (C8) | 6.2 | 93 | 62 | 76 | 14 | 79 | 36 | 6 | − | 0-0-0- |
| | | GGGAAAUCG | | | | | | | | | | | | 3-38 |
| E49K | SpBE3 | GCCGUCCUCCU | (AGG) | 20 (C9) | 7.0 | 94 | 53 | 78 | 24 | 62 | 50 | 7 | − | 0-0-1- |
| | | CGGAACGCA | | | | | | | | | | | | 1-28 |
| R29C | EQR- | CCCGCGGGCGC | (GGAG) | 20 (C13) | 4.3 | 92 | — | 80 | 3 | 44 | 69 | 4 | + | 0-0-0- |
| | SpBE3 | CCGUGCGCA | | | | | | | | | | | | 3-35 |
| A68T | SpBE3 | CACCUUGGCGC | (AGG) | 20 (C9) | 4.9 | 88 | 46 | 83 | 2 | 33 | 57 | 4 | + | 0-0-0- |
| | | AGCGGUGGA | | | | | | | | | | | | 8-73 |
| A53V | EQR- | UGGCCGAAGC | (GGAA) | 20 (C4) | 8.0 | 94 | — | 60 | 10 | 76 | 67 | 8 | + | 0-0-0- |
| | SpBE3 | ACCCGAGCAC | | | | | | | | | | | | 1-50 |
| H226 | St3BE3 | AGUCAUGGCA | (AGGG | 20 (C4) | 4.9 | 85 | 49 | 85 | 4 | 49 | 50 | 4 | + | 0-0-0- |
| | | CCCACCUGGC | G) | | | | | | | | | | | 1-54 |
| R194W | SpBE3 | ACCACCGGGAA | (AGG) | 20 (C6) | 5.9 | 94 | 52 | 75 | 0 | 73 | 39 | 5 | + | 0-0-0- |
| | | AUCGAGGGC | | | | | | | | | | | | 1-48 |
| H193Y | SpBE3 | CCACCGGGAAA | (GGG) | 20 (C2) | 4.5 | 94 | 52 | 75 | 0 | 73 | 39 | 5 | + | 0-0-0- |
| | | UCGAGGGCA | | | | | | | | | | | | 1-48 |
| C375Y | VQR- | GCAGUCGCUG | (TGAT) | 20 (C2) | 5.4 | 83 | — | 85 | 32 | 84 | 80 | 5 | − | 0-0-0- |
| | SpBE3 | GAGGCACCAA | | | | | | | | | | | | 5-89 |
| R237R | SpBE3 | CGGGAUGCCGG | (GGG) | 20 (C1) | 7.6 | 83 | 41 | 60 | 10 | 62 | 85 | 7 | + | 0-0-0- |
| | | CGUGGCCAA | | | | | | | | | | | | 4-50 |
| R237Q | SpBE3 | CGGGAUGCCGG | (GGG) | 20 (C1) | 7.6 | 83 | 41 | 60 | 10 | 62 | 85 | 7 | + | 0-0-0- |
| | | CGUGGCCAA | | | | | | | | | | | | 4-50 |
| S47F | SpBE3 | GCCUUGCGUU | (CGG) | 20 (C6) | 4.4 | 82 | 68 | 85 | 27 | 68 | 49 | 4 | + | 0-0-0- |
| | | CCGAGGAGGA | | | | | | | | | | | | 3-75 |
| R46L | SpBE3 | GCCUUGCGUU | (CGG) | 20 (C7) | 4.4 | 82 | 68 | 85 | 27 | 68 | 49 | 4 | + | 0-0-0- |
| | | CCGAGGAGGA | | | | | | | | | | | | 3-75 |
| R46L | SpBE3 | GCCUUGCGUU | (CGG) | 20 (C7) | 4.4 | 82 | 68 | 85 | 27 | 68 | 49 | 4 | + | 0-0-0- |
| | | CCGAGGAGGA | | | | | | | | | | | | 3-75 |
| A53V | SpBE3 | CUGGCCGAAGC | (CGG) | 20 (C5) | 4.4 | 88 | 58 | 79 | 4 | 53 | 61 | 4 | + | 0-0-0- |
| | | ACCCGAGCA | | | | | | | | | | | | 3-87 |
| R46H | SpBE3 | UCGGAACGCA | (CAG) | 20 (C7) | 5.1 | 90 | 63 | 24 | 32 | 77 | 63 | 5 | − | 0-0-0- |
| | | AGGCUAGCAC | | | | | | | | | | | | 4-25 |
| R29C | VRER- | CGUGCGCAGGA | (GGCG) | 21 (C−1) | 5.9 | 98 | — | 53 | 2 | 60 | 68 | 5 | + | 0-0-0- |
| | SpBE3 | GGACGAGGAC | | | | | | | | | | | | 0-17 |
| G452D | SaBE3 | GCCAACCUGCA | (TGGG | 20 (C6) | 7.2 | 95 | 37 | 53 | 11 | 71 | 10 | 7 | + | 0-0-0- |
| | | AAAAGGGCC | AT) | | | | | | | | | | | 0-34 |
| R194W | KKH- | CGGGAAAUCG | (CATG | 20 (C1) | 5.9 | 93 | — | 13 | 6 | 69 | 73 | 5 | + | 0-0-0- |
| | SaBE3 | AGGGCAGGGU | GT) | | | | | | | | | | | 2-26 |
| A443T | St3BE3 | GGGCAGGGCGG | (TGGG | 20 (C9) | 4.2 | 79 | 34 | 82 | 3 | 76 | 85 | 4 | + | 0-0-1- |
| | | CCACCAGGU | G) | | | | | | | | | | | 13-127 |
| R237R | VRER- | UGGUCAGCGG | (GGCG) | 20 (C12) | 6.7 | 98 | — | 41 | 1 | 23 | 66 | 6 | + | 0-0-0- |
| | SpBE3 | CCGGGAUGCC | | | | | | | | | | | | 1-8 |
| R237Q | VRER- | UGGUCAGCGG | (GGCG) | 20 (C12) | 6.7 | 98 | — | 41 | 1 | 23 | 66 | 6 | + | 0-0-0- |
| | SpBE3 | CCGGGAUGCC | | | | | | | | | | | | 1-8 |
| R46L | SpBE3 | GCGUUCCGAG | (TGG) | 20 (C2) | 4.8 | 85 | 48 | 78 | 13 | 72 | 43 | 4 | + | 0-0-0- |
| | | GAGGACGGCC | | | | | | | | | | | | 5-58 |
| S47F | SpBE3 | GCGUUCCGAG | (TGG) | 20 (C5) | 4.8 | 85 | 48 | 78 | 13 | 72 | 43 | 4 | + | 0-0-0- |
| | | GAGGACGGCC | | | | | | | | | | | | 5-58 |
| A220V | KKH- | UCGAGCAGGCC | (GACA | 20 (C10) | 7.7 | 89 | — | 41 | 12 | 66 | 73 | 7 | − | 0-0-1- |
| | SaBE3 | AGCAAGUGU | GT) | | | | | | | | | | | 0-20 |
| A443T | SaBE3 | GGCAGGGCGGC | (GGGG | 20 (C7) | 5.5 | 84 | 24 | 28 | 0 | 58 | 78 | 5 | − | 0-0-0- |
| | | CACCAGGUU | GT) | | | | | | | | | | | 4-64 |
| L253F | SpBE3 | CGUGCUCAAC | (AGG) | 20 (C5) | 6.0 | 78 | 52 | 73 | 6 | 84 | 39 | 6 | − | 0-0-0- |
| | | UGCCAAGGGA | | | | | | | | | | | | 7-82 |
| A68T | KKH- | GCGCAGCGGUG | (TGTG | 20 (C2) | 5.5 | 91 | 27 | 71 | 1 | 44 | 53 | 5 | + | 0-0-0- |
| | SaBE3 | GAAGGUGGC | GT) | | | | | | | | | | | 2-37 |
| R29C | VQR- | GCGGGCGCCCG | (GGAC) | 20 (C10) | 7.5 | 83 | — | 78 | 29 | 78 | 67 | 7 | + | 0-0-1- |
| | SpBE3 | UGCGCAGGA | | | | | | | | | | | | 13-60 |
| A220T | SpBE3 | UGGCCUGCUCG | (AGG) | 20 (C4) | 6.0 | 88 | 56 | 73 | 21 | 62 | 49 | 6 | − | 0-0-0- |
| | | ACGAACACA | | | | | | | | | | | | 6-49 |
| E49K | SpBE3 | GGCCGUCCUCC | (AAG) | 20 (C10) | 6.0 | 96 | 46 | 53 | 5 | 65 | 30 | 6 | + | 0-0-0- |
| | | UCGGAACGC | | | | | | | | | | | | 1-27 |
| R93C | SpBE3 | AGCGCACUGCC | (CAG) | 20 (C3) | 8.7 | 78 | 36 | 83 | 2 | 59 | 67 | 8 | + | 0-0-1- |
| | | CGCCGCCUG | | | | | | | | | | | | 9-104 |
| L253F | SpBE3 | GCGUGCUCAAC | (AAG) | 20 (C6) | 4.8 | 75 | 54 | 80 | 16 | 84 | 63 | 4 | +GG | 0-0-0- |
| | | UGCCAAGGG | | | | | | | | | | | | 5-93 |
| S153N | SaBE3 | AGCAUCCCGUG | (GCGG | 20 (C3) | 5.4 | 93 | — | 66 | 20 | 51 | 53 | 5 | + | 0-0-0- |
| | | GAACCUGGA | AT) | | | | | | | | | | | 3-21 |
| R29C | VQR- | GCCCGUGCGCA | (GGAC) | 20 (C4) | 7.7 | 81 | — | 76 | 28 | 77 | 60 | 7 | + | 0-0-0- |
| | SpBE3 | GGAGGACGA | | | | | | | | | | | | 4-91 |
| R29C | EQR- | GGCGCCCGUGC | (CGAG) | 20 (C7) | 4.0 | 68 | — | 90 | 6 | 70 | 62 | 4 | + | 0-0-2- |
| | SpBE3 | GCAGGAGGA | | | | | | | | | | | | 11-115 |
| S373N, | KKH- | GUGCUGCAGU | (ACCA | 20 | 6.6 | 90 | — | 68 | 4 | 64 | 62 | 6 | + | 0-0-0- |
| D374N | SaBE3 | CGCUGGAGGC | AT) | (C11/7) | | | | | | | | | | 3-30 |
| S153N | SpBE3 | AGAGCAUCCCG | (GAG) | 20 (C5) | 7.1 | 75 | 59 | 71 | 19 | 83 | 72 | 7 | − | 0-0-2- |
| | | UGGAACCUG | | | | | | | | | | | | 9-100 |
| R29C | St3BE3 | CGUGCGCAGGA | (CGGC | 20 (C1) | 6.7 | 76 | 58 | 81 | 27 | 73 | 70 | 6 | + | 0-0-0- |
| | | GGACGAGGA | G) | | | | | | | | | | | 4-127 |
| R237R | SpBE3 | CAGCGGCCGGG | (TGG) | 20 (C8) | 5.3 | 77 | 58 | 80 | 3 | 74 | 78 | 5 | + | 0-0-0- |
| | | AUGCCGGCG | | | | | | | | | | | | 15-170 |
| R237Q | SpBE3 | CAGCGGCCGGG | (TGG) | 20 (C8) | 5.3 | 77 | 58 | 80 | 3 | 74 | 78 | 5 | + | 0-0-0- |
| | | AUGCCGGCG | | | | | | | | | | | | 15-170 |
| T77I | SaBE3 | GCAGCACCUGC | (CAGA | 20 (C7) | 5.6 | 90 | — | 19 | 28 | 66 | 47 | 5 | − | 0-0-1- |
| | | UUUGUGUCA | GT) | | | | | | | | | | | 0-35 |
| T377I | SaBE3 | GCAGCACCUGC | (CAGA | 20 (C7) | 5.6 | 90 | — | 19 | 28 | 66 | 47 | 5 | − | 0-0-1- |
| | | UUUGUGUCA | GT) | | | | | | | | | | | 0-35 |
| C378Y | St3BE3 | AAAGCAGGUG | (TGGA | 20 (C5) | 5.1 | 86 | 43 | 39 | 1 | 70 | 61 | 5 | + | 0-0-1- |
| | | CUGCAGUCGC | G) | | | | | | | | | | | 11-50 |
| S376N | St3BE3 | AAAGCAGGUG | (TGGA | 20 (C13) | 5.1 | 86 | 43 | 39 | 1 | 70 | 61 | 5 | + | 0-0-1- |
| | | CUGCAGUCGC | G) | | | | | | | | | | | 11-50 |
| A220T | SpBE3 | CUGGCCUGCUC | (AAG) | 20 (C5) | 4.5 | 98 | 48 | 43 | 8 | 55 | 57 | 4 | − | 0-0-0- |
| | | GACGAACAC | | | | | | | | | | | | 2-29 |
| A68T | VQR- | ACCUUGGCGCA | (GGTG) | 20 (C8) | 7.5 | 97 | — | 30 | 10 | 58 | 55 | 7 | − | 0-0-0- |
| | SpBE3 | GCGGUGGAA | | | | | | | | | | | | 1-1 |
| M1I | EQR- | CGGUGCCCAUG | (GGAG) | 20 (C8) | 6.2 | 57 | — | 97 | 33 | 65 | 68 | 6 | +GG | 0-0-6- |
| | SpBE3 | AGGGCCAGG | | | | | | | | | | | | 18-117 |
| P12L | EQR- | AGCGGCCACCA | (GGAG) | 20 (C6) | 8.2 | 82 | — | 51 | 2 | 72 | 57 | 8 | + | 0-0-1- |
| | SpBE3 | GGACCGCCU | | | | | | | | | | | | 9-94 |
| A443T | St3BE3 | GGCAGGGCGGC | (GGGG | 20 (C8) | 5.5 | 76 | 24 | 28 | 0 | 58 | 78 | 5 | − | 0-0-0- |
| | | CACCAGGUU | G) | | | | | | | | | | | 7-131 |
| E57K | SpBE3 | CGUGCUCGGG | (AGG) | 20 (C7) | 7.1 | 94 | 48 | 53 | 3 | 60 | 50 | 7 | + | 0-0-0- |
| | | UGCUUCGGCC | | | | | | | | | | | | 2-33 |
| R194W | SpBE3 | CCACCGGGAAA | (GGG) | 20 (C5) | 4.5 | 83 | 59 | 63 | 31 | 70 | 66 | 4 | + | 0-0-1- |
| | | UCGAGGGCA | | | | | | | | | | | | 9-66 |
| A53V | SpBE3 | ACGGCCUGGCC | (GAG) | 20 (C10) | 6.9 | 77 | 60 | 76 | 6 | 72 | 60 | 6 | + | 0-0-2- |
| | | GAAGCACCC | | | | | | | | | | | | 11-91 |
| L253F | SpBE3 | UGCGCGUGCUC | (GGG) | 20 (C9) | 3.7 | 85 | 52 | 67 | 50 | 60 | 53 | 3 | − | 0-0-1- |
| | | AACUGCCAA | | | | | | | | | | | | 25-90 |
| G27D | EQR- | ACGGGCGCCCG | (GGAG) | 20 (C8) | 8.3 | 71 | — | 81 | 7 | 72 | 76 | 8 | + | 0-0-1- |
| | SpBE3 | CGGGACCCA | | | | | | | | | | | | 16-40 |
| S386 | SpBE3 | AUCACAGGCU | (TGG) | 20 (C3) | 5.1 | 61 | 59 | 91 | 16 | 43 | 70 | 5 | + | 0-0-3- |
| | | GCUGCCCACG | | | | | | | | | | | | 13-177 |
| G27D | St3BE3 | CACGGGCGCCC | (AGGA | 20 (C9) | 6.3 | 87 | 35 | 65 | 1 | 43 | 59 | 6 | + | 0-0-0- |
| | | GCGGGACCC | G) | | | | | | | | | | | 1-52 |
| R237R | SaBE3 | GCCGGGAUGCC | (AAGG | 20 (C3) | 7.8 | 96 | — | 43 | 2 | 54 | 55 | 7 | + | 0-0-0- |
| | | GGCGUGGCC | GT) | | | | | | | | | | | 0-17 |
| R237Q | SaBE3 | GCCGGGAUGCC | (AAGG | 20 (C3) | 7.8 | 96 | — | 43 | 2 | 54 | 55 | 7 | + | 0-0-0- |
| | | GGCGUGGCC | GT) | | | | | | | | | | | 0-17 |
| M1I | EQR- | GUGCCCAUGA | (AGAG) | 20 (C6) | 6.2 | 57 | — | 92 | 9 | 88 | 79 | 6 | +GG | 0-0-0- |
| | SpBE3 | GGGCCAGGGG | | | | | | | | | | | | 23-227 |
| R194Q | St3BE3 | CCGGUGGUCAC | (TGGT | 20 (C2) | 6.4 | 95 | 50 | 10 | 9 | 54 | 42 | 6 | − | 0-0-0- |
| | | UCUGUAUGC | G) | | | | | | | | | | | 1-17 |
| R237Q | St3BE3 | GUGGUCAGCG | (CGGC | 20 (C13) | 5.0 | 89 | 40 | 54 | 2 | 49 | 60 | 5 | + | 0-0-0- |
| | | GCCGGGAUGC | G) | | | | | | | | | | | 5-55 |
| R29C | SpBE3 | CGCCCGUGCGC | (AGG) | 20 (C5) | 4.4 | 64 | 43 | 85 | 10 | 60 | 49 | 4 | + | 0-0-1- |
| | | AGGAGGACG | | | | | | | | | | | | 15-154 |
| S153N | St3BE3 | CCAGAGCAUCC | (TGGA | 20 (C7) | 8.6 | 90 | 45 | 59 | 3 | 41 | 32 | 8 | + | 0-0-1- |
| | | CGUGGAACC | G) | | | | | | | | | | | 2-68 |
| M1I | SpBE3 | ACGGUGCCCAU | (GGG) | 20 (C9) | 5.1 | 54 | 59 | 81 | 10 | 77 | 92 | 5 | + | 0-0-6- |
| | | GAGGGCCAG | | | | | | | | | | | | 24-136 |
| D186 | SpBE3 | CUAGGAGAUA | (AGG) | 20 (C1) | 4.3 | 75 | 63 | 66 | 70 | 66 | 39 | 4 | + | 0-0-0- |
| | | CACCUCCACC | | | | | | | | | | | | 14-90 |
| H193Y | EQR- | CAGAGUGACC | (CGAG) | 20 (C10) | 7.6 | 83 | — | 40 | 3 | 31 | 62 | 7 | − | 0-0-0- |
| | SpBE3 | ACCGGGAAAU | | | | | | | | | | | | 7-134 |
| G452D | SpBE3 | CCAACCUGCAA | (GGG) | 20 (C5) | 4.9 | 69 | 46 | 68 | 41 | 75 | 39 | 4 | + | 0-0-1- |
| | | AAAGGGCCU | | | | | | | | | | | | 18-136 |
| G106R | SpBE3 | GGUAUCCCCGG | (TGG) | 20 (C7) | 5.7 | 67 | 28 | 77 | 3 | 53 | 23 | 5 | + | 0-0-2- |
| | | CGGGCAGCC | | | | | | | | | | | | 9-108 |
| R29C | SpBE3 | GCGCCCGUGCG | (GAG) | 20 (C6) | 8.3 | 77 | 31 | 66 | 5 | 57 | 67 | 8 | + | 0-0-0- |
| | | CAGGAGGAC | | | | | | | | | | | | 6-85 |
| A68T | SpBE3 | CUUGGCGCAGC | (TGG) | 20 (C6) | 7.7 | 62 | 54 | 81 | 9 | 61 | 78 | 7 | +GG | 0-0-2- |
| | | GGUGGAAGG | | | | | | | | | | | | 23-187 |
| G106R | SpBE3 | GUAUCCCCGGC | (GGG) | 20 (C6) | 5.9 | 71 | 37 | 49 | 6 | 72 | 57 | 5 | + | 0-0-2- |
| | | GGGCAGCCU | | | | | | | | | | | | 16-83 |
| A53V | EQR- | GACGGCCUGGC | (CGAG) | 20 (C11) | 6.2 | 86 | — | 57 | 2 | 52 | 55 | 6 | + | 0-0-0- |
| | SpBE3 | CGAAGCACC | | | | | | | | | | | | 10-48 |
| L253F | SpBE3 | CUGCGCGUGCU | (AGG) | 20 (C10) | 7.9 | 84 | 50 | 34 | 7 | 59 | 44 | 7 | + | 0-0-1- |
| | | CAACUGCCA | | | | | | | | | | | | 26-105 |
| C378Y | EQR- | AAGCAGGUGC | (GGAG) | 20 (C4) | 7.4 | 85 | — | 38 | 23 | 52 | 56 | 7 | + | 0-0-0- |
| | SpBE3 | UGCAGUCGCU | | | | | | | | | | | | 13-118 |
| C375Y | EQR- | AAGCAGGUGC | (GGAG) | 20 (C12) | 7.4 | 85 | — | 38 | 23 | 52 | 56 | 7 | + | 0-0-0- |
| | SpBE3 | UGCAGUCGCU | | | | | | | | | | | | 13-118 |
| S376N | EQR- | AAGCAGGUGC | (GGAG) | 20 (C10) | 7.4 | 85 | — | 38 | 23 | 52 | 56 | 7 | + | 0-0-0- |
| | SpBE3 | UGCAGUCGCU | | | | | | | | | | | | 13-118 |
| A290V | VRER- | CCCUGGCGGGU | (CGCG) | 20 (C7) | 5.9 | 99 | — | 42 | 0 | 32 | 42 | 5 | − | 0-0-0- |
| | SpBE3 | GGGUACAGC | | | | | | | | | | | | 0-16 |
| S373N, | KKH- | CUGCAGUCGC | (AATG | 20 (C8/ | 7.8 | 90 | — | 15 | 1 | 28 | 51 | 7 | + | 0-0-1- |
| D374N | SaBE3 | UGGAGGCACC | AT) | 4) | | | | | | | | | | 1-33 |
| M1I | St3BE3 | UGACGGUGCCC | (AGGG | 20 (C10) | 5.5 | 83 | 42 | 32 | 2 | 56 | 34 | 5 | + | 0-0-1- |
| | | AUGAGGGCC | G) | | | | | | | | | | | 6-47 |
| G452D | SpBE3 | GCCAACCUGCA | (TGG) | 20 (C6) | 7.2 | 68 | 37 | 53 | 11 | 71 | 10 | 7 | + | 0-0-7- |
| | | AAAAGGGCC | | | | | | | | | | | | 12-130 |
| E57K | SpBE3 | GGUUCCGUGC | (CGG) | 20 (C12) | 9.1 | 88 | 49 | 34 | 18 | 43 | 39 | 9 | − | 0-0-0- |
| | | UCGGGUGCUU | | | | | | | | | | | | 4-46 |
| C378Y | SpBE3 | AAAGCAGGUG | (TGG) | 20 (C5) | 5.1 | 65 | 43 | 39 | 1 | 70 | 61 | 5 | + | 0-0-3- |
| | | CUGCAGUCGC | | | | | | | | | | | | 35-165 |
| S376N | SpBE3 | AAAGCAGGUG | (TGG) | 20 (C11) | 5.1 | 65 | 43 | 39 | 1 | 70 | 61 | 5 | + | 0-0-3- |
| | | CUGCAGUCGC | | | | | | | | | | | | 35-165 |
| R194Q | VQR- | CGGUGGUCAC | (GGTG) | 20 (C1) | 6.1 | 100 | — | 3 | 3 | 33 | 35 | 6 | − | 0-0-0- |
| | SpBE3 | UCUGUAUGCU | | | | | | | | | | | | 0-0 |
| E57K | SpBE3 | CCGUGCUCGGG | (CAG) | 20 (C8) | 6.1 | 88 | 39 | 4 | 2 | 40 | 46 | 6 | + | 0-0-0- |
| | | UGCUUCGGC | | | | | | | | | | | | 3-53 |
| M1I | SpBE3 | GACGGUGCCCA | (GGG) | 20 (C10) | 7.8 | 48 | 51 | 47 | 21 | 83 | 60 | 7 | + | 0-1-3- |
| | | UGAGGGCCA | | | | | | | | | | | | 22-128 |
| S153N | EQR- | CAGAGCAUCCC | (GGAG) | 20 (C6) | 6.4 | 77 | — | 35 | 10 | 47 | 54 | 6 | − | 0-0-2- |
| | SpBE3 | GUGGAACCU | | | | | | | | | | | | 6-98 |
| L253F | SpBE3 | GUGCUCAACU | (GGG) | 20 (C3) | 4.3 | 53 | 56 | 60 | 41 | 74 | 72 | 4 | − | 0-0-3- |
| | | GCCAAGGGAA | | | | | | | | | | | | 40-225 |
| S153N | SpBE3 | CCAGAGCAUCC | (TGG) | 20 (C7) | 8.6 | 68 | 45 | 59 | 3 | 41 | 32 | 8 | + | 0-0-4- |
| | | CGUGGAACC | | | | | | | | | | | | 14-201 |
| P12L | SpBE3 | CAGCGGCCACC | (TGG) | 20 (C8) | 6.6 | 61 | 43 | 63 | 17 | 53 | 48 | 6 | + | 0-1-0- |
| | | AGGACCGCC | | | | | | | | | | | | 28-213 |
| P14S | SpBE3 | CAGCGGCCACC | (TGG) | 20 (C1) | 6.6 | 61 | 43 | 63 | 17 | 53 | 48 | 6 | + | 0-1-0- |
| | | AGGACCGCC | | | | | | | | | | | | 28-213 |
| G27D | SpBE3 | CACGGGCGCCC | (AGG) | 20 (C9) | 6.3 | 59 | 35 | 65 | 1 | 43 | 59 | 6 | + | 0-0-2- |
| | | GCGGGACCC | | | | | | | | | | | | 17-172 |
| T77I | EQR- | CAGCACCUGCU | (AGAG) | 20 (C6) | 7.6 | 58 | — | 5 | 2 | 23 | 61 | 7 | − | 0-0-2- |
| | SpBE3 | UUGUGUCAC | | | | | | | | | | | | 33-235 |
| T377I | EQR- | CAGCACCUGCU | (AGAG) | 20 (C6) | 7.6 | 58 | — | 5 | 2 | 23 | 61 | 7 | − | 0-0-2- |
| | SpBE3 | UUGUGUCAC | | | | | | | | | | | | 33-235 |
| R194Q | SpBE3 | CCGGUGGUCAC | (TGG) | 20 (C2) | 6.4 | 62 | 50 | 10 | 9 | 54 | 42 | 6 | − | 0-0-1- |
| | | UCUGUAUGC | | | | | | | | | | | | 7-168 |
| G263S | SpBE3 | CGCUAACCGUG | (TGG) | 20 (C1) | 4.8 | 71 | 40 | 7 | 8 | 43 | 42 | 4 | − | 0-0-1- |
| | | CCCUUCCCU | | | | | | | | | | | | 8-65 |
| R46L | VQR- | CUAGCCUUGC | (GGAC) | 20 (C10) | 7.1 | 64 | — | 28 | 21 | 47 | 45 | 7 | + | 0-0-1- |
| | SpBE3 | GUUCCGAGGA | | | | | | | | | | | | 29-728 |
| P616S/ | St3BE3 | AAUCCCGGCCC | (AGGT | 20 | 6.6 | 40 | 51 | 44 | 12 | 60 | 40 | 6 | + | 0-0-0- |
| L | | CUCAGGAGC | G) | (C4/5) | | | | | | | | | | 39-583 |

*Guide sequences (the portion of the guide RNA that targets the nucleobase editor to the target sequence) are provided, which may be used with any tracrRNA framework sequences provided herein to generate the full guide RNA sequence
^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.
^{b}Efficiency score, based on Housden et al (Science Signaling, 2015, 8(393): r59).
^{c}Specificity scores based on Hsu et al (Nature biotechnology, 2013, 31(9): 827-832), Fusi et al (bioRxiv 021568; doi: http://dx.doi.org/10.1101/021568), Chari et al (Nature Methods, 2015, 12(9): 823-6), Doench et al (Nature Biotechnology, 2014, 32(12): 1262-7), Wang et al (Science, 2014, 343(6166): 80-4), Moreno-Mateos et al (Nature Methods, 2015, 12(10): 982-8), Housden et al (Science Signaling, 2015, 8(393): r59), and the “Prox/GC” column shows “+” if the proximal 6 bp to the PAM has a GC count >= 4, and GG if the guide ends with GG, based on Farboud et al (Genetics, 2015, 199(4): 959-71).
^{d}Number of predicted off-target binding sites in the human genome allowing up to 0, 1, 2, 3 or 4 mismatches, respectively shown in the format 0-1-2-3-4. Algorithm used: Haeussler et al, Genome Biol. 2016; 17: 148.

TABLE 10
Efficiency and Specificity Scores for gRNAs for PCSK9 Variants to Destabilize Protein
Folding. Guide sequences correspond to SEQ ID NOs: 1438-1620 from top to bottom.

| | BE | | | gRNA size | | | | | | | M.- | Hous | Prox/ | Off- |
|---|---|---|---|---|---|---|---|---|---|---|---|---|---|---|
| Variants | type^{a} | guidesequence | PAM | (C edited) | Eff.^{b} | Hsu^{c} | Fusi | C. | Doench | W. | M. | den | GC | targets |
| P163S/L | VRER- | AUUACCCCUCCA | (GGCG) | 20 | 6.5 | 100 | — | 97 | 70 | 72 | 33 | 6 | + | 0-0-0- |
| and/or | SpBE3 | CGGUACCG | | (C7,8,10,11) | | | | | | | | | | 0-0 |
| P164S/L | | | | | | | | | | | | | | |
| P163S/L | SaBE3 | UUACCCCUCCAC | (GCGGAT) | 20 | 7.8 | 100 | — | 97 | 46 | 83 | 62 | 7 | +GG | 0-0-0- |
| and/or | | GGUACCGG | | (C6,7,9,10) | | | | | | | | | | 0-2 |
| P164S/L | | | | | | | | | | | | | | |
| P138S/L | St3BE3 | GCCCCAUGUCGA | (AGGAG) | 20 (C2/3) | 6.5 | 99 | 73 | 96 | 24 | 79 | 26 | 6 | − | 0-0-0- |
| | | CUACAUCG | | | | | | | | | | | | 0-5 |
| P138S/L | SpBE3 | GCCCCAUGUCGA | (AGG) | 20 (C2/3) | 6.5 | 98 | 73 | 96 | 24 | 79 | 26 | 6 | − | 0-0-0- |
| | | CUACAUCG | | | | | | | | | | | | 0-16 |
| P585S/L | VQR- | CGAGGUCAGCCC | (CGTG) | 20 (C10/11) | 7.5 | 99 | — | 94 | 4 | 58 | 78 | 7 | + | 0-0-0- |
| and/or | SpBE3 | AACCAGUG | | | | | | | | | | | | 0-1 |
| C558Y | | | | | | | | | | | | | | |
| P581S/L | VQR- | GCCACGAGGUCA | (AGTG) | 20 (C2/3) | 5.2 | 99 | — | 93 | 1 | 54 | 41 | 5 | + | 0-0-0- |
| | SpBE3 | GCCCAACC | | | | | | | | | | | | 0-7 |
| P404S/L | SaBE3 | CGAGCCGGAGCU | (CCGAGT) | 20 (C5/6) | 5.5 | 96 | — | 95 | 25 | 78 | 85 | 5 | +GG | 0-0-0- |
| | | CACCCUGG | | | | | | | | | | | | 1-12 |
| P75S/L | St3BE3 | GUUGCCUGGCAC | (TGGTG) | 20 (C5/6) | 9.4 | 98 | 73 | 88 | 15 | 92 | 60 | 9 | +GG | 0-0-0- |
| | | CUACGUGG | | | | | | | | | | | | 0-14 |
| P585S/L | VRER- | CACGAGGUCAGC | (TGCG) | 20 (C12/13) | 4.4 | 100 | — | 87 | 20 | 90 | 69 | 4 | − | 0-0-0- |
| and/or | SpBE3 | CCAACCAG | | | | | | | | | | | | 0-5 |
| C558Y | | | | | | | | | | | | | | |
| P56S/L | SpBE3 | AGCACCCGAGCA | (CAG) | 20 (C5/6) | 4.0 | 93 | 56 | 97 | 36 | 70 | 38 | 4 | − | 0-0-0- |
| | | CGGAACCA | | | | | | | | | | | | 2-46 |
| P155S/L | VRER- | GAGCAUCCCGUG | (AGCG) | 20 (C7/8) | 4.2 | 98 | — | 90 | 46 | 84 | 65 | 4 | +GG | 0-0-0- |
| | SpBE3 | GAACCUGG | | | | | | | | | | | | 1-3 |
| P163S/L | SaBE3 | CCCUCCACGGUA | (ATGAAT) | 20 (C2,3,5,6) | 5.3 | 99 | — | 88 | 7 | 70 | 56 | 5 | +GG | 0-0-0- |
| and/or | | CCGGGCGG | | | | | | | | | | | | 0-6 |
| P164S/L | | | | | | | | | | | | | | |
| P445S/L | KKH- | UGCCCCCCAGCA | (GCAGGT) | 20 (C3,4,6,7) | 4.4 | 91 | — | 96 | 7 | 66 | 61 | 4 | +GG | 0-0-0- |
| and/or | SaBE3 | CCCAUGGG | | | | | | | | | | | | 3-38 |
| P446S/L | | | | | | | | | | | | | | |
| C255Y | VRER- | GCAGUUGAGCAC | (TGCG) | 20 (C2) | 8.2 | 99 | — | 85 | 6 | 79 | 20 | 8 | + | 0-0-0- |
| | SpBE3 | GCGCAGGC | | | | | | | | | | | | 0-7 |
| G516R/E | VQR- | ACCCUCACCCCC | (TGTG) | 20 (C10/11) | 5.6 | 100 | — | 24 | 9 | 83 | 20 | 5 | − | 0-0-0- |
| | SpBE3 | AAAAGCGU | | | | | | | | | | | | 0-3 |
| P581S/L | KKH- | GAGGCCACGAGG | (ACCAGT) | 20 (C5/6) | 4.6 | 96 | — | 61 | 12 | 87 | 81 | 4 | + | 0-0-0- |
| | SaBE3 | UCAGCCCA | | | | | | | | | | | | 1-18 |
| P75S/L | SpBE3 | GUUGCCUGGCAC | (TGG) | 20 (C5/6) | 9.4 | 90 | 73 | 88 | 15 | 92 | 60 | 9 | +GG | 0-0-0- |
| | | CUACGUGG | | | | | | | | | | | | 4-63 |
| P163S/L | SpBE3 | UACCCCUCCACG | (CGG) | 20 (C5,6,8,9) | 5.6 | 97 | 70 | 85 | 72 | 79 | 67 | 5 | +GG | 0-0-0- |
| and/or | | GUACCGGG | | | | | | | | | | | | 0-24 |
| P164S/L | | | | | | | | | | | | | | |
| P163S/L | VQR- | CCUCCACGGUAC | (TGAA) | 20 (C1,2,4,5) | 6.4 | 96 | — | 86 | 2 | 46 | 60 | 6 | + | 0-0-0- |
| and/or | SpBE3 | CGGGCGGA | | | | | | | | | | | | 1-26 |
| P164S/L | | | | | | | | | | | | | | |
| P288S/L | SaBE3 | GGUGCUGCUGCC | (GTGGGT) | 20 (C11/12) | 4.3 | 89 | — | 86 | 13 | 93 | 83 | 4 | +GG | 0-0-1- |
| | | CCUGGCGG | | | | | | | | | | | | 8-76 |
| P616S/L | KKH- | GGAAUCCCGGCC | (GCAGGT) | 20 (C7/8) | 4.0 | 94 | — | 86 | 23 | 87 | 53 | 4 | − | 0-0-0- |
| and/or | SaBE3 | CCUCAGGA | | | | | | | | | | | | 1-26 |
| P618S/L | | | | | | | | | | | | | | |
| C601Y | VRER- | CCUGGGGCAUGG | (AGCG) | 20 (C12) | 4.5 | 91 | — | 89 | 22 | 71 | 54 | 4 | + | 0-0-0- |
| | SpBE3 | CAGCAGGA | | | | | | | | | | | | 0-41 |
| C655Y | SpBE3 | CACACGUGUUGU | (TAG) | 20 (C3) | 5.4 | 98 | 58 | 71 | 22 | 82 | 36 | 5 | + | 0-0-0- |
| | | CUACGGCG | | | | | | | | | | | | 2-21 |
| G337R/E | KKH- | CCCCAACUGUGA | (AAAGGT) | 20 (C3/4) | 4.6 | 94 | — | 85 | 13 | 60 | 50 | 4 | +GG | 0-0-0- |
| | SaBE3 | UGACCUGG | | | | | | | | | | | | 3-20 |
| P25S/L | VRER- | CUGGGUCCCGCG | (TGCG) | 20 (C7/8) | 5.8 | 90 | — | 70 | 1 | 55 | 88 | 5 | + | 0-0-0- |
| | SpBE3 | GGCGCCCG | | | | | | | | | | | | 1-60 |
| C67Y | St3BE3 | CACCUUGGCGCA | (AGGTG) | 20 (C11) | 4.9 | 95 | 46 | 83 | 2 | 33 | 57 | 4 | + | 0-0-0- |
| | | GCGGUGGA | | | | | | | | | | | | 2-33 |
| P467S/L | SpBE3 | ACACUCGGGGCC | (TGG) | 20 (C11/12) | 5.3 | 96 | 57 | 82 | 3 | 73 | 46 | 5 | + | 0-0-0- |
| | | UACACGGA | | | | | | | | | | | | 3-24 |
| P75S/L | VQR- | AGGUUGCCUGGC | (GGTG) | 20 (C7/8) | 4.2 | 100 | — | 23 | 17 | 77 | 71 | 4 | − | 0-0-0- |
| | SpBE3 | ACCUACGU | | | | | | | | | | | | 0-3 |
| P540S/L | St3BE3 | UCCACCAGCUGA | (TGGGG) | 20 (C2,3,5,6) | 4.7 | 83 | 50 | 94 | 5 | 44 | 35 | 4 | + | 0-0-0- |
| and/or | | GGCCAGCA | | | | | | | | | | | | 8-70 |
| P541S/L | | | | | | | | | | | | | | |
| C255Y | SpBE3 | CCUUGGCAGUUG | (CAG) | 20 (C7) | 6.3 | 88 | 49 | 88 | 38 | 56 | 54 | 6 | + | 0-0-1- |
| | | AGCACGCG | | | | | | | | | | | | 6-46 |
| P75S/L | KKH- | AGGUUGCCUGGC | (GGTGGT) | 20 (C7/8) | 4.2 | 98 | 49 | 23 | 17 | 77 | 71 | 4 | − | 0-0-0- |
| | SaBE3 | ACCUACGU | | | | | | | | | | | | 1-16 |
| C223Y | VQR- | ACACUUGCUGGC | (CG) | 20 (C2) | 5.8 | 91 | — | 84 | 40 | 69 | 56 | 5 | + | 0-0-0- |
| | SpBE3 | CUGCUCGA | | | | | | | | | | | | 0-85 |
| C526Y | KKH- | CAUGGCACCCAC | (GGTGGT) | 20 (C12/9) | 10.1 | 85 | 47 | 90 | 14 | 77 | 57 | 10 | +GG | 0-0-0- |
| and/or | SaBE3 | CUGGCAGG | | | | | | | | | | | | 4-45 |
| C527Y | | | | | | | | | | | | | | |
| P604S/L | KKH- | CAUGCCCCAGGU | (CAAAGT) | 20 (C7/8) | 7.2 | 94 | — | 81 | 15 | 43 | 74 | 7 | − | 0-0-0- |
| | SaBE3 | CUGGAAUG | | | | | | | | | | | | 1-41 |
| P585S/L | SpBE3 | GGUCAGCCCAAC | (GGG) | 20 (C4,7,8) | 4.8 | 86 | 62 | 59 | 44 | 88 | 34 | 4 | + | 0-0-2- |
| and/or | | CAGUGCGU | | | | | | | | | | | | 6-51 |
| C558Y | | | | | | | | | | | | | | |
| C255Y | SpBE3 | CUUGGCAGUUGA | (AGG) | 20 (C6) | 5.4 | 94 | 51 | 69 | 43 | 79 | 44 | 5 | + | 0-0-0- |
| | | GCACGCGC | | | | | | | | | | | | 1-46 |
| C526Y | VQR- | GCAGCACCUGGC | (AGAC) | 20 (C5/2) | 3.8 | 84 | — | 54 | 46 | 89 | 59 | 3 | + | 0-0-2- |
| and/or | SpBE3 | AAUGGCGU | | | | | | | | | | | | 6-92 |
| C527Y | | | | | | | | | | | | | | |
| P25S/L | EQR- | CCCGCGGGCGCC | (GGAG) | 20 (C1/2) | 4.3 | 92 | — | 80 | 3 | 44 | 69 | 4 | + | 0-0-0- |
| | SpBE3 | CGUGCGCA | | | | | | | | | | | | 3-35 |
| P75S/L | St3BE3 | GAGGUUGCCUGG | (TGGTG) | 20 (C8/9) | 4.8 | 89 | 71 | 83 | 19 | 75 | 68 | 4 | + | 0-0-1- |
| | | CACCUACG | | | | | | | | | | | | 1-28 |
| P25S/L | SpBE3 | GUCCCGCGGGCG | (CAG) | 20 (C3/4) | 5.2 | 78 | 40 | 94 | 2 | 55 | 67 | 5 | + | 0-0-1- |
| | | CCCGUGCG | | | | | | | | | | | | 8-100 |
| C67Y | SpBE3 | CACCUUGGCGCA | (AGG) | 20 (C11) | 4.9 | 88 | 46 | 83 | 2 | 33 | 57 | 4 | + | 0-0-0- |
| | | GCGGUGGA | | | | | | | | | | | | 8-73 |
| P327S/L | KKH- | CCCCAGCCUCAG | (GTAGGT) | 20 (C3/4) | 8.3 | 87 | — | 84 | 34 | 67 | 64 | 8 | + | 0-0-1- |
| | SaBE3 | CUCCCGAG | | | | | | | | | | | | 6-48 |
| P56S/L | VQR- | UGGCCGAAGCAC | (GGAA) | 20 (C12/13) | 8.0 | 94 | — | 60 | 10 | 76 | 67 | 8 | + | 0-0-0- |
| | SpBE3 | CCGAGCAC | | | | | | | | | | | | 1-50 |
| P75S/L | VQR- | UUGCCUGGCACC | (GGTG) | 20 (C4/5) | 4.7 | 100 | — | 41 | 7 | 33 | 70 | 4 | + | 0-0-0- |
| | SpBE3 | UACGUGGU | | | | | | | | | | | | 0-4 |
| P173S/L | VQR- | CCCCCCGGUAAG | (TGTG) | 21 (C1,−1, | 4.6 | 99 | — | 71 | 3 | 29 | 27 | 4 | + | 0-0-0- |
| and/or | SpBE3 | ACCCCCAUC | | 3,4) | | | | | | | | | | 0-4 |
| P174S/L | | | | | | | | | | | | | | |
| C358Y | KKH- | AGGUCCACACAG | (GTTGGT) | 20 (C10) | 7.4 | 94 | — | 76 | 41 | 48 | 46 | 7 | − | 0-0-0- |
| | SaBE3 | CGGCCAAA | | | | | | | | | | | | 1-28 |
| P75S/L | KKH- | UGGAGGUUGCCU | (CGTGGT) | 20 (C10/11) | 8.2 | 93 | 40 | 36 | 7 | 43 | 76 | 8 | − | 0-0-0- |
| | SaBE3 | GGCACCUA | | | | | | | | | | | | 2-44 |
| P209S/L | VQR- | GAAUGUGCCCGA | (GGAC) | 20 (C8/9) | 6.9 | 82 | — | 87 | 32 | 87 | 52 | 6 | + | 0-0-1- |
| | SpBE3 | GGAGGACG | | | | | | | | | | | | 2-79 |
| P279S/L | St3BE3 | CCAGCCUGUGGG | (TGGTG) | 20 (C5/6) | 5.4 | 85 | 48 | 84 | 10 | 78 | 66 | 5 | +GG | 0-0-3- |
| | | GCCACUGG | | | | | | | | | | | | 7-79 |
| G232R/E | SaBE3 | CCGCUGACCACC | (GTGGGT) | 20 (C11/12) | 4.1 | 87 | — | 73 | 12 | 81 | 81 | 4 | + | 0-0-1- |
| | | CCUGCCAG | | | | | | | | | | | | 1-28 |
| C301Y | SpBE3 | GGCGCUGGCAGG | (AGG) | 20 (C9) | 4.9 | 74 | 49 | 94 | 11 | 68 | 67 | 4 | + | 0-0-1- |
| | | CGGCGUUG | | | | | | | | | | | | 23-216 |
| C358Y | KKH- | CAGCGGCCAAAG | (CAAAGT) | 20 (C1) | 6.7 | 97 | — | 18 | 12 | 47 | 71 | 6 | + | 0-0-0- |
| | SaBE3 | UUGGUCCC | | | | | | | | | | | | 1-12 |
| G384R/E | St3BE3 | CCCACUCUGUGA | (AGGTG) | 20 (C2/3) | 5.0 | 88 | 58 | 80 | 19 | 44 | 34 | 5 | − | 0-0-0- |
| | | CACAAAGC | | | | | | | | | | | | 8-66 |
| C301Y | VRER- | CUGGCAGGCGGC | (CGCG) | 20 (C5) | 6.7 | 97 | — | 63 | 11 | 65 | 70 | 6 | − | 0-0-0- |
| | SpBE3 | GUUGAGGA | | | | | | | | | | | | 3-22 |
| P331S/L | VQR- | CAGCCUCAGCUC | (GGTG) | 20 (C12/13) | 7.2 | 100 | — | 66 | 5 | 46 | 64 | 7 | − | 0-0-0- |
| | SpBE3 | CCGAGGUA | | | | | | | | | | | | 2-7 |
| G213R/E | SpBE3 | GAAGCGGGUCCC | (CGG) | 20 (C10/11) | 8.9 | 80 | 42 | 85 | 2 | 69 | 69 | 8 | + | 0-0-1- |
| | | GUCCUCCU | | | | | | | | | | | | 8-95 |
| G232R/E | St3BE3 | GCUGACCACCCC | (GGGTG) | 20 (C9/10) | 6.2 | 83 | 58 | 82 | 8 | 68 | 60 | 6 | + | 0-0-1- |
| | | UGCCAGGU | | | | | | | | | | | | 5-55 |
| G292R/E | SpBE3 | CGGCUGUACCCA | (GGG) | 20 (C10/11) | 6.4 | 79 | 60 | 86 | 19 | 78 | 82 | 6 | + | 0-0-0- |
| | | CCCGCCAG | | | | | | | | | | | | 11-86 |
| C301Y | VQR- | GCGCUGGCAGGC | (GGAC) | 20 (C8) | 5.3 | 90 | — | 58 | 10 | 50 | 75 | 5 | − | 0-0-0- |
| | SpBE3 | GGCGUUGA | | | | | | | | | | | | 8-48 |
| P331S/L | St3BE3 | UCAGCUCCCGAG | (TGGGG) | 20 (C7/8) | 6.9 | 90 | 34 | 14 | 15 | 75 | 36 | 6 | + | 0-0-0- |
| | | GUAGGUGC | | | | | | | | | | | | 6-43 |
| C655Y | SpBE3 | ACACGUGUUGUC | (AGG) | 20 (C2) | 4.5 | 99 | 61 | 26 | 14 | 66 | 59 | 4 | + | 0-0-0- |
| | | UACGGCGU | | | | | | | | | | | | 1-10 |
| C323Y | KKH- | GUAGAGGCAGGC | (GGAAGT) | 20 (C12) | 6.4 | 96 | 52 | 61 | 26 | 69 | 68 | 6 | + | 0-0-0- |
| | SaBE3 | AUCGUCCC | | | | | | | | | | | | 0-20 |
| P345S/L | SpBE3 | AAGACCAGCCGG | (GGG) | 20 (C9/10) | 6.3 | 66 | 67 | 96 | 19 | 79 | 68 | 6 | + | 0-0-1- |
| | | UGACCCUG | | | | | | | | | | | | 13-143 |
| C477Y | SpBE3 | AUCUGGGGCGCA | (CGG) | 20 (C11) | 5.1 | 84 | 45 | 78 | 17 | 73 | 75 | 5 | + | 0-0-0- |
| | | GCGGGCGA | | | | | | | | | | | | 2-112 |
| C67Y | KKH- | GCGCAGCGGUGG | (TGTGGT) | 20 (C4) | 5.5 | 91 | 27 | 71 | 1 | 44 | 53 | 5 | + | 0-0-0- |
| | SaBE3 | AAGGUGGC | | | | | | | | | | | | 2-37 |
| P138S/L | EQR- | UUGCCCCAUGUC | (CGAG) | 20 (C4/5) | 5.2 | 94 | — | 38 | 20 | 29 | 67 | 5 | − | 0-0-0- |
| | SpBE3 | GACUACAU | | | | | | | | | | | | 1-45 |
| C678Y | SpBE3 | GCAGAUGGCAAC | (CGG) | 20 (C2) | 5.4 | 82 | 50 | 57 | 14 | 79 | 56 | 5 | − | 0-0-1- |
| and/or | | GGCUGUCA | | | | | | | | | | | | 9-101 |
| C679Y | | | | | | | | | | | | | | |
| P173S/L | VQR- | UGAAUACCAGCC | (AGAC) | 20 (C11/12) | 3.7 | 97 | — | 63 | 2 | 59 | 62 | 3 | + | 0-0-0- |
| and/or | SpBE3 | CCCCGGUA | | | | | | | | | | | | 1-31 |
| P174S/L | | | | | | | | | | | | | | |
| P364S/L | KKH- | UUGCCCCAGGGG | (ATTGGT) | 20 (C6/7) | 6.2 | 91 | — | 69 | 1 | 15 | 65 | 6 | − | 0-0-0- |
| | SaBE3 | AGGACAUC | | | | | | | | | | | | 4-31 |
| G516R/E | SpBE3 | CCUCACCCCCAA | (TGG) | 20 (C9/10) | 7.5 | 78 | 57 | 82 | 13 | 52 | 14 | 7 | + | 0-0-0- |
| | | AAGCGUUG | | | | | | | | | | | | 19-108 |
| C526Y | St3BE3 | UAGCAGGCAGCA | (TGGCG) | 20 (C8/5) | 3.1 | 79 | 55 | 44 | 19 | 81 | 68 | 3 | − | 0-0-1- |
| and/or | | CCUGGCAA | | | | | | | | | | | | 5-48 |
| C527Y | | | | | | | | | | | | | | |
| P585S/L | SpBE3 | AGGUCAGCCCAA | (TGG) | 20 (C5,8,9) | 7.2 | 83 | 56 | 70 | 36 | 77 | 37 | 7 | + | 0-0-2- |
| and/or | | CCAGUGCG | | | | | | | | | | | | 6-65 |
| C558Y | | | | | | | | | | | | | | |
| P75S/L | SpBE3 | GAGGUUGCCUGG | (TGG) | 20 (C8/9) | 4.8 | 76 | 71 | 83 | 19 | 75 | 68 | 4 | + | 0-0-1- |
| | | CACCUACG | | | | | | | | | | | | 7-118 |
| P163S/L | SpBE3 | GGAUUACCCCUC | (CGG) | 20 | 6.7 | 98 | 47 | 7 | 17 | 61 | 47 | 6 | + | 0-0-1- |
| and/or | | CACGGUAC | | (C9,10,12,13) | | | | | | | | | | 1-10 |
| P164S/L | | | | | | | | | | | | | | |
| G176R/E | VRER- | GGCUGCCUCCGU | (GGCG) | 20 (C9/10) | 8.5 | 99 | — | 51 | 52 | 60 | 45 | 8 | − | 0-0-0- |
| | SpBE3 | CUUUCCAA | | | | | | | | | | | | 0-6 |
| P364S/L | St3BE3 | GCCCCAGGGGAG | (TGGTG) | 20 (C4/5) | 6.6 | 92 | 40 | 60 | 8 | 54 | 67 | 6 | − | 0-0-0- |
| | | GACAUCAU | | | | | | | | | | | | 4-53 |
| P438S/L | SpBE3 | GCGGGUACUGAC | (TGG) | 20 (C12/13) | 4.7 | 90 | 58 | 45 | 16 | 65 | 69 | 4 | + | 0-0-0- |
| | | CCCCAACC | | | | | | | | | | | | 3-50 |
| P530S/L | VRER- | UGCUACCCCAGG | (AGCG) | 20 (C6/7) | 4.1 | 99 | — | 23 | 3 | 60 | 19 | 4 | − | 0-0-0- |
| | SpBE3 | CCAACUGC | | | | | | | | | | | | 1-5 |
| G670R/E | VQR- | GCUGUCACGGCC | (GGTG) | 20 (C13/14) | 5.2 | 100 | — | 40 | 11 | 59 | 32 | 5 | − | 0-0-0- |
| | SpBE3 | CCUUCGCU | | | | | | | | | | | | 1-2 |
| P279S/L | VQR- | GUCCAGCCUGUG | (GGTG) | 20 (C7/8) | 4.7 | 99 | — | 51 | 9 | 31 | 60 | 4 | + | 0-0-0- |
| | SpBE3 | GGGCCACU | | | | | | | | | | | | 0-8 |
| G292R/E | SpBE3 | CUGUACCCACCC | (CAG) | 20 (C7/8) | 7.2 | 74 | 52 | 70 | 23 | 81 | 85 | 7 | +GG | 0-0-0- |
| | | GCCAGGGG | | | | | | | | | | | | 10-154 |
| C526Y | VRER- | AGCAGGCAGCAC | (GGCG) | 20 (C10/7) | 10.6 | 98 | — | 60 | 3 | 39 | 57 | 10 | − | 0-0-0- |
| and/or | SpBE3 | CUGGCAAU | | | | | | | | | | | | 1-16 |
| C527Y | | | | | | | | | | | | | | |
| G365R/E | KKH- | GAUGUCCUCCCC | (AGAGGT) | 20 (C11/12) | 6.9 | 89 | 46 | 69 | 4 | 67 | 61 | 6 | + | 0-0-1- |
| | SaBE3 | UGGGGCAA | | | | | | | | | | | | 1-35 |
| P138S/L | EQR- | CCCCAUGUCGAC | (GGAG) | 20 (C1/2) | 4.5 | 95 | — | 62 | 55 | 53 | 40 | 4 | − | 0-0-0- |
| | SpBE3 | UACAUCGA | | | | | | | | | | | | 1-47 |
| G213R/E | SpBE3 | AAGCGGGUCCCG | (GGG) | 20 (C9/10) | 6.6 | 75 | 45 | 18 | 7 | 43 | 82 | 6 | + | 0-0-1- |
| | | UCCUCCUC | | | | | | | | | | | | 7-55 |
| P430S/L | SaBE3 | GCCUGGUUCCCU | (GCGGGT) | 20 (C10/11) | 6.4 | 94 | — | 62 | 25 | 58 | 47 | 6 | + | 0-0-0- |
| | | GAGGACCA | | | | | | | | | | | | 2-38 |
| C655Y | St3BE3 | GACUACACACGU | (CGGCG) | 20 (C8) | 8.3 | 99 | 57 | 32 | 24 | 44 | 41 | 8 | − | 0-0-0- |
| | | GUUGUCUA | | | | | | | | | | | | 0-6 |
| G337R/E | St3BE3 | CCAACUGUGAUG | (AGGTG) | 20 (C1/2) | 5.1 | 90 | 65 | 44 | 14 | 58 | 47 | 5 | − | 0-0-0- |
| | | ACCUGGAA | | | | | | | | | | | | 2-40 |
| G450R/E | St3BE3 | UACCUGCCCCAU | (GGGGG) | 20 (C9/10) | 7.5 | 88 | 43 | 53 | 4 | 67 | 50 | 7 | + | 0-0-1- |
| | | GGGUGCUG | | | | | | | | | | | | 4-45 |
| C67Y | VQR- | ACCUUGGCGCAG | (GGTG) | 20 (C10) | 7.5 | 97 | — | 30 | 10 | 58 | 55 | 7 | − | 0-0-0- |
| | SpBE3 | CGGUGGAA | | | | | | | | | | | | 1-1 |
| P25S/L | St3BE3 | UCCCGCGGGCGC | (AGGAG) | 20 (C2) | 7.6 | 94 | 38 | 60 | 0 | 56 | 48 | 7 | + | 0-0-0- |
| | | CCGUGCGC | | | | | | | | | | | | 3-42 |
| P163S/L | VQR- | ACCCCUCCACGG | (GGAT) | 20 (C4,5,7,8) | 5.7 | 94 | — | 47 | 7 | 60 | 54 | 5 | + | 0-0-0- |
| and/or | SpBE3 | UACCGGGC | | | | | | | | | | | | 1-30 |
| P164S/L | | | | | | | | | | | | | | |
| P279S/L | KKH- | CUGGUCCAGCCU | (ACTGGT) | 20 (C10/11) | 10.8 | 83 | — | 21 | 0 | 43 | 71 | 10 | + | 0-0-0- |
| | SaBE3 | GUGGGGCC | | | | | | | | | | | | 10-77 |
| P445S/L | St3BE3 | GCCCUGCCCCCC | (TGGGG) | 20 | 5.9 | 78 | 34 | 76 | 4 | 73 | 36 | 5 | + | 0-0-1- |
| and/or | | AGCACCCA | | (C7,8,10,11) | | | | | | | | | | 17-123 |
| P446S/L | | | | | | | | | | | | | | |
| C477Y | SpBE3 | GGCGCAGCGGGC | (TGG) | 20 (C5) | 6.5 | 76 | 35 | 76 | 3 | 78 | 64 | 6 | + | 0-0-3- |
| | | GACGGCUG | | | | | | | | | | | | 21-226 |
| C600Y | VRER- | GGGGCAUGGCAG | (GTGGAT) | 20 (C13/10) | 7.4 | 81 | — | 58 | 0 | 73 | 58 | 7 | + | 0-0-0- |
| and/or | SpBE3 | CAGGAAGC | | | | | | | | | | | | 13-76 |
| C601Y | | | | | | | | | | | | | | |
| P163S/L | St3BE3 | GAUUACCCCUCC | (GGGCG) | 20 | 5.1 | 99 | 54 | 48 | 9 | 32 | 38 | 5 | + | 0-0-0- |
| and/or | | ACGGUACC | | (C8,9,11,12) | | | | | | | | | | 0-3 |
| P164S/L | | | | | | | | | | | | | | |
| C255Y | VRER- | CUUCCCUUGGCA | (CGCG) | 20 (C11) | 6.9 | 97 | — | 56 | 18 | 34 | 27 | 6 | − | 0-0-0- |
| | SpBE3 | GUUGAGCA | | | | | | | | | | | | 0-16 |
| G257R/E | VRER- | CUUCCCUUGGCA | (CGCG) | 20 (C5/6) | 6.9 | 97 | — | 56 | 18 | 34 | 27 | 6 | − | 0-0-0- |
| | SpBE3 | GUUGAGCA | | | | | | | | | | | | 0-16 |
| C588Y | VQR- | GGCCCACGCACU | (TGAC) | 20 (C9) | 4.5 | 84 | — | 28 | 1 | 69 | 22 | 4 | + | 0-0-0- |
| | SpBE3 | GGUUGGGC | | | | | | | | | | | | 8-58 |
| P288S/L | St3BE3 | GUGGUGCUGCUG | (GGGTG) | 20 (C13/14) | 7.4 | 71 | 40 | 52 | 5 | 66 | 81 | 7 | + | 0-0-1- |
| | | CCCCUGGC | | | | | | | | | | | | 24-152 |
| G292R/E | St3BE3 | CGCGGCUGUACC | (AGGGG) | 20 (C12/13) | 4.7 | 94 | 44 | 58 | 5 | 40 | 54 | 4 | + | 0-0-0- |
| | | CACCCGCC | | | | | | | | | | | | 0-25 |
| P364S/L | VQR- | CCCCAGGGGAGG | (GGTG) | 20 (C3/4) | 4.8 | 99 | — | 25 | 1 | 23 | 53 | 4 | − | 0-0-0- |
| | SpBE3 | ACAUCAUU | | | | | | | | | | | | 1-3 |
| P576S/L | SpBE3 | CCGCCUGUGCUG | (AGG) | 20 (C1,2,4,5) | 7.9 | 59 | 63 | 93 | 54 | 42 | 53 | 7 | + | 0-0-2- |
| and/or | | AGGCCACG | | | | | | | | | | | | 14-197 |
| P577S/L | | | | | | | | | | | | | | |
| P331S/L | SpBE3 | UCAGCUCCCGAG | (TGG) | 20 (C7/8) | 6.9 | 76 | 34 | 14 | 15 | 75 | 36 | 6 | + | 0-0-1- |
| | | GUAGGUGC | | | | | | | | | | | | 18-133 |
| P279S/L | KKH- | GUCCAGCCUGUG | (GGTGGT) | 20 (C7/8) | 4.7 | 90 | 30 | 51 | 9 | 31 | 60 | 4 | + | 0-0-0- |
| | SaBE3 | GGGCCACU | | | | | | | | | | | | 6-28 |
| C477Y | VQR- | GGGGCGCAGCGG | (TGTG) | 20 (C7) | 8.5 | 66 | — | 84 | 2 | 81 | 47 | 8 | + | 0-0-7- |
| | SpBE3 | GCGACGGC | | | | | | | | | | | | 24-199 |
| P155S/L | St3BE3 | CCAGAGCAUCCC | (TGGAG) | 20 (C10) | 8.6 | 90 | 45 | 59 | 3 | 41 | 32 | 8 | + | 0-0-1- |
| | | GUGGAACC | | | | | | | | | | | | 2-68 |
| G176R/E | St3BE3 | AGGCUGCCUCCG | (AGGCG) | 20 (C9/10) | 5.3 | 92 | 55 | 15 | 22 | 57 | 39 | 5 | − | 0-0-0- |
| | | UCUUUCCA | | | | | | | | | | | | 3-50 |
| P345S/L | VQR- | AGACCAGCCGGU | (GGAC) | 20 (C8/9) | 5.9 | 62 | — | 87 | 40 | 77 | 72 | 5 | +GG | 0-0-3- |
| | SpBE3 | GACCCUGG | | | | | | | | | | | | 29-319 |
| P163S/L | SpBE3 | GAUUACCCCUCC | (GGG) | 20 | 5.1 | 94 | 54 | 48 | 9 | 32 | 38 | 5 | + | 0-0-1- |
| and/or | | ACGGUACC | | (C8,9,11,12) | | | | | | | | | | 1-24 |
| P164S/L | | | | | | | | | | | | | | |
| P279S/L | St3BE3 | GGUCCAGCCUGU | (TGGTG) | 20 (C8/9) | 6.6 | 85 | 36 | 39 | 2 | 50 | 63 | 6 | + | 0-0-0- |
| | | GGGGCCAC | | | | | | | | | | | | 13-49 |
| C301Y | EQR- | CAGGCGCUGGCA | (TGAG) | 20 (C11) | 6.1 | 73 | — | 50 | 0 | 75 | 69 | 6 | + | 0-0-2- |
| | SpBE3 | GGCGGCGU | | | | | | | | | | | | 25-102 |
| G337R/E | VQR- | AUUGGUGGCCCC | (TGAC) | 20 (C11/12) | 7.1 | 76 | — | 45 | 15 | 72 | 56 | 7 | − | 0-0-2- |
| | SpBE3 | AACUGUGA | | | | | | | | | | | | 9-106 |
| G450R/E | St3BE3 | CCCAUGGGUGCU | (GGGCG) | 20 (C2/3) | 5.2 | 55 | 41 | 47 | 1 | 35 | 93 | 5 | + | 0-0-3- |
| | | GGGGGGCA | | | | | | | | | | | | 17-226 |
| C323Y | VQR- | GUAGAGGCAGGC | (GGAA) | 20 (C12) | 6.4 | 78 | — | 61 | 26 | 69 | 68 | 6 | + | 0-0-7- |
| | SpBE3 | AUCGUCCC | | | | | | | | | | | | 9-93 |
| P345S/L | St3BE3 | GCCGGUGACCCU | (TGGGG) | 20 (C2/3) | 7.4 | 84 | 33 | 41 | 1 | 33 | 63 | 7 | − | 0-0-0- |
| | | GGGGACUU | | | | | | | | | | | | 4-69 |
| G505R/E | SaBE3 | CAGCUUGCCCCC | (TAGAGT) | 20 (C11/12) | 8.1 | 86 | — | 5 | 3 | 46 | 60 | 8 | + | 0-0-0- |
| | | UUGGGCCU | | | | | | | | | | | | 4-50 |
| G493R/E | St1BE3 | CCCCGCCGCUUC | (GGAGAAA) | 20 (C13/14) | 4.5 | 97 | — | 48 | 6 | 24 | 42 | 4 | − | 0-0-0- |
| | | CCACUCCU | | | | | | | | | | | | 1-11 |
| C588Y | SpBE3 | CACUGGUUGGGC | (TGG) | 20 (C1) | 4.8 | 88 | 54 | 57 | 6 | 54 | 23 | 4 | + | 0-0-0- |
| | | UGACCUCG | | | | | | | | | | | | 2-65 |
| C601Y | SpBE3 | GGGCAUGGCAGC | (TGG) | 20 (C9) | 4.6 | 47 | 59 | 97 | 54 | 80 | 64 | 4 | + | 0-0-4- |
| | | AGGAAGCG | | | | | | | | | | | | 38-411 |
| C67Y | SpBE3 | CUUGGCGCAGCG | (TGG) | 20 (C8) | 7.7 | 62 | 54 | 81 | 9 | 61 | 78 | 7 | +GG | 0-0-2- |
| | | GUGGAAGG | | | | | | | | | | | | 23-187 |
| P364S/L | VQR- | GACCUCUUUGCC | (GGAC) | 20 (C13/14) | 2.9 | 67 | — | 41 | 5 | 76 | 59 | 2 | + | 0-0-1- |
| | SpBE3 | CCAGGGGA | | | | | | | | | | | | 11-144 |
| P120S/L | KKH- | CUUCUUCCUGGC | (GAAGAT) | 20 (C1/2) | 6.4 | 85 | — | 27 | 12 | 27 | 57 | 6 | + | 0-0-0- |
| | SaBE3 | UUCCUGGU | | | | | | | | | | | | 15-83 |
| P327S/L | St3BE3 | CCAGCCUCAGCU | (AGGTG) | 20 (C1/2) | 4.0 | 88 | 54 | 26 | 7 | 50 | 53 | 4 | + | 0-0-0- |
| | | CCCGAGGU | | | | | | | | | | | | 8-205 |
| P404S/L | EQR- | GAGCCGGAGCUC | (CGAG) | 20 (C4/5) | 7.4 | 66 | — | 76 | 4 | 62 | 62 | 7 | + | 0-0-1- |
| | SpBE3 | ACCCUGGC | | | | | | | | | | | | 13-119 |
| P478S/L | EQR- | GCCCGCUGCGCC | (GGAG) | 20 (C13) | 3.1 | 81 | — | 61 | 3 | 57 | 38 | 3 | − | 0-0-0- |
| | SpBE3 | CCAGAUGA | | | | | | | | | | | | 5-73 |
| C534Y | St3BE3 | UGUGGACGCUGC | (TGGGG) | 20 (C12) | 5.1 | 92 | 28 | 21 | 3 | 50 | 38 | 5 | + | 0-0-0- |
| | | AGUUGGCC | | | | | | | | | | | | 2-57 |
| C588Y | VQR- | CGCACUGGUUGG | (CGTG) | 20 (C3) | 4.6 | 99 | — | 21 | 4 | 43 | 37 | 4 | − | 0-0-0- |
| | SpBE3 | GCUGACCU | | | | | | | | | | | | 0-4 |
| C223Y | VQR- | GUCACACUUGCU | (CGAC) | 20 (C5) | 5.3 | 72 | — | 43 | 3 | 25 | 69 | 5 | + | 0-0-0- |
| | SpBE3 | GGCCUGCU | | | | | | | | | | | | 5-161 |
| P288S/L | VRER- | CCCCUGGCCGGGU | (CGCG) | 21 (C1/−1) | 5.9 | 99 | — | 42 | 0 | 32 | 42 | 5 | − | 0-0-0- |
| | SpBE3 | GGGUACAGC | | | | | | | | | | | | 0-16 |
| C655Y | SpBE3 | GACUACACACGU | (CGG) | 20 (C8) | 8.3 | 84 | 57 | 32 | 24 | 44 | 41 | 8 | − | 0-0-0- |
| | | GUUGUCUA | | | | | | | | | | | | 9-34 |
| P530S/L | SpBE3 | CUGCUACCCCAG | (CAG) | 20 (C7/8) | 7.4 | 61 | 61 | 50 | 28 | 68 | 80 | 7 | − | 0-0-1- |
| | | GCCAACUG | | | | | | | | | | | | 25-215 |
| C534Y | SaBE3 | UGUGGACGCUGC | (TGGGGT) | 20 (C12) | 5.1 | 90 | 28 | 21 | 3 | 50 | 38 | 5 | + | 0-0-0- |
| | | AGUUGGCC | | | | | | | | | | | | 4-70 |
| G670R/E | SpBE3 | GGCUGUCACGGC | (TGG) | 20 (C12/13) | 4.6 | 80 | 37 | 60 | 2 | 51 | 25 | 4 | + | 0-0-1- |
| | | CCCUUCGC | | | | | | | | | | | | 12-104 |
| P25S/L | SpBE3 | UCCCGCGGGCGC | (AGG) | 20 (C2/3) | 7.6 | 79 | 38 | 60 | 0 | 56 | 48 | 7 | + | 0-0-2- |
| | | CCGUGCGC | | | | | | | | | | | | 12-133 |
| G337R/E | SpBE3 | UGGCCCCAACUG | (TGG) | 20 (C6/7) | 6.0 | 78 | 61 | 10 | 1 | 35 | 36 | 6 | − | 0-0-3- |
| | | UGAUGACC | | | | | | | | | | | | 6-136 |
| P639S/L | St3BE3 | CCUGGGACCUCC | (GGGGG) | 20 (C1/2) | 5.3 | 86 | 38 | 36 | 5 | 41 | 53 | 5 | + | 0-0-1- |
| | | CACGUCCU | | | | | | | | | | | | 14-53 |
| P345S/L | St3BE3 | CCAAGACCAGCC | (TGGGG) | 20 (C11/12) | 4.3 | 92 | 44 | 38 | 2 | 46 | 33 | 4 | + | 0-0-0- |
| | | GGUGACCC | | | | | | | | | | | | 6-53 |
| C509Y | SpBE3 | GCAGACCAGCUU | (GGG) | 20 (C2) | 8.4 | 68 | 41 | 66 | 18 | 62 | 70 | 8 | + | 0-0-1- |
| | | GCCCCCUU | | | | | | | | | | | | 14-153 |
| P279S/L | SpBE3 | CCAGCCUGUGGG | (TGG) | 20 (C5/6) | 5.4 | 53 | 48 | 84 | 10 | 78 | 66 | 5 | +GG | 0-0-8- |
| | | GCCACUGG | | | | | | | | | | | | 42-299 |
| C655Y | VRER- | ACUACACACGUG | (GGCG) | 20 (C7) | 6.8 | 100 | — | 37 | 10 | 29 | 35 | 6 | − | 0-0-0- |
| | SpBE3 | UUGUCUAC | | | | | | | | | | | | 0-0 |
| G516R/E | SpBE3 | CUCACCCCCAAA | (GGG) | 20 (C8/9) | 5.6 | 89 | 47 | 26 | 5 | 32 | 21 | 5 | − | 0-0-1- |
| | | AGCGUUGU | | | | | | | | | | | | 10-68 |
| C635Y | SpBE3 | GGAGGGCACUGC | (AGG) | 20 (C13) | 4.8 | 52 | 34 | 84 | 1 | 55 | 61 | 4 | + | 0-0-5- |
| | | AGCCAGUC | | | | | | | | | | | | 33-327 |
| G365R/E | EQR- | GAUGUCCUCCCC | (AGAG) | 20 (C11/12) | 6.9 | 66 | — | 69 | 4 | 67 | 61 | 6 | + | 0-0-0- |
| | SpBE3 | UGGGGCAA | | | | | | | | | | | | 21-139 |
| G450R/E | St3BE3 | CUUACCUGCCCC | (TGGGG) | 20 (C11/12) | 8.8 | 93 | 25 | 27 | 2 | 42 | 27 | 8 | + | 0-0-0- |
| | | AUGGGUGC | | | | | | | | | | | | 3-39 |
| G337R/E | VQR- | GGCCCCAACUGU | (GGAA) | 20 (C5/6) | 4.9 | 76 | — | 45 | 15 | 58 | 43 | 4 | − | 0-0-0- |
| | SpBE3 | GAUGACCU | | | | | | | | | | | | 10-96 |
| P576S/L | KKH- | AGCCGCCUGUGC | (CGAGGT) | 20 (C4,5,6,7) | 5.3 | 81 | 41 | 27 | 10 | 49 | 53 | 5 | + | 0-0-1- |
| and/or | SaBE3 | UGAGGCCA | | | | | | | | | | | | 7-46 |
| P577S/L | | | | | | | | | | | | | | |
| P430S/L | VQR- | CCCUGAGGACCA | (TGAC) | 20 (C2/3) | 7.6 | 87 | — | 21 | 0 | 26 | 46 | 7 | + | 0-0-0- |
| | SpBE3 | GCGGGUAC | | | | | | | | | | | | 7-75 |
| P639S/L | St3BE3 | CCCUGGGACCUC | (TGGGG) | 20 (C2/3) | 6.3 | 84 | 29 | 16 | 0 | 49 | 31 | 6 | + | 0-0-1- |
| | | CCACGUCC | | | | | | | | | | | | 11-68 |
| P155S/L | EQR- | CAGAGCAUCCCG | (GGAG) | 20 (C9/10) | 6.4 | 77 | — | 35 | 10 | 47 | 54 | 6 | − | 0-0-2- |
| | SpBE3 | UGGAACCU | | | | | | | | | | | | 6-98 |
| G232R/E | VQR- | GCUGACCACCCC | (GGG) | 20 (C9/10) | 6.2 | 49 | 58 | 82 | 8 | 68 | 60 | 6 | + | 0-0-5- |
| | SpBE3 | UGCCAGGU | | | | | | | | | | | | 30-182 |
| G450R/E | St3BE3 | UUACCUGCCCCA | (GGGGG) | 20 (C10/11) | 6.4 | 90 | 29 | 40 | 3 | 17 | 35 | 6 | + | 0-0-0- |
| | | UGGGUGCU | | | | | | | | | | | | 3-35 |
| G670R/E | KKH- | GCCCCUUCGCUG | (TGTAGT) | 20 (C4/5) | 8.9 | 90 | 36 | 40 | 14 | 30 | 24 | 8 | + | 0-0-1- |
| | SaBE3 | GUGCUGCC | | | | | | | | | | | | 6-27 |
| P71S/L | SpBE3 | CAGGAUCCGUGG | (TGG) | 20 (C7/8) | 5.5 | 77 | 42 | 16 | 3 | 23 | 52 | 5 | + | 0-0-1- |
| | | AGGUUGCC | | | | | | | | | | | | 9-124 |
| C486Y | St3BE3 | CAGCUCAGCAGC | (TGGGG) | 20 (C1) | 4.9 | 87 | 21 | 15 | 0 | 20 | 42 | 4 | − | 0-0-2- |
| | | UCCUCAUC | | | | | | | | | | | | 5-64 |
| C509Y | SpBE3 | GGCAGACCAGCU | (TGG) | 20 (C3) | 4.4 | 75 | 29 | 32 | 0 | 49 | 54 | 4 | + | 0-0-3- |
| | | UGCCCCCU | | | | | | | | | | | | 21-139 |
| P209S/L | SpBE3 | AGAAUGUGCCCG | (GGG) | 20 (C9/10) | 6.2 | 66 | 47 | 43 | 16 | 62 | 47 | 6 | + | 0-0-1- |
| | | AGGAGGAC | | | | | | | | | | | | 11-200 |
| P120S/L | KKH- | CAUGGCCUUCUU | (CCTGGT) | 20 (C7/8) | 7.2 | 67 | — | 2 | 6 | 36 | 60 | 7 | − | 0-0-3- |
| | SaBE3 | CCUGGCUU | | | | | | | | | | | | 12-77 |
| G516R/E | SpBE3 | CCCCAAAAGCGU | (CGG) | 20 (C3/4) | 6.7 | 84 | 38 | 3 | 1 | 22 | 42 | 6 | + | 0-0-0- |
| | | UGUGGGCC | | | | | | | | | | | | 3-81 |
| C323Y | SpBE3 | GGCAUCGUCCCG | (CGG) | 20 (C3) | 7.2 | 77 | 47 | 21 | 28 | 44 | 38 | 7 | − | 0-0-8- |
| | | GAAGUUGC | | | | | | | | | | | | 2-42 |
| C358Y | SpBE3 | GUCCACACAGCG | (TGG) | 20 (C8) | 4.1 | 72 | 52 | 36 | 3 | 52 | 39 | 4 | − | 0-0-2- |
| | | GCCAAAGU | | | | | | | | | | | | 16-85 |
| G493R/E | St3BE3 | CUUCCCACUCCU | (TGGAG) | 20 (C5/6) | 7.3 | 88 | 30 | 8 | 9 | 17 | 36 | 7 | − | 0-0-0- |
| | | GGAGAAAC | | | | | | | | | | | | 5-69 |
| P404S/L | SpBE3 | UGCCGAGCCGGA | (TGG) | 20 (C8/9) | 4.3 | 61 | 52 | 40 | 8 | 59 | 19 | 4 | + | 0-0-1- |
| | | GCUCACCC | | | | | | | | | | | | 18-117 |
| P540S/L | EQR- | GUCCACACAGCU | (TGAG) | 20 (C13) | 3.6 | 63 | — | 44 | 6 | 55 | 1 | 3 | + | 0-0-1- |
| and/or | SpBE3 | CCACCAGC | | | | | | | | | | | | 16-165 |
| P541S/L | | | | | | | | | | | | | | |
| G505R/E | EQR- | AGCUUGCCCCCU | (AGAG) | 20 (C10/11) | 6.9 | 75 | — | 10 | 0 | 21 | 42 | 6 | + | 0-0-0- |
| | SpBE3 | UGGGCCUU | | | | | | | | | | | | 8-120 |
| C534Y | SpBE3 | UGCAGUUGGCCU | (AGG) | 20 (C3) | 8.3 | 53 | 41 | 31 | 0 | 13 | 64 | 8 | + | 0-0-4- |
| | | GGGGUAGC | | | | | | | | | | | | 28-300 |
| P576S/L | EQR- | CACCCACAAGCC | (TGAG) | 20 (C11/12) | 4.6 | 80 | — | 23 | 0 | 37 | 24 | 4 | + | 0-0-2- |
| and/or | SpBE3 | GCCUGUGC | | | | | | | | | | | | 5-129 |
| P577S/L | | | | | | | | | | | | | | |
| P345S/L | SpBE3 | GCCGGUGACCCU | (TGG) | 20 (C2/3) | 7.4 | 52 | 33 | 41 | 1 | 33 | 63 | 7 | − | 0-0-6- |
| | | GGGGACUU | | | | | | | | | | | | 20-179 |
| P430S/L | VRER- | GGCCUGGUUCCC | (AGCG) | 20 (C11/12) | 5.8 | 63 | — | 14 | 0 | 51 | 44 | 5 | + | 0-1-0- |
| | SpBE3 | UGAGGACC | | | | | | | | | | | | 3-22 |
| G232R/E | VQR- | CCCCUGCCAGGU | (TGAC) | 20 (C2/3) | 4.7 | 56 | — | 32 | 11 | 46 | 57 | 4 | + | 0-0-2- |
| | SpBE3 | GGGUGCCA | | | | | | | | | | | | 32-272 |
| P279S/L | SpBE3 | GGUCCAGCCUGU | (TGG) | 20 (C8/9) | 6.6 | 50 | 36 | 39 | 2 | 50 | 63 | 6 | + | 0-0-3- |
| | | GGGGCCAC | | | | | | | | | | | | 39-270 |
| P478S/L | EQR- | CGCCCCAGAUGA | (TGAG) | 20 (C5/6) | 5.3 | 63 | — | 50 | 1 | 35 | 14 | 5 | + | 0-0-1- |
| | SpBE3 | GGAGCUGC | | | | | | | | | | | | 14-146 |
| P288S/L | SpBE3 | UGCUGCUGCCCC | (GGG) | 20 (C9/10) | 6.3 | 60 | 46 | 32 | 4 | 45 | 51 | 6 | + | 0-0-2- |
| | | UGGCGGGU | | | | | | | | | | | | 42-286 |
| C608Y | St3BE3 | UUGACUUUGCAU | (TGGGG) | 20 (C10) | 7.7 | 77 | 34 | 2 | 3 | 34 | 12 | 7 | + | 0-0-0- |
| | | UCCAGACC | | | | | | | | | | | | 6-141 |
| P364S/L | SpBE3 | GCCCCAGGGGAG | (TGG) | 20 (C4/5) | 6.6 | 41 | 40 | 60 | 8 | 54 | 67 | 6 | − | 0-1-2- |
| | | GACAUCAU | | | | | | | | | | | | 25-189 |
| C534Y | SpBE3 | UGUGGACGCUGC | (TGG) | 20 (C12) | 5.1 | 58 | 28 | 21 | 3 | 50 | 38 | 5 | + | 0-0-3- |
| | | AGUUGGCC | | | | | | | | | | | | 25-336 |
| G450R/E | SpBE3 | UUACCUGCCCCA | (GGG) | 20 (C10/11) | 6.4 | 67 | 29 | 40 | 3 | 17 | 35 | 6 | + | 0-0-1- |
| | | UGGGUGCU | | | | | | | | | | | | 12-141 |
| P639S/L | SpBE3 | CCCUGGGACCUC | (TGG) | 20 (C2/3) | 6.3 | 57 | 29 | 16 | 0 | 49 | 31 | 6 | + | 0-0-3- |
| | | CCACGUCC | | | | | | | | | | | | 38-294 |
| P576S/L | EQR- | AGCCGCCUGUGC | (CGAG) | 20 (C3,4,6,7) | 5.3 | 49 | — | 27 | 10 | 49 | 53 | 5 | + | 0-0-5- |
| and/or | SpBE3 | UGAGGCCA | | | | | | | | | | | | 26-182 |
| P577S/L | | | | | | | | | | | | | | |
| P616S/L | St3BE3 | AAUCCCGGCCCC | (AGGTG) | 20 | 6.6 | 40 | 51 | 44 | 12 | 60 | 40 | 6 | + | 0-0-0- |
| and/or | | UCAGGAGC | | (C5,6,11,12) | | | | | | | | | | 39-583 |
| P618S/L | | | | | | | | | | | | | | |
| C635Y | SpBE3 | CACUGCAGCCAG | (CAG) | 20 (C6) | 6.7 | 47 | 42 | 4 | 3 | 35 | 52 | 6 | + | 0-0-9- |
| | | UCAGGGUC | | | | | | | | | | | | 42-425 |
| P120S/L | St3BE3 | UGGCCUUCUUCC | (TGGTG) | 20 (C4/5) | 4.1 | 64 | 22 | 6 | 1 | 12 | 34 | 4 | + | 0-0-3- |
| | | UGGCUUCC | | | | | | | | | | | | 22-144 |

*Guide sequences (the portion of the guide RNA that targets the nucleobase editor to the target sequence) are provided, which may be used with any tracrRNA framework sequences provided herein to generate the full guide RNA sequence
^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.
^{b}Efficiency score, based on Housden et al (Science Signaling, 2015, 8(393): rs9).
^{c}Specificity scores based on Hsu et al (Nature biotechnology, 2013, 31(9): 827-832), Fusi et al (bioRxiv 021568; doi: http://dx.doi.org/10.1101/021568), Chari et al (Nature Methods, 2015, 12(9): 823-6), Doench et al (Nature Biotechnology, 2014, 32(12): 1262-7), Wang et al (Science, 2014, 343(6166): 80-4), Moreno-Mateos et al (Nature Methods, 2015, 12(10): 982-8), Housden et al (Science Signaling, 2015, 8(393): r59), and the “Prox/GC” column shows “+” if the proximal 6 bp to the PAM has a GC count >=4, and GG if the guide ends with GG, based on Farboud et al (Genetics, 2015, 199 (4): 959-71).
^{d}Number of predicted off-target binding sites in the human genome allowing up to 0, 1, 2, 3 or 4 mismatches, respectively shown in the format 0-1-2-3-4. Algorithm used: Haeussler et al, Genome Biol. 2016; 17: 148.

TABLE 11
Efficiency and Specificity Scores for gRNAs for Introducing Premature Stop Codon into PCSK9 Gene
via Base Editing. Guide sequences correspond to SEQ ID NOs: 1621-1700 from top to bottom.

| Target | | guide | | gRNA size | | | | | | | | Hous | Prox/ | Off- |
|---|---|---|---|---|---|---|---|---|---|---|---|---|---|---|
| codon | BE type^{a} | sequence | PAM | (C edited) | Eff.^{b} | Hsu^{c} | Fusi | C. | Doench | W. | M.-M. | den | GC | targets |
| R582 | VQR- | CGAGGUCAGCC | (CGTG) | 20 (C6/1) | 7.5 | 99 | — | 94 | 4 | 58 | 78 | 7 | + | 0-0-0- |
| and/or | SpBE3 | CAACCAGUG | | | | | | | | | | | | 0-1 |
| Q584 | | | | | | | | | | | | | | |
| R582 | VQR- | GCCACGAGGUC | (AGTG) | 20 (C11/5) | 5.2 | 99 | — | 93 | 1 | 54 | 41 | 5 | + | 0-0-0- |
| and/or | SpBE3 | AGCCCAACC | | | | | | | | | | | | 0-7 |
| Q584 | | | | | | | | | | | | | | |
| Q190 | KKH- | AGCAUACAGAG | (GGAAA | 20 (C7) | 6.0 | 98 | 83 | 93 | 52 | 84 | 60 | 6 | + | 0-0-0- |
| | SaBE3 | UGACCACCG | T) | | | | | | | | | | | 0-18 |
| R582 | VRER- | CACGAGGUCAG | (TGCG) | 20 (C9/3) | 4.4 | 100 | — | 87 | 20 | 90 | 69 | 4 | − | 0-0-0- |
| and/or | SpBE3 | CCCAACCAG | | | | | | | | | | | | 0-5 |
| Q584 | | | | | | | | | | | | | | |
| Q433 | KKH- | CAGCGGGUACU | (CCTGG | 20 (C1) | 6.6 | 97 | — | 60 | 30 | 59 | 92 | 6 | + | 0-0-0- |
| | SaBE3 | GACCCCCAA | T) | | | | | | | | | | | 1-8 |
| Q219 | KKH- | CAGACAGGUAA | (TCTGA | 20 (C5) | 5.1 | 99 | — | 77 | 38 | 89 | 62 | 5 | + | 0-0-0- |
| | SaBE3 | GCACGGCCG | T) | | | | | | | | | | | 0-16 |
| Q219 | VQR- | GACAGGUAAGC | (TGAT) | 20 (C3) | 3.8 | 97 | — | 90 | 5 | 41 | 42 | 3 | + | 0-0-0- |
| | SpBE3 | ACGGCCGUC | | | | | | | | | | | | 0-33 |
| Q342 | KKH- | GCCACCAAUGC | (GCCGG | 20 (C13) | 3.1 | 92 | — | 92 | 29 | 73 | 49 | 3 | − | 0-0-0- |
| and/or | SaBE3 | CCAAGACCA | T) | | | | | | | | | | | 2-29 |
| Q344 | | | | | | | | | | | | | | |
| R582 | KKH- | GAGGCCACGAG | (ACCAG | 20 (C8) | 4.6 | 96 | — | 61 | 12 | 87 | 79 | 4 | + | 0-0-0- |
| and/or | SaBE3 | GUCAGCCCA | T) | | | | | | | | | | | 1-18 |
| R584 | | | | | | | | | | | | | | |
| Q342 | VQR- | CAAUGCCCAAG | (TGAC) | 20 (C8) | 4.3 | 86 | — | 94 | 13 | 89 | 56 | 4 | +GG | 0-0-0- |
| and/or | SpBE3 | ACCAGCCGG | | | | | | | | | | | | 9-83 |
| Q344 | | | | | | | | | | | | | | |
| Q454 | KKH- | GCAGCUGUUUU | (TATGG | 20 (C2) | 4.3 | 89 | — | 91 | 18 | 81 | 50 | 4 | + | 0-0-0- |
| | SaBE3 | GCAGGACUG | T) | | | | | | | | | | | 3-64 |
| Q256 | KKH- | CUCAACUGCCA | (CACGG | 20 (C10) | 7.1 | 84 | — | 95 | 9 | 72 | 49 | 7 | +GG | 0-0-0- |
| | SaBE3 | AGGGAAGGG | T) | | | | | | | | | | | 5-65 |
| Q387 | KKH- | CACAGGCUGCU | (GCTGG | 20 (C3) | 7.7 | 95 | — | 81 | 4 | 56 | 73 | 7 | + | 0-0-0- |
| | SaBE3 | GCCCACGUG | T) | | | | | | | | | | | 3-23 |
| R582 | SpBE3 | GGUCAGCCCAA | (GGG) | 20 (C4/13) | 4.8 | 86 | 62 | 59 | 44 | 88 | 34 | 4 | + | 0-0-2- |
| and/or | | CCAGUGCGU | | | | | | | | | | | | 6-51 |
| Q584 | | | | | | | | | | | | | | |
| Q101X | EQR- | AGGCCCAGGCU | (GGAT) | 20 (C6) | 7.9 | 79 | — | 92 | 3 | 80 | 94 | 7 | +GG | 0-0-0- |
| | SpBE3 | GCCCGCCGG | | | | | | | | | | | | 24-153 |
| Q99X | SaBE3 | GCAGGCCCAGG | (GGGGA | 20 (C2/8) | 4.9 | 94 | 26 | 77 | 8 | 53 | 74 | 4 | + | 0-0-0- |
| and/or | | CUGCCCGCC | T) | | | | | | | | | | | 6-43 |
| Q101X | | | | | | | | | | | | | | |
| Q587 | St3BE3 | CAACCAGUGCG | (GGGAG) | 20 (C5) | 8.5 | 91 | 55 | 79 | 23 | 37 | 60 | 8 | + | 0-0-0- |
| | | UGGGCCACA | | | | | | | | | | | | 1-32 |
| Q503 | KKH- | UCUAAGGCCCA | (GCTGG | 20 (C10) | 7.7 | 94 | — | 75 | 17 | 72 | 61 | 7 | + | 0-0-0- |
| | SaBE3 | AGGGGGCAA | T) | | | | | | | | | | | 0-30 |
| Q278 | St3BE3 | CCAGCCUGUGG | (TGGTG) | 20 (C2) | 5.4 | 85 | 48 | 84 | 10 | 78 | 66 | 5 | +GG | 0-0-3- |
| and/or | | GGCCACUGG | | | | | | | | | | | | |
| Q275 | | | | | | | | | | | | | | |
| Q554 | KKH- | ACCAACAGGGC | (ACAGG | 20 (C3/6) | 5.3 | 97 | — | 71 | 0 | 29 | 49 | 5 | + | 0-0-0- |
| and/or | SaBE3 | CACGUCCUC | T) | | | | | | | | | | | 0-18 |
| Q555 | | | | | | | | | | | | | | |
| Q31 | VRER- | GUGCGCAGGAG | (GGCG) | 20 (C6) | 5.9 | 98 | — | 53 | 2 | 60 | 68 | 5 | + | 0-0-0- |
| | SpBE3 | GACGAGGAC | | | | | | | | | | | | 0-17 |
| W453 | SaBE3 | GCCAACCUGCA | (TGGGA | 20 (C2/3) | 7.2 | 95 | 37 | 53 | 11 | 71 | 10 | 7 | + | 0-0-0- |
| | | AAAAGGGCC | T) | | | | | | | | | | | 0-34 |
| Q302 | VRER- | AACGCCGCCUG | (GGCG) | 20 (C13) | 5.0 | 97 | — | 59 | 13 | 68 | 41 | 5 | + | 0-0-0- |
| | SpBE3 | CCAGCGCCU | | | | | | | | | | | | 0-14 |
| Q256 | VRER- | GCCAAGGGAAG | (AGCG) | 20 (C3) | 4.1 | 97 | — | 66 | 6 | 67 | 57 | 4 | − | 0-0-0- |
| | SpBE3 | GGCACGGUU | | | | | | | | | | | | 2-18 |
| Q302 | EQR- | CGCCGCCUGCC | (CGAG) | 20 (C11) | 8.6 | 71 | — | 93 | 11 | 54 | 52 | 8 | +GG | 0-0-0- |
| | SpBE3 | AGCGCCUGG | | | | | | | | | | | | 15-115 |
| Q275 | VQR- | AAAAGCCAGCU | (TGTG) | 20 (C7) | 9.7 | 95 | — | 67 | 1 | 50 | 46 | 9 | + | 0-0-0- |
| | SpBE3 | GGUCCAGCC | | | | | | | | | | | | 0-32 |
| Q621 | EQR- | GGAGCAGGUGA | (TGAG) | 20 (C5) | 6.2 | 62 | — | 99 | 56 | 93 | 69 | 6 | + | 0-0-2- |
| | SpBE3 | AGAGGCCCG | | | | | | | | | | | | 24-248 |
| Q172 | VQR- | UGAAUACCAGC | (AGAC) | 20 (C8) | 3.7 | 97 | — | 63 | 2 | 59 | 62 | 3 | + | 0-0-0- |
| | SpBE3 | CCCCCGGUA | | | | | | | | | | | | 1-31 |
| Q172 | SpBE3 | AUGAAUACCAG | (AAG) | 20 (C9) | 4.4 | 90 | 64 | 61 | 32 | 70 | 56 | 4 | + | 0-0-0- |
| | | CCCCCCGGU | | | | | | | | | | | | 6-48 |
| Q99X | St3BE3 | UGCAGGCCCAG | (CGGGG) | 20 (C3/9) | 6.2 | 85 | 34 | 70 | 17 | 75 | 51 | 6 | + | 0-0-0- |
| and/or | | GCUGCCCGC | | | | | | | | | | | | 3-96 |
| Q101X | | | | | | | | | | | | | | |
| Q584 | SpBE3 | AGGUCAGCCCA | (TGG) | 20 (C5) | 7.2 | 83 | 56 | 70 | 36 | 77 | 37 | 7 | + | 0-0-2- |
| | | ACCAGUGCG | | | | | | | | | | | | 6-65 |
| Q621 | SpBE3 | AGCAGGUGAAG | (AGG) | 20 (C3) | 5.2 | 62 | 61 | 98 | 23 | 58 | 69 | 5 | + | 0-0-1- |
| | | AGGCCCGUG | | | | | | | | | | | | 28-271 |
| Q531 | VQR- | UGCUACCCCAG | (AGCG) | 20 (C9) | 4.1 | 99 | — | 23 | 3 | 60 | 19 | 4 | − | 0-0-0- |
| | SpBE3 | GCCAACUGC | | | | | | | | | | | | 1-5 |
| W428 | KKH- | UCCUCAGGGAA | (ATTGA | 20 (C11/12) | 6.3 | 88 | — | 70 | 0 | 42 | 63 | 6 | + | 0-0-0- |
| | SaBE3 | CCAGGCCUC | T) | | | | | | | | | | | 3-45 |
| Q31 | VQR- | GCCCGUGCGCA | (GGAC) | 20 (C10) | 7.7 | 81 | — | 76 | 28 | 77 | 60 | 7 | + | 0-0-0- |
| | SpBE3 | GGAGGACGA | | | | | | | | | | | | 4-91 |
| Q275 | St3BE3 | AAGCCAGCUGG | (TGGGG) | 20 (C5) | 4.6 | 80 | 51 | 56 | 3 | 73 | 78 | 4 | + | 0-0-0- |
| | | UCCAGCCUG | | | | | | | | | | | | 7-79 |
| Q31 | EQR- | GGCGCCCGUGC | (CGAG) | 20 (C13) | 4.0 | 68 | — | 90 | 6 | 70 | 62 | 4 | + | 0-0-2- |
| | SpBE3 | GCAGGAGGA | | | | | | | | | | | | 11-115 |
| W10 | St3BE3 | CCAGGACCGCC | (CGGTG) | 20 (C−1) | 8.0 | 80 | 55 | 23 | 25 | 60 | 77 | 8 | − | 0-0-0- |
| and/or | | UGGAGCUGA | | | | | | | | | | | | 9-71 |
| W11 | | | | | | | | | | | | | | |
| Q31 | St3BE3 | CGUGCGCAGGA | (CGGCG | 20 (C7) | 6.7 | 76 | 58 | 81 | 27 | 73 | 70 | 6 | + | 0-0-0- |
| | | GGACGAGGA | | | | | | | | | | | | 4-127 |
| Q686 | St3BE3 | GCACCUGGCGC | (CAGGA | 19 (C11) | 7.6 | 60 | 38 | 97 | 9 | 56 | 59 | 4 | + | 0-1-0- |
| | | AGGCCUCC | G) | | | | | | | | | | | 12-76 |
| Q152 | VQR- | CUUUGCCCAGA | (GGAA) | 20 (C7) | 5.1 | 75 | — | 55 | 81 | 67 | 47 | 5 | + | 0-0-2- |
| | SpBE3 | GCAUCCCGU | | | | | | | | | | | | 8-120 |
| Q152 | VQR- | UGUCUUUGCCC | (CGTG) | 20 (C10) | 6.6 | 98 | — | 56 | 4 | 31 | 6 | 6 | + | 0-0-0- |
| | SpBE3 | AGAGCAUCC | | | | | | | | | | | | 2-19 |
| Q584 | SpBE3 | GGCCACGAGGU | (CAG) | 20 (C12) | 5.9 | 85 | 40 | 64 | 13 | 25 | 69 | 5 | + | 0-0-1- |
| | | CAGCCCAAC | | | | | | | | | | | | 4-70 |
| Q278 | KKH- | CUGGUCCAGCC | (ACTGG | 20 (C7) | 10.8 | 83 | — | 21 | 0 | 43 | 71 | 10 | + | 0-0-0- |
| and/or | SaBE3 | UGUGGGGCC | T) | | | | | | | | | | | 10-77 |
| Q275 | | | | | | | | | | | | | | |
| W10 | EQR- | AGCGGCCACCA | (GGAG) | 20 | 8.2 | 82 | — | 51 | 2 | 72 | 57 | 8 | + | 0-0-1- |
| and/or | SpBE3 | GGACCGCCU | | (C9,10,6,7) | | | | | | | | | | 9-94 |
| W11 | | | | | | | | | | | | | | |
| Q587 | EQR- | AACCAGUGCGU | (GGAG) | 20 (C4) | 4.0 | 64 | — | 90 | 15 | 67 | 70 | 4 | + | 0-0-2- |
| | SpBE3 | GGGCCACAG | | | | | | | | | | | | 15-149 |
| W10 | St3BE3 | CAGCGGCCACC | (TGGAG) | 20 | 6.6 | 90 | 43 | 63 | 17 | 53 | 48 | 6 | + | 0-0-0- |
| and/or | | AGGACCGCC | | (C10,11,7,8) | | | | | | | | | | 6-55 |
| W11 | | | | | | | | | | | | | | |
| W630 | KKH- | GUCCAGCCCUC | (CACGG | 20 (C3/4) | 3.3 | 95 | — | 52 | 7 | 57 | 32 | 3 | + | 0-0-0- |
| | SaBE3 | CUCGCAGGC | T) | | | | | | | | | | | 3-43 |
| Q152 | SpBE3 | UCUUUGCCCAG | (TGG) | 20 (C9) | 4.8 | 63 | 66 | 89 | 73 | 87 | 44 | 4 | + | 0-0-5- |
| | | AGCAUCCCG | | | | | | | | | | | | 18-163 |
| Q387 | SpBE3 | AUCACAGGCUG | (TGG) | 20 (C5) | 5.1 | 61 | 59 | 91 | 16 | 43 | 70 | 5 | + | 0-0-3- |
| | | CUGCCCACG | | | | | | | | | | | | 13-177 |
| Q342 | St3BE3 | CACCAAUGCCC | (CGGTG) | 20 (C11) | 5.0 | 94 | 53 | 57 | 39 | 42 | 20 | 5 | + | 0-0-0- |
| and/or | | AAGACCAGC | | | | | | | | | | | | 1-42 |
| Q344 | | | | | | | | | | | | | | |
| Q302 | SaBE3 | UGCCAGCGCCU | (TGGGG | 20 (C4) | 6.8 | 94 | 20 | 38 | 1 | 57 | 27 | 6 | + | 0-0-0- |
| | | GGCGAGGGC | T) | | | | | | | | | | | 3-48 |
| Q278 | KKH- | GUCCAGCCUGU | (GGTGG | 20 (C4) | 4.7 | 90 | 30 | 51 | 9 | 31 | 60 | 4 | + | 0-0-0- |
| and/or | SaBE3 | GGGGCCACU | T) | | | | | | | | | | | 6-28 |
| Q275 | | | | | | | | | | | | | | |
| Q554 | SpBE3 | CAACAGGGCCA | (AGG) | 20 (C1/4) | 9.6 | 74 | 58 | 76 | 7 | 50 | 70 | 9 | + | 0-0-1- |
| and/or | | CGUCCUCAC | | | | | | | | | | | | 17-125 |
| Q555 | | | | | | | | | | | | | | |
| Q152 | St3BE3 | CCAGAGCAUCC | (TGGAG) | 20 (C1) | 8.6 | 90 | 45 | 59 | 3 | 41 | 32 | 8 | + | 0-0-1- |
| | | CGUGGAACC | | | | | | | | | | | | 2-68 |
| Q302 | SpBE3 | CGCCUGCCAGC | (GGG) | 20 (C8) | 3.0 | 78 | 36 | 31 | 21 | 71 | 56 | 3 | + | 0-0-0- |
| | | GCCUGGCGA | | | | | | | | | | | | 13-129 |
| Q31 | SpBE3 | CGCCCGUGCGC | (AGG) | 20 (C11) | 4.4 | 64 | 43 | 85 | 10 | 60 | 49 | 4 | + | 0-0-1- |
| | | AGGAGGACG | | | | | | | | | | | | 15-154 |
| Q278 | St3BE3 | GGUCCAGCCUG | (TGGTG) | 20 (C5) | 6.6 | 85 | 36 | 39 | 2 | 50 | 63 | 6 | + | 0-0-0- |
| and/or | | UGGGGCCAC | | | | | | | | | | | | 13-49 |
| Q275 | | | | | | | | | | | | | | |
| Q190 | VQR- | AGCAUACAGAG | (GGAA) | 20 (C7) | 6.0 | 83 | — | 40 | 3 | 31 | 62 | 7 | − | 0-0-0- |
| | SpBE3 | UGACCACCG | | | | | | | | | | | | 7-134 |
| Q190 | EQR- | CAGAGUGACCA | (CGAG) | 20 (C1) | 7.6 | 83 | — | 40 | 3 | 31 | 62 | 7 | − | 0-0-0- |
| | SpBE3 | CCGGGAAAU | | | | | | | | | | | | 7-134 |
| Q686 | SaBE3 | GGCGCAGGCCU | (TCCAG | 20 (C5) | 6.3 | 69 | — | 32 | 5 | 75 | 44 | 6 | + | 0-0-1- |
| | | CCCAGGAGC | T) | | | | | | | | | | | 6-74 |
| W10 | KKH- | CACCAGGACCG | (GACGG | 20 (C3,4,1) | 7.9 | 86 | — | 56 | 1 | 39 | 50 | 7 | + | 0-0-1- |
| and/or | SaBE3 | CCUGGAGCU | T) | | | | | | | | | | | 10-41 |
| W11 | | | | | | | | | | | | | | |
| W453 | SpBE3 | GCCAACCUGCA | (TGG) | 20 (C2/3) | 7.2 | 68 | 37 | 53 | 11 | 71 | 10 | 7 | + | 0-0-7- |
| | | AAAAGGGCC | | | | | | | | | | | | 12-130 |
| Q342 | St3BE3 | CCAAGACCAGC | (TGGGG) | 20 (C2/8) | 4.3 | 92 | 44 | 38 | 2 | 46 | 33 | 4 | + | 0-0-0- |
| and/or | | CGGUGACCC | | | | | | | | | | | | 6-53 |
| Q344 | | | | | | | | | | | | | | |
| Q302 | St3BE3 | UGCCAGCGCCU | (TGGGG) | 20 (C4) | 6.8 | 80 | 20 | 38 | 1 | 57 | 27 | 6 | + | 0-0-1- |
| | | GGCGAGGGC | | | | | | | | | | | | 13-110 |
| Q587 | SpBE3 | CAACCAGUGCG | (GGG) | 20 (C5) | 8.5 | 57 | 55 | 79 | 23 | 37 | 60 | 8 | + | 0-0-0- |
| | | UGGGCCACA | | | | | | | | | | | | 34-114 |
| Q302 | SpBE3 | CCGCCUGCCAG | (AGG) | 20 (C9) | 5.4 | 63 | 40 | 72 | 6 | 72 | 50 | 5 | + | 0-0-2- |
| | | CGCCUGGCG | | | | | | | | | | | | 20-225 |
| W156 | SpBE3 | CCAGGUUCCAC | (TGG) | 20 (C8/9) | 4.0 | 71 | 29 | 4 | 2 | 63 | 33 | 4 | − | 0-0-1- |
| | | GGGAUGCUC | | | | | | | | | | | | 14-147 |
| Q433 | VQR- | CCCUGAGGACC | (TGAC) | 20 (C11) | 7.6 | 87 | — | 21 | 0 | 26 | 46 | 7 | + | 0-0-0- |
| | SpBE3 | AGCGGGUAC | | | | | | | | | | | | 7-75 |
| Q454 | VQR- | AGGUUGGCAGC | (GGAC) | 20 (C8) | 6.7 | 71 | — | 19 | 49 | 50 | 62 | 6 | − | 0-0-1- |
| | SpBE3 | UGUUUUGCA | | | | | | | | | | | | 17-178 |
| Q503 | SpBE3 | UAAGGCCCAAG | (TGG) | 20 (C8) | 5.1 | 64 | 51 | 69 | 5 | 53 | 34 | 5 | + | 0-0-0- |
| | | GGGGCAAGC | | | | | | | | | | | | 14-168 |
| W156 | VQR- | CCACGGGAUGC | (AGAC) | 20 (C1/2) | 6.4 | 60 | — | 62 | 3 | 62 | 71 | 6 | + | 0-0-3- |
| | SpBE3 | UCUGGGCAA | | | | | | | | | | | | 26-128 |
| W630 | SpBE3 | CAGGGUCCAGC | (AGG) | 20 (C7/8) | 6.3 | 63 | 55 | 66 | 2 | 55 | 60 | 6 | + | 0-0-3- |
| | | CCUCCUCGC | | | | | | | | | | | | 23-318 |
| Q31 | VQR- | GCGCAGGAGGA | (CGAC) | 20 (C4) | 6.2 | 29 | — | 99 | 54 | 91 | 90 | 6 | +GG | 0-0-4- |
| | SpBE3 | CGAGGACGG | | | | | | | | | | | | 59-1094 |
| Q587 | SpBE3 | CCAACCAGUGC | (AGG) | 20 (C6) | 4.7 | 60 | 42 | 68 | 0 | 38 | 62 | 4 | + | 0-0-7- |
| | | GUGGGCCAC | | | | | | | | | | | | 5-103 |
| Q99X | SpBE3 | CAGGCCCAGGC | (GGG) | 20 (C1/7) | 6.6 | 37 | 50 | 90 | 6 | 80 | 89 | 6 | + | 0-1-2- |
| and/or | | UGCCCGCCG | | | | | | | | | | | | 66-344 |
| Q101X | | | | | | | | | | | | | | |
| Q99X | SpBE3 | UGCAGGCCCAG | (CGG) | 20 (C3/9) | 6.2 | 52 | 34 | 70 | 17 | 75 | 51 | 6 | + | 0-0-2- |
| and/or | | GCUGCCCGC | | | | | | | | | | | | 45-342 |
| Q101X | | | | | | | | | | | | | | |
| W10 | SpBE3 | CAGCGGCCACC | (TGG) | 20 | 6.6 | 61 | 43 | 63 | 17 | 53 | 48 | 6 | + | 0-1-0- |
| and/or | | AGGACCGCC | | (C10,11,7,8) | | | | | | | | | | 28-213 |
| W11 | | | | | | | | | | | | | | |
| W630 | SpBE3 | UCAGGGUCCAG | (CAG) | 20 (C8/9) | 4.0 | 44 | 63 | 74 | 41 | 77 | 35 | 4 | + | 0-0-0- |
| | | CCCUCCUCG | | | | | | | | | | | | 47-393 |
| W10 | VQR- | CCACCAGGACC | (TGAC) | 20 | 5.7 | 55 | — | 32 | 3 | 60 | 29 | 5 | + | 0-0-6- |
| and/or | SpBE3 | GCCUGGAGC | | (C4,5,1,2) | | | | | | | | | | 37-179 |
| W11 | | | | | | | | | | | | | | |

*Guide sequences (the portion of the guide RNA that targets the nucleobase editor to the target sequence) are prov.ded, which may be used with any tracrRNA framework sequences provided herein to generate the full guide RNA sequence
aBE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.
bEfficiency score, based on Housden et al (Science Signaling, 2015, 8(393): rs9).
cSpecificity scores based on Hsu et al (Nature biotechnology, 2013, 31 (9): 827-832), Fusi et al (bioRxiv 021568; doi: http://dx.doi.org/10.1101/021568), Chari et al (Nature Methods, 2015, 12 (9): 823-6), Doench et al (Nature Biotechnology, 2014, 32 (12): 1262-7), Wang et al (Science, 2014, 343 (6166): 80-4), Moreno-Mateos et al (Nature Methods, 2015, 12 (10): 982-8), Housden et al (Science Signaling, 2015, 8 (393): rs9), and the “Prox/GC” column shows “+” if the proximal 6 bp to the PAM has a GC count >=4, and GG if the guide ends with GG, based on Farboud et al (Genetics, 2015, 199 (4): 959-71).
dNumber of predicted off-target binding sites in the human genome allowing up to 0, 1, 2, 3 or 4 mismatches, respectively shown in the format 0-1-2-3-4. Algorithm used: Haeussler et al, Genome Biol. 2016; 17: 148.

TABLE 12
Efficiency and Specificity Scores for gRNAs for Alteration of Intron/Exon Junctions in PCSK9 Gene
via Base Editing. Guide sequences correspond to SEQ ID NOs: 1701-1768 from top to bottom.

| | | | | gRNA | | | | | | | | | | |
|---|---|---|---|---|---|---|---|---|---|---|---|---|---|---|
| Target | | guide | | size | | | | | | | M. | Hous | Prox/ | Off- |
| intron | BE type^{a} | sequence | PAM | (C edited) | Eff.^{b} | Hsu^{c} | Fusi | Ch. | Doench | W. | M.- | den | GC | targets^{d} |
| intron 1, | KKH- | CGCACCUUGGC | (GAAGGT) | 20 (C5/6) | 5.1 | 98 | — | 85 | 2 | 48 | 53 | 5 | + | 0-0-0- |
| donor | SaBE3 | GCAGCGGUG | | | | | | | | | | | | 0-10 |
| site | | | | | | | | | | | | | | |
| intron | VQR- | GGUCACCUGCC | (GGAA) | 20 (C7) | 8.0 | 81 | — | 99 | 78 | 85 | 55 | 8 | + | 0-0-0- |
| 11, | SpBE3 | AGAGCCCGA | | | | | | | | | | | | 14-113 |
| acceptor | | | | | | | | | | | | | | |
| site | | | | | | | | | | | | | | |
| intron 6, | St3BE3 | GAUGACCUGGA | (AGGTG) | 20 (C7) | 6.3 | 81 | 73 | 98 | 52 | 88 | 52 | 6 | +GG | 0-0-2- |
| acceptor | | AAGGUGAGG | | | | | | | | | | | | 6-98 |
| site | | | | | | | | | | | | | | |
| intron 1, | VQR- | CCGCACCUUGG | (GGAA) | 20 (C6/7) | 5.2 | 93 | — | 39 | 4 | 45 | 85 | 5 | + | 0-0-0- |
| donor | SpBE3 | CGCAGCGGU | | | | | | | | | | | | 5-28 |
| site | | | | | | | | | | | | | | |
| intron 1, | St3BE3 | CACCUUGGCGC | (AGGTG) | 20 (C3/4) | 4.9 | 95 | 46 | 83 | 2 | 33 | 57 | 4 | + | 0-0-0- |
| donor | | AGCGGUGGA | | | | | | | | | | | | 2-33 |
| site | | | | | | | | | | | | | | |
| intron 1, | St3BE3 | ACACCCGCACC | (CGGTG) | 20 | 6.7 | 93 | 64 | 83 | 41 | 75 | 43 | 6 | + | 0-0-0- |
| donor | | UUGGCGCAG | | (C10/11) | | | | | | | | | | 0-26 |
| site | | | | | | | | | | | | | | |
| intron 1, | VRER- | CUACACCCGCA | (AGCG) | 20 | 9.0 | 99 | — | 27 | 23 | 77 | 31 | 9 | + | 0-0-0- |
| donor | SpBE3 | CCUUGGCGC | | (C12/13) | | | | | | | | | | 0-7 |
| site | | | | | | | | | | | | | | |
| intron 4, | VQR- | ACACUUGCUGG | (CGAA) | 20 (C13) | 5.8 | 91 | — | 84 | 40 | 69 | 56 | 5 | + | 0-0-0- |
| acceptor | SpBE3 | CCUGCUCGA | | | | | | | | | | | | 0-85 |
| site | | | | | | | | | | | | | | |
| intron 7, | SaBE3 | CUGCAAUGCCU | (GTGAAT) | 20 (C10) | 8.0 | 88 | — | 85 | 40 | 66 | 72 | 8 | +GG | 0-0-2- |
| acceptor | | GGUGCAGGG | | | | | | | | | | | | 5-52 |
| site | | | | | | | | | | | | | | |
| intron 6, | SaBE3 | UGACCUGGAAA | (GTGGGT) | 20 (C5) | 7.6 | 78 | — | 95 | 38 | 80 | 65 | 7 | + | 0-0-1- |
| acceptor | | GGUGAGGAG | | | | | | | | | | | | 8-99 |
| site | | | | | | | | | | | | | | |
| intron 1, | SpBE3 | CCCGCACCUUG | (TGG) | 20 (C7/8) | 4.3 | 89 | 50 | 70 | 16 | 83 | 64 | 4 | +GG | 0-0-0- |
| donor | | GCGCAGCGG | | | | | | | | | | | | 4-76 |
| site | | | | | | | | | | | | | | |
| intron 8, | St3BE3 | AUCCUGCUUAC | (GGGTG) | 20 | 4.3 | 92 | 47 | 38 | 7 | 39 | 80 | 4 | + | 0-0-0- |
| donor | | CUGCCCCAU | | (C11/12) | | | | | | | | | | 3-22 |
| site | | | | | | | | | | | | | | |
| intron 1, | SpBE3 | GCACCUUGGCG | (AGG) | 20 (C4/5) | 7.0 | 81 | 38 | 91 | 4 | 78 | 73 | 7 | +GG | 0-0-1- |
| donor | | CAGCGGUGG | | | | | | | | | | | | 11-110 |
| site | | | | | | | | | | | | | | |
| intron 1, | SpBE3 | CACCUUGGCGC | (AGG) | 20 (C3/4) | 4.9 | 88 | 46 | 83 | 2 | 33 | 57 | 4 | + | 0-0-0- |
| donor | | AGCGGUGGA | | | | | | | | | | | | 8-73 |
| site | | | | | | | | | | | | | | |
| intron | KKH- | ACCUGUGAGGA | (GTTGGT) | 20 (C2/3) | 9.0 | 96 | — | 62 | 3 | 47 | 72 | 9 | + | 0-0-0- |
| 10, | SaBE3 | CGUGGCCCU | | | | | | | | | | | | 2-20 |
| donor | | | | | | | | | | | | | | |
| site | | | | | | | | | | | | | | |
| intron 8, | SaBE3 | GCCAACCUGCA | (TGGGAT) | 20 (C7) | 7.2 | 95 | 37 | 53 | 11 | 71 | 10 | 7 | + | 0-0-0- |
| acceptor | | AAAAGGGCC | | | | | | | | | | | | 0-34 |
| site | | | | | | | | | | | | | | |
| intron 1, | SpBE3 | ACACCCGCACC | (CGG) | 20 | 6.7 | 82 | 64 | 83 | 41 | 75 | 43 | 6 | + | 0-0-0- |
| donor | | UUGGCGCAG | | (C10/11) | | | | | | | | | | 1-92 |
| site | | | | | | | | | | | | | | |
| intron 7, | KKH- | CAAUGCCUGGU | (AATGGT) | 20 (C7) | 6.0 | 85 | — | 79 | 1 | 53 | 80 | 6 | + | 0-0-0- |
| acceptor | SaBE3 | GCAGGGGUG | | | | | | | | | | | | 8-57 |
| site | | | | | | | | | | | | | | |
| intron | St1BE3 | CACCUGCCAGA | (AAAGAAA) | 20 (C4) | 3.8 | 98 | — | 53 | 4 | 64 | 49 | 3 | + | 0-0-0- |
| 11, | | GCCCGAGGA | | | | | | | | | | | | 0-13 |
| acceptor | | | | | | | | | | | | | | |
| site | | | | | | | | | | | | | | |
| intron | St3BE3 | CUGUGAGGACG | (TGGTG) | 20 (C1/-1) | 8.3 | 90 | 54 | 21 | 3 | 32 | 72 | 8 | + | 0-0-0- |
| 10, | | UGGCCCUGU | | | | | | | | | | | | 5-34 |
| donor | | | | | | | | | | | | | | |
| site | | | | | | | | | | | | | | |
| intron 3, | SpBE3 | UCUUUCCAAGG | (TGG) | 20 (C2) | 6.3 | 74 | 44 | 88 | 7 | 26 | 35 | 6 | − | 0-0-1- |
| acceptor | | CGACAUUUG | | | | | | | | | | | | 9-123 |
| site | | | | | | | | | | | | | | |
| intron 1, | SpBE3 | GAUCCUGGCCC | (AGG) | 20 (C5) | 8.1 | 62 | 70 | 99 | 65 | 78 | 49 | 8 | +GG | 0-0-3- |
| acceptor | | CAUGCAAGG | | | | | | | | | | | | 24-164 |
| site | | | | | | | | | | | | | | |
| intron 4, | SpBE3 | UGGCCUGCUCG | (AGG) | 20 (C5) | 6.0 | 88 | 56 | 73 | 21 | 62 | 49 | 6 | − | 0-0-0- |
| acceptor | | ACGAACACA | | | | | | | | | | | | 6-49 |
| site | | | | | | | | | | | | | | |
| intron 1, | St3BE3 | ACGGAUCCUGG | (AGGAG) | 20 (C8) | 4.4 | 93 | 53 | 65 | 6 | 61 | 65 | 4 | − | 0-0-0- |
| acceptor | | CCCCAUGCA | | | | | | | | | | | | 2-27 |
| site | | | | | | | | | | | | | | |
| intron 7, | SpBE3 | CUUACCAGCCA | (CAG) | 20 (C5/6) | 10.6 | 66 | 54 | 92 | 43 | 76 | 50 | 10 | + | 0-0-2- |
| donor | | CGUGGGCAG | | | | | | | | | | | | 17-161 |
| site | | | | | | | | | | | | | | |
| intron 6, | KKH- | GUGAUGACCUG | (GGAGGT) | 20 (C9) | 3.7 | 77 | 59 | 27 | 58 | 80 | 61 | 3 | − | 0-0-0- |
| acceptor | SaBE3 | GAAAGGUGA | | | | | | | | | | | | 7-93 |
| site | | | | | | | | | | | | | | |
| intron 6, | St3BE3 | UGUGAUGACCU | (AGGAG) | 20 (C10) | 7.2 | 75 | 73 | 80 | 15 | 77 | 51 | 7 | − | 0-0-0- |
| acceptor | | GGAAAGGUG | | | | | | | | | | | | 10-98 |
| site | | | | | | | | | | | | | | |
| intron 8, | St3BE3 | UACCUGCCCCA | (GGGGG) | 20 (C3/4) | 7.5 | 88 | 43 | 53 | 4 | 67 | 50 | 7 | + | 0-0-1- |
| donor | | UGGGUGCUG | | | | | | | | | | | | 4-45 |
| site | | | | | | | | | | | | | | |
| intron 7, | St3BE3 | AUGCCUGGUGC | (TGGTG) | 20 (C4) | 5.5 | 76 | 46 | 79 | 6 | 27 | 73 | 5 | − | 0-0-1- |
| acceptor | | AGGGGUGAA | | | | | | | | | | | | 9-108 |
| site | | | | | | | | | | | | | | |
| intron 8, | VQR- | UUACCUGCCCC | (GGGG) | 20 (C4/5) | 6.4 | 76 | 46 | 79 | 6 | 27 | 73 | 5 | − | 0-0-1- |
| donor | SpBE3 | AUGGGUGCU | | | | | | | | | | | | 9-108 |
| site | | | | | | | | | | | | | | |
| intron 1, | VQR- | ACCUUGGCGCA | (GGTG) | 20 (C2/3) | 7.5 | 97 | — | 30 | 10 | 58 | 55 | 7 | − | 0-0-0- |
| donor | SpBE3 | GCGGUGGAA | | | | | | | | | | | | 1-1 |
| site | | | | | | | | | | | | | | |
| intron 5, | KKH- | AGGCCUGGGAG | (CAAGGT) | 20 (C5) | 5.5 | 82 | — | 61 | 3 | 58 | 71 | 5 | − | 0-0-3- |
| acceptor | SaBE3 | GAACAAAGC | | | | | | | | | | | | 2-66 |
| site | | | | | | | | | | | | | | |
| intron 3, | SpBE3 | UGGGGGUCUUA | (TGG) | 20 | 5.2 | 81 | 42 | 8 | 1 | 69 | 58 | 5 | + | 0-0-0- |
| donor | | CCGGGGGGC | | (C12/13) | | | | | | | | | | 6-130 |
| site | | | | | | | | | | | | | | |
| intron | VQR- | CCUGCCAGAGC | (AGAA) | 20 (C2) | 4.6 | 72 | — | 78 | 10 | 50 | 56 | 4 | − | 0-0-2- |
| 11, | SpBE3 | CCGAGGAAA | | | | | | | | | | | | 18-206 |
| acceptor | | | | | | | | | | | | | | |
| site | | | | | | | | | | | | | | |
| intron | St3BE3 | AACCACAGCUC | (AGGGG) | 20 (C12) | 4.5 | 67 | 45 | 83 | 3 | 63 | 49 | 4 | + | 0-0-2- |
| 10, | | CUGGGGCAG | | | | | | | | | | | | 15-115 |
| acceptor | | | | | | | | | | | | | | |
| site | | | | | | | | | | | | | | |
| intron 1, | EQR- | CGGAUCCUGGC | (GGAG) | 20 (C7) | 5.0 | 79 | — | 37 | 18 | 69 | 69 | 5 | − | 0-0-1- |
| acceptor | SpBE3 | CCCAUGCAA | | | | | | | | | | | | 4-79 |
| site | | | | | | | | | | | | | | |
| intron | St3BE3 | GGCCUCUUCAC | (AGGGG) | 20 | 4.1 | 78 | 46 | 70 | 3 | 55 | 31 | 4 | + | 0-0-0- |
| 11, | | CUGCUCCUG | | (C11/12) | | | | | | | | | | 3-70 |
| donor | | | | | | | | | | | | | | |
| site | | | | | | | | | | | | | | |
| intron 6, | SpBE3 | AGCACCUACCU | (AGG) | 20 (C8/9) | 7.4 | 58 | 53 | 89 | 12 | 63 | 42 | 7 | + | 0-0-0- |
| donor | | CGGGAGCUG | | | | | | | | | | | | 11-200 |
| site | | | | | | | | | | | | | | |
| intron 1, | VQR- | CACCCGCACCU | (GGTG) | 20 (C9/10) | 7.7 | 98 | — | 43 | 0 | 24 | 49 | 7 | + | 0-0-0- |
| donor | SpBE3 | UGGCGCAGC | | | | | | | | | | | | 1-10 |
| site | | | | | | | | | | | | | | |
| intron 6, | EQR- | ACUGUGAUGAC | (TGAG) | 20 (C12) | 5.4 | 55 | — | 91 | 16 | 80 | 50 | 5 | −GG | 0-0-4- |
| acceptor | SpBE3 | CUGGAAAGG | | | | | | | | | | | | 24-240 |
| site | | | | | | | | | | | | | | |
| intron 4, | SaBE3 | GUGCUUACCUG | (GCGGGT) | 20 (C8/9) | 6.2 | 83 | — | 25 | 28 | 62 | 62 | 6 | − | 0-0-0- |
| donor | | UCUGUGGAA | | | | | | | | | | | | 7-69 |
| site | | | | | | | | | | | | | | |
| intron 9, | KKH- | UGGGCCUUAGA | (GGAAAT) | 20 (C6) | 4.2 | 82 | 62 | 16 | 60 | 50 | 54 | 4 | − | 0-0-2- |
| acceptor | SaBE3 | GUCAAAGAC | | | | | | | | | | | | 11-69 |
| site | | | | | | | | | | | | | | |
| intron 4, | VQR- | CGUGCUUACCU | (AGCG) | 20 (C9/10) | 5.9 | 99 | — | 31 | 3 | 44 | 31 | 5 | − | 0-0-0- |
| donor | SpBE3 | GUCUGUGGA | | | | | | | | | | | | 0-5 |
| site | | | | | | | | | | | | | | |
| intron 6, | St3BE3 | UACCUCGGGAG | (GGGAG) | 20 (C3) | 5.0 | 66 | 51 | 66 | 1 | 63 | 76 | 5 | + | 0-0-1- |
| donor | | CUGAGGCUG | | | | | | | | | | | | 8-135 |
| site | | | | | | | | | | | | | | |
| intron | SpBE3 | CGGUCACCUGC | (AGG) | 20 (C8) | 4.4 | 61 | 58 | 78 | 25 | 69 | 80 | 4 | + | 0-0-2- |
| 11, | | CAGAGCCCG | | | | | | | | | | | | 23-116 |
| acceptor | | | | | | | | | | | | | | |
| site | | | | | | | | | | | | | | |
| intron 7, | SpBE3 | UGGUGACUUAC | (GGG) | 20 | 4.3 | 69 | 68 | 47 | 19 | 66 | 71 | 4 | + | 0-0-2- |
| donor | | CAGCCACGU | | (C11/12) | | | | | | | | | | 15-47 |
| site | | | | | | | | | | | | | | |
| intron 8, | SpBE3 | GCCAACCUGCA | (TGG) | 20 (C7) | 7.2 | 68 | 37 | 53 | 11 | 71 | 10 | 7 | + | 0-0-7- |
| acceptor | | AAAAGGGCC | | | | | | | | | | | | 12-130 |
| site | | | | | | | | | | | | | | |
| intron 7, | SpBE3 | UGACUUACCAG | (CAG) | 20 (C8/9) | 4.6 | 56 | 64 | 83 | 59 | 68 | 66 | 4 | +GG | 0-0-2- |
| donor | | CCACGUGGG | | | | | | | | | | | | 11-269 |
| site | | | | | | | | | | | | | | |
| intron 2, | EQR- | UCAAGGCCUGC | (AGAG) | 20 (C8) | 4.7 | 41 | — | 97 | 35 | 82 | 68 | 4 | + | 0-0-5- |
| acceptor | SpBE3 | AGAAGCCAG | | | | | | | | | | | | 54-318 |
| site | | | | | | | | | | | | | | |
| intron 3, | St3BE3 | CUUUCCAAGGC | (GGGAG) | 20 (C2) | 5.4 | 96 | 40 | 20 | 9 | 23 | 36 | 5 | − | 0-0-0- |
| acceptor | | GACAUUUGU | | | | | | | | | | | | 2-18 |
| site | | | | | | | | | | | | | | |
| intron 6, | EQR- | GUGAUGACCUG | (GGAG) | 20 (C9) | 3.7 | 55 | — | 27 | 58 | 80 | 61 | 3 | − | 0-0-2- |
| acceptor | SpBE3 | GAAAGGUGA | | | | | | | | | | | | 27-250 |
| site | | | | | | | | | | | | | | |
| intron 8, | St3BE3 | CUUACCUGCCC | (TGGGG) | 20 (C5/6) | 8.8 | 93 | 25 | 27 | 2 | 42 | 27 | 8 | + | 0-0-0- |
| donor | | CAUGGGUGC | | | | | | | | | | | | 3-39 |
| site | | | | | | | | | | | | | | |
| intron 4, | SpBE3 | CCGUGCUUACC | (AAG) | 20 | 9.2 | 69 | 66 | 32 | 22 | 60 | 60 | 9 | +GG | 0-0-0- |
| donor | | UGUCUGUGG | | (C10/11) | | | | | | | | | | 15-84 |
| site | | | | | | | | | | | | | | |
| intron 2, | St3BE3 | CUGCAGAAGCC | (GGGGG) | 20 (C1) | 7.7 | 67 | 43 | 66 | 3 | 61 | 49 | 7 | + | 0-0-3- |
| acceptor | | AGAGAGGCC | | | | | | | | | | | | 9-205 |
| site | | | | | | | | | | | | | | |
| intron 6, | SpBE3 | CAGCACCUACC | (GAG) | 20 (C9/10) | 6.5 | 79 | 36 | 31 | 3 | 19 | 54 | 6 | + | 0-0-2- |
| donor | | UCGGGAGCU | | | | | | | | | | | | 6-144 |
| site | | | | | | | | | | | | | | |
| intron | SpBE3 | GCCUCCUACCU | (TGG) | 20 (C9/10) | 5.6 | 65 | 49 | 52 | 13 | 66 | 32 | 5 | + | 0-0-3- |
| 10, | | GUGAGGACG | | | | | | | | | | | | 12-123 |
| donor | | | | | | | | | | | | | | |
| site | | | | | | | | | | | | | | |
| intron 3, | VQR- | CGUCUUUCCAA | (TGTG) | 20 (C4) | 5.9 | 100 | — | 8 | 5 | 21 | 31 | 5 | − | 0-0-0- |
| acceptor | SpBE3 | GGCGACAUU | | | | | | | | | | | | 0-1 |
| site | | | | | | | | | | | | | | |
| intron 1, | SpBE3 | ACGGAUCCUGG | (AGG) | 20 (C8) | 4.4 | 65 | 53 | 65 | 6 | 61 | 65 | 4 | − | 0-0-0- |
| acceptor | | CCCCAUGCA | | | | | | | | | | | | 19-137 |
| site | | | | | | | | | | | | | | |
| intron 8, | St3BE3 | UUACCUGCCCC | (GGGGG) | 20 (C4/5) | 6.4 | 90 | 29 | 40 | 3 | 17 | 35 | 6 | + | 0-0-0- |
| donor | | AUGGGUGCU | | | | | | | | | | | | 3-35 |
| site | | | | | | | | | | | | | | |
| intron | VQR- | CACCUGCUCCU | (GGAT) | 20 (C3/4) | 6.4 | 58 | — | 69 | 34 | 65 | 55 | 6 | + | 0-0-4- |
| 11, | SpBE3 | GAGGGGCCG | | | | | | | | | | | | 29-225 |
| donor | | | | | | | | | | | | | | |
| site | | | | | | | | | | | | | | |
| intron 8, | VQR- | CCUGCAAAAAG | (TGAG) | 20 (C2) | 4.9 | 50 | — | 62 | 2 | 75 | 40 | 4 | + | 0-0-2- |
| acceptor | SpBE3 | GGCCUGGGA | | | | | | | | | | | | 46-268 |
| site | | | | | | | | | | | | | | |
| intron | SaBE3 | UUCACCUGCUC | (CGGGAT) | 20 (C5/6) | 5.4 | 82 | 32 | 16 | 1 | 41 | 42 | 3 | + | 0-0-1- |
| 11, | | CUGAGGGGC | | | | | | | | | | | | 5-59 |
| donor | | | | | | | | | | | | | | |
| site | | | | | | | | | | | | | | |
| intron 6, | St3BE3 | ACCUGGAAAGG | (GGGTG) | 20 (C3) | 5.3 | 55 | 58 | 62 | 6 | 41 | 51 | 5 | + | 0-0-4- |
| acceptor | | UGAGGAGGU | | | | | | | | | | | | 28-200 |
| site | | | | | | | | | | | | | | |
| intron 9, | SpBE3 | CCCCUUGGGCC | (AAG) | 20 (C9) | 7.1 | 66 | 51 | 25 | 1 | 34 | 41 | 7 | − | 0-0-1- |
| acceptor | | UUAGAGUCA | | | | | | | | | | | | 14-144 |
| site | | | | | | | | | | | | | | |
| intron 2, | St3BE3 | CCUGCAGAAGC | (CGGGG) | 20 (C2) | 4.3 | 49 | 39 | 64 | 3 | 49 | 46 | 4 | + | 0-1-5- |
| acceptor | | CAGAGAGGC | | | | | | | | | | | | 23-194 |
| site | | | | | | | | | | | | | | |
| intron 2, | EQR- | CUUCAAGGCCU | (AGAG) | 20 (C10) | 6.5 | 54 | — | 57 | 16 | 36 | 38 | 6 | + | 0-0-2- |
| acceptor | SpBE3 | GCAGAAGCC | | | | | | | | | | | | 41-331 |
| site | | | | | | | | | | | | | | |
| intron 8, | SpBE3 | CUUACCUGCCC | (TGG) | 20 (C5/6) | 8.8 | 65 | 25 | 27 | 2 | 42 | 27 | 8 | + | 0-0-1- |
| donor | | CAUGGGUGC | | | | | | | | | | | | 21-143 |
| site | | | | | | | | | | | | | | |
| intron 8, | SpBE3 | UUACCUGCCCC | (GGG) | 20 (C4/5) | 6.4 | 67 | 29 | 40 | 3 | 17 | 35 | 6 | + | 0-0-1- |
| donor | | AUGGGUGCU | | | | | | | | | | | | 12-141 |
| site | | | | | | | | | | | | | | |

^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.
^{b}Efficiency score, based on Housden et al (Science Signaling, 2015, 8(393): rs9).
^{c}Specificity scores based on Hsu et al (Nature biotechnology, 2013, 31(9): 827-832), Fusi et al (bioRxiv 021568; doi: http://dx.doi.org/10.1101/021568), Chari et al (Nature Methods, 2015, 12(9): 823-6), Doench et al (Nature Biotechnology, 2014, 32(12): 1262-7), Wang et al (Science, 2014, 343(6166): 80-4), Moreno-Mateos et al (Nature Methods, 2015, 12(10): 982-8), Housden et al (Science Signaling, 2015, 8(393): rs9), and the “Prox/GC” column shows “+” the proximal 6 bp to the PAM has a GC count >=4, and GG if the guide ends with GG, based on Farboud et al (Genetics, 2015, 199(4): 959-71).
^{d}Number of predicted off-target binding sites in the human genome allowing up to 0, 1, 2, 3 or 4 mismatches, respectively shown in the format 0-1-2-3-4. Algorithm used: Haeussler et al, Genome Biol. 2016; 17: 148

Other Protective Variants

The LDL-R mediated cholesterol clearance pathway involves multiple players. Non-limiting examples of protein factors involved in this pathway include: Apolipoprotein C3 (APOC3), LDL receptor (LDL-R), and Increased Degradation of LDL Receptor Protein (IDOL). These protein factors and their respective function are described in the art. Further, loss-of-function variants of these factors have been identified and characterized, and are determined to have cardio protective functions. See, e.g., Jørgensen et al., N Engl J Med 2014; 371:32-41 Jul. 3, 2014; Scholtz 1 et al., Hum. Mol. Genet. (1999) 8 (11): 2025-2030; De Castro-Orós et al., BMC Medical Genomics, 20147:17; and Gu et al., J Lipid Res. 2013, 54(12):3345-57, each of which are incorporated herein by reference.

Thus, some aspects of the present disclosure provide the generation of loss-of-function variants of APOC3 (e.g., A43T and R19X), LDL-R, and IDOL (e.g., R266X) using the nucleobase editors and the strategies described herein. Non-limiting examples of such variants and the guide sequence that may be used to make them are provided in Table 13.

TABLE 13
Loss-of-Function Variants of APOC3, LDL-R, and IDOL

| | | | | | gRNA | | SEQ |
|---|---|---|---|---|---|---|---|
| | Codon | Effects of | | | size | BE | ID |
| Gene | Change | mutation | Guide sequence | PAM | (C edited) | type^{a} | NOs |
| APOC3 | A43T | Lowers triglyceride | UGCAUCCUUGGCGGUCUUGG | (TGG) | 20 (C12) | SpBE3 | 1769-1773 |
| | | levels in vivo | AUCCUUGGCGGUCUUGGUGG | (CGTG) | 20 (C9) | VQR- | |
| | | | GCAUCCUUGGCGGUCUUGGU | (GGCG) | 20 (C11) | SpBE3 | |
| | | | UGCAUCCUUGGCGGUCUUGG | (TGG) | 20 (C13) | VRER- | |
| | | | UGCAUCCUUGGCGGUCUUGG | (TGGCG) | 20 (C12) | SpBE3 | |
| | | | | | | SpBE3 | |
| | | | | | | St3BE3 | |
| APOC3 | R19C | Cardioprotective, | CUCUGCCCGUAAGCACUUGG | (TGG) | 20 (C8) | SpBE3 | 1774-1780 |
| | | lower triglyceride | GGCCUCUGCCCGUAAGCACU | (TGGTG) | 20 (C11) | St3BE3 | |
| | | levels | CUGGCCUCUGCCCGUAAGCA | (CTTGGT) | 20 (C13) | KKH- | |
| | | | UCUGCCCGUAAGCACUUGGU | (GGG) | 20 (C7) | SaBE3 | |
| | | | CUGCCCGUAAGCACUUGGUG | (GGAC) | 20 (C6) | SpBE3 | |
| | | | GCCUCUGCCCGUAAGCACUU | (GGTG) | 20 (C10) | VQR- | |
| | | | GGCCUCUGCCCGUAAGCACU | (TGG) | 20 (C11) | SpBE3 | |
| | | | | | | VQR- | |
| | | | | | | SpBE3 | |
| | | | | | | SpBE3 | |
| APOC3 | Splicing | Associated with | UGCUUACGGGCAGAGGCCAG | (GAG) | 20 (C7) | SpBE3 | 1781-1787 |
| | variant | lower triglyceride | AGUGCUUACGGGCAGAGGCC | (AGGAG) | 20 (C9) | St3BE3 | |
| | IVS2 G | levels | GUGCUUACGGGCAGAGGCCA | (GGAG) | 20 (C9) | St3BE3 | |
| | to A | | AAGUGCUUACGGGCAGAGGC | (CAG) | 20 (C10) | SpBE3 | |
| | | | AGUGCUUACGGGCAGAGGCC | (AGG) | 20 (C9) | SpBE3 | |
| | | | CGGGCAGAGGCCAGGAGCGC | (CAG) | 20 (C1) | SpBE3 | |
| | | | GCUUACGGGCAGAGGCCAGG | (AGCG) | 20 (C6) | VRER- | |
| | | | | | | SpBE3 | |
| IDOL | R266Q | Loss-of-function | GGCUCUACCGAGCGAUAACA | (GAG) | 20 (C9) | SpBE3 | 1788-1791 |
| | | variant that lowers | CGGGCUCUACCGAGCGAUAA | (CAG) | 20 (C11) | SpBE3 | |
| | | LDL cholesterol | GGGCUCUACCGAGCGAUAAC | (AGAG) | 20 (C10) | EQR- | |
| | | levels | GCUCUACCGAGCGAUAACAG | (AGAC) | 20 (C8) | SpBE3 | |
| | | | | | | VQR- | |
| | | | | | | SpBE3 | |
| LDL-R | −124 C to T | Increased | UUAAAAAGCCGAUGUCACAU | (CGG) | 20 (C9) | SpBE3 | 1792, |
| | | transcription by 1.6 | CCGAUGUCACAUCGGCCGUU | (CGAA) | 20 (C1) | VQR- | 1793 |
| | | fold | | | | SpBE3 | |
| LDL-R | g. 3131 | Increased | AUAAACGUUGCAGCAGCUCC | (TAG) | 20 (C6) | SpBE3 | 1794-1796- |
| | T to C | transcription by 2.5 | UAAACGUUGCAGCAGCUCCU | (AGAA) | 20 (C5) | VQR- | |
| | | fold | UAUAAACGUUGCAGCAGCUC | (CTAGAAC) | 20 (C7) | SpBE3 | |
| | | | | | | St1BE3 | |
| LDL-R | D299N | Contacts PCSK9 | GUUGUUGUCCAAGCAUUCGU | (TGG) | 20 (C9) | SpBE3 | 1797-1799 |
| | | S153 N-terminal | UCCAAGCAUUCGUUGGUCCC | (TGCG) | 20 (C2) | VRER- | |
| | | amine | CCGUUGUUGUCCAAGCAUUC | (GTTGGT) | 20 (C11) | SpBE3 | |
| | | | | | | KKH- | |
| | | | | | | SaBE3 | |

*Guide sequences (the portion of the guide RNA that targets the nucleobase editor to the target sequence) are provided, which may be used with any tracrRNA framework sequences provided herein to generate the full guide RNA sequence
^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1- SaCas9n-UGI; KKH-SaBE3 = APOBEC1-KKH-SaCas9n-UGI; St3BE3 = APOBEC1-St3Cas9n-UGI; St1BE3 = APOBEC1-St1 Cas9n-UGI.

APOC3 Amino Acid Sequence (NC_000011.9 GRCh37.p5, SEQ ID NO: 1800) MQPRVLLVVALLALLASARASEAEDASLLSFMQGYMKHATKTAKDALSSVQESQVAQ QARGWVTDGFSSLKDYWSTVKDKFSEFWDLDPEVRPTSAVAA

APOC3 cDNA sequence showing amino acid residues assigned to the corresponding codons. Examples of residues targeted for base editing are underlined (nucleotide sequence: SEQ ID NO: 1801, protein sequence: SEQ ID NO: 1802).

```
gctcagttcatccctagaggcagctgctccaggaacagaggtgccatgcagccccgggta
                                                                                            M Q P R V
ctccttgttgttgccctcctggcgctcctggcctctgcccgagcttcagaggccgaggat
  L L V V A L L A L L A S A R A S E A E D
gcctcccttctcagcttcatgcagggttacatgaagcacgccaccaagaccgccaaggat
  A S L L S F M Q G Y M K H A T K T A K D
gcactgagcagcgtgcaggagtcccaggtggcccagcaggccaggggctgggtgaccgat
  A L S S V Q E S Q V A Q Q A R G W V T D
ggcttcagttccctgaaagactactggagcaccgttaaggacaagttctctgagttctgg
  G F S S L K D Y W S T V K D K F S E F W
gatttggaccctgaggtcagaccaacttcagccgtggctgcctgagacctcaatacccca
  D L D P E V R P T S A V A A -
```

APOC3 genomic sequence (SEQ ID NO: 1803) showing non-coding regions and introns (lowercase) as well as exons (uppercase). Examples of bases involved in splicing targeted for base editing are underlined.

gtgggcccaggggacatctcagccccgagaagggtcagcggcccctcctg
gaccaccgactccccgcagaactcctctgtgccctctcctcaccagacct
tgttcctcccagttgctcccacagccagggggcagtgagggctgctcttc
ccccagccccactgaggaacccaggaaggtgaacgagagaatcagtcctg
gtgggggctggggagggccccagacatgagaccagctcctcccccagggg
atgttatcagtgggtccagagggcaaaatagggagcctggtggagggagg
ggcaaaggcctcgggctctgagcggccttggcccttctccaccaacccct
gccctacactaagggggaggcagcggggggcacacagggtgggggcgggt
ggggggctgctgggtgagcagcactcgcctgcctggattgaaacccagag
atggaggtgctgggaggggctgtgagagctcagccctgtaaccaggcctt
gccggagccactgatgcctggtcttctgtgcctttactccaaacaccccc
cagcccaagccacccacttgttctcaagtctgaagaagcccctcacccct
ctactccaggctgtgttcagggcttggggctggtggagggaggggcctga
aattccagtgtgaaaggctgagatgggcccgaggcccctggcctatgtcc
aagccatttcccctctcaccagcctctccctggggagccagtcagctagg
aaggaatgagggctccccaggcccacccccagttcctgagctcatctggg
ctgcagggctggcgggacagcagcgtggactcagtctcctagggatttcc
caactctcccgcccgcttgctgcatctggacaccctgcctcaggccctca
tctccactggtcagcaggtgacctttgcccagcgccctgggtcctcagtg
cctgctgccctggagatgatataaaacaggtcagaaccctcctgcctgtc
TGCTCAGTTCATCCCTAGAGGCAGCTGCTCCAGgtaatgccctctgggga
ggggaaagaggaggggaggaggatgaagaggggcaagaggagctccctgc
ccagcccagccagcaagcctggagaagcacttgctagagctaaggaagcc
tcggagctggacgggtgccccccacccctcatcataacctgaagaacatg
gaggcccgggaggggtgtcacttgcccaaagctacacagggggtggggct
ggaagtggctccaagtgcaggttcccccctcattcttcaggcttagggct
ggaggaagccttagacagcccagtcctaccccagacagggaaactgaggc
ctggagagggccagaaatcacccaaagacacacagcatgttggctggact
ggacggagatcagtccagaccgcaggtgccttgatgttcagtctggtggg
ttttctgctccatcccacccacctccctttgggcctcgatccctcgcccc
tcaccagtcccccttctgagagcccgtattagcagggagccggcccctac
tccttctggcagacccagctaaggttctaccttaggggccacgccacctc
cccagggaggggtccagaggcatggggacctggggtgcccctcacaggac
acttccttgcagGAACAGAGGTGCCATGCAGCCCCGGGTACTCCTTGTTG
TTGCCCTCCTGGCGCTCCTGGCCTCTGCCCgtaagcacttggtgggactg
ggctgggggcagggtggaggcaacttggggatcccagtcccaatgggtgg
tcaagcaggagcccagggctcgtccagaggccgatccaccccactcagcc
ctgctctttcctcagGAGCTTCAGAGGCCGAGGATGCCTCCCTTCTCAGC
TTCATGCAGGGTTACATGAAGCACGCCACCAAGACCGCCAAGGATGCACT
GAGCAGCGTGCAGGAGTCCCAGGTGGCCCAGCAGGCCAGgtacacccgct
ggcctccctccccatcccccctgccagctgcctccattcccacccgcccc
tgccctggtgagatcccaacaatggaatggaggtgctccagcctcccctg
ggcctgtgcctcttcagcctcctctttcctcacagggcctttgtcaggct
gctgcgggagagatgacagagttgagactgcattcctcccaggtccctcc
tttctccccggagcagtcctagggcgtgccgttttagccctcatttccat
tttcctttcctttccctttctttctctttctatttctttctttctttctt
tctttctttctttctttctttctttctttctttctttctttctttctttc
ctttctttctttcctttctttctttcctttctttctttctttcctttctt
tctctttctttctttctttcctttttctttctttccctctcttcctttct
ctctttctttcttcttcttttttttttaatggagtctccctctgtcacct
aggctggagtgcagtggtgccatctcggctcactgcaacctccgtctccc
gggttcaacccattctcctgcctcagcctcccaagtagctgggattacag
gcacgcgccaccacacccagctaatttttgtatttttagcagagatgggg
tttcaccatgttggccaggttggtcttgaattcctgacctcaggggatcc
tcctgcctcggcctcccaaagtgctgggattacaggcatgagccactgcg
cctggccccattttccttttctgaaggtctggctagagcagtggtcctca
gcctttttggcaccagggaccagttttgtggtggacaatttttccatggg
ccagcggggatggttttgggatgaagctgttccacctcagatcatcaggc
attagattctcataaggagccctccacctagatccctggcatgtgcagtt
cacaatagggttcacactcctatgagaatgtaaggccacttgatctgaca
ggaggcggagctcaggcggtattgctcactcacccaccactcacttcgtg
ctgtgcagcccggctcctaacagtccatggaccagtacctatctatgact
tgggggttggggacccctgggctaggggtttgccttgggaggccccacct
gacccaattcaagcccgtgagtgcttctgctttgttctaagacctggggc
cagtgtgagcagaagtgtgtccttcctctcccatcctgcccctgcccatc
agtactctcctctcccctactcccttctccacctcaccctgactggcatt
agctggcatagcagaggtgttcataaacattcttagtccccagaaccggc
tttggggtaggtgttattttctcactttgcagatgagaaaattgaggctc
agagcgattaggtgacctgccccagatcacacaactaatcaatcctccaa
tgactttccaaatgagaggctgcctccctctgtcctaccctgctcagagc
caccaggttgtgcaactccaggcggtgctgtttgcacagaaaacaatgac
agccttgacctttcacatctccccaccctgtcactttgtgcctcaggccc
aggggcataaacatctgaggtgacctggagatggcagggtttgacttgtg
ctggggttcctgcaaggatatctcttctcccagggtggcagctgtggggg
attcctgcctgaggtctcagggctgtcgtccagtgaagttgagagggtgg
tgtggtcctgactggtgtcgtccagtggggacatgggtgtgggtcccatg
gttgcctacagaggagttctcatgccctgctctgttgcttcccctgactg
atttagGGGCTGGGTGACCGATGGCTTCAGTTCCCTGAAAGACTACTGGA
GCACCGTTAAGGACAAGTTCTCTGAGTTCTGGGATTTGGACCCTGAGGTC
AGACCAACTTCAGCCGTGGCTGCCTGAGACCTCAATACCCCAAGTCCACC
TGCCTATCCATCCTGCGAGCTCCTTGGGTCCTGCAATCTCCAGGGCTGCC
CCTGTAGGTTGCTTAAAAGGGACAGTATTCTCAGTGCTCTCCTACCCCAC
CTCATGCCTGGCCCCCCTCCAGGCATGCTGGCCTCCCAATAAAGCTGGAC
AAGAAGCTGCTATGagtgggccgtcgcaagtgtgccatctgtgtctgggc
atgggaaagggccgaggctgttctgtgggtgggcactggacagactccag
gtcaggcaggcatggaggccagcgctctatccaccttctggtagctgggc
agtctctgggcctcagtttcttcatctctaaggtaggaatcaccctccgt
accctgccttccttgacagctttgtgcggaaggtcaaacaggacaataag
tttgctgatactttgataaactgttaggtgctgcacaacatgacttgagt
gtgtgccccatgccagccactatgcctggcacttaagttgtcatcagagt
tgagactgtgtgtgtttactcaaaactgtggagctgacctcccctatcca
ggccccctagccctcttaggcgcacgtgaagggaggaggccggatgggct
agaggttggagtaagatgcaacgaggcactattcttggctccaccacttg
atatcagcctcagtttcttacatgtaaagtggatacaaccgtaccccctc
caccgtaggtttgccgtgagattgaaatgagagagcgttcgaaccgtttg
gcacagcacctgcacgtaaagatgcttgatcaatgttgtcatgattacag
ttgagctgactgggcccttgggacccggactggagtggtggggggcagtg
tcctgggaccaaaaagaagcacaaggtctcccaatagaggctgcttcctt
tgtgtccccaccacccgaaagatgtcaggtcagagagcccgagagctgca
gatggcttgagtagggctccactcttcagatcaaaaaactgtggcccgga
gaggcgaaggcacttggccagcatcacagagccagcacgtggcagggcca
gaccttgagcccaggtcagctgcgtgtattctgctcagttggtgcagaaa
acagttttgtcactcctatgtcaggtgttagggactcctttacagatctc
agtggcatcagtac
IDOL Amino Acid Sequence

(SEQ ID NO: 1804)

MLCYVTRPDAVLMEVEVEAKANGEDCLNQVCRRLGIIEVDYFGLQFTGSK
GESLWLNLRNRISQQMDGLAPYRLKLRVKFFVEPHLILQEQTRHIFFLHI
KEALLAGHLLCSPEQAVELSALLAQTKFGDYNQNTAKYNYEELCAKELSS
ATLNSIVAKHKELEGTSQASAEYQVLQIVSAMENYGIEWHSVRDSEGQKL
LIGVGPEGISICKDDFSPINRIAYPVVQMATQSGKNVYLTVTKESGNSIV
LLFKMISTRAASGLYRAITETHAFYRCDTVTSAVMMQYSRDLKGHLASLF
LNENINLGKKYVFDIKRTSKEVYDHARRALYNAGVVDLVSRNNQSPSHSP
LKSSESSMNCSSCEGLSCQQTRVLQEKLRKLKEAMLCMVCCEEEINSTFC
PCGHTVCCESCAAQLQSCPVCRSRVEHVQHVYLPTHTSLLNLTVI
LDL-R Amino Acid Sequence

(SEQ ID NO: 1805)

AVGDRCERNEFQCQDGKCISYKWVCDGSAECQDGSDESQETCLSVTCKSG
DFSCGGRVNRCIPQFWRCDGQVDCDNGSDEQGCPPKTCSQDEFRCHDGKC
ISRQFVCDSDRDCLDGSDEASCPVLTCGPASFQCNSSTCIPQLWACDNDP
DCEDGSDEWPQRCRGLYVFQGDSSPCSAFEFHCLSGECIHSSWRCDGGPD
CKDKSDEENCAVATCRPDEFQCSDGNCIHGSRQCDREYDCKDMSDEVGCV
NVTLCEGPNKFKCHSGECITLDKVCNMARDCRDWSDEPIKECGTNECLDN
NGGCSHVCNDLKIGYECLCPDGFQLVAQRRCEDIDECQDPDTCSQLCVNL
EGGYKCQCEEGFQLDPHTKACKAVGSIAYLFFTNRHEVRKMTLDRSEYTS
LIPNLRNVVALDTEVASNRIYWSDLSQRMICSTQLDRAHGVSSYDTVISR
DIQAPDGLAVDWIHSNIYWTDSVLGTVSVADTKGVKRKTLFRENGSKPRA
IVVDPVHGFMYWTDWGTPAKIKKGGLNGVDIYSLVTENIQWPNGITLDLL
SGRLYWVDSKLHSISSIDVNGGNRKTILEDEKRLAHPFSLAVFEDKVFWT
DIINEAIFSANRLTGSDVNLLAENLLSPEDMVLFHNLTQPRGVNWCERTT
LSNGGCQYLCLPAPQINPHSPKFTCACPDGMLLARDMRSCLTEAEAAVAT
QETSTVRLKVSSTAVRTQHTTTRPVPDTSRLPGATPGLTTVEIVTMSHQA
LGDVAGRGNEKKPSSVRALSIVLPIVLLVFLCLGVFLLWKNWRLKNINSI
NFDNPVYQKTTEDEVHICHNQDGYSYPSRQMVSLEDDVA

Loss-of-function mutations that may be made in APOC3 gene using the nucleobased editors described herein are also provided. The strategies to generate loss-of-function mutation are similar to that used for PCSK9 (e.g., premature stop codons, destabilizing mutations, altering splicing, etc.) APOC3 mutations and guide RNA sequences are listed in Tables 14-16.

TABLE 14
Exemplary APOC3 Protective Loss-of-Function Mutations via Codon Change
and Premature STOP Codons

| | | Location | | | | | |
|---|---|---|---|---|---|---|---|
| Residue | Codon | of | | | gRNA size | | SEG |
| Change | Change | mutation | guide sequence | (PAM) | (C edited) | BE type^{a} | ID NOs |
| A43T | GCC | ACC | UGCAUCCUUGGCGGUCUUGG | (TGG) | 20 (C12) | SpBE3 | 1806-1809 |
| | | | AUCCUUGGCGGUCUUGGUGG | (CGTG) | 20 (C9) | VQR-SpBE3 | |
| | | | GCAUCCUUGGCGGUCUUGGU | (GGCG) | 20 (C11) | VRER-SpBE3 | |
| | | | UGCAUCCUUGGCGGUCUUGG | (TGGCG) | 20 (C12) | St3BE3 | |
| R19X | CGA | TGA | CUCUGCCCGUAAGCACUUGG | (TGG) | 20 (C8) | SpBE3 | 1810-1816 |
| | | | GGCCUCUGCCCGUAAGCACU | (TGGTG) | 20 (C11) | St3BE3 | |
| | | | CUGGCCUCUGCCCGUAAGCA | (CTTGGT) | 20 (C13) | KKH-SaBE3 | |
| | | | UCUGCCCGUAAGCACUUGGU | (GGG) | 20 (C7) | SpBE3 | |
| | | | CUGCCCGUAAGCACUUGGUG | (GGAC) | 20 (C6) | VQR-SpBE3 | |
| | | | GCCUCUGCCCGUAAGCACUU | (GGTG) | 20 (C10) | VQR-SpBE3 | |
| | | | GGCCUCUGCCCGUAAGCACU | (TGG) | 20 (C11) | SpBE3 | |
| W62X | TGG | TAG, TGA, | CAGCCCCUAAAUCAGUCAGG | (GGAA) | 20 (C1/−1) | VQR-SpBE3 | 1817-1824 |
| | | or TAA | CCAGCCCCUAAAUCAGUCAG | (GGG) | 20 (C1/2) | SpBE3 | |
| | | | CCCAGCCCCUAAAUCAGUCA | (GGG) | 20 (C2/3) | SpBE3 | |
| | | | ACCCAGCCCCUAAAUCAGUC | (AGG) | 20 (C3/4) | SpBE3 | |
| | | | CACCCAGCCCCUAAAUCAGU | (CAG) | 20 (C4/5) | SpBE3 | |
| | | | CGGUCACCCAGCCCCUAAAU | (CAG) | 20 (C8/9) | SpBE3 | |
| | | | AUCGGUCACCCAGCCCCUAA | (ATCAGT) | 20 (C11/12) | KKH-SaBE3 | |
| | | | ACCCAGCCCCUAAAUCAGUC | (AGGGG) | 20 (C3/4) | St3BE3 | |
| W74X | TGG | TAG, TGA, | AGUAGUCUUUCAGGGAACUG | (AAG) | 20 (C−1/−2) | SpBE3 | 1825-1830 |
| | | or TAA | CCAGUAGUCUUUCAGGGAAC | (TGAA) | 20 (C1/2) | VQR-SpBE3 | |
| | | | GUGCUCCAGUAGUCUUUCAG | (GGAA) | 20 (C6/7) | VQR-SpBE3 | |
| | | | GGUGCUCCAGUAGUCUUUCA | (GGG) | 20 (C7/8) | SpBE3 | |
| | | | CGGUGCUCCAGUAGUCUUUC | (AGG) | 20 (C8/9) | SpBE3 | |
| | | | ACGGUGCUCCAGUAGUCUUU | (CAG) | 20 (C9/10) | SpBE3 | |
| W85X | TGG | TAG, TGA, | GUCCAAAUCCCAGAACUCAG | (AGAA) | 20 (C10/11) | VQR-SpBE3 | 1831-1832 |
| | | or TAA | GGGUCCAAAUCCCAGAACUC | (AGAGAAC) | 20 (C12/13) | St1BE3 | |
| Q2 | CAG | TAG | CAGAGGUGCCAUGCAGCCCC | (GGG) | 20 (C14) | SpBE3 | 1833 |
| Q33 | CAG | TAG | CAGCUUCAUGCAGGGUUACA | (TGAA) | 20 (C11) | VQR-SpBE3 | 1834-1835 |
| | | | GCUUCAUGCAGGGUUACAUG | (AAG) | 20 (C9) | SpBE3 | |
| Q51 | CAG | TAG | UGAGCAGCGUGCAGGAGUCC | (CAG) | 20 (C12) | SpBE3 | 1836-1842 |
| | | | GAGCAGCGUGCAGGAGUCCC | (AGG) | 20 (C11) | SpBE3 | |
| | | | AGCAGCGUGCAGGAGUCCCA | (GGTG) | 20 (C10) | VQR-SpBE3 | |
| | | | CAGCGUGCAGGAGUCCCAGG | (TGG) | 20 (C8) | SpBE3 | |
| | | | UGCAGGAGUCCCAGGUGGCC | (CAG) | 20 (C3) | SpBE3 | |
| | | | CUGAGCAGCGUGCAGGAGUC | (CCAGGT) | 20 (C13) | KKH-SaBE3 | |
| | | | GAGCAGCGUGCAGGAGUCCC | (AGGTG) | 20 (C11) | St3BE3 | |
| Q54 and | CAG | TAG | AGGAGUCCCAGGUGGCCCAG | (CAG) | 20 (C9/−1) | SpBE3 | 1843-1847 |
| Q57 | | | GGAGUCCCAGGUGGCCCAGC | (AGG) | 20 (C8) | SpBE3 | |
| | | | UCCCAGGUGGCCCAGCAGGC | (CAG) | 20 (C4/13) | SpBE3 | |
| | | | CCCAGGUGGCCCAGCAGGCC | (AGG) | 20 (C3/12) | SpBE3 | |
| | | | GUCCCAGGUGGCCCAGCAGG | (CCAGGT) | 20 (C5) | KKH-SaBE3 | |
| Q58 | CAG | TAG | AGCAGGCCAGGUACACCCGC | (TGG) | 20 (C3) | SpBE3 | 1848 |
| P89US | CCT | TCT, CTT, | UGGGAUUUGGACCCUGAGGU | (CAG) | 20 (C13/14) | SpBE3 | 1849-1851 |
| | | or TTT | GGGAUUUGGACCCUGAGGUC | (AGAC) | 20 (C12/13) | VQR-SpBE3 | |
| | | | CCCUGAGGUCAGACCAACUU | (CAG) | 20 (C2/3) | SpBE3 | |
| P93L/S | CCA | TCA, CTA, | GAGGUCAGACCAACUUCAGC | (CGTG) | 20 (C10/11) | VQR-SpBE3 | 1852-1853 |
| | | or TTA | GGUCAGACCAACUUCAGCCG | (TGG) | 20 (C8/9) | SpBE3 | |
| M1I | ATG | ATA | AUGGCACCUCUGUUCCUGCA | (AGG) | 20 (C−1) | SpBE3 | 1854-1855 |
| | | | CAUGGCACCUCUGUUCCUGC | (AAG) | 20 (C1) | SpBE3 | |

*Guide sequences (the portion of the guide RNA that targets the nucleobase editor to the target sequence) are provided, which may be used with any tracrRNA framework sequences provided herein to generate the full guide RNA sequence
^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.

TABLE 15
Alteration of Intron/Exon Junctions in APOC3 Gene via Base Editing

| | | | | | | Guide |
|---|---|---|---|---|---|---|
| Target | Genome target | | | gRNA size | | RNA SEQ |
| site | sequence | guide sequence | (PAM) | (C edited) | BE type^{a} | ID NO |
| Intron 1 | GCTCAGTTCATCCCTA | CCUGGAGCAGCUGCCUCUAG | (GGAT) | 20 (C1/2) | VQR-SpBE3 | 1856-1860 |
| donor | GAGGCAGCTGCTCCAG | ACCUGGAGCAGCUGCCUCUA | (GGG) | 20 (C2/3) | SpBE3 | |
| site | gtaatgcc (SEQ ID | UACCUGGAGCAGCUGCCUCU | (AGG) | 20 (C3/4) | SpBE3 | |
| | NO: 1907) | UUACCUGGAGCAGCUGCCUC | (TAG) | 20 (C4/5) | SpBE3 | |
| | | UACCUGGAGCAGCUGCCUCU | (AGGGAT) | 20 (C3/4) | SaBE3 | |
| Intron 1 | caggacacttccttgc | CUGCAAGGAAGUGUCCUGUG | (AGG) | 20 (C1/−1) | SpBE3 | 1861-1869 |
| acceptor | agGAACAGAGGTGCCA | CCUGCAAGGAAGUGUCCUGU | (GAG) | 20 (C1/2) | SpBE3 | |
| site | TGCA (SEQ ID | GUUCCUGCAAGGAAGUGUCC | (TGTG) | 20 (C4/5) | VQR-SpBE3 | |
| | NO: 1908) | CUGCAAGGAAGUGUCCUGUG | (AGGGG) | 20 (C1/−1) | St3BE3 | |
| | | GACACUUCCUUGCAGGAACA | (GAG) | 20 (C13) | SpBE3 | |
| | | ACACUUCCUUGCAGGAACAG | (AGG) | 20 (C12) | SpBE3 | |
| | | CACUUCCUUGCAGGAACAGA | (GGTG) | 20 (C10) | VQR-SpBE3 | |
| | | GCAGGAACAGAGGUGCCAUG | (CAG) | 20 (C2) | SpBE3 | |
| | | ACACUUCCUUGCAGGAACAG | (AGGTG) | 20 (C12) | St3BE3 | |
| Intron 2 | GGCGCTCCTGGCCTCT | GGGCAGAGGCCAGGAGCGCC | (AGG) | 20 (C−1) | SpBE3 | 1870-1878 |
| donor | GCCCgtaagcacttgg | CGGGCAGAGGCCAGGAGCGC | (CAG) | 20 (C1) | SpBE3 | |
| site | tgggact (SEQ ID | GCUUACGGGCAGAGGCCAGG | (AGCG) | 20 (C6) | VRER-SpBE3 | |
| | NO: 1909) | UGCUUACGGGCAGAGGCCAG | (GAG) | 20 (C7) | SpBE3 | |
| | | GUGCUUACGGGCAGAGGCCA | (GGAG) | 20 (C8) | EQR-SpBE3 | |
| | | AGUGCUUACGGGCAGAGGCC | (AGG) | 20 (C9) | SpBE3 | |
| | | AAGUGCUUACGGGCAGAGGC | (CAG) | 20 (C10) | SpBE3 | |
| | | GGGCAGAGGCCAGGAGCGCC | (AGGAG) | 20 (C−1) | St3BE3 | |
| | | AGUGCUUACGGGCAGAGGCC | (AGGAG) | 20 (C9) | St3BE3 | |
| Intron 2 | cagccctgctctttcc | CUGAGGAAAGAGCAGGGCUG | (AGTG) | 20 (C1/−1) | VQR-SpBE3 | 1879-1894 |
| acceptor | tcagGAGCTTCAGAGG | CCUGAGGAAAGAGCAGGGCU | (GAG) | 20 (C1/2) | SpBE3 | |
| site | CCGAGGATGCCTC | AAGCUCCUGAGGAAAGAGCA | (GGG) | 20 (C6/7) | SpBE3 | |
| | (SEQ ID NO: | GAAGCUCCUGAGGAAAGAGC | (AGG) | 20 (C7/8) | SpBE3 | |
| | 1910) | UGAAGCUCCUGAGGAAAGAG | (CAG) | 20 (C8/9) | SpBE3 | |
| | | CUCUGAAGCUCCUGAGGAAA | (GAG) | 20 (C11/12) | SpBE3 | |
| | | CUCCUGAGGAAAGAGCAGGG | (CTGAGT) | 20 (C3/4) | SaBE3 | |
| | | UGCUCUUUCCUCAGGAGCUU | (CAG) | 20 (C12) | SpBE3 | |
| | | GCUCUUUCCUCAGGAGCUUC | (AGAG) | 20 (C11/12) | EQR-SpBE3 | |
| | | CUCUUUCCUCAGGAGCUUCA | (GAG) | 20 (C10) | SpBE3 | |
| | | UCUUUCCUCAGGAGCUUCAG | (AGG) | 20 (C9) | SpBE3 | |
| | | UCCUCAGGAGCUUCAGAGGC | (CGAG) | 20 (C5) | EQR-SpBE3 | |
| | | CCUCAGGAGCUUCAGAGGCC | (GAG) | 20 (C4) | SpBE3 | |
| | | CUCAGGAGCUUCAGAGGCCG | (AGG) | 20 (C3) | SpBE3 | |
| | | UCAGGAGCUUCAGAGGCCGA | (GGAT) | 20 (C2) | VQR-SpBE3 | |
| | | CCUCAGGAGCUUCAGAGGCC | (GAGGAT) | 20 (C4) | SaBE3 | |
| Intron 3 | CAGGTGGCCCAGCAGG | CUGGCCUGCUGGGCCACCUG | (GGAC) | 20 (C1/−1) | VQR-SpBE3 | 1895-1899 |
| donor | CCAGgtacacccgctg | CCUGGCCUGCUGGGCCACCU | (GGG) | 20 (C1/2) | SpBE3 | |
| site | gcctccctcc (SEQ | ACCUGGCCUGCUGGGCCACC | (TGG) | 20 (C2/3) | SpBE3 | |
| | ID NO: 1911) | GCGGGUGUACCUGGCCUGCU | (GGG) | 20 (C10/11) | SpBE3 | |
| | | AGCGGGUGUACCUGGCCUGC | (TGG) | 20 (C11/12) | SpBE3 | |
| Intron 3 | cccctgactgatttag | GCCCCUAAAUCAGUCAGGGG | (AAG) | 20 (C4/5) | SpBE3 | 1900-1906 |
| acceptor | GGGCTGGGTGACCGA | CAGCCCCUAAAUCAGUCAGG | (GGAA) | 20 (C6/7) | VQR-SpBE3 | |
| site | (SEQ ID NO: | CCAGCCCCUAAAUCAGUCAG | (GGG) | 20 (C7/8) | SpBE3 | |
| | 1912) | CCCAGCCCCUAAAUCAGUCA | (GGG) | 20 (C8/9) | SpBE3 | |
| | | ACCCAGCCCCUAAAUCAGUC | (AGG) | 20 (C9/10) | SpBE3 | |
| | | CACCCAGCCCCUAAAUCAGU | (CAG) | 20 (C10/11) | SpBE3 | |
| | | ACCCAGCCCCUAAAUCAGUC | (AGGGG) | 20 (C9/10) | St3BE3 | |

*Guide sequences (the portion of the guide RNA that targets the nucleobase editor to the target sequence) are provided, which may be used with any tracrRNA framework sequences provided herein to generate the full guide RNA sequence
^{a}BE types: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI; St3BE3 = APOBEC1-St3Cas9n-UGI; St1BE3 = APOBEC1-St1Cas9n-UGI.

TABLE 16
Efficiency and Specificity Scores for gRNAs for APOC3 Protective Loss-of-Function Mutations via Codon Change. The
guidesequences correspond to SEQ ID NOs: 1913-1987 from top to bottom.

| | | | | gRNA size | | | | | | | | | Prox/ | |
|---|---|---|---|---|---|---|---|---|---|---|---|---|---|---|
| Target variants | BE type^{a} | guidesequence | PAM | (C edited) | Eff^{b} | Hsu^{c} | Fusi | Chari | Doench | Wang | M.-M. | Housden | GC | Off-targets^{d} |
| Intron 2 donor | VRER-SpBE3 | GCUUACGGGCAGAGGCCAGG | (AGCG) | 20 (C6) | 8.5 | 88 | −1 | 99 | 19 | 79 | 49 | 8 | +GG | 0-0-1-2- |
| | | | | | | | | | | | | | | 16 |
| P93L/S | SpBE3 | GGUCAGACCAACUUCAGCCG | (TGG) | 20 (C8/9) | 6.5 | 91 | 65 | 78 | 81 | 94 | 39 | 6 | + | 0-0-0-6- |
| | | | | | | | | | | | | | | 38 |
| W85X | St1BE3 | GGGUCCAAAUCCCAGAACUC | (AGAGAAC) | 20 (C12/13) | 4.5 | 96 | −1 | 86 | 10 | 60 | 34 | 4 | − | 0-0-0-1- |
| | | | | | | | | | | | | | | 18 |
| Intron 1 acceptor | St3BE3 | ACACUUCCUUGCAGGAACAG | (AGGTG) | 20 (C12) | 4.3 | 88 | 66 | 93 | 72 | 79 | 47 | 4 | − | 0-0-1-1- |
| | | | | | | | | | | | | | | 39 |
| W62X | KKH-SaBE3 | AUCGGUCACCCAGCCCCUAA | (ATCAGT) | 20 (C11/12) | 7.4 | 97 | −1 | 81 | 8 | 41 | 55 | 7 | − | 0-0-0-0- |
| | | | | | | | | | | | | | | 15 |
| P93L/S | VQR-SpBE3 | GAGGUCAGACCAACUUCAGC | (CGTG) | 20 (C10/11) | 5.9 | 99 | −1 | 64 | 11 | 77 | −2 | 5 | − | 0-0-0-0-8 |
| Intron 2 acceptor | SaBE3 | CUCCUGAGGAAAGAGCAGGG | (CTGAGT) | 20 (C3/4) | 5.9 | 78 | −1 | 98 | 14 | 76 | 62 | 5 | +GG | 0-0-0- |
| | | | | | | | | | | | | | | 12-116 |
| Q51 | KKH-SaBE3 | CUGAGCAGCGUGCAGGAGUC | (CCAGGT) | 20 (C13) | 5.0 | 94 | −1 | 36 | 2 | 19 | 77 | 5 | + | 0-0-0-1- |
| | | | | | | | | | | | | | | 28 |
| Intron 1 acceptor | St3BE3 | CUGCAAGGAAGUGUCCUGUG | (AGGGG) | 20 (C1/−1) | 7.6 | 87 | 62 | 83 | 5 | 39 | 84 | 7 | + | 0-0-0-3- |
| | | | | | | | | | | | | | | 46 |
| A43T | St3BE3 | UGCAUCCUUGGCGGUCUUGG | (TGGCG) | 20 (C12) | 5.3 | 92 | 45 | 76 | 5 | 45 | 54 | 5 | −GG | 0-0-0-6- |
| | | | | | | | | | | | | | | 28 |
| Q51 | VQR-SpBE3 | AGCAGCGUGCAGGAGUCCCA | (GGTG) | 20 (C10) | 9.1 | 98 | −1 | 70 | 31 | 62 | 58 | 9 | + | 0-0-0-1- |
| | | | | | | | | | | | | | | 11 |
| Intron 1 acceptor | VQR-SpBE3 | CACUUCCUUGCAGGAACAGA | (GGTG) | 20 (C10) | 4.5 | 95 | −1 | 73 | 9 | 53 | 42 | 4 | − | 0-0-0-5-7 |
| W62X | VQR-SpBE3 | CAGCCCCUAAAUCAGUCAGG | (GGAA) | 20 (C1/−1) | 5.7 | 74 | −1 | 91 | 66 | 70 | 62 | 5 | +GG | 0-0-1- |
| | | | | | | | | | | | | | | 14-130 |
| Q58 | SpBE3 | AGCAGGCCAGGUACACCCGC | (TGG) | 20 (C3) | 4.3 | 87 | 54 | 50 | 15 | 78 | 41 | 4 | + | 0-0-0- |
| | | | | | | | | | | | | | | 14-142 |
| Intron 3 acceptor | VQR-SpBE3 | CAGCCCCUAAAUCAGUCAGG | (GGAA) | 20 (C6/7) | 5.7 | 74 | −1 | 91 | 66 | 70 | 62 | 5 | +GG | 0-0-1- |
| | | | | | | | | | | | | | | 14-130 |
| A43T | VQR-SpBE3 | AUCCUUGGCGGUCUUGGUGG | (CGTG) | 20 (C9) | 6.3 | 100 | −1 | 40 | 7 | 63 | 64 | 6 | +GG | 0-0-0-0-5 |
| R19X | VQR-SpBE3 | CUGCCCGUAAGCACUUGGUG | (GGAC) | 20 (C6) | 4.7 | 92 | −1 | 62 | 29 | 58 | 72 | 4 | − | 0-0-0-1- |
| | | | | | | | | | | | | | | 45 |
| Q51 | St3BE3 | GAGCAGCGUGCAGGAGUCCC | (AGGTG) | 20 (C11) | 4.3 | 83 | 51 | 80 | 7 | 56 | 72 | 4 | + | 0-0-1-4- |
| | | | | | | | | | | | | | | 68 |
| Q54 and Q57 | KKH-SaBE3 | GUCCCAGGUGGCCCAGCAGG | (CCAGGT) | 20 (C5) | 4.2 | 69 | −1 | 93 | 14 | 78 | 88 | 4 | +GG | 0-1-1-6- |
| | | | | | | | | | | | | | | 49 |
| R19X | KKH-SaBE3 | CUGGCCUCUGCCCGUAAGCA | (CTTGGT) | 20 (C13) | 3.4 | 98 | −1 | 32 | 5 | 50 | 59 | 3 | − | 0-0-0-4- |
| | | | | | | | | | | | | | | 27 |
| R19X | VQR-SpBE3 | GCCUCUGCCCGUAAGCACUU | (GGTG) | 20 (C10) | 6.3 | 100 | −1 | 57 | 15 | 46 | 38 | 6 | − | 0-0-0-0-4 |
| Intron 1 acceptor | VQR-SpBE3 | GUUCCUGCAAGGAAGUGUCC | (TGTG) | 20 (C4/5) | 4.6 | 99 | −1 | 27 | 9 | 58 | 21 | 4 | + | 0-0-0-0-9 |
| Intron 2 donor | St3BE3 | AGUGCUUACGGGCAGAGGCC | (AGGAG) | 20 (C9) | 4.8 | 87 | 47 | 65 | 16 | 69 | 46 | 4 | + | 0-0-0-2- |
| | | | | | | | | | | | | | | 49 |
| Intron 2 donor | St3BE3 | GGGCAGAGGCCAGGAGCGCC | (AGGAG) | 20 (C−1) | 7.5 | 76 | 40 | 79 | 1 | 57 | 70 | 7 | + | 0-0-0- |
| | | | | | | | | | | | | | | 26-196 |
| W62X | St3BE3 | ACCCAGCCCCUAAAUCAGUC | (AGGGG) | 20 (C3/4) | 5.1 | 98 | 45 | 56 | 4 | 35 | 13 | 5 | − | 0-0-0-2- |
| | | | | | | | | | | | | | | 11 |
| Intron 3 acceptor | St3BE3 | ACCCAGCCCCUAAAUCAGUC | (AGGGG) | 20 (C9/10) | 5.1 | 98 | 45 | 56 | 4 | 35 | 13 | 5 | − | 0-0-0-2- |
| | | | | | | | | | | | | | | 11 |
| A43T | SpBE3 | UGCAUCCUUGGCGGUCUUGG | (TGG) | 20 (C12) | 5.3 | 75 | 45 | 76 | 5 | 45 | 54 | 5 | −GG | 0-0-0 |
| | | | | | | | | | | | | | | 12-115 |
| A43T | VRER-SpBE3 | GCAUCCUUGGCGGUCUUGGU | (GGCG) | 20 (C11) | 7.3 | 97 | −1 | 47 | 18 | 54 | 39 | 7 | − | 0-0-0-1- |
| | | | | | | | | | | | | | | 10 |
| W62X | SpBE3 | CCAGCCCCUAAAUCAGUCAG | (GGG) | 20 (C1/2) | 4.8 | 69 | 70 | 79 | 58 | 82 | 70 | 4 | − | 0-0-1- |
| | | | | | | | | | | | | | | 13-128 |
| Intron 3 acceptor | SpBE3 | CCAGCCCCUAAAUCAGUCAG | (GGG) | 20 (C7/8) | 4.8 | 69 | 70 | 79 | 58 | 82 | 70 | 4 | − | 0-0-1- |
| | | | | | | | | | | | | | | 13-128 |
| Intron 1 acceptor | SpBE3 | ACACUUCCUUGCAGGAACAG | (AGG) | 20 (C12) | 4.3 | 57 | 66 | 93 | 72 | 79 | 47 | 4 | − | 0-0-4- |
| | | | | | | | | | | | | | | 27-191 |
| R19X | SpBE3 | CUCUGCCCGUAAGCACUUGG | (TGG) | 20 (C8) | 6.7 | 84 | 44 | 65 | 7 | 47 | 45 | 6 | −GG | 0-0-0-9- |
| | | | | | | | | | | | | | | 70 |
| R19X | SpBE3 | UCUGCCCGUAAGCACUUGGU | (GGG) | 20 (C7) | 5.6 | 85 | 58 | 61 | 30 | 59 | 48 | 5 | − | 0-0-0-5- |
| | | | | | | | | | | | | | | 56 |
| W74X | VQR-SpBE3 | GUGCUCCAGUAGUCUUUCAG | (GGAA) | 20 (C6/7) | 5.6 | 75 | −1 | 63 | 48 | 71 | 65 | 5 | − | 0-0-0- |
| | | | | | | | | | | | | | | 10-107 |
| Q51 | SpBE3 | CAGCGUGCAGGAGUCCCAGG | (TGG) | 20 (C8) | 7.2 | 49 | 68 | 95 | 22 | 74 | 82 | 7 | +GG | 0-0-6- |
| | | | | | | | | | | | | | | 32-258 |
| R19X | St3BE3 | GGCCUCUGCCCGUAAGCACU | (TGGTG) | 20 (C11) | 5.6 | 97 | 45 | 14 | 13 | 34 | 36 | 5 | − | 0-0-0-0- |
| | | | | | | | | | | | | | | 28 |
| W74X | SpBE3 | GGUGCUCCAGUAGUCUUUCA | (GGG) | 20 (C7/8) | 7.1 | 75 | 55 | 67 | 25 | 47 | 37 | 7 | − | 0-0-3-8- |
| | | | | | | | | | | | | | | 88 |
| Q51 | SpBE3 | GAGCAGCGUGCAGGAGUCCC | (AGG) | 20 (C11) | 4.3 | 62 | 51 | 80 | 7 | 56 | 72 | 4 | + | 0-0-4- |
| | | | | | | | | | | | | | | 17-237 |
| Intron 3 donor | SpBE3 | GCGGGUGUACCUGGCCUGCU | (GGG) | 20 (C10/11) | 7.9 | 59 | 47 | 50 | 9 | 31 | 83 | 7 | + | 0-0-0- |
| | | | | | | | | | | | | | | 18-130 |
| W74X | SpBE3 | ACGGUGCUCCAGUAGUCUUU | (CAG) | 20 (C9/10) | 7.4 | 92 | 35 | 8 | 17 | 34 | 49 | 7 | − | 0-0-0-2- |
| | | | | | | | | | | | | | | 40 |
| W85X | VQR-SpBE3 | GUCCAAAUCCCAGAACUCAG | (AGAA) | 20 (C10/11) | 6.1 | 44 | −1 | 97 | 69 | 73 | 28 | 6 | − | 0-0-2- |
| | | | | | | | | | | | | | | 44-375 |
| Q33 | VQR-SpBE3 | CAGCUUCAUGCAGGGUUACA | (TGAA) | 20 (C11) | 4.8 | 74 | −1 | 66 | 12 | 16 | 53 | 4 | − | 0-0-2-9- |
| | | | | | | | | | | | | | | 124 |
| Intron 1 acceptor | SpBE3 | CUGCAAGGAAGUGUCCUGUG | (AGG) | 20 (C1/−1) | 7.6 | 56 | 62 | 83 | 5 | 39 | 84 | 7 | + | 0-0-6- |
| | | | | | | | | | | | | | | 20-210 |
| P89L/S | VQR-SpBE3 | GGGAUUUGGACCCUGAGGUC | (AGAC) | 20 (C12/13) | 6.7 | 71 | −1 | 51 | 2 | 68 | 59 | 6 | + | 0-0-0- |
| | | | | | | | | | | | | | | 10-190 |
| W62X | SpBE3 | CGGUCACCCAGCCCCUAAAU | (CAG) | 20 (C8/9) | 4.6 | 82 | 44 | 11 | 19 | 38 | 56 | 4 | − | 0-0-1-4- |
| | | | | | | | | | | | | | | 69 |
| W62X | SpBE3 | ACCCAGCCCCUAAAUCAGUC | (AGG) | 20 (C3/4) | 5.1 | 81 | 45 | 56 | 4 | 35 | 13 | 5 | − | 0-0-2-9- |
| | | | | | | | | | | | | | | 96 |
| Intron 1 donor | SaBE3 | UACCUGGAGCAGCUGCCUCU | (AGGGAT) | 20 (C3/4) | 9.5 | 87 | 50 | 50 | 2 | 47 | 35 | 9 | + | 0-0-0-3- |
| | | | | | | | | | | | | | | 52 |
| Intron 3 acceptor | SpBE3 | ACCCAGCCCCUAAAUCAGUC | (AGG) | 20 (C9/10) | 5.1 | 81 | 45 | 56 | 4 | 35 | 13 | 5 | − | 0-0-2-9- |
| | | | | | | | | | | | | | | 96 |
| Intron 2 donor | EQR-SpBE3 | GUGCUUACGGGCAGAGGCCA | (GGAG) | 20 (C8) | 4.5 | 59 | −1 | 45 | 27 | 75 | 71 | 4 | + | 0-0-0- |
| | | | | | | | | | | | | | | 20-161 |
| Intron 2 acceptor | SpBE3 | GAAGCUCCUGAGGAAAGAGC | (AGG) | 20 (C7/8) | 4.7 | 42 | 52 | 58 | 19 | 91 | 31 | 4 | − | 0-0-4- |
| | | | | | | | | | | | | | | 45-382 |
| Intron 2 donor | SpBE3 | AGUGCUUACGGGCAGAGGCC | (AGG) | 20 (C9) | 4.8 | 63 | 47 | 65 | 16 | 69 | 46 | 4 | + | 0-0-0- |
| | | | | | | | | | | | | | | 16-158 |
| Intron 2 acceptor | SpBE3 | UCUUUCCUCAGGAGCUUCAG | (AGG) | 20 (C9) | 5.4 | 46 | 56 | 84 | 56 | 58 | 50 | 5 | − | 0-0-3- |
| | | | | | | | | | | | | | | 55-263 |
| Intron 3 donor | VQR-SpBE3 | CUGGCCUGCUGGGCCACCUG | (GGAC) | 20 (C1/−1) | 5.9 | 48 | −1 | 82 | 3 | 62 | 76 | 5 | + | 0-0-2- |
| | | | | | | | | | | | | | | 45-302 |
| R19X | SpBE3 | GGCCUCUGCCCGUAAGCACU | (TGG) | 20 (C11) | 5.6 | 82 | 45 | 14 | 13 | 34 | 36 | 5 | − | 0-0-1- |
| | | | | | | | | | | | | | | 12-105 |
| W62X | SpBE3 | CCCAGCCCCUAAAUCAGUCA | (GGG) | 20 (C2/3) | 7.0 | 66 | 59 | 36 | 18 | 61 | 42 | 7 | − | 0-0-3- |
| | | | | | | | | | | | | | | 23-153 |
| Intron 3 acceptor | SpBE3 | CCCAGCCCCUAAAUCAGUCA | (GGG) | 20 (C8/9) | 7.0 | 66 | 59 | 36 | 18 | 61 | 42 | 7 | − | 0-0-3- |
| | | | | | | | | | | | | | | 23-153 |
| Intron 3 acceptor | SpBE3 | CACCCAGCCCCUAAAUCAGU | (CAG) | 20 (C10/11) | 6.0 | 71 | 52 | 10 | 16 | 44 | 28 | 6 | − | 0-0-2- |
| | | | | | | | | | | | | | | 12-132 |
| M1I | SpBE3 | AUGGCACCUCUGUUCCUGCA | (AGG) | 20 (C−1) | 8.0 | 56 | 63 | 35 | 18 | 43 | 61 | 8 | + | 0-0-4- |
| | | | | | | | | | | | | | | 42-212 |
| Intron 1 donor | SpBE3 | ACCUGGAGCAGCUGCCUCUA | (GGG) | 20 (C2/3) | 4.4 | 43 | 46 | 76 | 8 | 34 | 63 | 4 | − | 0-1-5- |
| | | | | | | | | | | | | | | 40-232 |
| P89L/S | SpBE3 | CCCUGAGGUCAGACCAACUU | (CAG) | 20 (C2/3) | 6.8 | 62 | 54 | 16 | 22 | 36 | 56 | 6 | − | 0-0-3- |
| | | | | | | | | | | | | | | 22-198 |
| Intron 2 acceptor | SaBE3 | CCUCAGGAGCUUCAGAGGCC | (GAGGAT) | 20 (C4) | 7.9 | 69 | −1 | 44 | 6 | 49 | 48 | 7 | + | 0-1-1-6- |
| | | | | | | | | | | | | | | 66 |
| Intron 2 donor | SpBE3 | GGGCAGAGGCCAGGAGCGCC | (AGG) | 20 (C−1) | 7.5 | 36 | 40 | 79 | 1 | 57 | 70 | 7 | + | 0-0-15- |
| | | | | | | | | | | | | | | 70-641 |
| Q54 and Q57 | SpBE3 | GGAGUCCCAGGUGGCCCAGC | (AGG) | 20 (C8) | 5.9 | 42 | 46 | 71 | 10 | 68 | 57 | 5 | + | 0-0-1- |
| | | | | | | | | | | | | | | 50-378 |
| W74X | SpBE3 | CGGUGCUCCAGUAGUCUUUC | (AGG) | 20 (C8/9) | 5.1 | 81 | 13 | 1 | 1 | 13 | 31 | 5 | − | 0-0-1-6- |
| | | | | | | | | | | | | | | 64 |
| Intron 2 acceptor | SpBE3 | AAGCUCCUGAGGAAAGAGCA | (GGG) | 20 (C6/7) | 4.6 | 35 | 64 | 56 | 76 | 65 | 74 | 4 | − | 0-0-9- |
| | | | | | | | | | | | | | | 55-389 |
| Intron 1 donor | VQR-SpBE3 | CCUGGAGCAGCUGCCUCUAG | (GGAT) | 20 (C1/2) | 6.4 | 47 | −1 | 47 | 11 | 40 | 63 | 6 | − | 0-1-5- |
| | | | | | | | | | | | | | | 31-251 |
| W74X | VQR-SpBE3 | CCAGUAGUCUUUCAGGGAAC | (TGAA) | 20 (C1/2) | 5.5 | 63 | −1 | 5 | 9 | 42 | 41 | 5 | + | 0-0-2- |
| | | | | | | | | | | | | | | 17-150 |
| Intron 3 donor | SpBE3 | AGCGGGUGUACCUGGCCUGC | (TGG) | 20 (C11/12) | 4.4 | 60 | 31 | 33 | 1 | 44 | 17 | 4 | + | 0-0-0- |
| | | | | | | | | | | | | | | 16-131 |
| Q54 and Q57 | SpBE3 | UCCCAGGUGGCCCAGCAGGC | (CAG) | 20 (C4/13) | 4.5 | 24 | 37 | 78 | 3 | 42 | 44 | 4 | + | 0-2-5- |
| | | | | | | | | | | | | | | 55-501 |
| Intron 1 donor | SpBE3 | UUACCUGGAGCAGCUGCCUC | (TAG) | 20 (C4/5) | 4.6 | 31 | 29 | 68 | 4 | 35 | 41 | 4 | + | 0-1-3- |
| | | | | | | | | | | | | | | 56-283 |
| Intron 1 donor | SpBE3 | UACCUGGAGCAGCUGCCUCU | (AGG) | 20 (C3/4) | 9.5 | 35 | 50 | 50 | 2 | 47 | 35 | 9 | + | 0-0-14- |
| | | | | | | | | | | | | | | 36-265 |
| Q54 and Q57 | SpBE3 | CCCAGGUGGCCCAGCAGGCC | (AGG) | 20 (C3/12) | 7.1 | 27 | 38 | 41 | 0 | 41 | 54 | 7 | + | 0-1-10- |
| | | | | | | | | | | | | | | 104-583 |
| Intron 3 donor | SpBE3 | ACCUGGCCUGCUGGGCCACC | (TGG) | 20 (C2/3) | 5.6 | 40 | 24 | 39 | 2 | 20 | 37 | 5 | + | 0-0-10- |
| | | | | | | | | | | | | | | 41-318 |
| Intron 2 acceptor | EQR-SpBE3 | UCCUCAGGAGCUUCAGAGGC | (CGAG) | 20 (C5) | 3.5 | 39 | −1 | 22 | 6 | 37 | 37 | 3 | + | 0-0-4- |
| | | | | | | | | | | | | | | 52-319 |
| Intron 2 acceptor | EQR-SpBE3 | GCUCUUUCCUCAGGAGCUUC | (AGAG) | 20 (C11/12) | 4.6 | 42 | −1 | 24 | 6 | 22 | 30 | 4 | − | 0-1-4- |
| | | | | | | | | | | | | | | 27-243 |

*Guide sequences (the portion of the guide RNA that targets the nucleobase editor to the target sequence) are provided, which may be used with any tracrRNA framework sequences provided herein to generate the full guide RNA sequence

In some embodiments, simultaneous introduction of loss-of-function mutations into more than one protein factors in the LDL-mediated cholesterol clearance pathway are provided. For example, in some embodiments, a loss-of-function mutation may be simultaneously introduced into PCSK9 and APOC3. In some embodiments, a loss-of-function mutation may be simultaneously introduced into PCSK9 and LDL-R. In some embodiments, a loss-of-function mutation may be simultaneously introduced into PCSK9 and IODL. In some embodiments, a loss-of-function mutation may be simultaneously introduced into APOC3 and IODL. In some embodiments, a loss-of-function mutation may be simultaneously introduced into LDL-R and APOC3. In some embodiments, a loss-of-function mutation may be simultaneously introduced into LDL-R and IDOL. In some embodiments, a loss-of-function mutation may be simultaneously introduced into PCSK9, APOC3, LDL-R and IDOL. To simultaneous introduce of loss-of-function mutations into more than one protein, multiple guide nucleotide sequences are used.

Further provided herein are methods for the generation of novel and uncharacterized mutations in any of the protein factors involved in the LDL-R mediated cholesterol clearance pathway described herein. For example, libraries of guide nucleotide sequences may be designed for all possible PAM sequences in the genomic site of these protein factors, and used to generate mutations in these proteins. The function of the protein variants may be evaluated. If a loss-of-function variant is identified, the specific gRNA used for making the mutation may be identified via sequencing of the edited genomic site, e.g., via DNA deep sequencing.

### Nucleobase Editors

The methods of generating loss-of-function PCSK9 variants described herein, are enabled by the use of the nucleobase editors. As described herein, a nucleobase editor is a fusion protein comprising: (i) a programmable DNA binding protein domain; and (ii) a deaminase domain. It is to be understood that any programmable DNA binding domain may be used in the based editors.

In some embodiments, the programmable DNA binding protein domain comprises the DNA binding domain of a zinc finger nuclease (ZFN) or a transcription activator-like effector domain (TALE). In some embodiments, the programmable DNA binding protein domain may be programmed by a guide nucleotide sequence, and is thus referred as a “guide nucleotide sequence-programmable DNA binding-protein domain.” In some embodiments, the guide nucleotide sequence-programmable DNA binding protein is a nuclease inactive Cas9, or dCas9. A dCas9 as used herein, encompasses a Cas9 that is completely inactive in its nuclease activity, or partially inactive in its nuclease activity (e.g., a Cas9 nickase). Thus, in some embodiments, the guide nucleotide sequence-programmable DNA binding protein is a Cas9 nickase. In some embodiments, the guide nucleotide sequence-programmable DNA binding protein is a nuclease inactive Cpf1. In some embodiments, the guide nucleotide sequence-programmable DNA binding protein is a nuclease inactive Argonaute.

In some embodiments, the guide nucleotide sequence-programmable DNA binding protein is a dCas9 domain. In some embodiments, the guide nucleotide sequence-programmable DNA binding protein is a Cas9 nickase. In some embodiments, the dCas9 domain comprises the amino acid sequence of SEQ ID NO: 2 or SEQ ID NO: 3. In some embodiments, the dCas9 domain comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 11-260), and comprises mutations corresponding to D10X (X is any amino acid except for D) and/or H840X (X is any amino acid except for H) in SEQ ID NO: 1. In some embodiments, the dCas9 domain comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 11-260), and comprises mutations corresponding to D10A and/or H840A in SEQ ID NO: 1. In some embodiments, the Cas9 nickase comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 11-260), and comprises mutations corresponding to D10X (X is any amino acid except for D) in SEQ ID NO: 1 and a histidine at a position correspond to position 840 in SEQ ID NO: 1. In some embodiments, the Cas9 nickase comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 11-260), and comprises mutations corresponding to D10A in SEQ ID NO: 1 and a histidine at a position correspond to position 840 in SEQ ID NO: 1. In some embodiments, variants or homologues of dCas9 or Cas9 nickase (e.g., variants of SEQ ID NO: 2 or SEQ ID NO: 3, respectively) are provided which are at least about 70% identical, at least about 80% identical, at least about 90% identical, at least about 95% identical, at least about 98% identical, at least about 99% identical, at least about 99.5% identical, or at least about 99.9% to SEQ ID NO: 2 or SEQ ID NO: 3, respectively, and comprises mutations corresponding to D10A and/or H840A in SEQ ID NO: 1. In some embodiments, variants of Cas9 (e.g., variants of SEQ ID NO: 2) are provided having amino acid sequences which are shorter, or longer than SEQ ID NO: 2, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more, provided that the dCas9 variants comprise mutations corresponding to D10A and/or H840A in SEQ ID NO: 1. In some embodiments, variants of Cas9 nickase (e.g., variants of SEQ ID NO: 3) are provided having amino acid sequences which are shorter, or longer than SEQ ID NO: 3, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more, provided that the dCas9 variants comprise mutations corresponding to D10A and comprises a histidine at a position corresponding to position 840 in SEQ ID NO: 1.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, D10A/D839A/H840A/N863A mutant domains (See, e.g., Prashant et al., Nature Biotechnology. 2013; 31(9): 833-838, which are incorporated herein by reference), or K603R (See, e.g., Chavez et al., Nature Methods 12, 326-328, 2015, which is incorporated herein by reference.

In some embodiments, the nucleobase editors described herein comprise a Cas9 domain with decreased electrostatic interactions between the Cas9 domain and a sugar-phosphate backbone of a DNA, as compared to a wild-type Cas9 domain. In some embodiments, a Cas9 domain comprises one or more mutations that decreases the association between the Cas9 domain and a sugar-phosphate backbone of a DNA. In some embodiments, the nucleobase editors described herein comprises a dCas9 (e.g., with D10A and H840A mutations) or a Cas9 nickase (e.g., with D10A mutation), wherein the dCas9 or the Cas9 nickase further comprises one or more of a N497X, a R661X, a Q695X, and/or a Q926X mutation of the amino acid sequence provided in SEQ ID NO: 1, or a corresponding mutation in any of the amino acid sequences provided in SEQ ID NOs: 11-260, wherein X is any amino acid. In some embodiments, the nucleobase editors described herein comprises a dCas9 (e.g., with D10A and H840A mutations) or a Cas9 nickase (e.g., with D10A mutation), wherein the dCas9 or the Cas9 nickase further comprises one or more of a N497A, a R661A, a Q695A, and/or a Q926A mutation of the amino acid sequence provided in SEQ ID NO: 1, or a corresponding mutation in any of the amino acid sequences provided in SEQ ID NOs: 11-260. In some embodiments, the dCas9 domain (e.g., of any of the nucleobase editors provided herein) comprises the amino acid sequence as set forth in any one of SEQ ID NOs: 2-9. In some embodiments, the nucleobase editor comprises the amino acid sequence as set forth in any one of SEQ ID NOs: 293-302 and 321. In some embodiments, the Cas9 domain (e.g., of any of the fusion proteins provided herein) comprises the amino acid sequence as set forth in SEQ ID NO: 9. In some embodiments, the fusion protein comprises the amino acid sequence as set forth in SEQ ID NO: 321. Cas9 domains with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference.

It should be appreciated that the base editors provided herein, for example, base editor 2 (BE2) or base editor 3 (BE3), may be converted into high fidelity base editors by modifying the Cas9 domain as described herein to generate high fidelity base editors, for example, high fidelity base editor 2 (HF-BE2) or high fidelity base editor 3 (HF-BE3). In some embodiments, base editor 2 (BE2) comprises a deaminase domain, a dCas9 domain, and a UGI domain. In some embodiments, base editor 3 (BE3) comprises a deaminase domain, a nCas9 domain, and a UGI domain.

Cas9 variant with decreased electrostatic
interactions between the Cas9 and DNA backbone.
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTAFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGALSRKLINGIRDKQSGKTILDFLKSDGFANRNFMALIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRAITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD (SEQ ID NO: 9, mutations
relative to SEQ ID NO: 1 are bolded
and underlined)
High fidelity nucleobase editor (HF-BE3)

(SEQ ID NO: 321)

MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLKSGSETPGTSESATPESDKKYS
IGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLA
RGNSRFAWMTRKSEETITPWNFEEVVDKGASAQSFIERMTAFDKNLPNEK
VLPKHSLLYEYFTVYNELTKVKYVTEGMRKPAFLSGEQKKAIVDLLFKTN
RKVTVKQLKEDYFKKIECFDSVETSGVEDRFNASLGTYHDLLKIIKDKDF
LDNEENEDILEDIVLTLTLFEDREMIEERLKTYAHLFDDKVMKQLKRRRY
TGWGALSRKLINGIRDKQSGKTILDFLKSDGFANRNFMALIHDDSLTFKE
DIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERG
GLSELDKAGFIKRQLVETRAITKHVAQILDSRMNTKYDENDKLIREVKVI
TLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLY
ETRIDLSQLGGD

Cas9 recognizes a short motif (PAM motif) in the CRISPR repeat sequences in the target DNA sequence. A “PAM motif,” or “protospacer adjacent motif,” as used herein, refers a DNA sequence immediately following the DNA sequence targeted by the Cas9 nuclease in the CRISPR bacterial adaptive immune system. PAM is a component of the invading virus or plasmid, but is not a component of the bacterial CRISPR locus. Naturally, Cas9 will not successfully bind to or cleave the target DNA sequence if it is not followed by the PAM sequence. PAM is an essential targeting component (not found in the bacterial genome) which distinguishes bacterial self from non-self DNA, thereby preventing the CRISPR locus from being targeted and destroyed by nuclease.

Wild-type Streptococcus pyogenes Cas9 recognizes a canonical PAM sequence (5′-NGG-3′). Other Cas9 nucleases (e.g., Cas9 from Streptococcus thermophiles, Staphylococcus aureus, Neisseria meningitidis, or Treponema denticolaor) and Cas9 variants thereof have been described in the art to have different, or more relaxed PAM requirements. For example, in Kleinstiver et al., Nature 523, 481-485, 2015; Klenstiver et al., Nature 529, 490-495, 2016; Ran et al., Nature, April 9; 520(7546): 186-191, 2015; Kleinstiver et al., Nat Biotechnol, 33(12):1293-1298, 2015; Hou et al., Proc Natl Acad Sci USA, 110(39):15644-9, 2014; Prykhozhij et al., PLoS One, 10(3): e0119372, 2015; Zetsche et al., Cell 163, 759-771, 2015; Gao et al., Nature Biotechnology, doi:10.1038/nbt.3547, 2016; Want et al., Nature 461, 754-761, 2009; Chavez et al., doi: dx.doi.org/10.1101/058974; Fagerlund et al., Genome Biol. 2015; 16: 25, 2015; Zetsche et al., Cell, 163, 759-771, 2015; and Swarts et al., Nat Struct Mol Biol, 21(9):743-53, 2014, each of which is incorporated herein by reference.

Thus, the guide nucleotide sequence-programmable DNA-binding protein of the present disclosure may recognize a variety of PAM sequences including, without limitation: NGG, NGAN, NGNG, NGAG, NGCG, NNGRRT, NGRRN, NNNRRT, NNNGATT, NNAGAAW, NAAAC, TTN, TTTN, and YTN, wherein Y is a pyrimidine, and N is any nucleobase.

One example of an RNA-programmable DNA-binding protein that has different PAM specificity is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells.

Also useful in the present disclosure are nuclease-inactive Cpf1 (dCpf1) variants that may be used as a guide nucleotide sequence-programmable DNA-binding protein domain. The Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 (SEQ ID NO: 10) inactivates Cpf1 nuclease activity. In some embodiments, the dCpf1 of the present disclosure comprises mutations corresponding to D917A, E1006A, D1255A, D917A/E1006A, D917A/D1255A, E1006A/D1255A, or D917A/E1006A/D1255A in SEQ ID NO: 10. It is to be understood that any mutations, e.g., substitution mutations, deletions, or insertions that inactivates the RuvC domain of Cpf1 may be used in accordance with the present disclosure.

Thus, in some embodiments, the guide nucleotide sequence-programmable DNA binding protein is a nuclease inactive Cpf1 (dCpf1). In some embodiments, the dCpf1 comprises the amino acid sequence of any one SEQ ID NOs: 261-267 or 2007-2014. In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to SEQ ID NO: 10, and comprises mutations corresponding to D917A, E1006A, D1255A, D917A/E1006A, D917A/D1255A, E1006A/D1255A, or D917A/E1006A/D1255A in SEQ ID NO: 10. Cpf1 from other bacterial species may also be used in accordance with the present disclosure.

Wild type Francisella novicida Cpf1 (SEQ ID NO:
10) (D917, E1006, and D1255 are bolded and
underlined)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN
Francisella novicida Cpf1 D917A (SEQ ID NO: 261)
(A917, E1006, and D1255 are bolded and underlined)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN
Francisella novicida Cpf1 E1006A (SEQ ID NO: 262)
(D917, A1006, and D1255 are bolded and underlined)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN
Francisella novicida Cpf1 D1255A (SEQ ID NO: 263)
(D917, E1006, and A1255 are bolded and underlined)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN
Francisella novicida Cpf1 D917A/E1006A (SEQ ID
NO: 264) (A917, A1006, and D1255 are bolded
and underlined)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN
Francisella novicida Cpf1 D917A/D1255A (SEQ ID
NO: 265) (A917, E1006, and A1255 are bolded
and underlined)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN
Francisella novicida Cpf1 E1006A/D1255A (SEQ
ID NO: 266) (D917, A1006, and A1255 are bolded
and underlined)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN
Francisella novicida Cpf1 D917A/E1006A/D1255A (SEQ
ID NO: 267) (A917, A1006, and A1255 are bolded
and underlined)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN

In some embodiments, the guide nucleotide sequence-programmable DNA binding protein is a Cpf1 protein from an Acidaminoccous species (AsCpf1). Cpf1 proteins form Acidaminococcus species have been described previously and would be apparent to the skilled artisan. Exemplary Acidaminococcus Cpf1 proteins (AsCpf1) include, without limitation, any of the AsCpf1 proteins provided herein.

Wild-type AsCpf1-Residue R912 is indicated in bold
underlining and residues 661-667 are indicated
in italics and underlining.

(SEQ ID NO: 2007)

TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN
AsCpf1(R912A)-Residue A912 is indicated in bold
underlining and residues 661-667 are indicated
in italics and underlining.

(SEQ ID NO: 2008)

TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGEANLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

In some embodiments, the guide nucleotide sequence-programmable DNA binding protein is a Cpf1 protein from a Lachnospiraceae species (LbCpf1). Cpf1 proteins form Lachnospiraceae species have been described previously and would be apparent to the skilled artisan. Exemplary Lachnospiraceae Cpf1 proteins (LbCpf1) include, without limitation, any of the AsCpf1 proteins provided herein.

Wild-type LbCpf1-Residues R836 and R1138 is
indicated in bold underlining.

(SEQ ID NO: 2009)

MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSFNGFTTA
FTGFFDNRENMFSEEAKSTSIAFRCINENLTRYISNMDIFEKVDAIFDKH
EVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYELHIPI
AINKCPKNIFKINTEVRVLLKHDDNPYVIGIDRGERNLLYIVVVDGKGNI
VEQYSLNEIINNFNGIRIKTDYHSLLDKKEKERFEARQNWTSIENIKELK
AGYISQVVHKICELVEKYDAVIALEDLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIFRNPKKNNVFDWEEVCLTSAYKELFN
KYGINYQQGDIRALLCEQSDKAFYSSFMALMSLMLQMRNSITGRTDVDFL
ISPVKNSDGIFYDSRNYEAQENAILPKNADANGAYNIARKVLWAIGQFKK
AEDEKLDKVKIAISNKEWLEYAQTSVKH
LbCpf1 (R836A)-Residue A836 is indicated in
bold underlining.
(SEQ ID NO: 2010)
MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSFNGFTTA
FTGFFDNRENMFSEEAKSTSIAFRCINENLTRYISNMDIFEKVDAIFDKH
EVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYELHIPI
AINKCPKNIFKINTEVRVLLKHDDNPYVIGIDRGEANLLYIVVVDGKGNI
VEQYSLNEIINNFNGIRIKTDYHSLLDKKEKERFEARQNWTSIENIKELK
AGYISQVVHKICELVEKYDAVIALEDLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIFRNPKKNNVFDWEEVCLTSAYKELFN
KYGINYQQGDIRALLCEQSDKAFYSSFMALMSLMLQMRNSITGRTDVDFL
ISPVKNSDGIFYDSRNYEAQENAILPKNADANGAYNIARKVLWAIGQFKK
AEDEKLDKVKIAISNKEWLEYAQTSVKH
LbCpf1 (R1138A)-Residue A1138 is indicated in
bold underlining.

(SEQ ID NO: 2011)

MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSFNGFTTA
FTGFFDNRENMFSEEAKSTSIAFRCINENLTRYISNMDIFEKVDAIFDKH
EVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYELHIPI
AINKCPKNIFKINTEVRVLLKHDDNPYVIGIDRGERNLLYIVVVDGKGNI
VEQYSLNEIINNFNGIRIKTDYHSLLDKKEKERFEARQNWTSIENIKELK
AGYISQVVHKICELVEKYDAVIALEDLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIFRNPKKNNVFDWEEVCLTSAYKELFN
KYGINYQQGDIRALLCEQSDKAFYSSFMALMSLMLQMANSITGRTDVDFL
ISPVKNSDGIFYDSRNYEAQENAILPKNADANGAYNIARKVLWAIGQFKK
AEDEKLDKVKIAISNKEWLEYAQTSVKH

In some embodiments, the Cpf1 protein is a crippled Cpf1 protein. As used herein, a “crippled Cpf1” protein is a Cpf1 protein having diminished nuclease activity as compared to a wild-type Cpf1 protein. In some embodiments, the crippled Cpf1 protein preferentially cuts the target strand more efficiently than the non-target strand. For example, the Cpf1 protein preferentially cuts the strand of a duplexed nucleic acid molecule in which a nucleotide to be edited resides. In some embodiments, the crippled Cpf1 protein preferentially cuts the non-target strand more efficiently than the target strand. For example, the Cpf1 protein preferentially cuts the strand of a duplexed nucleic acid molecule in which a nucleotide to be edited does not reside. In some embodiments, the crippled Cpf1 protein preferentially cuts the target strand at least 5% more efficiently than it cuts the non-target strand. In some embodiments, the crippled Cpf1 protein preferentially cuts the target strand at least 5%, at least 10%, at least 15%, at least 20%, at least 25%, at least 30%, at least 35%, at least 40%, at least 50%, at least 60%, at least 70%, at least 80%, at least 90%, or at least 100% more efficiently than it cuts the non-target strand.

In some embodiments, a crippled Cpf1 protein is a non-naturally occurring Cpf1 protein. In some embodiments, the crippled Cpf1 protein comprises one or more mutations relative to a wild-type Cpf1 protein. In some embodiments, the crippled Cpf1 protein comprises 1, 2, 3, 4, 5, 6, 7, 8, 9, 10, 11, 12, 13, 14, 15, 16, 17, 18, 19, or 20 mutations relative to a wild-type Cpf1 protein. In some embodiments, the crippled Cpf1 protein comprises an R836A mutation mutation as set forth in SEQ ID NO: 2009, or in a corresponding amino acid in another Cpf1 protein. It should be appreciated that a Cpf1 comprising a homologous residue (e.g., a corresponding amino acid) to R836A of SEQ ID NO: 2009 could also be mutated to achieve similar results. In some embodiments, the crippled Cpf1 protein comprises a R1138A mutation as set forth in SEQ ID NO: 2009, or in a corresponding amino acid in another Cpf1 protein. In some embodiments, the crippled Cpf1 protein comprises an R912A mutation mutation as set forth in SEQ ID NO: 2007, or in a corresponding amino acid in another Cpf1 protein. Without wishing to be bound by any particular theory, residue R838 of SEQ ID NO: 2009 (LbCpf1) and residue R912 of SEQ ID NO: 2007 (AsCpf1) are examples of corresponding (e.g., homologous) residues. For example, a portion of the alignment between SEQ ID NO: 2007 and 2009 shows that R912 and R838 are corresponding residues.

| | |
|---|---|
| AsCpf1 | YQAANSPSKFNQRVNAYLKEHPETPIIGIDRGERNLIYITVIDSTGKILEQRSLNTIQ-- |
| LbCpf1 | KCPKN-IFKINTEVRVLLKHDDNPYVIGIDRGERNLLYIVVVDGKGNIVEQYSLNEIINN |
| | * *:* .*.. **.. : :**********:**.*:*..*:*:** *** * |

In some embodiments, any of the Cpf1 proteins provided herein comprises one or more amino acid deletions. In some embodiments, any of the Cpf1 proteins provided herein comprises 1, 2, 3, 4, 5, 6, 7, 8, 9, 10, 11, 12, 13, 14, 15, 16, 17, 18, 19, or 20 amino acid deletions. Without wishing to be bound by any particular theory, there is a helical region in Cpf1, which includes residues 661-667 of AsCpf1 (SEQ ID NO: 2007), that may obstruct the function of a deaminase (e.g., APOBEC) that is fused to the Cpf1. This region comprises the amino acid sequence KKTGDQK. Accordingly, aspects of the disclosure provide Cpf1 proteins comprising mutations (e.g., deletions) that disrupt this helical region in Cpf1. In some embodiments, the Cpf1 protein comprises one or more deletions of the following residues in SEQ ID NO: 2007, or one or more corresponding deletions in another Cpf1 protein: K661, K662, T663, G664, D665, Q666, and K667. In some embodiments, the Cpf1 protein comprises a T663 and a D665 deletion in SEQ ID NO: 2007, or corresponding deletions in another Cpf1 protein. In some embodiments, the Cpf1 protein comprises a K662, T663, D665, and Q666 deletion in SEQ ID NO: 2007, or corresponding deletions in another Cpf1 protein. In some embodiments, the Cpf1 protein comprises a K661, K662, T663, D665, Q666 and K667 deletion in SEQ ID NO: 2007, or corresponding deletions in another Cpf1 protein.

AsCpf1 (deleted T663 and D665)

(SEQ ID NO: 2012)

TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKGQKGYREALCKWIDFTRDFLSKYTKTTSIDLSSLRPSS
QYKDLGEYYAELNPLLYHISFQRIAEKEIMDAVETGKLYLFQIYNKDFAK
GHHGKPNLHTLYWTGLFSPENLAKTSIKLNGQAELFYRPKSRMKRMAHRL
GEKMLNKKLKDQKTPIPDTLYQELYDYVNHRLSHDLSDEARALLPNVITK
EVSHEIIKDRRFTSDKFFFHVPITLNYQAANSPSKFNQRVNAYLKEHPET
PIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSK
RTGIAEKAVYQQFEKMLIDKLNCLVLKDYPAEKVGGVLNPYQLTDQFTSF
AKMGTQSGFLFYVPAPYTSKIDPLTGFVDPFVWKTIKNHESRKHFLEGFD
FLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPK
LLENDDSHAIDTMVALIRSVLQMRNSNAATGEDYINSPVRDLNGVCFDSR
FQNPEWPMDADANGAYHIALKGQLLLNHLKESKDLKLQNGISNQDWLAYI
QELRN
AsCpf1 (deleted K662, T663, D665, and Q666)

(SEQ ID NO: 2013)

TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKGKGYREALCKWIDFTRDFLSKYTKTTSIDLSSLRPSSQY
KDLGEYYAELNPLLYHISFQRIAEKEIMDAVETGKLYLFQIYNKDFAKGH
HGKPNLHTLYWTGLFSPENLAKTSIKLNGQAELFYRPKSRMKRMAHRLGE
KMLNKKLKDQKTPIPDTLYQELYDYVNHRLSHDLSDEARALLPNVITKEV
SHEIIKDRRFTSDKFFFHVPITLNYQAANSPSKFNQRVNAYLKEHPETPI
IGIDRGERNLIYITVIDSTGKILEQRSLNTIQQFDYQKKLDNREKERVAA
RQAWSVVGTIKDLKQGYLSQVIHEIVDLMIHYQAVVVLENLNFGFKSKRT
GIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFL
HYDVKTGDFILHFKMNRNLSFQRGLPGFMPAWDIVFEKNETQFDAKGTPF
IAGKRIVPVIENHRFTGRYRDLYPANELIALLEEKGIVFRDGSNILPKLL
ENDDSHAIDTMVALIRSVLQMRNSNAATGEDYINSPVRDLNGVCFDSRFQ
NPEWPMDADANGAYHIALKGQLLLNHLKESKDLKLQNGISNQDWLAYIQE
LRN
AsCpf1 (deleted K661, K662, T663, D665, Q666,
and K667)

(SEQ ID NO: 2014)

TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAGGYREALCKWIDFTRDFLSKYTKTTSIDLSSLRPSSQYKD
LGEYYAELNPLLYHISFQRIAEKEIMDAVETGKLYLFQIYNKDFAKGHHG
KPNLHTLYWTGLFSPENLAKTSIKLNGQAELFYRPKSRMKRMAHRLGEKM
LNKKLKDQKTPIPDTLYQELYDYVNHRLSHDLSDEARALLPNVITKEVSH
EIIKDRRFTSDKFFFHVPITLNYQAANSPSKFNQRVNAYLKEHPETPIIG
IDRGERNLIYITVIDSTGKILEQRSLNTIQQFDYQKKLDNREKERVAARQ
AWSVVGTIKDLKQGYLSQVIHEIVDLMIHYQAVVVLENLNFGFKSKRTGI
AEKAVYQQFEKMLIDKLNCLVLKDYPAEKVGGVLNPYQLTDQFTSFAKMG
TQSGFLFYVPAPYTSKIDPLTGFVDPFVWKTIKNHESRKHFLEGFDFLHY
DVKTGDFILHFKMNRNLSFQRGLPGFMPAWDIVFEKNETQFDAKGTPFIA
GKRIVPVIENHRFTGRYRDLYPANELIALLEEKGIVFRDGSNILPKLLEN
DDSHAIDTMVALIRSVLQMRNSNAATGEDYINSPVRDLNGVCFDSRFQNP
EWPMDADANGAYHIALKGQLLLNHLKESKDLKLQNGISNQDWLAYIQELR
N

In some embodiments, the guide nucleotide sequence-programmable DNA-binding protein domain of the present disclosure has no requirements for a PAM sequence. One example of such guide nucleotide sequence-programmable DNA-binding protein may be an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at gDNA site. In contrast to Cas9, the NgAgo-gDNA system does not require a protospacer-adjacent motif (PAM). Using a nuclease inactive NgAgo (dNgAgo) can greatly expand the codons that may be targeted. The characterization and use of NgAgo have been described in Gao et al., Nat Biotechnol. Epub 2016 May 2. PubMed PMID: 27136078; Swarts et al., Nature. 507(7491) (2014):258-61; and Swarts et al., Nucleic Acids Res. 43(10) (2015):5120-9, each of which are incorporated herein by reference. The sequence of Natronobacterium gregoryi Argonaute is provided in SEQ ID NO: 270.

Wild type Natronobacterium gregoryi Argonaute

(SEQ ID NO: 270)

MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

In some embodiments, the guide nucleotide sequence-programmable DNA-binding protein is a prokaryotic homolog of an Argonaute protein. Prokaryotic homologs of Argonaute proteins are known and have been described, for example, in Makarova et al., “Prokaryotic homologs of Argonaute proteins are predicted to function as key components of a novel system of defense against mobile genetic elements”, Biol. Direct. 2009 Aug. 25; 4:29. doi: 10.1186/1745-6150-4-29, which is incorporated herein by reference. In some embodiments, the guide nucleotide sequence-programmable DNA-binding protein is a Marinitoga piezophila Argunaute (MpAgo) protein. The CRISPR-associated Marinitoga piezophila Argonaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other Argonaute proteins may be used in any of the fusion proteins (e.g., base editors) described herein, for example, to guide a deaminase (e.g., cytidine deaminase) to a target nucleic acid (e.g., ssRNA).

In some embodiments, the guide nucleotide sequence-programmable DNA-binding protein is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which are herein incorporated by reference. Effectors of two of the systems, C2c1 and C2c3, contain RuvC-like endonuclease domains related to Cpf1. A third system, C2c2 contains an effector with two predicted HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programmed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector,” Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See, e.g., Liu et al., “C2c1-sgRNA Complex Structure Reveals RNA-Guided DNA Cleavage Mechanism”, Mol. Cell, 2017 Jan. 19; 65(2):310-322, incorporated herein by reference. The crystal structure has also been reported for Alicyclobacillus acidoterrestris C2c1 bound to target DNAs as ternary complexes. See, e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the guide nucleotide sequence-programmable DNA-binding protein of any of the fusion proteins provided herein is a C2c1, a C2c2, or a C2c3 protein. In some embodiments, the guide nucleotide sequence-programmable DNA-binding protein is a C2c1 protein. In some embodiments, the guide nucleotide sequence-programmable DNA-binding protein is a C2c2 protein. In some embodiments, the guide nucleotide sequence-programmable DNA-binding protein is a C2c3 protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the guide nucleotide sequence-programmable DNA-binding protein is a naturally-occurring C2c1, C2c2, or C2c3 protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 2015-2017. In some embodiments, the guide nucleotide sequence-programmable DNA-binding protein comprises an amino acid sequence of any one SEQ ID NOs: 2015-2017. It should be appreciated that C2c1, C2c2, or C2c3 from other bacterial species may also be used in accordance with the present disclosure.

C2c1 (uniprot.org/uniprot/T0D7A2#)
sp|T0D7A2|C2C1_ALIAG CRISPR-associated endonuclease C2c1
OS = Alicyclobacillus acidoterrestris (strain ATCC 49025/DSM
3922/CIP 106132/NCIMB 13137/GD3B) GN = c2c1 PE = 1 SV = 1
(SEQ ID NO: 2015)
MAVKSIKVKLRLDDMPEIRAGLWKLHKEVNAGVRYYTEWLSLLRQENLYRRSPNGDG
EQECDKTAEECKAELLERLRARQVENGHRGPAGSDDELLQLARQLYELLVPQAIGAKG
DAQQIARKFLSPLADKDAVGGLGIAKAGNKPRWVRMREAGEPGWEEEKEKAETRKSA
DRTADVLRALADFGLKPLMRVYTDSEMSSVEWKPLRKGQAVRTWDRDMFQQAIERM
MSWESWNQRVGQEYAKLVEQKNRFEQKNFVGQEHLVHLVNQLQQDMKEASPGLESK
EQTAHYVTGRALRGSDKVFEKWGKLAPDAPFDLYDAEIKNVQRRNTRRFGSHDLFAKL
AEPEYQALWREDASFLTRYAVYNSILRKLNHAKMFATFTLPDATAHPIWTRFDKLGGN
LHQYTFLFNEFGERRHAIRFHKLLKVENGVAREVDDVTVPISMSEQLDNLLPRDPNEPIA
LYFRDYGAEQHFTGEFGGAKIQCRRDQLAHMHRRRGARDVYLNVSVRVQSQSEARGE
RRPPYAAVFRLVGDNHRAFVHFDKLSDYLAEHPDDGKLGSEGLLSGLRVMSVDLGLRT
SASISVFRVARKDELKPNSKGRVPFFFPIKGNDNLVAVHERSQLLKLPGETESKDLRAIRE
ERQRTLRQLRTQLAYLRLLVRCGSEDVGRRERSWAKLIEQPVDAANHMTPDWREAFEN
ELQKLKSLHGICSDKEWMDAVYESVRRVWRHMGKQVRDWRKDVRSGERPKIRGYAK
DVVGGNSIEQIEYLERQYKFLKSWSFFGKVSGQVIRAEKGSRFAITLREHIDHAKEDRLK
KLADRIIMEALGYVYALDERGKGKWVAKYPPCQLILLEELSEYQFNNDRPPSENNQLM
QWSHRGVFQELINQAQVHDLLVGTMYAAFSSRFDARTGAPGIRCRRVPARCTQEHNPE
PFPWWLNKFVVEHTLDACPLRADDLIPTGEGEIFVSPFSAEEGDFHQIHADLNAAQNLQ
QRLWSDFDISQIRLRCDWGEVDGELVLIPRLTGKRTADSYSNKVFYTNTGVTYYERERG
KKRRKVFAQEKLSEEEAELLVEADEAREKSVVLMRDPSGIINRGNWTRQKEFWSMVNQ
RIEGYLVKQIRSRVPLQDSACENTGDI
C2c2 (uniprot.org/uniprot/P0DOC6)
>sp|P0DOC6|C2C2_LEPSD CRISPR-associated endoribonuclease
C2c2 OS = Leptotrichia shahii (strain DSM 19757/CCUG 47503/CIP
107916/JCM 16776/LB37) GN = c2c2 PE = 1 SV = 1
(SEQ ID NO: 2016)
MGNLFGHKRWYEVRDKKDFKIKRKVKVKRNYDGNKYILNINENNNKEKIDNNKFIRKY
INYKKNDNILKEFTRKFHAGNILFKLKGKEGIIRIENNDDFLETEEVVLYIEAYGKSEKLK
ALGITKKKIIDEAIRQGITKDDKKIEIKRQENEEEIEIDIRDEYTNKTLNDCSIILRIIENDELE
TKKSIYEIFKNINMSLYKIIEKIIENETEKVFENRYYEEHLREKLLKDDKIDVILTNFMEIRE
KIKSNLEILGFVKFYLNVGGDKKKSKNKKMLVEKILNINVDLTVEDIADFVIKELEFWNI
TKRIEKVKKVNNEFLEKRRNRTYIKSYVLLDKHEKFKIERENKKDKIVKFFVENIKNNSI
KEKIEKILAEFKIDELIKKLEKELKKGNCDTEIFGIFKKHYKVNFDSKKFSKKSDEEKELY
KIIYRYLKGRIEKILVNEQKVRLKKMEKIEIEKILNESILSEKILKRVKQYTLEHIMYLGKL
RHNDIDMTTVNTDDFSRLHAKEELDLELITFFASTNMELNKIFSRENINNDENIDFFGGDR
EKNYVLDKKILNSKIKIIRDLDFIDNKNNITNNFIRKFTKIGTNERNRILHAISKERDLQGT
QDDYNKVINIIQNLKISDEEVSKALNLDVVFKDKKNIITKINDIKISEENNNDIKYLPSFSK
VLPEILNLYRNNPKNEPFDTIETEKIVLNALIYVNKELYKKLILEDDLEENESKNIFLQELK
KTLGNIDEIDENIIENYYKNAQISASKGNNKAIKKYQKKVIECYIGYLRKNYEELFDFSDF
KMNIQEIKKQIKDINDNKTYERITVKTSDKTIVINDDFEYIISIFALLNSNAVINKIRNRFFA
TSVWLNTSEYQNIIDILDEIMQLNTLRNECITENWNLNLEEFIQKMKEIEKDFDDFKIQTK
KEIFNNYYEDIKNNILTEFKDDINGCDVLEKKLEKIVIFDDETKFEIDKKSNILQDEQRKLS
NINKKDLKKKVDQYIKDKDQEIKSKILCRIIFNSDFLKKYKKEIDNLIEDMESENENKFQE
IYYPKERKNELYIYKKNLFLNIGNPNFDKIYGLISNDIKMADAKFLFNIDGKNIRKNKISEI
DAILKNLNDKLNGYSKEYKEKYIKKLKENDDFFAKNIQNKNYKSFEKDYNRVSEYKKIR
DLVEFNYLNKIESYLIDINWKLAIQMARFERDMHYIVNGLRELGIIKLSGYNTGISRAYPK
RNGSDGFYTTTAYYKFFDEESYKKFEKICYGFGIDLSENSEINKPENESIRNYISHFYIVRN
PFADYSIAEQIDRVSNLLSYSTRYNNSTYASVFEVFKKDVNLDYDELKKKFKLIGNNDIL
ERLMKPKKVSVLELESYNSDYIKNLIIELLTKIENTNDTL
C2c3, translated from >CEPX01008730.1 marine metagenome
genome assembly TARA_037_MES_0.1-0.22, contig
TARA_037_MES_0.1-0.22_scaffold22115_1, whole
genome shotgun sequence.
(SEQ ID NO: 2017)
MRSNYHGGRNARQWRKQISGLARRTKETVFTYKFPLETDAAEIDFDKAVQTYGIAEGV
GHGSLIGLVCAFHLSGFRLFSKAGEAMAFRNRSRYPTDAFAEKLSAIMGIQLPTLSPEGL
DLIFQSPPRSRDGIAPVWSENEVRNRLYTNWTGRGPANKPDEHLLEIAGEIAKQVFPKFG
GWDDLASDPDKALAAADKYFQSQGDFPSIASLPAAIMLSPANSTVDFEGDYIAIDPAAET
LLHQAVSRCAARLGRERPDLDQNKGPFVSSLQDALVSSQNNGLSWLFGVGFQHWKEKS
PKELIDEYKVPADQHGAVTQVKSFVDAIPLNPLFDTTHYGEFRASVAGKVRSWVANYW
KRLLDLKSLLATTEFTLPESISDPKAVSLFSGLLVDPQGLKKVADSLPARLVSAEEAIDRL
MGVGIPTAADIAQVERVADEIGAFIGQVQQFNNQVKQKLENLQDADDEEFLKGLKIELP
SGDKEPPAINRISGGAPDAAAEISELEEKLQRLLDARSEHFQTISEWAEENAVTLDPIAAM
VELERLRLAERGATGDPEEYALRLLLQRIGRLANRVSPVSAGSIRELLKPVFMEEREFNL
FFHNRLGSLYRSPYSTSRHQPFSIDVGKAKAIDWIAGLDQISSDIEKALSGAGEALGDQLR
DWINLAGFAISQRLRGLPDTVPNALAQVRCPDDVRIPPLLAMLLEEDDIARDVCLKAFN
LYVSAINGCLFGALREGFIVRTRFQRIGTDQIHYVPKDKAWEYPDRLNTAKGPINAAVSS
DWIEKDGAVIKPVETVRNLSSTGFAGAGVSEYLVQAPHDWYTPLDLRDVAHLVTGLPV
EKNITKLKRLTNRTAFRMVGASSFKTHLDSVLLSDKIKLGDFTIIIDQHYRQSVTYGGKV
KISYEPERLQVEAAVPVVDTRDRTVPEPDTLFDHIVAIDLGERSVGFAVFDIKSCLRTGEV
KPIHDNNGNPVVGTVAVPSIRRLMKAVRSHRRRRQPNQKVNQTYSTALQNYRENVIGD
VCNRIDTLMERYNAFPVLEFQIKNFQAGAKQLEIVYGS

In some embodiments, the guide nucleotide sequence-programmable DNA-binding protein of any of the fusion proteins provided herein is a Cas9 from archaea (e.g. nanoarchaea), which constitute a domain and kingdom of single-celled prokaryotic microbes. In some embodiments, the guide nucleotide sequence-programmable DNA-binding protein is CasX or CasY, which have been described in, for example, Burstein et al., “New CRISPR-Cas systems from uncultivated microbes.” Cell Res. 2017 February 21. doi: 10.1038/cr.2017.21, which is incorporated herein by reference. Using genome-resolved metagenomics, a number of CRISPR-Cas systems were identified, including the first reported Cas9 in the archaeal domain of life. This divergent Cas9 protein was found in nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a guide nucleotide sequence-programmable DNA-binding protein and are within the scope of this disclosure.

In some embodiments, the guide nucleotide sequence-programmable DNA-binding protein of any of the fusion proteins provided herein is a CasX or CasY protein. In some embodiments, the guide nucleotide sequence-programmable DNA-binding protein is a CasX protein. In some embodiments, the guide nucleotide sequence-programmable DNA-binding protein is a CasY protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the guide nucleotide sequence-programmable DNA-binding protein is a naturally-occurring CasX or CasY protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 2018-2020. In some embodiments, the guide nucleotide sequence-programmable DNA-binding protein comprises an amino acid sequence of any one of SEQ ID NOs: 2018-2020. It should be appreciated that CasX and CasY from other bacterial species may also be used in accordance with the present disclosure.

CasX (uniprot.org/uniprot/F0NN87; uniprot.org/uniprot/F0NH53)
>tr|F0NN87|F0NN87_SULIH CRISPR-associated Casx protein
OS = Sulfolobus islandicus (strain HVE10/4) GN = SiH_0402
PE = 4 SV = 1
(SEQ ID NO: 2018)
MEVPLYNIFGDNYIIQVATEAENSTIYNNKVEIDDEELRNVLNLAYKIAKNNEDAAAERR
GKAKKKKGEEGETTTSNIILPLSGNDKNPWTETLKCYNFPTTVALSEVFKNFSQVKECEE
VSAPSFVKPEFYEFGRSPGMVERTRRVKLEVEPHYLIIAAAGWVLTRLGKAKVSEGDYV
GVNVFTPTRGILYSLIQNVNGIVPGIKPETAFGLWIARKVVSSVTNPNVSVVRIYTISDAV
GQNPTTINGGFSIDLTKLLEKRYLLSERLEAIARNALSISSNMRERYIVLANYIYEYLTGSK
RLEDLLYFANRDLIMNLNSDDGKVRDLKLISAYVNGELIRGEG
>tr|F0NH53|F0NH53_SULIR CRISPR associated protein, Casx
OS = Sulfolobus islandicus (strain REY15A) GN = SiRe_0771
PE = 4 SV = 1
(SEQ ID NO: 2019)
MEVPLYNIFGDNYIIQVATEAENSTIYNNKVEIDDEELRNVLNLAYKIAKNNEDAAAERR
GKAKKKKGEEGETTTSNIILPLSGNDKNPWTETLKCYNFPTTVALSEVFKNFSQVKECEE
VSAPSFVKPEFYKFGRSPGMVERTRRVKLEVEPHYLIMAAAGWVLTRLGKAKVSEGDY
VGVNVFTPTRGILYSLIQNVNGIVPGIKPETAFGLWIARKVVSSVTNPNVSVVSIYTISDA
VGQNPTTINGGFSIDLTKLLEKRDLLSERLEAIARNALSISSNMRERYIVLANYIYEYLTGS
KRLEDLLYFANRDLIMNLNSDDGKVRDLKLISAYVNGELIRGEG
CasY (ncbi.nlm.nih.gov/protein/APG80656.1)
>APG80656.1 CRISPR-associated protein CasY [uncultured
Parcubacteria group bacterium]
(SEQ ID NO: 2020)
MSKRHPRISGVKGYRLHAQRLEYTGKSGAMRTIKYPLYSSPSGGRTVPREIVSAINDDY
VGLYGLSNFDDLYNAEKRNEEKVYSVLDFWYDCVQYGAVFSYTAPGLLKNVAEVRGG
SYELTKTLKGSHLYDELQIDKVIKFLNKKEISRANGSLDKLKKDIIDCFKAEYRERHKDQ
CNKLADDIKNAKKDAGASLGERQKKLFRDFFGISEQSENDKPSFTNPLNLTCCLLPFDTV
NNNRNRGEVLFNKLKEYAQKLDKNEGSLEMWEYIGIGNSGTAFSNFLGEGFLGRLREN
KITELKKAMMDITDAWRGQEQEEELEKRLRILAALTIKLREPKFDNHWGGYRSDINGKL
SSWLQNYINQTVKIKEDLKGHKKDLKKAKEMINRFGESDTKEEAVVSSLLESIEKIVPDD
SADDEKPDIPAIAIYRRFLSDGRLTLNRFVQREDVQEALIKERLEAEKKKKPKKRKKKSD
AEDEKETIDFKELFPHLAKPLKLVPNFYGDSKRELYKKYKNAAIYTDALWKAVEKIYKS
AFSSSLKNSFFDTDFDKDFFIKRLQKIFSVYRRFNTDKWKPIVKNSFAPYCDIVSLAENEV
LYKPKQSRSRKSAAIDKNRVRLPSTENIAKAGIALARELSVAGFDWKDLLKKEEHEEYID
LIELHKTALALLLAVTETQLDISALDFVENGTVKDFMKTRDGNLVLEGRFLEMFSQSIVF
SELRGLAGLMSRKEFITRSAIQTMNGKQAELLYIPHEFQSAKITTPKEMSRAFLDLAPAEF
ATSLEPESLSEKSLLKLKQMRYYPHYFGYELTRTGQGIDGGVAENALRLEKSPVKKREIK
CKQYKTLGRGQNKIVLYVRSSYYQTQFLEWFLHRPKNVQTDVAVSGSFLIDEKKVKTR
WNYDALTVALEPVSGSERVFVSQPFTIFPEKSAEEEGQRYLGIDIGEYGIAYTALEITGDS
AKILDQNFISDPQLKTLREEVKGLKLDQRRGTFAMPSTKIARIRESLVHSLRNRIHHLALK
HKAKIVYELEVSRFEEGKQKIKKVYATLKKADVYSEIDADKNLQTTVWGKLAVASEISA
SYTSQFCGACKKLWRAEMQVDETITTQELIGTVRVIKGGTLIDAIKDFMRPPIFDENDTPF
PKYRDFCDKHHISKKMRGNSCLFICPFCRANADADIQASQTIALLRYVKEEKKVEDYFE
RFRKLKNIKVLGQMKKI

Cas9 Domains with Reduced PAM Exclusivity

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a four base region (e.g., a “deamination window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence and has relaxed PAM requirements (PAMless Cas9). PAMless Cas9 exhibits an increased activity on a target sequence that does not include a canonical PAM (e.g., NGG) at its 3′-end as compared to Streptococcus pyogenes Cas9 as provided by SEQ ID NO: 1, e.g., increased activity by at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference. See also U.S. Provisional Applications 62/245,828, 62/279,346, 62/311,763, 62/322,178, and 62/357,332, each of which is incorporated herein by reference. In some embodiments, the dCas9 or Cas9 nickase useful in the present disclosure may further comprise mutations that relax the PAM requirements, e.g., mutations that correspond to A262T, K294R, S409I, E480K, E543D, M694I, or E1219V in SEQ ID NO: 1.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises the amino acid sequence SEQ ID NO: 2021. In some embodiments, the SaCas9 comprises a N579X mutation of SEQ ID NO: 2021, or a corresponding mutation in any of the amino acid sequences provided in any of the Cas9 proteins disclosed herein including, but not limited to, SEQ ID NOs: 1-260, 2004, or 2006, wherein X is any amino acid except for N. In some embodiments, the SaCas9 comprises a N579A mutation of SEQ ID NO: 2021, or a corresponding mutation in any of the amino acid sequences provided in SEQ ID NOs: 1-260, 2004, or 2006. In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT PAM sequence. In some embodiments, the SaCas9 domain comprises one or more of a E781X, a N967X, and a R1014X mutation of SEQ ID NO: 2021, or a corresponding mutation in any of the Cas9 amino acid sequences provided herein, including but not limited to in SEQ ID NOs: 1-260, 2004, or 2006, wherein X is any amino acid. In some embodiments, the SaCas9 domain comprises one or more of a E781K, a N967K, and a R1014H mutation of SEQ ID NO: 2021, or one or more corresponding mutation in any of the Cas9 amino acid sequences provided herein, including but not limited to in SEQ ID NOs: 1-260, 2004, or 2006. In some embodiments, the SaCas9 domain comprises a E781K, a N967K, or a R1014H mutation of SEQ ID NO: 2021, or one or more corresponding mutation in any of the Cas9 amino acid sequences provided herein, including but not limited to in SEQ ID NOs: 1-260, 2004, or 2006.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 2021-2024 or 268. In some embodiments, the Cas9 domain of any of the fusion proteins provided herein comprises the amino acid sequence of any one of SEQ ID NOs: 2021-2024 or 268. In some embodiments, the Cas9 domain of any of the fusion proteins provided herein consists of the amino acid sequence of any one of SEQ ID NOs: 2021-2024 or 268.

Exemplary SaCas9 sequence
(SEQ ID NO: 2021)
KRNYILGLDIGITSVGYGIIDYETRDVIDAGVRLFKEANVENNEGRRSKRGARRLKRRRR
HRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKE
AKQLLKVQKAYHQLDQSFIDTYIDLLETRRTYYEGPGEGSPFGWKDIKEWYEMLMGHC
TYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
Residue N579 of SEQ ID NO: 2021, which is underlined and in
bold, may be mutated (e.g., to a A579) to yield a SaCas9
nickase.
Exemplary SaCas9d sequence
(SEQ ID NO: 2022)
KRNYILGLAIGITSVGYGIIDYETRDVIDAGVRLFKEANVENNEGRRSKRGARRLKRRRR
HRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKE
AKQLLKVQKAYHQLDQSFIDTYIDLLETRRTYYEGPGEGSPFGWKDIKEWYEMLMGHC
TYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
Residue A10 of SEQ ID NO: 2022, which can be mutated from
D10 of SEQ ID NO: E1 to yield a nuclease inactive SaCas9d,
is underlined and in bold.
Exemplary SaCas9n sequence
(SEQ ID NO: 2023)
KRNYILGLDIGITSVGYGIIDYETRDVIDAGVRLFKEANVENNEGRRSKRGARRLKRRRR
HRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKE
AKQLLKVQKAYHQLDQSFIDTYIDLLETRRTYYEGPGEGSPFGWKDIKEWYEMLMGHC
TYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEEASKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
Residue A579 of SEQ ID NO: 2023, which can be mutated from
N579 of SEQ ID NO: 2021 to yield a SaCas9 nickase, is
underlined and in bold.
Exemplary SaKKH Cas9
(SEQ ID NO: 2024)
KRNYILGLDIGITSVGYGIIDYETRDVIDAGVRLFKEANVENNEGRRSKRGARRLKRRRR
HRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKE
AKQLLKVQKAYHQLDQSFIDTYIDLLETRRTYYEGPGEGSPFGWKDIKEWYEMLMGHC
TYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEEASKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
KLINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYKNDLIKINGELYRVIGVNNDLLNRIEVNMIDITYREYLENMNDKRPPH
IIKTIASKTQSIKKYSTDILGNLYEVKSKKHPQIIKKG.
Residue A579 of SEQ ID NO: 2024, which can be mutated from
N579 of SEQ ID NO: 2021 to yield a SaCas9 nickase, is
underlined and in bold. Residues K781, K967, and H1014 of SEQ
ID SEQ ID NO: 2024, which can be mutated from E781, N967,
and R1014 of SEQ ID NO: 2021 to yield a SaKKH Cas9
are underlined and initalics.
KKH-nCas9 (D10A/E782K/N968K/R1015H) S. aureus Cas9 Nickase
(SEQ ID NO: 268)
MKRNYILGLAIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
KLINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYKNDLIKINGELYRVIGVNNDLLNRIEVNMIDITYREYLENMNDKRPPH
IIKTIASKTQSIKKYSTDILGNLYEVKSKKHPQIIKKG

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises the amino acid sequence SEQ ID NO: 2025. In some embodiments, the SpCas9 comprises a D9X mutation of SEQ ID NO: 2025, or a corresponding mutation in any of the Cas9 amino acid sequences provided herein, including but not limited to SEQ ID NOs: 1-260, 2004, or 2006, wherein X is any amino acid except for D. In some embodiments, the SpCas9 comprises a D9A mutation of SEQ ID NO: 2025, or a corresponding mutation in any of the Cas9 amino acid sequences provided herein, including but not limited to SEQ ID NOs: 1-260, 2004, or 2006. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having a NGG, a NGA, or a NGCG PAM sequence. In some embodiments, the SpCas9 domain comprises one or more of a D1134X, a R1334X, and a T1336X mutation of SEQ ID NO: 2025, or a corresponding mutation in any of the Cas9 amino acid sequences provided herein, including but not limited to SEQ ID NOs: 1-260, 2004, or 2006, wherein X is any amino acid. In some embodiments, the SpCas9 domain comprises one or more of a D1134E, R1334Q, and T1336R mutation of SEQ ID NO: 2025, or a corresponding mutation in any of the Cas9 amino acid sequences provided herein, including but not limited to SEQ ID NOs: 1-260, 2004, or 2006. In some embodiments, the SpCas9 domain comprises a D1134E, a R1334Q, and a T1336R mutation of SEQ ID NO: 2025, or a corresponding mutation in any of the Cas9 amino acid sequences provided herein, including but not limited to SEQ ID NOs: 1-260, 2004, or 2006. In some embodiments, the SpCas9 domain comprises one or more of a D1134X, a R1334X, and a T1336X mutation of SEQ ID NO: 2025, or a corresponding mutation in any of the Cas9 amino acid sequences provided herein, including but not limited to SEQ ID NOs: 1-260, 2004, or 2006, wherein X is any amino acid. In some embodiments, the SpCas9 domain comprises one or more of a D1134V, a R1334Q, and a T1336R mutation of SEQ ID NO: 2025, or a corresponding mutation in any of the Cas9 amino acid sequences provided herein, including but not limited to SEQ ID NOs: 1-260, 2004, or 2006. In some embodiments, the SpCas9 domain comprises a D1134V, a R1334Q, and a T1336R mutation of SEQ ID NO: 2025, or a corresponding mutation in any of the Cas9 amino acid sequences provided herein, including but not limited to SEQ ID NOs: 1-260, 2004, or 2006. In some embodiments, the SpCas9 domain comprises one or more of a D1134X, a G1217X, a R1334X, and a T1336X mutation of SEQ ID NO: 2025, or a corresponding mutation in any of the Cas9 amino acid sequences provided herein, including but not limited to SEQ ID NOs: 1-260, 2004, or 2006, wherein X is any amino acid. In some embodiments, the SpCas9 domain comprises one or more of a D1134V, a G1217R, a R1334Q, and a T1336R mutation of SEQ ID NO: 2025, or a corresponding mutation in any of the Cas9 amino acid sequences provided herein, including but not limited to SEQ ID NOs: 1-260, 2004, or 2006. In some embodiments, the SpCas9 domain comprises a D1134V, a G1217R, a R1334Q, and a T1336R mutation of SEQ ID NO: 2025, or a corresponding mutation in any of the Cas9 amino acid sequences provided herein, including but not limited to SEQ ID NOs: 1-260, 2004, or 2006.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 2025-2029 or 2000-2002. In some embodiments, the Cas9 domain of any of the fusion proteins provided herein comprises the amino acid sequence of any one of SEQ ID NOs: 2025-2029 or 2000-2002. In some embodiments, the Cas9 domain of any of the fusion proteins provided herein consists of the amino acid sequence of any one of SEQ ID NOs: 2025-2029 or 2000-2002.

Exemplary SpCas9
(SEQ ID NO: 2025)
DKKYSIGLDIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGGD
Exemplary SpCas9n
(SEQ ID NO: 2026)
DKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGGD
VRER-Cas9 (D1135V/G1218R/R1335E/T1337R) S. pyogenes Cas9
(SEQ ID NO: 2027)
MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFVSPTVAYSVLVVAKVEKGKSKKLKSVKELLGITIMERSSFEKNPIDFLEAKGYKE
VKKDLIIKLPKYSLFELENGRKRMLASARELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKEYRSTKEVLDATLIHQSITGLYETRIDLSQLGGD
(single underline: HNH domain; double underline: RuvC domain)
VRER-nCas9 (D10A/D1135V/G1218R/R1335E/T1337R)
S. pyogenes Cas9 Nickase
(SEQ ID NO: 2000)
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFVSPTVAYSVLVVAKVEKGKSKKLKSVKELLGITIMERSSFEKNPIDFLEAKGYKE
VKKDLIIKLPKYSLFELENGRKRMLASARELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKEYRSTKEVLDATLIHQSITGLYETRIDLSQLGGD
(single underline: HNH domain; double underline: RuvC domain)
VQR-Cas9 (D1135V/R1335Q/T1337R) S. pyogenes Cas9
(SEQ ID NO: 2028)
MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFVSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKQYRSTKEVLDATLIHQSITGLYETRIDLSQLGGD
(single underline: HNH domain; double underline: RuvC domain)
VQR-nCas9 (D10A/D1135V/R1335Q/T1337R) S. pyogenes
Cas9 Nickase
(SEQ ID NO: 2001)
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFVSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKQYRSTKEVLDATLIHQSITGLYETRIDLSQLGGD
(single underline: HNH domain; double underline: RuvC domain)
EQR-Cas9 (D1135E/R1335Q/T1337R) S. pyogenes Cas9
(SEQ ID NO: 2029)
MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFESPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENII
HLFTLTNLGAPAAFKYFDTTIDRKQYRSTKEVLDATLIHQSITGLYETRIDLSQLGGD
(single underline: HNH domain; double underline: RuvC domain)
EQR-nCas9 (D10A/D1135E/R1335Q/T1337R) S. pyogenes
Cas9 Nickase
(SEQ ID NO: 2002)
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFESPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENII
HLFTLTNLGAPAAFKYFDTTIDRKQYRSTKEVLDATLIHQSITGLYETRIDLSQLGGD
(single underline: HNH domain; double underline: RuvC domain)

Other on-limiting, exemplary Cas9 variants (including dCas9, Cas9 nickase, and Cas9 variants with alternative PAM requirements) suitable for use in the nucleobase editors described herein and their respective sequence are provided below.

Streptococcus thermophilus CRISPR1 Cas9 (St1Cas9)
Nickase (D9A)
(SEQ ID NO: 269)
MSDLVLGLAIGIGSVGVGILNKVTGEIIHKNSRIFPAAQAENNLVRRTNRQGRRLTRRKK
HRRVRLNRLFEESGLITDFTKISINLNPYQLRVKGLTDELSNEELFIALKNMVKHRGISYL
DDASDDGNSSIGDYAQIVKENSKQLETKTPGQIQLERYQTYGQLRGDFTVEKDGKKHRL
INVFPTSAYRSEALRILQTQQEFNPQITDEFINRYLEILTGKRKYYHGPGNEKSRTDYGRY
RTSGETLDNIFGILIGKCTFYPDEFRAAKASYTAQEFNLLNDLNNLTVPTETKKLSKEQK
NQIINYVKNEKAMGPAKLFKYIAKLLSCDVADIKGYRIDKSGKAEIHTFEAYRKMKTLE
TLDIEQMDRETLDKLAYVLTLNTEREGIQEALEHEFADGSFSQKQVDELVQFRKANSSIF
GKGWHNFSVKLMMELIPELYETSEEQMTILTRLGKQKTTSSSNKTKYIDEKLLTEEIYNP
VVAKSVRQAIKIVNAAIKEYGDFDNIVIEMARETNEDDEKKAIQKIQKANKDEKDAAML
KAANQYNGKAELPHSVFHGHKQLATKIRLWHQQGERCLYTGKTISIHDLINNSNQFEVD
HILPLSITFDDSLANKVLVYATANQEKGQRTPYQALDSMDDAWSFRELKAFVRESKTLS
NKKKEYLLTEEDISKFDVRKKFIERNLVDTRYASRVVLNALQEHFRAHKIDTKVSVVRG
QFTSQLRRHWGIEKTRDTYHHHAVDALIIAASSQLNLWKKQKNTLVSYSEDQLLDIETG
ELISDDEYKESVFKAPYQHFVDTLKSKEFEDSILFSYQVDSKFNRKISDATIYATRQAKV
GKDKADETYVLGKIKDIYTQDGYDAFMKIYKKDKSKFLMYRHDPQTFEKVIEPILENYP
NKQINEKGKEVPCNPFLKYKEEHGYIRKYSKKGNGPEIKSLKYYDSKLGNHIDITPKDSN
NKVVLQSVSPWRADVYFNKTTGKYEILGLKYADLQFEKGTGTYKISQEKYNDIKKKEG
VDSDSEFKFTLYKNDLLLVKDTETKEQQLFRFLSRTMPKQKHYVELKPYDKQKFEGGE
ALIKVLGNVANSGQCKKGLGKSNISIYKVRTDVLGNQHIIKNEGDKPKLDF
Streptococcus thermophilus CRISPR3Cas9 (St3Cas9)
Nickase (D10A)
(SEQ ID NO: 1999)
MTKPYSIGLAIGTNSVGWAVITDNYKVPSKKMKVLGNTSKKYIKKNLLGVLLFDSGITA
EGRRLKRTARRRYTRRRNRILYLQEIFSTEMATLDDAFFQRLDDSFLVPDDKRDSKYPIF
GNLVEEKVYHDEFPTIYHLRKYLADSTKKADLRLVYLALAHMIKYRGHFLIEGEFNSKN
NDIQKNFQDFLDTYNAIFESDLSLENSKQLEEIVKDKISKLEKKDRILKLFPGEKNSGIFSE
FLKLIVGNQADFRKCFNLDEKASLHFSKESYDEDLETLLGYIGDDYSDVFLKAKKLYDAI
LLSGFLTVTDNETEAPLSSAMIKRYNEHKEDLALLKEYIRNISLKTYNEVFKDDTKNGYA
GYIDGKTNQEDFYVYLKNLLAEFEGADYFLEKIDREDFLRKQRTFDNGSIPYQIHLQEMR
AILDKQAKFYPFLAKNKERIEKILTFRIPYYVGPLARGNSDFAWSIRKRNEKITPWNFEDV
IDKESSAEAFINRMTSFDLYLPEEKVLPKHSLLYETFNVYNELTKVRFIAESMRDYQFLD
SKQKKDIVRLYFKDKRKVTDKDIIEYLHAIYGYDGIELKGIEKQFNSSLSTYHDLLNIIND
KEFLDDSSNEAIIEEIIHTLTIFEDREMIKQRLSKFENIFDKSVLKKLSRRHYTGWGKLSAK
LINGIRDEKSGNTILDYLIDDGISNRNFMQLIHDDALSFKKKIQKAQIIGDEDKGNIKEVV
KSLPGSPAIKKGILQSIKIVDELVKVMGGRKPESIVVEMARENQYTNQGKSNSQQRLKRL
EKSLKELGSKILKENIPAKLSKIDNNALQNDRLYLYYLQNGKDMYTGDDLDIDRLSNYD
IDHIIPQAFLKDNSIDNKVLVSSASNRGKSDDFPSLEVVKKRKTFWYQLLKSKLISQRKFD
NLTKAERGGLLPEDKAGFIQRQLVETRQITKHVARLLDEKFNNKKDENNRAVRTVKIIT
LKSTLVSQFRKDFELYKVREINDFHHAHDAYLNAVIASALLKKYPKLEPEFVYGDYPKY
NSFRERKSATEKVYFYSNIMNIFKKSISLADGRVIERPLIEVNEETGESVWNKESDLATVR
RVLSYPQVNVVKKVEEQNHGLDRGKPKGLFNANLSSKPKPNSNENLVGAKEYLDPKK
YGGYAGISNSFAVLVKGTIEKGAKKKITNVLEFQGISILDRINYRKDKLNFLLEKGYKDIE
LIIELPKYSLFELSDGSRRMLASILSTNNKRGEIHKGNQIFLSQKFVKLLYHAKRISNTINE
NHRKYVENHKKEFEELFYYILEFNENYVGAKKNGKLLNSAFQSWQNHSIDELCSSFIGP
TGSERKGLFELTSRGSAADFEFLGVKIPRYRDYTPSSLLKDATLIHQSVTGLYETRIDLAK
LGEG

### Deaminase Domains

In some embodiments, the nucleobase editors useful in the present disclosure comprises: (i) a guide nucleotide sequence-programmable DNA-binding protein domain; and (ii) a deaminase domain. In some embodiments, the deaminase domain of the fusion protein is a cytosine deaminase. In some embodiments, the deaminase is an APOBEC1 deaminase. In some embodiments, the deaminase is a rat APOBEC1. In some embodiments, the deaminase is a human APOBEC1. In some embodiments, the deaminase is an APOBEC2 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a Lamprey CDA1 (pmCDA1). In some embodiments, the deaminase is a human APOBEC3G or a functional fragment thereof. In some embodiments, the deaminase is an APOBEC3G variant comprising mutations correspond to the D316R/D317R mutations in the human APOBEC3G. Exemplary, non-limiting cytosine deaminase sequences that may be used in accordance with the methods of the present disclosure are provided in Example 1 below.

In some embodiments, the cytosine deaminase is a wild type deaminase or a deaminase as set forth in SEQ ID NOs: 271-292 and 303. In some embodiments, the cytosine deaminase domains of the fusion proteins provided herein include fragments of deaminases and proteins homologous to a deaminase. For example, in some embodiments, a deaminase domain may comprise a fragment of the amino acid sequence set forth in any of SEQ ID NOs: 271-292 and 303. In some embodiments, a deaminase domain comprises an amino acid sequence homologous to the amino acid sequence set forth in any of SEQ ID NOs: 271-292 and 303 or an amino acid sequence homologous to a fragment of the amino acid sequence set forth in any of SEQ ID NOs: 271-292 and 303. In some embodiments, proteins comprising a deaminase, a fragments of a deaminase, or homologs of a deaminase or a deaminase are referred to as “deaminase variants.” A deaminase variant shares homology to a deaminase, or a fragment thereof. For example a deaminase variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild type deaminase or a deaminase as set forth in any of SEQ ID NOs: 271-292 and 303. In some embodiments, the deaminase variant comprises a fragment of the deaminase, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of wild type deaminase or a deaminase as set forth in any of SEQ ID NOs: 271-292 and 303. In some embodiments, the cytosine deaminase is at least at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to an APOBEC3G variant as set forth in SEQ ID NO: 291 or SEQ ID NO: 292, and comprises mutations corresponding to the D316E/D317R mutations in SEQ ID NO: 290.

In some embodiments, the cytosine deaminase domain is fused to the N-terminus of the guide nucleotide sequence-programmable DNA-binding protein domain. For example, the fusion protein may have an architecture of NH₂-[cytosine deaminase]-[guide nucleotide sequence-programmable DNA-binding protein domain]-COOH. The “]-[” used in the general architecture above indicates the presence of an optional linker sequence. The term “linker,” as used herein, refers to a chemical group or a molecule linking two molecules or moieties, e.g., two domains of a fusion protein, such as, for example, a dCas9 domain and a cytosine deaminase domain. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

In some embodiments, the cytosine deaminase domain and the Cas9 domain are fused to each other via a linker. Various linker lengths and flexibilities between the deaminase domain (e.g., APOBEC1) and the Cas9 domain can be employed (e.g., ranging from very flexible linkers of the form (GGGS)ₙ (SEQ ID NO: 1998), (GGGGS)ₙ (SEQ ID NO: 308), (GGS)ₙ, and (G)ₙ to more rigid linkers of the form (EAAAK)ₙ (SEQ ID NO: 309), SGSETPGTSESATPES (SEQ ID NO: 310) (see, e.g., Guilinger et, al., Nat. Biotechnol. 2014; 32(6): 577-82; the entire contents are incorporated herein by reference), (XP)ₙ, or a combination of any of these, wherein X is any amino acid and n is independently an integer between 1 and 30, in order to achieve the optimal length for deaminase activity for the specific application. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. In some embodiments, the linker comprises a (GGS)ₙ motif, wherein n is 1, 2, 3, 4, 5, 6, 7, 8, 9, 10, 11, 12, 13, 14 or 15. In some embodiments, the linker comprises a (GGS)ₙ motif, wherein n is 1, 3, or 7. In some embodiments, the linker comprises the amino acid sequence SGSETPGTSESATPES (SEQ ID NO: 310), also referred to as the XTEN linker. In some embodiments, the linker comprises an amino acid sequence chosen from the group including, but not limited to, AGVF, GFLG, FK, AL, ALAL, or ALALA. In some embodiments, suitable linker motifs and configurations include those described in Chen et al., Fusion protein linkers: property, design and functionality. Adv Drug Deliv Rev. 2013; 65(10):1357-69, which is incorporated herein by reference. In some embodiments, the linker may comprise any of the following amino acid sequences: VPFLLEPDNINGKTC (SEQ ID NO: 311), GSAGSAAGSGEF (SEQ ID NO: 312), SIVAQLSRPDPA (SEQ ID NO: 313), MKIIEQLPSA (SEQ ID NO: 314), VRHKLKRVGS (SEQ ID NO: 315), GHGTGSTGSGSS (SEQ ID NO: 316), MSRPDPA (SEQ ID NO: 317), GSAGSAAGSGEF (SEQ ID NO: 312), SGSETPGTSESA (SEQ ID NO: 318), SGSETPGTSESATPEGGSGGS (SEQ ID NO: 319), or GGSM (SEQ ID NO: 320). Additional suitable linker sequences will be apparent to those of skill in the art based on the instant disclosure.

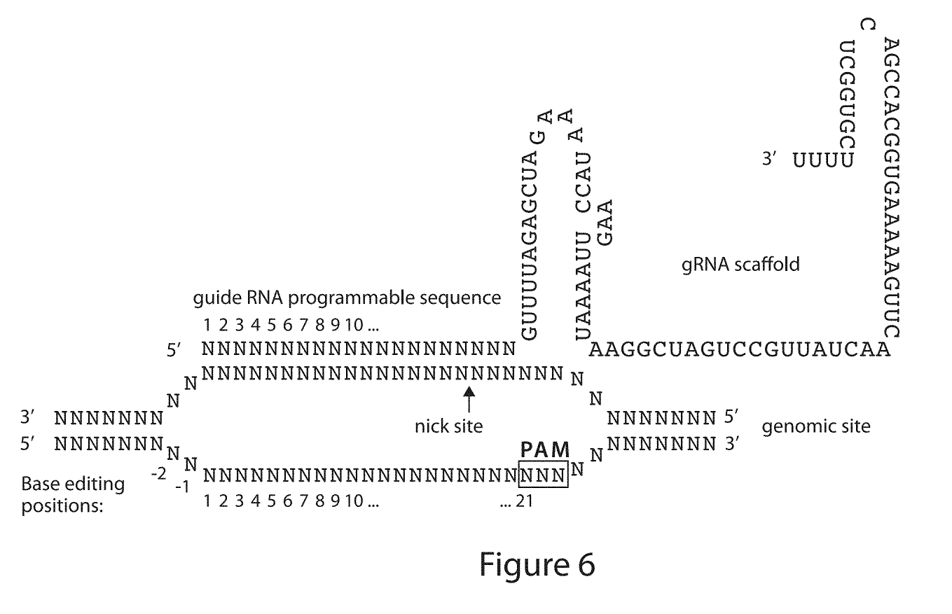

To successfully edit the desired target C base, the linker between Cas9 and APOBEC may be optimized, as described in Komor et al., Nature, 533, 420-424 (2016), which is incorporated herein by reference. The numbering scheme for base editing is based on the predicted location of the target C within the single stranded stretch of DNA (R-loop) displaced by a programmable guide RNA sequence occurring when a DNA-binding domain (e.g. Cas9, nCas9, dCas9) binds a genomic site (see FIG. 6). Conveniently, the sequence immediately surrounding the target C also matches the sequence of the guide RNA. The numbering scheme for base editing is based on a standard 20-mer programmable sequence, and defines position “21” as the first DNA base of the PAM sequence, resulting in position “1” assigned to the first DNA base matching the 5′-end of the 20-mer programmable guide RNA sequence. Therefore, for all Cas9 variants, position “21” is defined as the first base of the PAM sequence (e.g. NGG, NGAN, NGNG, NGAG, NGCG, NNGRRT, NGRRN, NNNRRT, NNNGATT, NNAGAA, NAAAC). When a longer programmable guide RNA sequence is used (e.g. 21-mer) the 5′-end bases are assigned a decreasing negative number starting at “−1”. For other DNA-binding domains that differ in the position of the PAM sequence, or that require no PAM sequence, the programmable guide RNA sequence is used as a reference for numbering. A 3-aa linker gives a 2-5 base editing window (e.g., positions 2, 3, 4, or 5 relative to the PAM sequence at position 21). A 9-aa linker gives a 3-6 base editing window (e.g., positions 3, 4, 5, or 6 relative to the PAM sequence at position 21). A 16-aa linker (e.g., the SGSETPGTSESATPES (SEQ ID NO: 310) linker) gives a 4-7 base editing window (e.g., positions 4, 5, 6, or 7 relative to the PAM sequence at position 21). A 21-aa linker gives a 5-8 base editing window (e.g., positions 5, 6, 7, 8 relative to the PAM sequence at position 21). Each of these windows can be useful for editing different targeted C bases. For example, the targeted C bases may be at different distances from the adjacent PAM sequence, and by varying the linker length, the precise editing of the desired C base is ensured. One skilled in the art, based on the teachings of CRISPR/Cas9 technology, in particular the teachings of U.S. Provisional Application Ser. Nos. 62/245,828, 62/279,346, 62/311,763, 62/322,178, 62/357,352, 62/370,700, and 62/398,490, and in Komor et al., Nature, Programmable editing of a target base in genomic DNA without double-stranded DNA cleavage, 533, 420-424 (2016), each of which is incorporated herein by reference, will be able to determine the window of editing for his/her purpose, and properly design the linker of the cytosine deaminase-dCas9 protein for the precise targeting of the desired C base.

To successfully edit the desired target C base, approporiate Cas9 domain may be selected to attached to the deaminase domain (e.g., APOBEC1), since different Cas9 domains may lead to different editing windows, as described in U.S. Provisional Application Ser. Nos. 62/245,828, 62/279,346, 62/311,763, 62/322,178, 62/357,352, 62/370,700, and 62/398,490, and in Komor et al., Nature, 533, 420-424 (2016), each of which is incorporated herein by reference. For example, APOBEC1-XTEN-SaCas9n-UGI gives a 1-12 base editing window (e.g., positions 1, 2, 3, 4, 5, 6, 7, 8, 9, 10, 11, or 12 relative to the NNNRRT PAM sequence in positions 20-26). One skilled in the art, based on the teachings of CRISPR/Cas9 technology, will be able to determine the editing window for his/her purpose, and properly determine the required Cas9 homolog and linker attached to the cytosine deaminase for the precise targeting of the desired C base.

In some embodiments, the fusion protein useful in the present disclosure further comprises a uracil glycosylase inhibitor (UGI) domain. A “uracil glycosylase inhibitor” refers to a protein that inhibits the activity of uracil-DNA glycosylase. The C to T base change induced by deamination results in a U:G heteroduplex, which triggers cellular DNA-repair response. Uracil DNA glycosylase (UDG) catalyzes removal of U from DNA in cells and initiates base excision repair, with reversion of the U:G pair to a C:G pair as the most common outcome. Thus, such cellular DNA-repair response may be responsible for the decrease in nucleobase editing efficiency in cells. Uracil DNA Glycosylase Inhibitor (UGI) is known in the art to potently blocks human UDG activity. As described in Komor et al., Nature (2016), fusing a UGI domain to the cytidine deaminase-dCas9 fusion protein reduced the activity of UDG and significantly enhanced editing efficiency.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), each of which is incorporated herein by reference. In some embodiments, the UGI comprises the following amino acid sequence: Bacillus phage PBS2 (Bacteriophage PBS2) Uracil-DNA glycosylase inhibitor MTNLSDIIEKETGKQLVIQESILMLPEEVEEVIGNKPESDILVHTAYDESTDENVMLLTSDAPEYKPWALVIQ DSNGENKIKML (SEQ ID NO: 304)

In some embodiments, the UGI protein comprises a wild type UGI or a UGI as set forth in SEQ ID NO: 304. In some embodiments, the UGI proteins useful in the present disclosure include fragments of UGI and proteins homologous to a UGI or a UGI fragment. For example, in some embodiments, a UGI comprises a fragment of the amino acid sequence set forth in SEQ ID NO: 304. In some embodiments, a UGI comprises an amino acid sequence homologous to the amino acid sequence set forth in SEQ ID NO: 304 or an amino acid sequence homologous to a fragment of the amino acid sequence set forth in SEQ ID NO: 304. In some embodiments, proteins comprising UGI or fragments of UGI or homologs of UGI or UGI fragments are referred to as “UGI variants.” A UGI variant shares homology to UGI, or a fragment thereof. For example a UGI variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild type UGI or a UGI as set forth in SEQ ID NO: 304. In some embodiments, the UGI variant comprises a fragment of UGI, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of wild type UGI or a UGI as set forth in SEQ ID NO: 304.

It should be appreciated that additional proteins may be uracil glycosylase inhibitors. For example, other proteins that are capable of inhibiting (e.g., sterically blocking) a uracil-DNA glycosylase base-excision repair enzyme are within the scope of this disclosure. In some embodiments, a uracil glycosylase inhibitor is a protein that binds DNA. In some embodiments, a uracil glycosylase inhibitor is a protein that binds single-stranded DNA. For example, a uracil glycosylase inhibitor may be a Erwinia tasmaniensis single-stranded binding protein. In some embodiments, the single-stranded binding protein comprises the amino acid sequence (SEQ ID NO: 305). In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. For example, a uracil glycosylase inhibitor is a UdgX. In some embodiments, the UdgX comprises the amino acid sequence (SEQ ID NO: 306). As another example, a uracil glycosylase inhibitor is a catalytically inactive UDG. In some embodiments, a catalytically inactive UDG comprises the amino acid sequence (SEQ ID NO: 307). It should be appreciated that other uracil glycosylase inhibitors would be apparent to the skilled artisan and are within the scope of this disclosure. In some embodiments, the fusion protein comprises a guide nucleotide sequence-programmable DNA-binding protein, a cytidine deaminase domain, a Gam protein, and a UGI domain. In some embodiments, any of the fusion proteins provided herein that comprise a guide nucleotide sequence-programmable DNA-binding protein (e.g., a Cas9 domain), a cytidine deaminase, and a Gam protein may be further fused to a UGI domain either directly or via a linker. This disclosure also contemplates a fusion protein comprising a Cas9 nickase-nucleic acid editing domain fused to a cytidine deaminase, and a Gam protein, which is further fused to a UGI domain.

Erwinia tasmaniensis SSB (themostable single-
stranded DNA binding protein)

(SEQ ID NO: 305)

MASRGVNKVILVGNLGQDPEVRYMPNGGAVANITLATSESWRDKQTGETK
EKTEWHRVVLFGKLAEVAGEYLRKGSQVYIEGALQTRKWTDQAGVEKYTT
EVVVNVGGTMQMLGGRSQGGGASAGGQNGGSNNGWGQPQQPQGGNQFSGG
AQQQARPQQQPQQNNAPANNEPPIDFDDDIP
UdgX (binds to Uracil in DNA but does not excise)

(SEQ ID NO: 306)

MAGAQDFVPHTADLAELAAAAGECRGCGLYRDATQAVFGAGGRSARIMMI
GEQPGDKEDLAGLPFVGPAGRLLDRALEAADIDRDALYVTNAVKHFKFTR
AAGGKRRIHKTPSRTEVVACRPWLIAEMTSVEPDVVVLLGATAAKALLGN
DFRVTQHRGEVLHVDDVPGDPALVATVHPSSLLRGPKEERESAFAGLVDD
LRVAADVRP
UDG (catalytically inactive human UDG, binds to
Uracil in DNAbut does not excise)

(SEQ ID NO: 307)

MIGQKTLYSFFSPSPARKRHAPSPEPAVQGTGVAGVPEESGDAAAIPAKK
APAGQEEPGTPPSSPLSAEQLDRIQRNKAAALLRLAARNVPVGFGESWKK
HLSGEFGKPYFIKLMGFVAEERKHYTVYPPPHQVFTWTQMCDIKDVKVVI
LGQEPYHGPNQAHGLCFSVQRPVPPPPSLENIYKELSTDIEDFVHPGHGD
LSGWAKQGVLLLNAVLTVRAHQANSHKERGWEQFTDAVVSWLNQNSNGLV
FLLWGSYAQKKGSAIDRKRHHVLQTAHPSPLSVYRGFFGCRHFSKTNELL
QKSGKKPIDWKEL

In some embodiments, the UGI domain is fused to the C-terminus of the dCas9 domain in the fusion protein. Thus, the fusion protein would have an architecture of NH₂-[cytosine deaminase]-[guide nucleotide sequence-programmable DNA-binding protein domain]-[UGI]-COOH. In some embodiments, the UGI domain is fused to the N-terminus of the cytosine deaminase domain. As such, the fusion protein would have an architecture of NH₂-[UGI]-[cytosine deaminase]-[guide nucleotide sequence-programmable DNA-binding protein domain]-COOH. In some embodiments, the UGI domain is fused between the guide nucleotide sequence-programmable DNA-binding protein domain and the cytosine deaminase domain. As such, the fusion protein would have an architecture of NH₂-[cytosine deaminase]-[UGI]-[guide nucleotide sequence-programmable DNA-binding protein domain]-COOH. The linker sequences described herein may also be used for the fusion of the UGI domain to the cytosine deaminase-dCas9 fusion proteins.

In some embodiments, the fusion protein comprises the structure:

[cytosine deaminase]-[optional linker sequence]-[guide nucleotide sequence-programmable DNA binding protein]-[optional linker sequence]-[UGI];
[cytosine deaminase]-[optional linker sequence]-[UGI]-[optional linker sequence]-[guide nucleotide sequence-programmable DNA binding protein];
[UGI]-[optional linker sequence]-[cytosine deaminase]-[optional linker sequence]-[guide nucleotide sequence-programmable DNA binding protein];
[UGI]-[optional linker sequence]-[guide nucleotide sequence-programmable DNA binding protein]-[optional linker sequence]-[cytosine deaminase];
[guide nucleotide sequence-programmable DNA binding protein]-[optional linker sequence]-[cytosine deaminase]-[optional linker sequence]-[UGI]; or
[guide nucleotide sequence-programmable DNA binding protein]-[optional linker sequence]-[UGI]-[optional linker sequence]-[cytosine deaminase].

In some embodiments, the fusion protein comprises the structure:

[cytosine deaminase]-[optional linker sequence]-[Cas9 nickase]-[optional linker sequence]-[UGI];
[cytosine deaminase]-[optional linker sequence]-[UGI]-[optional linker sequence]-[Cas9 nickase];
[UGI]-[optional linker sequence]-[cytosine deaminase]-[optional linker sequence]-[Cas9 nickase];
[UGI]-[optional linker sequence]-[Cas9 nickase]-[optional linker sequence]-[cytosine deaminase];
[Cas9 nickase]-[optional linker sequence]-[cytosine deaminase]-[optional linker sequence]-[UGI]; or
[Cas9 nickase]-[optional linker sequence]-[UGI]-[optional linker sequence]-[cytosine deaminase].

In some embodiments, fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the UGI protein. In some embodiments, the NLS is fused to the C-terminus of the UGI protein. In some embodiments, the NLS is fused to the N-terminus of the guide nucleotide sequence-programmable DNA-binding protein domain. In some embodiments, the NLS is fused to the C-terminus of the guide nucleotide sequence-programmable DNA-binding protein domain. In some embodiments, the NLS is fused to the N-terminus of the cytosine deaminase. In some embodiments, the NLS is fused to the C-terminus of the deaminase. In some embodiments, the NLS is fused to the fusion protein via one or more linkers. In some embodiments, the NLS is fused to the fusion protein without a linker. Non-limiting, exemplary NLS sequences may be PKKKRKV (SEQ ID NO: 1988) or MDSLLMNRRKFLYQFKNVRWAKGRRETYLC (SEQ ID NO: 1989).

Some aspects of the present disclosure provide nucleobase editors described herein associated with a guide nucleotide sequence (e.g., a guide RNA or gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as a single RNA species comprise two domains: (1) a domain that shares homology to a target nucleic acid (e.g., and directs binding of the Cas9 complex to the target); and (2) a domain that binds the Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof,” and U.S. Provisional Patent Application, U.S. Ser. No. 61/874,746, filed Sep. 6, 2013, entitled “Delivery System For Functional Nucleases,” each are hereby incorporated by reference in their entirety. The gRNA comprises a nucleotide sequence that complements a target site, which mediates binding of the nuclease/RNA complex to said target site, providing the sequence specificity of the nuclease:RNA complex. These proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Science 339, 819-823 (2013); Mali, P. et al. Science 339, 823-826 (2013); Hwang, W. Y. et al. Nature biotechnology 31, 227-229 (2013); Jinek, M. et al. eLife 2, e00471 (2013); Dicarlo, J. E. et al. Nucleic acids research (2013); Jiang, W. et al. Nature biotechnology 31, 233-239 (2013); each of which are incorporated herein by reference). In particular, examples of guide nucleotide sequences (e.g., sgRNAs) that may be used to target the fusion protein of the present disclosure to its target sequence to deaminate the targeted C bases are described in Komor et al., Nature, 533, 420-424 (2016), which is incorporated herein by reference.

The specific structure of the guide nucleotide sequences (e.g., sgRNAs) depends on its target sequence and the relative distance of a PAM sequence downstream of the target sequence. One skilled in the art will understand, that no unifying structure of guide nucleotide sequence is given, for that he target sequences are different for each and every C targeted to be deaminated.

However, the present disclosure provides guidance in how to design the guide nucleotide sequence, e.g., an sgRNA, so that one skilled in the art may use such teaching to a target sequence of interest. An gRNA typically comprises a tracrRNA framework allowing for Cas9 binding, and a guide sequence, which confers sequence specificity to fusion proteins disclosed herein. In some embodiments, the guide RNA comprises a structure 5′-[guide sequence]-tracrRNA-3′. Non-limiting, exemplary tracrRNA sequences are shown in Table 17.

TABLE 17
TracrRNA othologues and sequences

| | | SEQ |
|---|---|---|
| | | ID |
| Organism | tracrRNA sequence | NO |
| S. pyogenes | GUUUAAGAGCUAUGCUGGAAAGCCACGGUGAA | 322 |
| | AAAGUUCAACUAUUGCCUGAUCGGAAUAAAUU | |
| | UGAACGAUACGACAGUCGGUGCUUUUUUU | |
| S. pyogenes | GUUUAAGAGCUAGAAAUAGCAAGUUUAAAUAA | 323 |
| | GGCUAGUCCGUUAUCAACUUGAAAAAGUGGCAC | |
| | CGAGUCGGUGCUUUUUU | |
| S. thermophilus CRISPR1 | GUUUUUGUACUCUCAAGAUUCAAUAAUCUUGC | 324 |
| | AGAAGCUACAAAGAUAAGGCUUCAUGCCGAAA | |
| | UCAACACCCUGUCAUUUUAUGGCAGGGUGUUUU | |
| S. thermophilus CRISPR3 | GUUUUAGAGCUGUGUUGUUUGUUAAAACAACA | 325 |
| | CAGCGAGUUAAAAUAAGGCUUAGUCCGUACUCA | |
| | ACUUGAAAAGGUGGCACCGAUUCGGUGUUUUU | |
| C. jejuni | AAGAAAUUUAAAAAGGGACUAAAAUAAAGAGU | 326 |
| | UUGCGGGACUCUGCGGGGUUACAAUCCCCUAAA | |
| | ACCGCUUUU | |
| F. novicida | AUCUAAAAUUAUAAAUGUACCAAAUAAUUAAU | 327 |
| | GCUCUGUAAUCAUUUAAAAGUAUUUUGAACGG | |
| | ACCUCUGUUUGACACGUCUGAAUAACUAAAA | |
| S. thermophilus2 | UGUAAGGGACGCCUUACACAGUUACUUAAAUCU | 328 |
| | UGCAGAAGCUACAAAGAUAAGGCUUCAUGCCGA | |
| | AAUCAACACCCUGUCAUUUUAUGGCAGGGUGUU | |
| | UUCGUUAUUU | |
| M. mobile | UGUAUUUCGAAAUACAGAUGUACAGUUAAGAA | 329 |
| | UACAUAAGAAUGAUACAUCACUAAAAAAAGGC | |
| | UUUAUGCCGUAACUACUACUUAUUUUCAAAAU | |
| | AAGUAGUUUUUUUU | |
| L. innocua | AUUGUUAGUAUUCAAAAUAACAUAGCAAGUUA | 330 |
| | AAAUAAGGCUUUGUCCGUUAUCAACUUUUAAU | |
| | UAAGUAGCGCUGUUUCGGCGCUUUUUUU | |
| S. pyogenes | GUUGGAACCAUUCAAAACAGCAUAGCAAGUUA | 331 |
| | AAAUAAGGCUAGUCCGUUAUCAACUUGAAAAA | |
| | GUGGCACCGAGUCGGUGCUUUUUUU | |
| S. mutans | GUUGGAAUCAUUCGAAACAACACAGCAAGUUA | 332 |
| | AAAUAAGGCAGUGAUUUUUAAUCCAGUCCGUA | |
| | CACAACUUGAAAAAGUGCGCACCGAUUCGGUGC | |
| | UUUUUUAUUU | |
| S. thermophilus | UUGUGGUUUGAAACCAUUCGAAACAACACAGCG | 333 |
| | AGUUAAAAUAAGGCUUAGUCCGUACUCAACUU | |
| | GAAAAGGUGGCACCGAUUCGGUGUUUUUUUU | |
| N. meningitidis | ACAUAUUGUCGCACUGCGAAAUGAGAACCGUUG | 334 |
| | CUACAAUAAGGCCGUCUGAAAAGAUGUGCCGCA | |
| | ACGCUCUGCCCCUUAAAGCUUCUGCUUUAAGGG | |
| | GCA | |
| P. multocida | GCAUAUUGUUGCACUGCGAAAUGAGAGACGUU | 335 |
| | GCUACAAUAAGGCUUCUGAAAAGAAUGACCGU | |
| | AACGCUCUGCCCCUUGUGAUUCUUAAUUGCAAG | |
| | GGGCAUCGUUUUU | |

The guide sequence of the gRNA comprises a sequence that is complementary to the target sequence. The guide sequence is typically about 20 nucleotides long. For example, the guide sequence may be 15-25 nucleotides long. In some embodiments, the guide sequence is 15, 16, 17, 18, 19, 20, 21, 22, 23, 24, or 25 nucleotides long. In some embodiments, the guide sequence is more than 25 nucleotides long. Such suitable guide RNA sequences typically comprise guide sequences that are complementary to a nucleic sequence within 50 nucleotides upstream or downstream of the target nucleotide to be edited.

In some embodiments, the guide RNA is about 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

To edit the genes in the LDLR mediated cholesterol clearance pathway using the methods described herein, the nucleobase editor and/or the guide nucleotide sequence is introduced into the cell (e.g., a liver cell) where the editing occurs. In some embodiments, nucleic acid molecules (e.g., expression vectors) encoding the nucleobase editors and/or the guide nucleotide sequences are delivered into the cell, resulting in co-expression of nucleobase editors and/or the guide nucleotide sequences in the cell. The nucleic acid molecules encoding the nucleobase editors and/or the guide nucleotide sequences may be delivered into the cell using any known methods in the art, e.g., transfection (e.g., transfection mediated by cationic liposomes), transduction (e.g., via viral infection) and electroporation. In some embodiments, an isolated nucleobase editor/gRNA complex is delivered. Methods of delivering an isolated protein to a cell is familiar to those skilled in the art. For example, the isolated nucleobase editor in complex with a gRNA be associated with a supercharged, cell-penetrating protein or peptide, which facilitates its entry into a cell (e.g., as described in PCT Application Publication WO2010129023 and US Patent Application Publication US20150071906, incorporated herein by reference). In some embodiments, the isolated nucleobase editor incomplex with a gRNA may be delivered by a cationic transfection reagent, e.g., the Lipofectamine CRISPRMAX Cas9 Transfection Reagent from Thermofisher Scientific. In some embodiments, the nucleobase editor and the gRNA may be delivered separately. One skilled in the art is familiar with methods of delivering a nucleic acid molecule or an isolated protein.

### Fusion Proteins Comprising Gam

Some aspects of the disclosure provide fusion proteins comprising a Gam protein. Some aspects of the disclosure provide base editors that further comprise a Gam protein. Base editors are known in the art and have been described previously, for example, in U.S. Patent Application Publication Nos.: US-2015-0166980, published Jun. 18, 2015; US-2015-0166981, published Jun. 18, 2015; US-2015-0166984, published Jun. 18, 2015; US-2015-01669851, published Jun. 18, 2015; US-2016-0304846, published Oct. 20, 2016; US-2017-0121693-A1, published May 4, 2017; and PCT Application publication Nos.: WO 2015/089406, published Jun. 18, 2015; and WO 2017/070632, published Apr. 27, 2017; the entire contents of each of which are hereby incorporated by reference. A skilled artisan would understand, based on the disclosure, how to make and use base editors that further comprise a Gam protein.

In some embodiments, the disclosure provides fusion proteins comprising a guide nucleotide sequence-programmable DNA-binding protein and a Gam protein. In some embodiments, the disclosure provides fusion proteins comprising a cytidine deaminase domain and a Gam protein. In some embodiments, the disclosure provides fusion proteins comprising a UGI domain and a Gam protein. In some embodiments, the disclosure provides fusion proteins comprising a guide nucleotide sequence-programmable DNA-binding protein, a cytidine deaminase domain and a Gam protein. In some embodiments, the disclosure provides fusion proteins comprising a guide nucleotide sequence-programmable DNA-binding protein, a cytidine deaminase domain a Gam protein and a UGI domain.

In some embodiments, the Gam protein is a protein that binds to double strand breaks in DNA and prevents or inhibits degradation of the DNA at the double strand breaks. In some embodiments, the Gam protein is encoded by the bacteriophage Mu, which binds to double stranded breaks in DNA. Without wishing to be bound by any particular theory, Mu transposes itself between bacterial genomes and uses Gam to protect double stranded breaks in the transposition process. Gam can be used to block homologous recombination with sister chromosomes to repair double strand breaks, sometimes leading to cell death. The survival of cells exposed to UV is similar for cells expression Gam and cells where the recB is mutated. This indicates that Gam blocks DNA repair (Cox, 2013). The Gam protein can thus promote Cas9-mediated killing (Cui et al., 2016). GamGFP is used to label double stranded breaks, although this can be difficult in eukaryotic cells as the Gam protein competes with similar eukaryotic protein Ku (Shee et al., 2013).

Gam is related to Ku70 and Ku80, two eukaryotic proteins involved in non-homologous DNA end-joining (Cui et al., 2016). Gam has sequence homology with both subunits of Ku (Ku70 and Ku80), and can have a similar structure to the core DNA-binding region of Ku. Orthologs to Mu Gam are present in the bacterial genomes of Haemophilus influenzae, Salmonella typhi, Neisseria meningitidis and the enterohemorrhagic O157:H7 strain of E. coli (d'Adda di Fagagna et al., 2003). Gam proteins have been described previously, for example, in Cox, Proteins pinpoint double strand breaks. eLife. 2013; 2: e01561.; Cui et al., Consequences of Cas9 cleavage in the chromosome of Escherichia coli. Nucleic Acids Res. 2016 May 19; 44(9):4243-51. doi: 10.1093/nar/gkw223. Epub 2016 Apr. 8.; d'Adda di Fagana et al., The Gam protein of bacteriophage Mu is an orthologue of eukaryotic Ku. EMBO Rep. 2003 January; 4(1):47-52.; and Shee et al., Engineered proteins detect spontaneous DNA breakage in human and bacterial cells. Elife. 2013 Oct. 29; 2:e01222. doi: 10.7554/eLife.01222; the contents of each of which are incorporated herein by reference.

In some embodiments, the Gam protein is a protein that binds double strand breaks in DNA and prevents or inhibits degradation of the DNA at the double strand breaks. In some embodiments, the Gam protein is a naturally occurring Gam protein from any organism (e.g., a bacterium), for example, any of the organisms provided herein. In some embodiments, the Gam protein is a variant of a naturally-occurring Gam protein from an organism. In some embodiments, the Gam protein does not occur in nature. In some embodiments, the Gam protein is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring Gam protein. In some embodiments, the Gam protein is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to any of the Gam proteins provided herein (e.g., SEQ ID NO: 2030). Exemplary Gam proteins are provided below. In some embodiments, the Gam protein comprises the amino acid sequence of any one of SEQ ID NOs: 2030-2058. In some embodiments, the Gam protein is a truncated version of any of the Gam proteins provided herein. In some embodiments, the truncated Gam protein is missing 1, 2, 3, 4, 5, 6, 7, 8, 9, 10, 11, 12, 13, 14, 15, 6, 17, 18, 19, or 20 N-terminal amino acid residues relative to a full-length Gam protein. In some embodiments, the truncated Gam protein may be missing 1, 2, 3, 4, 5, 6, 7, 8, 9, 10, 11, 12, 13, 14, 15, 6, 17, 18, 19, or 20 C-terminal amino acid residues relative to a full-length Gam protein. In some embodiments, the Gam protein does not comprise an N-terminal methionine.

In some embodiments, the Gam protein comprises an amino acid sequence that is at least 60%, 65%, 70%, 75%, 80%, 85%, 90%, 95, 98%, 99%, or 99.5% identical to any of the Gam proteins provided herein. In some embodiments, the Gam protein comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the Gam proteins provided herein. In some embodiments, the Gam protein comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any of the Gam proteins provided herein. In some embodiments, the Gam protein comprises the amino acid sequence of any of the Gam proteins provided herein. In some embodiments, the Gam protein consists of the amino acid sequence of any one of SEQ ID NOs: 2030-2058.

Gam from Bacteriophage Mu

(SEQ ID NO: 2030)

AKPAKRIKSAAAAYVPQNRDAVITDIKRIGDLQREASRLETEMNDAIAEI
TEKFAARIAPIKTDIETLSKGVQGWCEANRDELTNGGKVKTANLVTGDV
SWRVRPPSVSIRGMDAVMETLERLGLQRFIRTKQEINKEAILLEPKAVA
GVAGITVKSGIEDFSIIPFEQEAGI

>WP_001107930.1 MULTISPECIES: host-nuclease inhibitor protein Gam [Enterobacteriaceae]

(SEQ ID NO: 2031)

MAKPAKRIKSAAAAYVPQNRDAVITDIKRIGDLQREASRLETEMNDAIA
EITEKFAARIAPIKTDIETLSKGVQGWCEANRDELTNGGKVKTANLVTG
DVSWRVRPPSVSIRGMDAVMETLERLGLQRFIRTKQEINKEAILLEPKA
VAGVAGITVKSGIEDFSIIPFEQEAGI

>CAA27978.1 unnamed protein product [Escherichia virus Mu]

(SEQ ID NO: 2058)

MAKPAKRIKSAAAAYVPQNRDAVITDIKRIGDLQREASRLETEMNDAIA
EITEKFAARIAPIKTDIETLSKGVQGWCEANRDELTNGGKVKTANLVTG
DVSWRVRPPSVSIRGMDAVMETLERLGLQRFVRTKQEINKEAILLEPKA
VAGVAGITVKSGIEDFSIIPFEQEAGI

>WP_001107932.1 host-nuclease inhibitor protein Gam [Escherichia coli]

(SEQ ID NO: 2032)

MAKPAKRIKSAAAAYVPQNRDAVITDIKRIGDLQREASRLETEMNDAIA
EITEKFAARIAPLKTDIETLSKGVQGWCEANRDELTNGGKVKTANLVTG
DVSWRVRPPSVSIRGMDAVMETLERLGLQRFIRTKQEINKEAILLEPKA
VAGVAGITVKSGIEDFSIIPFEQEAGI

>WP_061335739.1 host-nuclease inhibitor protein Gam [Escherichia coli]

(SEQ ID NO: 2033)

MAKPAKRIKSAAAAYVPQNRDAVITDIKRIGDLQREASRLETEMNDAIA
EITEKFAARIAPIKTDIETLSKGVQGWCEANRDELTNGGKVKTANLITG
DVSWRVRPPSVSIRGMDAVMETLERLGLQRFIRTKQEINKEAILLEPKA
VAGVAGITVKSGIEDFSIIPFEQEAGI

>WP_001107937.1 MULTISPECIES: host-nuclease inhibitor protein Gam [Enterobacteriaceae] >EJL11163.1 bacteriophage Mu Gam like family protein [Shigella sonnei str. Moseley] >CSO81529.1 host-nuclease inhibitor protein [Shigella sonnei] >OCE38605.1 host-nuclease inhibitor protein Gam [Shigella sonnei] >SJK50067.1 host-nuclease inhibitor protein [Shigella sonnei] >SJK19110.1 host-nuclease inhibitor protein [Shigella sonnei] >SIY81859.1 host-nuclease inhibitor protein [Shigella sonnei] >SJJ34359.1 host-nuclease inhibitor protein [Shigella sonnei] >SJK07688.1 host-nuclease inhibitor protein [Shigella sonnei] >SJI95156.1 host-nuclease inhibitor protein [Shigella sonnei] >SIY86865.1 host-nuclease inhibitor protein [Shigella sonnei] >SJJ67303.1 host-nuclease inhibitor protein [Shigella sonnei] >SJJ18596.1 host-nuclease inhibitor protein [Shigella sonnei] >SIX52979.1 host-nuclease inhibitor protein [Shigella sonnei] >SJD05143.1 host-nuclease inhibitor protein [Shigella sonnei] >SJD37118.1 host-nuclease inhibitor protein [Shigella sonnei] >SJE51616.1 host-nuclease inhibitor protein [Shigella sonnei]

(SEQ ID NO: 2034)

MAKPAKRIRNAAAAYVPQSRDAVVCDIRRIGDLQREAARLETEMNDAIA
EITEKYASQIAPLKTSIETLSKGVQGWCEANRDELTNGGKVKTANLVTG
DVSWRQRPPSVSIRGVDAVMETLERLGLQRFIRTKQEINKEAILLEPKA
VAGVAGITVKSGIEDFSIIPFEQEAGI

>WP_001107930.1 MULTISPECIES: host-nuclease inhibitor protein Gam [Enterobacteriaceae]

(SEQ ID NO: 2035)

MAKPAKRIKSAAAAYVPQNRDAVITDIKRIGDLQREASRLETEMNDAIA
EITEKFAARIAPIKTDIETLSKGVQGWCEANRDELTNGGKVKTANLVTG
DVSWRVRPPSVSIRGMDAVMETLERLGLQRFIRTKQEINKEAILLEPKA
VAGVAGITVKSGIEDFSIIPFEQEAGI

>CAA27978.1 unnamed protein product [Escherichia virus Mu]

(SEQ ID NO: 2036)

MAKPAKRIKSAAAAYVPQNRDAVITDIKRIGDLQREASRLETEMNDAIA
EITEKFAARIAPIKTDIETLSKGVQGWCEANRDELTNGGKVKTANLVTG
DVSWRVRPPSVSIRGMDAVMETLERLGLQRFVRTKQEINKEAILLEPKA
VAGVAGITVKSGIEDFSIIPFEQEAGI

>WP_001107932.1 host-nuclease inhibitor protein Gam [Escherichia coli]

(SEQ ID NO: 2037)

MAKPAKRIKSAAAAYVPQNRDAVITDIKRIGDLQREASRLETEMNDAIA
EITEKFAARIAPLKTDIETLSKGVQGWCEANRDELTNGGKVKTANLVTG
DVSWRVRPPSVSIRGMDAVMETLERLGLQRFIRTKQEINKEAILLEPKA
VAGVAGITVKSGIEDFSIIPFEQEAGI

>WP_061335739.1 host-nuclease inhibitor protein Gam [Escherichia coli]

(SEQ ID NO: 2038)

MAKPAKRIKSAAAAYVPQNRDAVITDIKRIGDLQREASRLETEMNDAIA
EITEKFAARIAPIKTDIETLSKGVQGWCEANRDELTNGGKVKTANLITG
DVSWRVRPPSVSIRGMDAVMETLERLGLQRFIRTKQEINKEAILLEPKA
VAGVAGITVKSGIEDFSIIPFEQEAGI

>WP_089552732.1 host-nuclease inhibitor protein Gam [Escherichia coli]

(SEQ ID NO: 2039)

MAKPAKRIKNAAAAYVPQSRDAVVCDIRRIGDLQREAARLETEMNDAIA
EITEKYASQIAPLKTSIETISKGVQGWCEANRDELTNGGKVKTANLVTG
DVSWRQRPPSVSIRGVDAVMETLERLGLQRFIRTKQEINKEAILLEPKA
VAGVAGITVKSGIEDFSIIPFEQEAGI

>WP_042856719.1 host-nuclease inhibitor protein Gam [Escherichia coli] >CDL02915.1 putative host-nuclease inhibitor protein [Escherichia coli IS35]

(SEQ ID NO: 2040)

MAKPAKRIKNAAAAYVPQSRDAVVCDIRRIGDLQREAARLETEMNDAIA
DITEKYASQIAPLKTSIETLSKGVQGWCEANRDELTNGGKVKTANLVTG
DVSWRQRPPSVSIRGVDAVMETLERLGLQRFIRTKQEINKEAILLEPKA
VAGVAGITVKSGIEDFSIIPFEQEAGI

>WP_001129704.1 host-nuclease inhibitor protein Gam [Escherichia coli] >EDU62392.1 bacteriophage Mu Gam like protein [Escherichia coli 53638]

(SEQ ID NO: 2041)

MAKSAKRIRNAAAAYVPQSRDAVVCDIRRIGNLQREAARLETEMNDAIA
EITEKFAARIAPLKTDIETLSKGVQGWCEANRDELTNGGKVKTANLVTG
DVSWRQRPPSVSIRGVDAVMETLERLGLQRFIRTKQEINREAILLEPKA
VAGVAGITVKSGIEDFSIIPFEQDAGI

>WP_001107936.1 MULTISPECIES: host-nuclease inhibitor protein Gam [Enterobacteriaceae] >EGI94970.1 host-nuclease inhibitor protein gam [Shigella boydii 5216-82] >CSR34065.1 host-nuclease inhibitor protein [Shigella sonnei] >CSQ65903.1 host-nuclease inhibitor protein [Shigella sonnei] >CSQ94361.1 host-nuclease inhibitor protein [Shigella sonnei] >SJK23465.1 host-nuclease inhibitor protein [Shigella sonnei] >SJB59111.1 host-nuclease inhibitor protein [Shigella sonnei] >SJI55768.1 host-nuclease inhibitor protein [Shigella sonnei] >SJI56601.1 host-nuclease inhibitor protein [Shigella sonnei] >SJJ20109.1 host-nuclease inhibitor protein [Shigella sonnei] >SJJ54643.1 host-nuclease inhibitor protein [Shigella sonnei] >SJI29650.1 host-nuclease inhibitor protein [Shigella sonnei] >SIZ53226.1 host-nuclease inhibitor protein [Shigella sonnei] >SJA65714.1 host-nuclease inhibitor protein [Shigella sonnei] >SJJ21793.1 host-nuclease inhibitor protein [Shigella sonnei] >SJD61405.1 host-nuclease inhibitor protein [Shigella sonnei] >SJJ14326.1 host-nuclease inhibitor protein [Shigella sonnei] >SIZ57861.1 host-nuclease inhibitor protein [Shigella sonnei] >SJD58744.1 host-nuclease inhibitor protein [Shigella sonnei] >SJD84738.1 host-nuclease inhibitor protein [Shigella sonnei] >SJJ51125.1 host-nuclease inhibitor protein [Shigella sonnei] >SJD01353.1 host-nuclease inhibitor protein [Shigella sonnei] >SJE63176.1 host-nuclease inhibitor protein [Shigella sonnei]

(SEQ ID NO: 2042)

MAKPAKRIRNAAAAYVPQSRDAVVCDIRRIGDLQREAARLETEMNDAIA
EITEKYASQIAPLKTSIETLSKGVQGWCEANRDELTNGGKVKTANLVTG
DVSWRQRPPSVSIRGVDAVMETLERLGLQRFIRTKQEINKEAILLEPKA
VAGVAGITVKSGIEDFSIIPFEQDAGI

>WP_050939550.1 host-nuclease inhibitor protein Gam [Escherichia coli] >KNF77791.1 host-nuclease inhibitor protein Gam [Escherichia coli]

(SEQ ID NO: 2043)

MAKPAKRIKNAAAAYVPQSRDAVVCDIRRIGDLQREAARLETEMNDAIA
EITEKYASQIAPLKTSIETLSKGVQGWCEANRDELTNGGKVKTANLVTG
DVSWRLRPPSVSIRGVDAVMETLERLGLQRFICTKQEINKEAILLEPKV
VAGVAGITVKSGIEDFSIIPFEQEAGI

>WP_085334715.1 host-nuclease inhibitor protein Gam [Escherichia coli] >OSC16757.1 host-nuclease inhibitor protein Gam [Escherichia coli]

(SEQ ID NO: 2044)

MAKPVKRIRNAAAAYVPQSRDAVVCDIRRIGDLQREAARLETEMNDAIAE
ITEKYASQIAPLKTSIETLSKGIQGWCEANRDELTNGGKVKTANLVTGDV
SWRQRPPSVSIRGVDAVMETLERLGLQRFIRTKQEINKEAILLEPKAVAG
VAGITVKSGIEDFSIIPFEQEAGI

>WP_065226797.1 host-nuclease inhibitor protein Gam [Escherichia coli] >ANO88858.1 host-nuclease inhibitor protein Gam [Escherichia coli] >ANO89006.1 host-nuclease inhibitor protein Gam [Escherichia coli]

(SEQ ID NO: 2045)

MAKPAKRIRNAAAAYVPQSRDAVVCDIRWIGDLQREAVRLETEMNDAIAE
ITEKYASRIAPLKTRIETLSKGVQGWCEANRDELTNGGKVKTANLVTGDV
SWRQRPPSVSIRGVDAVMETLERLGLQRFIRTKQEINKEAILLEPKAVAG
VAGITVKSGIEDFSIIPFEQEAGI

>WP_032239699.1 host-nuclease inhibitor protein Gam [Escherichia coli] >KDU26235.1 bacteriophage Mu Gam like family protein [Escherichia coli 3-373-03_S4_C2] >KDU49057.1 bacteriophage Mu Gam like family protein [Escherichia coli 3-373-03_S4_C1] >KEL21581.1 bacteriophage Mu Gam like family protein [Escherichia coli 3-373-03_S4_C3]

(SEQ ID NO: 2046)

MAKSAKRIRNAAATYVPQSRDAVVCDIRRIGDLQREAARLETEMNDAIAE
ITEKYASQIAPLKTSIETLSKGIQGWCEANRDELTNGGKVKTANLVTGDV
SWRQRPPSVSIRGVDAVMETLERLGLQRFIRTKQEINKEAILLEPKAVAG
VAGITVKSGIEDFSIIPFEQEAGI

>WP_080172138.1 host-nuclease inhibitor protein Gam [Salmonella enterica]

(SEQ ID NO: 2047)

MAKSAKRIKSAAATYVPQSRDAVVCDIRRIGDLQREAARLETEMNDAIAE
ITEKYASQIAPLKTSIETLSKGVQGWCEANRDELTNGGKVKSANLVTGDV
QWRQRPPSVSIRGVDAVMETLERLGLQRFIRTKQEINKEAILLEPKAVAG
VAGITVKSGIEDFSIIPFEQEAGI

>WP_077134654.1 host-nuclease inhibitor protein Gam [Shigella sonnei] >SIZ51898.1 host-nuclease inhibitor protein [Shigella sonnei] >SJK07212.1 host-nuclease inhibitor protein [Shigella sonnei]

(SEQ ID NO: 2048)

MAKSAKRIRNAAAAYVPQSRDAVVCDIRRIGNLQREAARLETEMNDAIAE
ITEKYASQIAPLKTSIETLSKGVQGWCEANRDELTNGGKVKTANLVTGDV
SWRQRPPSVSIRGVDAVMETLERLGLQRFIRTKQEINKEAILLEPKAVAG
VAGITVKSGIEDFSIIPFEQDAGI

>WP_000261565.1 host-nuclease inhibitor protein Gam [Shigella flexneri] >EGK20651.1 host-nuclease inhibitor protein gam [Shigella flexneri K-272] >EGK34753.1 host-nuclease inhibitor protein gam [Shigella flexneri K-227]

(SEQ ID NO: 2049)

MVVSAIASTPHDAVVCDIRRIGDLQREAARLETEMNDAIAEITEKDASQI
APLKTSIETLSKGVQGWCEANRDELTNGGKVKTANLVTGDVSWRQRPPSV
SIRGVDAVMETLERLGLQRFIRTKQEINKEAILLEPKAVAGVAGITVKSG
IEDFSIIPFEQEAGI

>ASG63807.1 host-nuclease inhibitor protein Gam [Kluyvera georgiana]

(SEQ ID NO: 2050)

MVSKPKRIKAAAANYVSQSRDAVITDIRKIGDLQREATRLESAMNDEIAV
ITEKYAGLIKPLKADVEMLSKGVQGWCEANRDDLTSNGKVKTANLVTGDI
QWRIRPPSVSVRGPDAVMETLTRLGLSRFIRTKQEINKEAILNEPLAVAG
VAGITVKSGIEDFSIIPFEQTADI

>WP_078000363.1 host-nuclease inhibitor protein Gam [Edwardsiella tarda]

(SEQ ID NO: 2051)

MASKPKRIKSAAANYVSQSRDAVIIDIRKIGDLQREATRLESAMNDEIAV
ITEKYAGLIKPLKADVEMLSKGVQGWCEANRDELTCNGKVKTANLVTGDI
QWRIRPPSVSVRGPDSVMETLLRLGLSRFIRTKQEINKEAILNEPLAVAG
VAGITVKTGVEDFSIIPFEQTADI

>WP_047389411.1 host-nuclease inhibitor protein Gam [Citrobacter freundii] >KGY86764.1 host-nuclease inhibitor protein Gam [Citrobacter freundii] >OIZ37450.1 host-nuclease inhibitor protein Gam [Citrobacter freundii]

(SEQ ID NO: 2052)

MVSKPKRIKAAAANYVSQSKEAVIADIRKIGDLQREATRLESAMNDEIAV
ITEKYAGLIKPLKTDVEILSKGVQGWCEANRDELTSNGKVKTANLVTGDI
QWRIRPPSVAVRGPDAVMETLLRLGLSRFIRTKQEINKEAILNEPLAVAG
VAGITVKSGVEDFSIIPFEQTADI

>WP_058215121.1 host-nuclease inhibitor protein Gam [Salmonella enterica] >KSU39322.1 host-nuclease inhibitor protein Gam [Salmonella enterica subsp. enterica] >OHJ24376.1 host-nuclease inhibitor protein Gam [Salmonella enterica] >ASG15950.1 host-nuclease inhibitor protein Gam [Salmonella enterica subsp. enterica serovar Macclesfield str. S-1643]

(SEQ ID NO: 2053)

MASKPKRIKAAAALYVSQSREDVVRDIRMIGDFQREIVRLETEMNDQIAA
VTLKYADKIKPLQEQLKTLSEGVQNWCEANRSDLTNGGKVKTANLVTGDV
QWRVRPPSVTVRGVDSVMETLRRLGLSRFIRIKEEINKEAILNEPGAVAG
VAGITVKSGVEDFSIIPFEQSATN

>WP_016533308.1 phage host-nuclease inhibitor protein Gam [Pasteurella multocida] >EPE65165.1 phage host-nuclease inhibitor protein Gam [Pasteurella multocida P1933] >ESQ71800.1 host-nuclease inhibitor protein Gam [Pasteurella multocida subsp. multocida P1062] >ODS44103.1 host-nuclease inhibitor protein Gam [Pasteurella multocida] >OPC87246.1 host-nuclease inhibitor protein Gam [Pasteurella multocida subsp. multocida] >OPC98402.1 host-nuclease inhibitor protein Gam [Pasteurella multocida subsp. multocida]

(SEQ ID NO: 2054)

MAKKATRIKTTAQVYVPQSREDVASDIKTIGDLNREITRLETEMNDKIAE
ITESYKGQFSPIQERIKNLSTGVQFWAEANRDQITNGGKTKTANLITGEV
SWRVRNPSVKITGVDSVLQNLKIHGLTKFIRVKEEINKEAILNEKHEVAG
IAGIKVVSGVEDFVITPFEQEI

>WP_005577487.1 host-nuclease inhibitor protein Gam [Aggregatibacter actinomycetemcomitans] >EHK90561.1 phage host-nuclease inhibitor protein Gam [Aggregatibacter actinomycetemcomitans RhAA1] >KNE77613.1 host-nuclease inhibitor protein Gam [Aggregatibacter actinomycetemcomitans RhAA1]

(SEQ ID NO: 2055)

MAKSATRVKATAQIYVPQTREDAAGDIKTIGDLNREVARLEAEMNDKIAA
ITEDYKDKFAPLQERIKTLSNGVQYWSEANRDQITNGGKTKTANLVTGEV
SWRVRNPSVKVTGVDSVLQNLRIHGLERFIRTKEEINKEAILNEKSAVAG
IAGIKVITGVEDFVITPFEQEAA

>WP_090412521.1 host-nuclease inhibitor protein Gam [Nitrosomonas halophila] >SDX89267.1 Mu-like prophage host-nuclease inhibitor protein Gam [Nitrosomonas halophila]

(SEQ ID NO: 2056)

MARNAARLKTKSIAYVPQSRDDAAADIRKIGDLQRQLTRTSTEMNDAIAA
ITQNFQPRMDAIKEQINLLQAGVQGYCEAHRHALTDNGRVKTANLITGEV
QWRQRPPSVSIRGQQVVLETLRRLGLERFIRTKEEVNKEAILNEPDEVRG
VAGLNVITGVEDFVITPFEQEQP

>WP_077926574.1 host-nuclease inhibitor protein Gam [Wohlfahrtiimonas larvae]

(SEQ ID NO: 2057)

MAKKRIKAAATVYVPQSKEEVQNDIREIGDISRKNERLETEMNDRIAEIT
NEYAPKFEVNKVRLELLTKGVQSWCEANRDDLTNSGKVKSANLVTGKVEW
RQRPPSISVKGMDAVIEWLQDSKYQRFLRTKVEVNKEAMLNEPEDAKTIP
GITIKSGIEDFAITPFEQEAGV

### Compositions

Aspects of the present disclosure relate to compositions that may be used for editing PCSK9-encoding polynucleotides. In some embodiments, the editing is carried out in vitro. In some embodiments, the editing is carried out in cultured cell. In some embodiments, the editing is carried out in vivo. In some embodiments, the editing is carried out in a mammal. In some embodiments, the mammal is a human. In some embodiments, the mammal may be a rodent. In some embodiments, the editing is carried out ex vivo.

In some embodiments, the composition comprises: (i) a fusion protein comprising: (a) a guide nucleotide sequence-programmable DNA binding protein domain; and (b) a cytosine deaminase domain; and (ii) a guide nucleotide sequence targeting the fusion protein of (i) to a polynucleotide encoding a Proprotein Convertase subtilisin/Kexin Type 9 (PCSK9) protein. In some embodiments, the fusion protein of (i) further comprises a Gam protein.

In some embodiments, the composition comprises: (i) a fusion protein comprising: (a) a guide nucleotide sequence-programmable DNA binding protein domain; and (b) a cytosine deaminase domain; (ii) a guide nucleotide sequence targeting the fusion protein of (i) to a polynucleotide encoding a Proprotein Convertase subtilisin/Kexin Type 9 (PCSK9) protein; and (ii) a guide nucleotide sequence targeting the fusion protein of (i) to a polynucleotide encoding an Apolipoprotein C3 protein. In some embodiments, the fusion protein of (i) further comprises a Gam protein.

In some embodiments, the composition comprises: (i) a fusion protein comprising: (a) a guide nucleotide sequence-programmable DNA binding protein domain; and (b) a cytosine deaminase domain; (ii) a guide nucleotide sequence targeting the fusion protein of (i) to a nucleic acid molecule polynucleotide encoding a Proprotein Convertase subtilisin/Kexin Type 9 (PCSK9) protein; (iii) a guide nucleotide sequence targeting the fusion protein of (i) to a polynucleotide encoding an Apolipoprotein C3 protein; and (iv) a guide nucleotide sequence targeting the fusion protein of (i) to a nucleic acid molecule polynucleotide encoding Low-Density Lipoprotein Receptor protein. In some embodiments, the fusion protein of (i) further comprises a Gam protein.

In some embodiments, the composition comprises: (i) a fusion protein comprising (a) a guide nucleotide sequence-programmable DNA binding protein domain; and (b) a cytosine deaminase domain; (ii) a guide nucleotide sequence targeting the fusion protein of (i) to a polynucleotide encoding a Proprotein Convertase subtilisin/Kexin Type 9 (PCSK9) protein; (iii) a guide nucleotide sequence targeting the fusion protein of (i) to a nucleic acid molecule polynucleotide encoding an Apolipoprotein C3 protein; (iv) a guide nucleotide sequence targeting the fusion protein of (i) to a polynucleotide encoding Low-Density Lipoprotein Receptor protein; and (v) a guide nucleotide sequence targeting the fusion protein of (i) to a polynucleotide encoding Inducible Degrader of the LDL receptor protein. In some embodiments, the fusion protein of (i) further comprises a Gam protein.

The guide nucleotide sequence used in the compositions described herein for editing the PCSK9-encoding polynucleotide is selected from SEQ ID NOs: 336-1309. The guide nucleotide sequence used in the compositions described herein for editing the APOC3-encoding polynucleotide is selected from SEQ ID NOs: 1806-1906. The guide nucleotide sequence used in the compositions described herein for editing the LDLR-encoding polynucleotide is selected from SEQ ID NOs: 1792-1799. The guide nucleotide sequence used in the compositions described herein for editing the IDOL-encoding polynucleotide is selected from SEQ ID NOs: 1788-1791. In some embodiments, the composition comprises a nucleic acid encoding a fusion protein described in and a guide nucleotide sequence described herein. In some embodiments, the composition described herein further comprises a pharmaceutically acceptable carrier. In some embodiments, the nucleobase editor (i.e., the fusion protein) and the gRNA are provided in two different compositions.

As used here, the term “pharmaceutically acceptable carrier” means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the nucleobase editors and the guide nucleotides of the present disclosure in a composition is administered by injection, by means of a catheter, by means of a suppository, or by means of an implant, the implant being of a porous, non-porous, or gelatinous material, including a membrane, such as a sialastic membrane, or a fiber. In some embodiments, the injection is directed to the liver.

In other embodiments, the nucleobase editors and the guide nucleotides are delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In typical embodiments, the pharmaceutical composition is formulated in accordance with routine procedures as a pharmaceutical composition adapted for intravenous or subcutaneous administration to a subject, e.g., a human. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757.

The pharmaceutical compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

In some embodiments, the nucleobase editors or the guide nucleotides described herein may be conjugated to a therapeutic moiety, e.g., an anti-inflammatory agent. Techniques for conjugating such therapeutic moieties to polypeptides, including e.g., Fc domains, are well known; see, e.g., Amon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al. (1982) “The Preparation And Cytotoxic Properties Of Antibody-Toxin Conjugates,” Immunol. Rev., 62:119-158.

Further, the compositions of the present disclosure may be assembled into kits. In some embodiments, the kit comprises nucleic acid vectors for the expression of the nucleobase editors described herein. In some embodiments, the kit further comprises appropriate guide nucleotide sequences (e.g., gRNAs) or nucleic acid vectors for the expression of such guide nucleotide sequences, to target the nucleobase editors to the desired target sequences.

The kit described herein may include one or more containers housing components for performing the methods described herein and optionally instructions of uses. Any of the kit described herein may further comprise components needed for performing the assay methods. Each component of the kits, where applicable, may be provided in liquid form (e.g., in solution), or in solid form, (e.g., a dry powder). In certain cases, some of the components may be reconstitutable or otherwise processible (e.g., to an active form), for example, by the addition of a suitable solvent or other species (for example, water or certain organic solvents), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

### Therapeutics

The compositions described herein, may be administered to a subject in need thereof, in a therapeutically effective amount, to treat conditions related to high circulating cholesterol levels. Conditions related to high circulating cholesterol level that may be treated using the compositions and methods described herein include, without limitation: hypercholesterolemia, elevated total cholesterol levels, elevated low-density lipoprotein (LDL) levels, elevated LDL-cholesterol levels, reduced high-density lipoprotein levels, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and combinations thereof. The compositions and kits are effective in reducing the circulating cholesterol level in the subject, thus treating the conditions.

“A therapeutically effective amount” as used herein refers to the amount of each therapeutic agent of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system.

Frequency of administration may be determined and adjusted over the course of therapy, and is generally, but not necessarily, based on treatment and/or suppression and/or amelioration and/or delay of a disease. Alternatively, sustained continuous release formulations of a polypeptide or a polynucleotide may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The dosing regimen (including the polypeptide used) can vary over time. In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the polypeptide or the polynucleotide (such as the half-life of the polypeptide or the polynucleotide, and other considerations well known in the art).

For the purpose of the present disclosure, the appropriate dosage of a therapeutic agent as described herein will depend on the specific agent (or compositions thereof) employed, the formulation and route of administration, the type and severity of the disease, whether the polypeptide or the polynucleotide is administered for preventive or therapeutic purposes, previous therapy, the subject's clinical history and response to the antagonist, and the discretion of the attending physician. Typically the clinician will administer a polypeptide until a dosage is reached that achieves the desired result.

Administration of one or more polypeptides or polynucleotides can be continuous or intermittent, depending, for example, upon the recipient's physiological condition, whether the purpose of the administration is therapeutic or prophylactic, and other factors known to skilled practitioners. The administration of a polypeptide may be essentially continuous over a preselected period of time or may be in a series of spaced dose, e.g., either before, during, or after developing a disease. As used herein, the term “treating” refers to the application or administration of a polypeptide or a polynucleotide or composition including the polypeptide or the polynucleotide to a subject in need thereof.

“A subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject has hypercholesterolemia. In some embodiments, the subject is a mammal. In some embodiments, the subject is a non-human primate. In some embodiments, the subject is human. Alleviating a disease includes delaying the development or progression of the disease, or reducing disease severity. Alleviating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

### Host Cells and Organisms

Other aspects of the present disclosure provide host cells and organisms for the production and/or isolation of the nucleobase editors, e.g., for in vitro editing. Host cells are genetically engineered to express the nucleobase editors and components of the translation system described herein. In some embodiments, host cells comprise vectors encoding the nucleobase editors and components of the translation system (e.g., transformed, transduced, or transfected), which can be, for example, a cloning vector or an expression vector. The vector can be, for example, in the form of a plasmid, a bacterium, a virus, a naked polynucleotide, or a conjugated polynucleotide. The vectors are introduced into cells and/or microorganisms by standard methods including electroporation, infection by viral vectors, high velocity ballistic penetration by small particles with the nucleic acid either within the matrix of small beads or particles, or on the surface (Klein et al., Nature 327, 70-73 (1987)). In some embodiments, the host cell is a prokaryotic cell. In some embodiments, the host cell is a eukaryotic cell. In some embodiments, the host cell is a bacterial cell. In some embodiments, the host cell is a yeast cell. In some embodiments, the host cell is a mammalian cell. In some embodiments, the host cell is a human cell. In some embodiments, the host cell is a cultured cell. In some embodiments, the host cell is within a tissue or an organism.

The engineered host cells can be cultured in conventional nutrient media modified as appropriate for such activities as, for example, screening steps, activating promoters or selecting transformants. These cells can optionally be cultured into transgenic organisms.

Several well-known methods of introducing target nucleic acids into bacterial cells are available, any of which can be used in the present disclosure. These include: fusion of the recipient cells with bacterial protoplasts containing the DNA, electroporation, projectile bombardment, and infection with viral vectors (discussed further, below), etc. Bacterial cells can be used to amplify the number of plasmids containing DNA constructs of the present disclosure. The bacteria are grown to log phase and the plasmids within the bacteria can be isolated by a variety of methods known in the art (see, for instance, Sambrook). In addition, a plethora of kits are commercially available for the purification of plasmids from bacteria, (see, e.g., EasyPrep™ FlexiPrep™, both from Pharmacia Biotech; StrataClean™, from Stratagene; and, QIAprep™ from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); and Schneider, B., et al., Protein Expr. Purifi 6435:10 (1995)).

Bacteriophages useful for cloning is provided, e.g., by the ATCC, e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA Second Edition Scientific American Books, NY.

Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell. Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla. In addition, essentially any nucleic acid (and virtually any labeled nucleic acid, whether standard or non-standard) can be custom or standard ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.), and many others.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The synthetic examples described in this application are offered to illustrate the compounds and methods provided herein and are not to be construed in any way as limiting their scope.

### Example 1: Guide Nucleotide Sequence Programmable DNA-Binding Protein Domains, Deaminases, and Base Editors

Non-limiting examples of suitable guide nucleotide sequence-programmable DNA-binding protein domain s are provided. The disclosure provides Cas9 variants, for example, Cas9 proteins from one or more organisms, which may comprise one or more mutations (e.g., to generate dCas9 or Cas9 nickase). In some embodiments, one or more of the amino acid residues, identified below by an asterek, of a Cas9 protein may be mutated. In some embodiments, the D10 and/or H840 residues of the amino acid sequence provided in SEQ ID NO: 1, or a corresponding mutation in any of the amino acid sequences provided in SEQ ID NOs: 11-260, are mutated. In some embodiments, the D10 residue of the amino acid sequence provided in SEQ ID NO: 1, or a corresponding mutation in any of the amino acid sequences provided in SEQ ID NOs: 11-260, is mutated to any amino acid residue, except for D. In some embodiments, the D10 residue of the amino acid sequence provided in SEQ ID NO: 1, or a corresponding mutation in any of the amino acid sequences provided in SEQ ID NOs: 11-260, is mutated to an A. In some embodiments, the H840 residue of the amino acid sequence provided in SEQ ID NO: 1, or a corresponding residue in any of the amino acid sequences provided in SEQ ID NOs: 11-260, is an H. In some embodiments, the H840 residue of the amino acid sequence provided in SEQ ID NO: 1, or a corresponding mutation in any of the amino acid sequences provided in SEQ ID NOs: 11-260, is mutated to any amino acid residue, except for H. In some embodiments, the H840 residue of the amino acid sequence provided in SEQ ID NO: 1, or a corresponding mutation in any of the amino acid sequences provided in SEQ ID NOs: 11-260, is mutated to an A. In some embodiments, the D10 residue of the amino acid sequence provided in SEQ ID NO: 1, or a corresponding residue in any of the amino acid sequences provided in SEQ ID NOs: 11-260, is a D.

A number of Cas9 sequences from various species were aligned to determine whether corresponding homologous amino acid residues of D10 and H840 of SEQ ID NO: 1 or SEQ ID NO: 11 can be identified in other Cas9 proteins, allowing the generation of Cas9 variants with corresponding mutations of the homologous amino acid residues. The alignment was carried out using the NCBI Constraint-based Multiple Alignment Tool (COBALT (accessible at st-va.ncbi.nlm.nih.gov/tools/cobalt), with the following parameters. Alignment parameters: Gap penalties −11, −1; End-Gap penalties −5, −1. CDD Parameters: Use RPS BLAST on; Blast E-value 0.003; Find Conserved columns and Recompute on. Query Clustering Parameters: Use query clusters on; Word Size 4; Max cluster distance 0.8; Alphabet Regular.

An exemplary alignment of four Cas9 sequences is provided below. The Cas9 sequences in the alignment are: Sequence 1 (S1): SEQ ID NO: 11|WP_010922251|gi 499224711|type II CRISPR RNA-guided endonuclease Cas9 [Streptococcus pyogenes]; Sequence 2 (S2): SEQ ID NO: 12|WP_039695303|gi 746743737|type II CRISPR RNA-guided endonuclease Cas9 [Streptococcus gallolyticus]; Sequence 3 (S3): SEQ ID NO: 13|WP_045635197|gi 782887988|type II CRISPR RNA-guided endonuclease Cas9 [Streptococcus mitis]; Sequence 4 (S4): SEQ ID NO: 14|5AXW_A|gi 924443546|Staphylococcus Aureus Cas9. The HNH domain (bold and underlined) and the RuvC domain (boxed) are identified for each of the four sequences. Amino acid residues 10 and 840 in S1 and the homologous amino acids in the aligned sequences are identified with an asterisk following the respective amino acid residue.

| | | | |
|---|---|---|---|
| S1 | 1 | --MDKK-YSIGLD*IGTNSVGWAVITDEYKVPSKKFKVLGNTDRHSIKKNLI--GALLFDSG--ETAEATRLKRTARRRYT | 73 |
| S2 | 1 | --MTKKNYSIGLD*IGTNSVGWAVITDDYKVPAKKMKVLGNTDKKYIKKNLL--GALLFDSG--ETAEATRLKRTARRRYT | 74 |
| S3 | 1 | --M-KKGYSIGLD*IGTNSVGFAVITDDYKVPSKEMKVLGNTDKRFIKKNLI--GALLFDEG--TTAEARRLKRTARRRYT | 73 |
| S4 | 1 | GSHMKRNYILGLD*IGITSVGYGII--DYET-----------------RDVIDAGVRIFKEANVENNEGRRSKRGARRLKR | 61 |
| S1 | 74 | RRKNRICYLQEIFSNEMAKVDDSFFHRLEESFLVEEDKKHERHPIFGNIVDEVAYHEKYPTIYHLRKKLVDSTDKADLRL | 153 |
| S2 | 75 | RRKNRLRYLQEIFANEIAKVDESFFQRLDESFLTDDDKTEDSHPIFGNKAEEDAYHQKFPTIYHLRKHLADSSEKADLRL | 154 |
| S3 | 74 | RRKNRLRYLQEIFSEEMSKVDSSFFHRLDDSFLIPEDKRESKYPIFATLTEEKEYHKQFPTIYHLRKQLADSKEKTDLRL | 153 |
| S4 | 62 | RRRHRIQRVKKLL--------------FDYNLLTD--------------------HSELSGINPYEARVKGLSQKLSEEE | 107 |
| S1 | 154 | IYLALAHMIKERGHFLIEGDLNPDNSDVDKLFIQLVQTYNQLFEENPINASGVDAKAILSARLSKSRRLENLIAQLPGEK | 233 |
| S2 | 155 | VYLALAHMIKFRGHFLIEGELNAENTDVQKIFADFVGVYNRTFDDSHLSEITVDVASILTEKISKSRRLENLIKYYPTEK | 234 |
| S3 | 154 | IYLALAHMIKYRGHFLYEEAFDIKNNDIQKIFNEFISIYDNTFEGSSLSGQNAQVEAIFTDKISKSAKRERVLKLEPDEK | 233 |
| S4 | 108 | FSAALLHLAKRRG----------------------VHNVNEVEEDT---------------------------------- | 131 |
| S1 | 234 | KNGLFGNLIALSLGLTPNEKSNFDLAEDAKLQLSKDTYDDDLDNLLAQIGDQYADLFLAAKNLSDAILLSDILRVNTEIT | 313 |
| S2 | 235 | KNTLFGNLIALALGLQPNEKTNFKLSEDAKLQFSKDTYEEDLEELLGKIGDDYADLFTSAKNLYDAILLSGILTVDDNST | 314 |
| S3 | 234 | STGLFSEFLKLIVGNQADFKKHFDLEDKAPLQFSKDTYDEDLENLLGQIGDDFTDLFVSAKKLYDAILLSGILTVTDPST | 313 |
| S4 | 132 | -----GNELS------------------TKEQISRN-------------------------------------------- | 144 |
| S1 | 314 | KAPLSASMIKRYDEHHQDLTLLKALVRQQLPEKYKEIFFDQSKNGYAGYIDGGASQEEFYKFIKPILEKM--DGTEELLV | 391 |
| S2 | 315 | KAPLSASMIKRYVEHHEDLEKLKEFIKANKSELYHDIFKDKNKNGYAGYIENGVKQDEFYKYLKNILSKIKIDGSDYFLD | 394 |
| S3 | 314 | KAPLSASMIERYENHQNDLAALKQFIKNNLPEKYDEVFSDQSKDGYAGYIDGKTTQETFYKYIKNLLSKF--EGTDYFLD | 391 |
| S4 | 145 | ----SKALEEKYVAELQ-------------------------------------------------LERLKKDG------ | 165 |
| S1 | 392 | KLNREDLLRKQRTFDNGSIPHQIHLGELHAILRRQEDFYPFLKDNREKIEKILTFRIPYYVGPLARGNSRFAWMTRKSEE | 471 |
| S2 | 395 | KIEREDFLRKQRTFDNGSIPHQIHLQEMHAILRRQGDYYPFLKEKQDRIEKILTFRIPYYVGPLVRKDSRFAWAEYRSDE | 474 |
| S3 | 392 | KIEREDFLRKQRTFDNGSIPHQIHLQEMNAILRRQGEYYPFLKDNKEKIEKILTFRIPYYVGPLARGNRDFAWLTRNSDE | 471 |
| S4 | 166 | --EVRGSINRFKTSD--------YVKEAKQLLKVQKAYHQLDQSFIDTYIDLLETRRTYYEGP--GEGSPFGW------K | 227 |
| S1 | 472 | TITPWNFEEVVDKGASAQSFIERMTNFDKNLPNEKVLPKHSLLYEYFTVYNELTKVKYVTEGMRKPAFLSGEQKKAIVDL | 551 |
| S2 | 475 | KITPWNFDKVIDKEKSAEKFITRMTLNDLYLPEEKVLPKHSHVYETYAVYNELTKIKYVNEQGKE-SFFDSNMKQEIFDH | 553 |
| S3 | 472 | AIRPWNFEEIVDKASSAEDFINKMTNYDLYLPEEKVLPKHSLLYETFAVYNELTKVKFIAEGLRDYQFLDSGQKKQIVNQ | 551 |
| S4 | 228 | DIKEW---------------YEMLMGHCTYFPEELRSVKYAYNADLYNALNDLNNLVITRDENEK---LEYYEKFQIIEN | 289 |
| S1 | 552 | LEKTNRKVTVKQLKEDYFKKIECFDSVEISGVEDR---FNASLGTYHDLLKIIKDKDFLDNEENEDILEDIVLTLTLFED | 628 |
| S2 | 554 | VFKENRKVTKEKLLNYLNKEFPEYRIKDLIGLDKENKSFNASLGTYHDLKKIL-DKAFLDDKVNEEVIEDIIKTLTLFED | 632 |
| S3 | 552 | LEKENRKVTEKDIIHYLHN-VDGYDGIELKGIEKQ---FNASLSTYHDLLKIIKDKEEMDDAKNEAILENIVHTLTIFED | 627 |
| S4 | 290 | VFKQKKKPTLKQIAKEILVNEEDIKGYRVTSTGKPEF---TNLKVYHDIKDITARKEII---ENAELLDQIAKILTIYQS | 363 |
| S1 | 629 | REMIEERLKTYAHLFDDKVMKQLKR-RRYTGWGRLSRKLINGIRDKQSGKTILDFLKSDGFANRNFMQLIHDDSLTFKED | 707 |
| S2 | 633 | KDMIHERLQKYSDIFTANQLKKLER-RHYTGWGRLSYKLINGIRNKENNKTILDYLIDDGSANRNFMQLINDDTLPFKQI | 711 |
| S3 | 628 | REMIKQRLAQYDSLFDEKVIKALTR-RHYTGWGKLSAKLINGICDKQTGNTILDYLIDDGKINRNFMQLINDDGLSFKEI | 706 |
| S4 | 364 | SEDIQEELTNLNSELTQEEIEQISNLKGYTGTHNLSLKAINLILDE------LWHTNDNQIAIFNRLKLVP--------- | 428 |
| S1 | 708 | 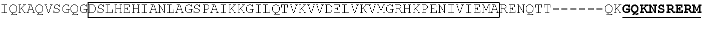 | 781 |
| S2 | 712 | 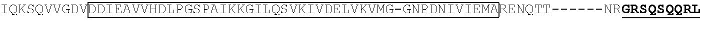 | 784 |
| S3 | 707 | 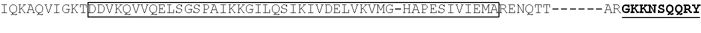 | 779 |
| S4 | 429 | 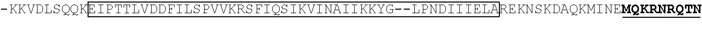 | 505 |
| S1 | 782 | KRIEEGIKELGSQIL-------KEHPVENTQLQNEKLYLYYLQNGRDMYVDQELDINRLSD----YDVDH*IVPQSFLKDD | 850 |
| S2 | 785 | KKLQNSLKELGSNILNEEKPSYIEDKVENSHLQNDQLFLYYIQNGKDMYTGDELDIDHLSD----YDIDH*IIPQAFIKDD | 860 |
| S3 | 780 | KRIEDSLKILASGL---DSNILKENPTDNNQLQNDRLFLYYLQNGKDMYTGEALDINQLSS----YDIDH*IIPQAFIKDD | 852 |
| S4 | 506 | ERIEEIIRTTGK---------------ENAKYLIEKIKLHDMQEGKCLYSLEAIPLEDLLNNPFNYEVDH*IIPRSVSFDN | 570 |
| S1 | 851 | 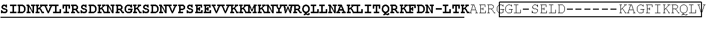 | 922 |
| S2 | 861 | 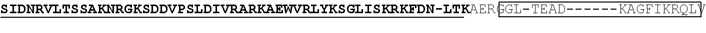 | 932 |
| S3 | 853 | 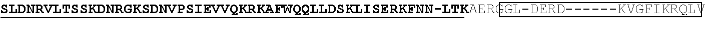 | 924 |
| S4 | 571 | 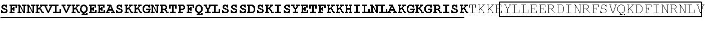 | 650 |
| S1 | 923 | 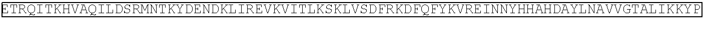 | 1002 |
| S2 | 933 | 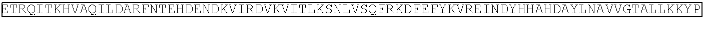 | 1012 |
| S3 | 925 | 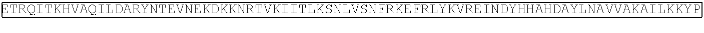 | 1004 |
| S4 | 651 | 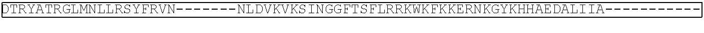 | 712 |
| S1 | 1003 | 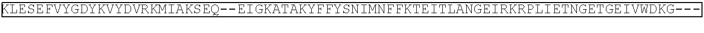 | 1077 |
| S2 | 1013 | 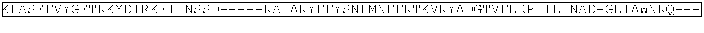 | 1083 |
| S3 | 1005 | 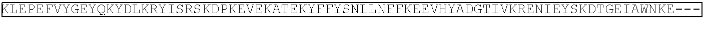 | 1081 |
| S4 | 713 | 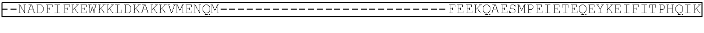 | 764 |
| S1 | 1078 | 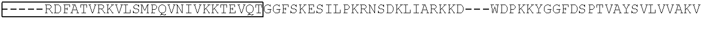 | 1149 |
| S2 | 1084 | 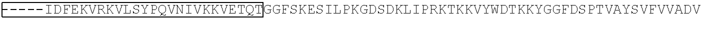 | 1158 |
| S3 | 1082 | 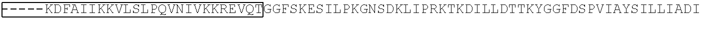 | 1156 |
| S4 | 765 | 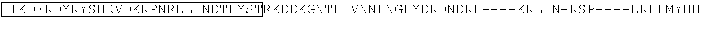 | 835 |
| S1 | 1150 | EKGKSKKLKSVKELLGITIMERSSFEKNPI-DFLEAKG------YKEVKKDLIIKLPKYSLFELENGRKRMLASAGELQKG | 1223 |
| S2 | 1159 | EKGKAKKLKTVKELVGISIMERSFFEENPV-EFLENKG------YHNIREDKLIKLPKYSLFEFEGGRRRLLASASELQKG | 1232 |
| S3 | 1157 | EKGKAKKLKTVKTLVGITIMEKAAFEENPI-TFLENKG------YHNVRKENILCLPKYSLFELENGRRRLLASAKELQKG | 1230 |
| S4 | 836 | DPQTYQKLK--------LIMEQYGDEKNPLYKYYEETGNYLTKYSKKDNGPVIKKIKYYGNKLNAHLDITDDYPNSRNKV | 907 |
| S1 | 1224 | NELALPSKYVNFLYLASHYEKLKGSPEDNEQKQLFVEQHKHYLDEIIEQISEFSKRVILADANLDKVLSAYNKH------ | 1297 |
| S2 | 1233 | NEMVLPGYLVELLYHAHRADNF-----NSTEYLNYVSEHKKEFEKVLSCVEDFANLYVDVEKNLSKIRAVADSM------ | 1301 |
| S3 | 1231 | NEIVLPVYLTTLLYHSKNVHKL-----DEPGHLEYIQKHRNEFKDLLNLVSEFSQKYVLADANLEKIKSLYADN------ | 1299 |
| S4 | 908 | VKLSLKPYRFD-VYLDNGVYKFV-----TVKNLDVIK--KENYYEVNSKAYEEAKKLKKISNQAEFIASFYNNDLIKING | 979 |
| S1 | 1298 | RDKPIREQAENIIHLFTLTNLGAPAAFKYFDTTIDRKRYTSTKEVLDATLIHQSIT--------GLYETRI----DLSQL | 1365 |
| S2 | 1302 | DNFSIEEISNSFINLLTLTALGAPADFNFLGEKIPRKRYTSTKECLNATLIHQSIT--------GLYETRI----DLSKL | 1369 |
| S3 | 1300 | EQADIEILANSFINLLTFTALGAPAAFKFFGKDIDRKRYTTVSEILNATLIHQSIT--------GLYETWI----DLSKL | 1367 |
| S4 | 980 | ELYRVIGVNNDLLNRIEVNMIDITYR-EYLENMNDKRPPRIIKTIASKT---QSIKKYSTDILGNLYEVKSKKHPQIIKK | 1055 |
| S1 | 1366 | GGD | 1368 |
| S2 | 1370 | GEE | 1372 |
| S3 | 1368 | GED | 1370 |
| S4 | 1056 | G-- | 1056 |

The alignment demonstrates that amino acid sequences and amino acid residues that are homologous to a reference Cas9 amino acid sequence or amino acid residue can be identified across Cas9 sequence variants, including, but not limited to Cas9 sequences from different species, by identifying the amino acid sequence or residue that aligns with the reference sequence or the reference residue using alignment programs and algorithms known in the art. This disclosure provides Cas9 variants in which one or more of the amino acid residues identified by an asterisk in SEQ ID NOs: 11-14 (e.g., 51, S2, S3, and S4, respectively) are mutated as described herein. The residues D10 and H840 in Cas9 of SEQ ID NO: 1 that correspond to the residues identified in SEQ ID NOs: 11-14 by an asterisk are referred to herein as “homologous” or “corresponding” residues. Such homologous residues can be identified by sequence alignment, e.g., as described above, and by identifying the sequence or residue that aligns with the reference sequence or residue. Similarly, mutations in Cas9 sequences that correspond to mutations identified in SEQ ID NO: 1 herein, e.g., mutations of residues 10, and 840 in SEQ ID NO: 1, are referred to herein as “homologous” or “corresponding” mutations. For example, the mutations corresponding to the D10A mutation in SEQ ID NO: 1 or 51 (SEQ ID NO: 11) for the four aligned sequences above are D11A for S2, D10A for S3, and D13A for S4; the corresponding mutations for H840A in SEQ ID NO: 1 or 51 (SEQ ID NO: 11) are H850A for S2, H842A for S3, and H560A for S4.

A total of 250 Cas9 sequences (SEQ ID NOs: 11-260) from different species are provided. Amino acid residues homologous to residues 10, and 840 of SEQ ID NO: 1 may be identified in the same manner as outlined above. All of these Cas9 sequences may be used in accordance with the present disclosure.

| | | |
|---|---|---|
| WP_010922251.1 | type II CRISPR RNA-guided endonuclease Cas9 [Streptococcus pyogenes] | SEQ ID NO: 11 |
| WP_039695303.1 | type II CRISPR RNA-guided endonuclease Cas9 [Streptococcus gallolyticus] | SEQ ID NO: 12 |
| WP_045635197.1 | type II CRISPR RNA-guided endonuclease Cas9 [Streptococcus mitis] | SEQ ID NO: 13 |
| 5AXW_A | Cas9, Chain A, Crystal Structure [Staphylococcus Aureus] | SEQ ID NO: 14 |
| WP_009880683.1 | type II CRISPR RNA-guided endonuclease Cas9 [Streptococcus pyogenes] | SEQ ID NO: 15 |
| WP_010922251.1 | type II CRISPR RNA-guided endonuclease Cas9 [Streptococcus pyogenes] | SEQ ID NO: 16 |
| WP_011054416.1 | type II CRISPR RNA-guided endonuclease Cas9 [Streptococcus pyogenes] | SEQ ID NO: 17 |
| WP_011284745.1 | type II CRISPR RNA-guided endonuclease Cas9 [Streptococcus pyogenes] | SEQ ID NO: 18 |
| WP_011285506.1 | type II CRISPR RNA-guided endonuclease Cas9 [Streptococcus pyogenes] | SEQ ID NO: 19 |
| WP_011527619.1 | type II CRISPR RNA-guided endonuclease Cas9 [Streptococcus pyogenes] | SEQ ID NO: 20 |
| WP_012560673.1 | type II CRISPR RNA-guided endonuclease Cas9 [Streptococcus pyogenes] | SEQ ID NO: 21 |
| WP_014407541.1 | type II CRISPR RNA-guided endonuclease Cas9 [Streptococcus pyogenes] | SEQ ID NO: 22 |
| WP_020905136.1 | type II CRISPR RNA-guided endonuclease Cas9 [Streptococcus pyogenes] | SEQ ID NO: 23 |
| WP_023080005.1 | type II CRISPR RNA-guided endonuclease Cas9 [Streptococcus pyogenes] | SEQ ID NO: 24 |
| WP_023610282.1 | type II CRISPR RNA-guided endonuclease Cas9 [Streptococcus pyogenes] | SEQ ID NO: 25 |
| WP_030125963.1 | type II CRISPR RNA-guided endonuclease Cas9 [Streptococcus pyogenes] | SEQ ID NO: 26 |
| WP_030126706.1 | type II CRISPR RNA-guided endonuclease Cas9 [Streptococcus pyogenes] | SEQ ID NO: 27 |
| WP_031488318.1 | type II CRISPR RNA-guided endonuclease Cas9 [Streptococcus pyogenes] | SEQ ID NO: 28 |
| WP_032460140.1 | type II CRISPR RNA-guided endonuclease Cas9 [Streptococcus pyogenes] | SEQ ID NO: 29 |
| WP_032461047.1 | type II CRISPR RNA-guided endonuclease Cas9 [Streptococcus pyogenes] | SEQ ID NO: 30 |
| WP_032462016.1 | type II CRISPR RNA-guided endonuclease Cas9 [Streptococcus pyogenes] | SEQ ID NO: 31 |
| WP_032462936.1 | type II CRISPR RNA-guided endonuclease Cas9 [Streptococcus pyogenes] | SEQ ID NO: 32 |
| WP_032464890.1 | type II CRISPR RNA-guided endonuclease Cas9 [Streptococcus pyogenes] | SEQ ID NO: 33 |
| WP_033888930.1 | type II CRISPR RNA-guided endonuclease Cas9 [Streptococcus pyogenes] | SEQ ID NO: 34 |
| WP_038431314.1 | type II CRISPR RNA-guided endonuclease Cas9 [Streptococcus pyogenes] | SEQ ID NO: 35 |
| WP_038432938.1 | type II CRISPR RNA-guided endonuclease Cas9 [Streptococcus pyogenes] | SEQ ID NO: 36 |
| WP_038434062.1 | type II CRISPR RNA-guided endonuclease Cas9 [Streptococcus pyogenes] | SEQ ID NO: 37 |
| BAQ51233.1 | CRISPR-associated protein, Csn1 family [Streptococcus pyogenes] | SEQ ID NO: 38 |
| KGE60162.1 | hypothetical protein MGAS2111_0903 [Streptococcus pyogenes MGAS2111] | SEQ ID NO: 39 |
| KGE60856.1 | CRISPR-associated endonuclease protein [Streptococcus pyogenes SS1447] | SEQ ID NO: 40 |
| WP_002989955.1 | MULTISPECIES: type II CRISPR RNA-guided endonuclease Cas9 [Streptococcus] | SEQ ID NO: 41 |
| WP_003030002.1 | MULTISPECIES: type II CRISPR RNA-guided endonuclease Cas9 [Streptococcus] | SEQ ID NO: 42 |
| WP_003065552.1 | MULTISPECIES: type II CRISPR RNA-guided endonuclease Cas9 [Streptococcus] | SEQ ID NO: 43 |
| WP_001040076.1 | type II CRISPR RNA-guided endonuclease Cas9 [Streptococcus agalactiae] | SEQ ID NO: 44 |
| WP_001040078.1 | type II CRISPR RNA-guided endonuclease Cas9 [Streptococcus agalactiae] | SEQ ID NO: 45 |
| WP_001040080.1 | type II CRISPR RNA-guided endonuclease Cas9 [Streptococcus agalactiae] | SEQ ID NO: 46 |
| WP_001040081.1 | type II CRISPR RNA-guided endonuclease Cas9 [Streptococcus agalactiae] | SEQ ID NO: 47 |
| WP_001040083.1 | type II CRISPR RNA-guided endonuclease Cas9 [Streptococcus agalactiae] | SEQ ID NO: 48 |
| WP_001040085.1 | type II CRISPR RNA-guided endonuclease Cas9 [Streptococcus agalactiae] | SEQ ID NO: 49 |
| WP_001040087.1 | type II CRISPR RNA-guided endonuclease Cas9 [Streptococcus agalactiae] | SEQ ID NO: 50 |
| WP_001040088.1 | type II CRISPR RNA-guided endonuclease Cas9 [Streptococcus agalactiae] | SEQ ID NO: 51 |
| WP_001040089.1 | type II CRISPR RNA-guided endonuclease Cas9 [Streptococcus agalactiae] | SEQ ID NO: 52 |
| WP_001040090.1 | type II CRISPR RNA-guided endonuclease Cas9 [Streptococcus agalactiae] | SEQ ID NO: 53 |
| WP_001040091.1 | type II CRISPR RNA-guided endonuclease Cas9 [Streptococcus agalactiae] | SEQ ID NO: 54 |
| WP_001040092.1 | type II CRISPR RNA-guided endonuclease Cas9 [Streptococcus agalactiae] | SEQ ID NO: 55 |
| WP_001040094.1 | type II CRISPR RNA-guided endonuclease Cas9 [Streptococcus agalactiae] | SEQ ID NO: 56 |
| WP_001040095.1 | type II CRISPR RNA-guided endonuclease Cas9 [Streptococcus agalactiae] | SEQ ID NO: 57 |
| WP_001040096.1 | type II CRISPR RNA-guided endonuclease Cas9 [Streptococcus agalactiae] | SEQ ID NO: 58 |
| WP_001040097.1 | type II CRISPR RNA-guided endonuclease Cas9 [Streptococcus agalactiae] | SEQ ID NO: 59 |
| WP_001040098.1 | type II CRISPR RNA-guided endonuclease Cas9 [Streptococcus agalactiae] | SEQ ID NO: 60 |
| WP_001040099.1 | type II CRISPR RNA-guided endonuclease Cas9 [Streptococcus agalactiae] | SEQ ID NO: 61 |
| WP_001040100.1 | type II CRISPR RNA-guided endonuclease Cas9 [Streptococcus agalactiae] | SEQ ID NO: 62 |
| WP_001040104.1 | type II CRISPR RNA-guided endonuclease Cas9 [Streptococcus agalactiae] | SEQ ID NO: 63 |
| WP_001040105.1 | type II CRISPR RNA-guided endonuclease Cas9 [Streptococcus agalactiae] | SEQ ID NO: 64 |
| WP_001040106.1 | type II CRISPR RNA-guided endonuclease Cas9 [Streptococcus agalactiae] | SEQ ID NO: 65 |
| WP_001040107.1 | type II CRISPR RNA-guided endonuclease Cas9 [Streptococcus agalactiae] | SEQ ID NO: 66 |
| WP_001040108.1 | type II CRISPR RNA-guided endonuclease Cas9 [Streptococcus agalactiae] | SEQ ID NO: 67 |
| WP_001040109.1 | type II CRISPR RNA-guided endonuclease Cas9 [Streptococcus agalactiae] | SEQ ID NO: 68 |
| WP_001040110.1 | type II CRISPR RNA-guided endonuclease Cas9 [Streptococcus agalactiae] | SEQ ID NO: 69 |
| WP_015058523.1 | type II CRISPR RNA-guided endonuclease Cas9 [Streptococcus agalactiae] | SEQ ID NO: 70 |
| WP_017643650.1 | type II CRISPR RNA-guided endonuclease Cas9 [Streptococcus agalactiae] | SEQ ID NO: 71 |
| WP_017647151.1 | type II CRISPR RNA-guided endonuclease Cas9 [Streptococcus agalactiae] | SEQ ID NO: 72 |
| WP_017648376.1 | type II CRISPR RNA-guided endonuclease Cas9 [Streptococcus agalactiae] | SEQ ID NO: 73 |
| WP_017649527.1 | type II CRISPR RNA-guided endonuclease Cas9 [Streptococcus agalactiae] | SEQ ID NO: 74 |
| WP_017771611.1 | type II CRISPR RNA-guided endonuclease Cas9 [Streptococcus agalactiae] | SEQ ID NO: 75 |
| WP_017771984.1 | type II CRISPR RNA-guided endonuclease Cas9 [Streptococcus agalactiae] | SEQ ID NO: 76 |
| CFQ25032.1 | CRISPR-associated protein [Streptococcus agalactiae] | SEQ ID NO: 77 |
| CFV16040.1 | CRISPR-associated protein [Streptococcus agalactiae] | SEQ ID NO: 78 |
| KLJ37842.1 | CRISPR-associated protein Csn1 [Streptococcus agalactiae] | SEQ ID NO: 79 |
| KLJ72361.1 | CRISPR-associated protein Csn1 [Streptococcus agalactiae] | SEQ ID NO: 80 |
| KLL20707.1 | CRISPR-associated protein Csn1 [Streptococcus agalactiae] | SEQ ID NO: 81 |
| KLL42645.1 | CRISPR-associated protein Csn1 [Streptococcus agalactiae] | SEQ ID NO: 82 |
| WP_047207273.1 | type II CRISPR RNA-guided endonuclease Cas9 [Streptococcus agalactiae] | SEQ ID NO: 83 |
| WP_047209694.1 | type II CRISPR RNA-guided endonuclease Cas9 [Streptococcus agalactiae] | SEQ ID NO: 84 |
| WP_050198062.1 | type II CRISPR RNA-guided endonuclease Cas9 [Streptococcus agalactiae] | SEQ ID NO: 85 |
| WP_050201642.1 | type II CRISPR RNA-guided endonuclease Cas9 [Streptococcus agalactiae] | SEQ ID NO: 86 |
| WP_050204027.1 | type II CRISPR RNA-guided endonuclease Cas9 [Streptococcus agalactiae] | SEQ ID NO: 87 |
| WP_050881965.1 | type II CRISPR RNA-guided endonuclease Cas9 [Streptococcus agalactiae] | SEQ ID NO: 88 |
| WP_050886065.1 | type II CRISPR RNA-guided endonuclease Cas9 [Streptococcus agalactiae] | SEQ ID NO: 89 |
| AHN30376.1 | CRISPR-associated protein Csn1 [Streptococcus agalactiae 138P] | SEQ ID NO: 90 |
| EAO78426.1 | reticulocyte binding protein [Streptococcus agalactiae H36B] | SEQ ID NO: 91 |
| CCW42055.1 | CRISPR-associated protein, SAG0894 family [Streptococcus agalactiae ILRI112] | SEQ ID NO: 92 |
| WP_003041502.1 | type II CRISPR RNA-guided endonuclease Cas9 [Streptococcus anginosus] | SEQ ID NO: 93 |
| WP_037593752.1 | type II CRISPR RNA-guided endonuclease Cas9 [Streptococcus anginosus] | SEQ ID NO: 94 |
| WP_049516684.1 | CRISPR-associated protein Csn1 [Streptococcus anginosus] | SEQ ID NO: 95 |
| GAD46167.1 | hypothetical protein ANG6_0662 [Streptococcus anginosus T5] | SEQ ID NO: 96 |
| WP_018363470.1 | type II CRISPR RNA-guided endonuclease Cas9 [Streptococcus caballi] | SEQ ID NO: 97 |
| WP_003043819.1 | type II CRISPR RNA-guided endonuclease Cas9 [Streptococcus canis] | SEQ ID NO: 98 |
| WP_006269658.1 | type II CRISPR RNA-guided endonuclease Cas9 [Streptococcus constellatus] | SEQ ID NO: 99 |
| WP_048800889.1 | type II CRISPR RNA-guided endonuclease Cas9 [Streptococcus constellatus] | SEQ ID NO: 100 |
| WP_012767106.1 | type II CRISPR RNA-guided endonuclease Cas9 [Streptococcus dysgalactiae] | SEQ ID NO: 101 |
| WP_014612333.1 | type II CRISPR RNA-guided endonuclease Cas9 [Streptococcus dysgalactiae] | SEQ ID NO: 102 |
| WP_015017095.1 | type II CRISPR RNA-guided endonuclease Cas9 [Streptococcus dysgalactiae] | SEQ ID NO: 103 |
| WP_015057649.1 | type II CRISPR RNA-guided endonuclease Cas9 [Streptococcus dysgalactiae] | SEQ ID NO: 104 |
| WP_048327215.1 | type II CRISPR RNA-guided endonuclease Cas9 [Streptococcus dysgalactiae] | SEQ ID NO: 105 |
| WP_049519324.1 | CRISPR-associated protein Csn1 [Streptococcus dysgalactiae] | SEQ ID NO: 106 |
| WP_012515931.1 | type II CRISPR RNA-guided endonuclease Cas9 [Streptococcus equi] | SEQ ID NO: 107 |
| WP_021320964.1 | type II CRISPR RNA-guided endonuclease Cas9 [Streptococcus equi] | SEQ ID NO: 108 |
| WP_037581760.1 | type II CRISPR RNA-guided endonuclease Cas9 [Streptococcus equi] | SEQ ID NO: 109 |
| WP_004232481.1 | type II CRISPR RNA-guided endonuclease Cas9 [Streptococcus equinus] | SEQ ID NO: 110 |
| WP_009854540.1 | type II CRISPR RNA-guided endonuclease Cas9 [Streptococcus gallolyticus] | SEQ ID NO: 111 |
| WP_012962174.1 | type II CRISPR RNA-guided endonuclease Cas9 [Streptococcus gallolyticus] | SEQ ID NO: 112 |
| WP_039695303.1 | type II CRISPR RNA-guided endonuclease Cas9 [Streptococcus gallolyticus] | SEQ ID NO: 113 |
| WP_014334983.1 | type II CRISPR RNA-guided endonuclease Cas9 [Streptococcus infantarius] | SEQ ID NO: 114 |
| WP_003099269.1 | type II CRISPR RNA-guided endonuclease Cas9 [Streptococcus iniae] | SEQ ID NO: 115 |
| AHY15608.1 | CRISPR-associated protein Csn1 [Streptococcus iniae] | SEQ ID NO: 116 |
| AHY17476.1 | CRISPR-associated protein Csn1 [Streptococcus iniae] | SEQ ID NO: 117 |
| ESR09100.1 | hypothetical protein IUSA1_08595 [Streptococcus iniae IUSA1] | SEQ ID NO: 118 |
| AGM98575.1 | CRISPR-associated protein Cas9/Csn1, subtype II/NMEMI [Streptococcus iniae SF1] | SEQ ID NO: 119 |
| ALF27331.1 | CRISPR-associated protein Csn1 [Streptococcus intermedius] | SEQ ID NO: 120 |
| WP_018372492.1 | type II CRISPR RNA-guided endonuclease Cas9 [Streptococcus massiliensis] | SEQ ID NO: 121 |
| WP_045618028.1 | type II CRISPR RNA-guided endonuclease Cas9 [Streptococcus mitis] | SEQ ID NO: 122 |
| WP_045635197.1 | type II CRISPR RNA-guided endonuclease Cas9 [Streptococcus mitis] | SEQ ID NO: 123 |
| WP_002263549.1 | type II CRISPR RNA-guided endonuclease Cas9 [Streptococcus mutans] | SEQ ID NO: 124 |
| WP_002263887.1 | type II CRISPR RNA-guided endonuclease Cas9 [Streptococcus mutans] | SEQ ID NO: 125 |
| WP_002264920.1 | type II CRISPR RNA-guided endonuclease Cas9 [Streptococcus mutans] | SEQ ID NO: 126 |
| WP_002269043.1 | type II CRISPR RNA-guided endonuclease Cas9 [Streptococcus mutans] | SEQ ID NO: 127 |
| WP_002269448.1 | type II CRISPR RNA-guided endonuclease Cas9 [Streptococcus mutans] | SEQ ID NO: 128 |
| WP_002271977.1 | type II CRISPR RNA-guided endonuclease Cas9 [Streptococcus mutans] | SEQ ID NO: 129 |
| WP_002272766.1 | type II CRISPR RNA-guided endonuclease Cas9 [Streptococcus mutans] | SEQ ID NO: 130 |
| WP_002273241.1 | type II CRISPR RNA-guided endonuclease Cas9 [Streptococcus mutans] | SEQ ID NO: 131 |
| WP_002275430.1 | type II CRISPR RNA-guided endonuclease Cas9 [Streptococcus mutans] | SEQ ID NO: 132 |
| WP_002276448.1 | type II CRISPR RNA-guided endonuclease Cas9 [Streptococcus mutans] | SEQ ID NO: 133 |
| WP_002277050.1 | type II CRISPR RNA-guided endonuclease Cas9 [Streptococcus mutans] | SEQ ID NO: 134 |
| WP_002277364.1 | type II CRISPR RNA-guided endonuclease Cas9 [Streptococcus mutans] | SEQ ID NO: 135 |
| WP_002279025.1 | type II CRISPR RNA-guided endonuclease Cas9 [Streptococcus mutans] | SEQ ID NO: 136 |
| WP_002279859.1 | type II CRISPR RNA-guided endonuclease Cas9 [Streptococcus mutans] | SEQ ID NO: 137 |
| WP_002280230.1 | type II CRISPR RNA-guided endonuclease Cas9 [Streptococcus mutans] | SEQ ID NO: 138 |
| WP_002281696.1 | type II CRISPR RNA-guided endonuclease Cas9 [Streptococcus mutans] | SEQ ID NO: 139 |
| WP_002282247.1 | type II CRISPR RNA-guided endonuclease Cas9 [Streptococcus mutans] | SEQ ID NO: 140 |
| WP_002282906.1 | type II CRISPR RNA-guided endonuclease Cas9 [Streptococcus mutans] | SEQ ID NO: 141 |
| WP_002283846.1 | type II CRISPR RNA-guided endonuclease Cas9 [Streptococcus mutans] | SEQ ID NO: 142 |
| WP_002287255.1 | type II CRISPR RNA-guided endonuclease Cas9 [Streptococcus mutans] | SEQ ID NO: 143 |
| WP_002288990.1 | type II CRISPR RNA-guided endonuclease Cas9 [Streptococcus mutans] | SEQ ID NO: 144 |
| WP_002289641.1 | type II CRISPR RNA-guided endonuclease Cas9 [Streptococcus mutans] | SEQ ID NO: 145 |
| WP_002290427.1 | type II CRISPR RNA-guided endonuclease Cas9 [Streptococcus mutans] | SEQ ID NO: 146 |
| WP_002295753.1 | type II CRISPR RNA-guided endonuclease Cas9 [Streptococcus mutans] | SEQ ID NO: 147 |
| WP_002296423.1 | type II CRISPR RNA-guided endonuclease Cas9 [Streptococcus mutans] | SEQ ID NO: 148 |
| WP_002304487.1 | type II CRISPR RNA-guided endonuclease Cas9 [Streptococcus mutans] | SEQ ID NO: 149 |
| WP_002305844.1 | type II CRISPR RNA-guided endonuclease Cas9 [Streptococcus mutans] | SEQ ID NO: 150 |
| WP_002307203.1 | type II CRISPR RNA-guided endonuclease Cas9 [Streptococcus mutans] | SEQ ID NO: 151 |
| WP_002310390.1 | type II CRISPR RNA-guided endonuclease Cas9 [Streptococcus mutans] | SEQ ID NO: 152 |
| WP_002352408.1 | type II CRISPR RNA-guided endonuclease Cas9 [Streptococcus mutans] | SEQ ID NO: 153 |
| WP_012997688.1 | type II CRISPR RNA-guided endonuclease Cas9 [Streptococcus mutans] | SEQ ID NO: 154 |
| WP_014677909.1 | type II CRISPR RNA-guided endonuclease Cas9 [Streptococcus mutans] | SEQ ID NO: 155 |
| WP_019312892.1 | type II CRISPR RNA-guided endonuclease Cas9 [Streptococcus mutans] | SEQ ID NO: 156 |
| WP_019313659.1 | type II CRISPR RNA-guided endonuclease Cas9 [Streptococcus mutans] | SEQ ID NO: 157 |
| WP_019314093.1 | type II CRISPR RNA-guided endonuclease Cas9 [Streptococcus mutans] | SEQ ID NO: 158 |
| WP_019315370.1 | type II CRISPR RNA-guided endonuclease Cas9 [Streptococcus mutans] | SEQ ID NO: 159 |
| WP_019803776.1 | type II CRISPR RNA-guided endonuclease Cas9 [Streptococcus mutans] | SEQ ID NO: 160 |
| WP_019805234.1 | type II CRISPR RNA-guided endonuclease Cas9 [Streptococcus mutans] | SEQ ID NO: 161 |
| WP_024783594.1 | type II CRISPR RNA-guided endonuclease Cas9 [Streptococcus mutans] | SEQ ID NO: 162 |
| WP_024784288.1 | type II CRISPR RNA-guided endonuclease Cas9 [Streptococcus mutans] | SEQ ID NO: 163 |
| WP_024784666.1 | type II CRISPR RNA-guided endonuclease Cas9 [Streptococcus mutans] | SEQ ID NO: 164 |
| WP_024784894.1 | type II CRISPR RNA-guided endonuclease Cas9 [Streptococcus mutans] | SEQ ID NO: 165 |
| WP_024786433.1 | type II CRISPR RNA-guided endonuclease Cas9 [Streptococcus mutans] | SEQ ID NO: 166 |
| WP_049473442.1 | CRISPR-associated protein Csn1 [Streptococcus mutans] | SEQ ID NO: 167 |
| WP_049474547.1 | CRISPR-associated protein Csn1 [Streptococcus mutans] | SEQ ID NO: 168 |
| EMC03581.1 | hypothetical protein SMU69_09359 [Streptococcus mutans NLML4] | SEQ ID NO: 169 |
| WP_000428612.1 | type II CRISPR RNA-guided endonuclease Cas9 [Streptococcus oralis] | SEQ ID NO: 170 |
| WP_000428613.1 | type II CRISPR RNA-guided endonuclease Cas9 [Streptococcus oralis] | SEQ ID NO: 171 |
| WP_049523028.1 | CRISPR-associated protein Csn1 [Streptococcus parasanguinis] | SEQ ID NO: 172 |
| WP_003107102.1 | type II CRISPR RNA-guided endonuclease Cas9 [Streptococcus parauberis] | SEQ ID NO: 173 |
| WP_054279288.1 | type II CRISPR RNA-guided endonuclease Cas9 [Streptococcus phocae] | SEQ ID NO: 174 |
| WP_049531101.1 | CRISPR-associated protein Csn1 [Streptococcus pseudopneumoniae] | SEQ ID NO: 175 |
| WP_049538452.1 | CRISPR-associated protein Csn1 [Streptococcus pseudopneumoniae] | SEQ ID NO: 176 |
| WP_049549711.1 | CRISPR-associated protein Csn1 [Streptococcus pseudopneumoniae] | SEQ ID NO: 177 |
| WP_007896501.1 | type II CRISPR RNA-guided endonuclease Cas9 [Streptococcus pseudoporcinus] | SEQ ID NO: 178 |
| EFR44625.1 | CRISPR-associated protein, Csn1 family [Streptococcus pseudoporcinus SPIN 20026] | SEQ ID NO: 179 |
| WP_002897477.1 | type II CRISPR RNA-guided endonuclease Cas9 [Streptococcus sanguinis] | SEQ ID NO: 180 |
| WP_002906454.1 | type II CRISPR RNA-guided endonuclease Cas9 [Streptococcus sanguinis] | SEQ ID NO: 181 |
| WP_009729476.1 | type II CRISPR RNA-guided endonuclease Cas9 [Streptococcus sp. F0441] | SEQ ID NO: 182 |
| CQR24647.1 | CRISPR-associated protein [Streptococcus sp. FF10] | SEQ ID NO: 183 |
| WP_000066813.1 | type II CRISPR RNA-guided endonuclease Cas9 [Streptococcus sp. M334] | SEQ ID NO: 184 |
| WP_009754323.1 | type II CRISPR RNA-guided endonuclease Cas9 [Streptococcus sp. taxon 056] | SEQ ID NO: 185 |
| WP_044674937.1 | type II CRISPR RNA-guided endonuclease Cas9 [Streptococcus suis] | SEQ ID NO: 186 |
| WP_044676715.1 | type II CRISPR RNA-guided endonuclease Cas9 [Streptococcus suis] | SEQ ID NO: 187 |
| WP_044680361.1 | type II CRISPR RNA-guided endonuclease Cas9 [Streptococcus suis] | SEQ ID NO: 188 |
| WP_044681799.1 | type II CRISPR RNA-guided endonuclease Cas9 [Streptococcus suis] | SEQ ID NO: 189 |
| WP_049533112.1 | CRISPR-associated protein Csn1 [Streptococcus suis] | SEQ ID NO: 190 |
| WP_029090905.1 | type II CRISPR RNA-guided endonuclease Cas9 [Brochothrix thermosphacta] | SEQ ID NO: 191 |
| WP_006506696.1 | type II CRISPR RNA-guided endonuclease Cas9 [Catenibacterium mitsuokai] | SEQ ID NO: 192 |
| AIT42264.1 | Cas9hc:NLS:HA [Cloning vector pYB196] | SEQ ID NO: 193 |
| WP_034440723.1 | type II CRISPR endonuclease Cas9 [Clostridiales bacterium S5-A11] | SEQ ID NO: 194 |
| AKQ21048.1 | Cas9 [CRISPR-mediated gene targeting vector p(bhsp68-Cas9)] | SEQ ID NO: 195 |
| WP_004636532.1 | type II CRISPR RNA-guided endonuclease Cas9 [Dolosigranulum pigrum] | SEQ ID NO: 196 |
| WP_002364836.1 | MULTISPECIES: type II CRISPR RNA-guided endonuclease Cas9 [Enterococcus] | SEQ ID NO: 197 |
| WP_016631044.1 | MULTISPECIES: type II CRISPR RNA-guided endonuclease Cas9 [Enterococcus] | SEQ ID NO: 198 |
| EMS75795.1 | hypothetical protein H318_06676 [Enterococcus durans IPLA 655] | SEQ ID NO: 199 |
| WP_002373311.1 | type II CRISPR RNA-guided endonuclease Cas9 [Enterococcus faecalis] | SEQ ID NO: 200 |
| WP_002378009.1 | type II CRISPR RNA-guided endonuclease Cas9 [Enterococcus faecalis] | SEQ ID NO: 201 |
| WP_002407324.1 | type II CRISPR RNA-guided endonuclease Cas9 [Enterococcus faecalis] | SEQ ID NO: 202 |
| WP_002413717.1 | type II CRISPR RNA-guided endonuclease Cas9 [Enterococcus faecalis] | SEQ ID NO: 203 |
| WP_010775580.1 | type II CRISPR RNA-guided endonuclease Cas9 [Enterococcus faecalis] | SEQ ID NO: 204 |
| WP_010818269.1 | type II CRISPR RNA-guided endonuclease Cas9 [Enterococcus faecalis] | SEQ ID NO: 205 |
| WP_010824395.1 | type II CRISPR RNA-guided endonuclease Cas9 [Enterococcus faecalis] | SEQ ID NO: 206 |
| WP_016622645.1 | type II CRISPR RNA-guided endonuclease Cas9 [Enterococcus faecalis] | SEQ ID NO: 207 |
| WP_033624816.1 | type II CRISPR RNA-guided endonuclease Cas9 [Enterococcus faecalis] | SEQ ID NO: 208 |
| WP_033625576.1 | type II CRISPR RNA-guided endonuclease Cas9 [Enterococcus faecalis] | SEQ ID NO: 209 |
| WP_033789179.1 | type II CRISPR RNA-guided endonuclease Cas9 [Enterococcus faecalis] | SEQ ID NO: 210 |
| WP_002310644.1 | type II CRISPR RNA-guided endonuclease Cas9 [Enterococcus faecium] | SEQ ID NO: 211 |
| WP_002312694.1 | type II CRISPR RNA-guided endonuclease Cas9 [Enterococcus faecium] | SEQ ID NO: 212 |
| WP_002314015.1 | type II CRISPR RNA-guided endonuclease Cas9 [Enterococcus faecium] | SEQ ID NO: 213 |
| WP_002320716.1 | type II CRISPR RNA-guided endonuclease Cas9 [Enterococcus faecium] | SEQ ID NO: 214 |
| WP_002330729.1 | type II CRISPR RNA-guided endonuclease Cas9 [Enterococcus faecium] | SEQ ID NO: 215 |
| WP_002335161.1 | type II CRISPR RNA-guided endonuclease Cas9 [Enterococcus faecium] | SEQ ID NO: 216 |
| WP_002345439.1 | type II CRISPR RNA-guided endonuclease Cas9 [Enterococcus faecium] | SEQ ID NO: 217 |
| WP_034867970.1 | type II CRISPR RNA-guided endonuclease Cas9 [Enterococcus faecium] | SEQ ID NO: 218 |
| WP_047937432.1 | type II CRISPR RNA-guided endonuclease Cas9 [Enterococcus faecium] | SEQ ID NO: 219 |
| WP_010720994.1 | type II CRISPR RNA-guided endonuclease Cas9 [Enterococcus hirae] | SEQ ID NO: 220 |
| WP_010737004.1 | type II CRISPR RNA-guided endonuclease Cas9 [Enterococcus hirae] | SEQ ID NO: 221 |
| WP_034700478.1 | type II CRISPR RNA-guided endonuclease Cas9 [Enterococcus hirae] | SEQ ID NO: 222 |
| WP_007209003.1 | type II CRISPR RNA-guided endonuclease Cas9 [Enterococcus italicus] | SEQ ID NO: 223 |
| WP_023519017.1 | type II CRISPR RNA-guided endonuclease Cas9 [Enterococcus mundtii] | SEQ ID NO: 224 |
| WP_010770040.1 | type II CRISPR RNA-guided endonuclease Cas9 [Enterococcus phoeniculicola] | SEQ ID NO: 225 |
| WP_048604708.1 | type II CRISPR RNA-guided endonuclease Cas9 [Enterococcus sp. AM1] | SEQ ID NO: 226 |
| WP_010750235.1 | type II CRISPR RNA-guided endonuclease Cas9 [Enterococcus villorum] | SEQ ID NO: 227 |
| AII16583.1 | Cas9 endonuclease [Expression vector pCas9] | SEQ ID NO: 228 |
| WP_029073316.1 | type II CRISPR RNA-guided endonuclease Cas9 [Kandleria vitulina] | SEQ ID NO: 229 |
| WP_031589969.1 | type II CRISPR RNA-guided endonuclease Cas9 [Kandleria vitulina] | SEQ ID NO: 230 |
| KDA45870.1 | CRISPR-associated protein Cas9/Csn1, subtype II/NMEMI [Lactobacillus animalis] | SEQ ID NO: 231 |
| WP_039099354.1 | type II CRISPR RNA-guided endonuclease Cas9 [Lactobacillus curvatus] | SEQ ID NO: 232 |
| AKP02966.1 | hypothetical protein ABB45_04605 [Lactobacillus farciminis] | SEQ ID NO: 233 |
| WP_010991369.1 | type II CRISPR RNA-guided endonuclease Cas9 [Listeria innocua] | SEQ ID NO: 234 |
| WP_033838504.1 | type II CRISPR RNA-guided endonuclease Cas9 [Listeria innocua] | SEQ ID NO: 235 |
| EHN60060.1 | CRISPR-associated protein, Csn1 family [Listeria innocua ATCC 33091] | SEQ ID NO: 236 |
| EFR89594.1 | crispr-associated protein, Csn1 family [Listeria innocua FSL S4-378] | SEQ ID NO: 237 |
| WP_038409211.1 | type II CRISPR RNA-guided endonuclease Cas9 [Listeria ivanovii] | SEQ ID NO: 238 |
| EFR95520.1 | crispr-associated protein Csn1 [Listeria ivanovii FSL F6-596] | SEQ ID NO: 239 |
| WP_003723650.1 | type II CRISPR RNA-guided endonuclease Cas9 [Listeria monocytogenes] | SEQ ID NO: 240 |
| WP_003727705.1 | type II CRISPR RNA-guided endonuclease Cas9 [Listeria monocytogenes] | SEQ ID NO: 241 |
| WP_003730785.1 | type II CRISPR RNA-guided endonuclease Cas9 [Listeria monocytogenes] | SEQ ID NO: 242 |
| WP_003733029.1 | type II CRISPR RNA-guided endonuclease Cas9 [Listeria monocytogenes] | SEQ ID NO: 243 |
| WP_003739838.1 | type II CRISPR RNA-guided endonuclease Cas9 [Listeria monocytogenes] | SEQ ID NO: 244 |
| WP_014601172.1 | type II CRISPR RNA-guided endonuclease Cas9 [Listeria monocytogenes] | SEQ ID NO: 245 |
| WP_023548323.1 | type II CRISPR RNA-guided endonuclease Cas9 [Listeria monocytogenes] | SEQ ID NO: 246 |
| WP_031665337.1 | type II CRISPR RNA-guided endonuclease Cas9 [Listeria monocytogenes] | SEQ ID NO: 247 |
| WP_031669209.1 | type II CRISPR RNA-guided endonuclease Cas9 [Listeria monocytogenes] | SEQ ID NO: 248 |
| WP_033920898.1 | type II CRISPR RNA-guided endonuclease Cas9 [Listeria monocytogenes] | SEQ ID NO: 249 |
| AKI42028.1 | CRISPR-associated protein [Listeria monocytogenes] | SEQ ID NO: 250 |
| AKI50529.1 | CRISPR-associated protein [Listeria monocytogenes] | SEQ ID NO: 251 |
| EFR83390.1 | crispr-associated protein Csn1 [Listeria monocytogenes FSL F2-208] | SEQ ID NO: 252 |
| WP_046323366.1 | type II CRISPR RNA-guided endonuclease Cas9 [Listeria seeligeri] | SEQ ID NO: 253 |
| AKE81011.1 | Cas9 [Plant multiplex genome editing vector pYLCRISPR/Cas9Pubi-H] | SEQ ID NO: 254 |
| CUO82355.1 | Uncharacterized protein conserved in bacteria [Roseburia hominis] | SEQ ID NO: 255 |
| WP_033162887.1 | type II CRISPR RNA-guided endonuclease Cas9 [Sharpea azabuensis] | SEQ ID NO: 256 |
| AGZ01981.1 | Cas9 endonuclease [synthetic construct] | SEQ ID NO: 257 |
| AKA60242.1 | nuclease deficient Cas9 [synthetic construct] | SEQ ID NO: 258 |
| AKS40380.1 | Cas9 [Synthetic plasmid pFC330] | SEQ ID NO: 259 |
| 4UN5_B | Cas9, Chain B, Crystal Structure | SEQ ID NO: 260 |

Non-limiting examples of suitable deaminase domains are provided.

Human AID
(SEQ ID NO: 303)
MDSLLMNRRKFLYQFKNVRWAKGRRETYLCYVVKRRDSATSFSLDFGYLRNKNGCHVELLFLRYISDWD
LDPGRCYRVTWFTSWSPCYDCARHVADFLRGNPNLSLRIFTARLYFCEDRKAEPEGLRRLHRAGVQIAIMT
FKDYFYCWNTFVENHERTFKAWEGLHENSVRLSRQLRRILLPLYEVDDLRDAFRTLGL
(underline: nuclear localization signal; double underline:
nuclear export signal)
Mouse AID
(SEQ ID NO: 271)
MDSLLMKQKKFLYHFKNVRWAKGRHETYLCYVVKRRDSATSCSLDFGHLRNKSGCHVELLFLRYISDWD
LDPGRCYRVTWFTSWSPCYDCARHVAEFLRWNPNLSLRIFTARLYFCEDRKAEPEGLRRLHRAGVQIGIMT
FKDYFYCWNTFVENRERTFKAWEGLHENSVRLTRQLRRILLPLYEVDDLRDAFRMLGF
(underline: nuclear localization signal; double underline:
nuclear export signal)
Dog AID
(SEQ ID NO: 272)
MDSLLMKQRKFLYHFKNVRWAKGRHETYLCYVVKRRDSATSFSLDFGHLRNKSGCHVELLFLRYISDWD
LDPGRCYRVTWFTSWSPCYDCARHVADFLRGYPNLSLRIFAARLYFCEDRKAEPEGLRRLHRAGVQIAIMT
FKDYFYCWNTFVENREKTFKAWEGLHENSVRLSRQLRRILLPLYEVDDLRDAFRTLGL
(underline: nuclear localization signal; double underline:
nuclear export signal)
Bovine AID
(SEQ ID NO: 273)
MDSLLKKQRQFLYQFKNVRWAKGRHETYLCYVVKRRDSPTSFSLDFGHLRNKAGCHVELLFLRYISDWD
LDPGRCYRVTWFTSWSPCYDCARHVADFLRGYPNLSLRIFTARLYFCDKERKAEPEGLRRLHRAGVQIAIM
TFKDYFYCWNTFVENHERTFKAWEGLHENSVRLSRQLRRILLPLYEVDDLRDAFRTLGL
(underline: nuclear localization signal; double underline:
nuclear export signal)
Mouse APOBEC-3
(SEQ ID NO: 274)
MGPFCLGCSHRKCYSPIRNLISQETFKFHFKNLGYAKGRKDTFLCYEVTRKDCDSPVSLHHGVFKNKDNIH
AEICFLYWFHDKVLKVLSPREEFKITWYMSWSPCFECAEQIVRFLATHHNLSLDIFSSRLYNVQDPETQQNLCR
LVQEGAQVAAMDLYEFKKCWKKFVDNGGRRFRPWKRLLTNFRYQDSKLQEILRPCYIPVPSSSSSTLSNIC
LTKGLPETRFCVEGRRMDPLSEEEFYSQFYNQRVKHLCYYHRMKPYLCYQLEQFNGQAPLKGCLLSEKGK
QHAEILFLDKIRSMELSQVTITCYLTWSPCPNCAWQLAAFKRDRPDLILHIYTSRLYFHWKRPFQKGLCSLWQ
SGILVDVMDLPQFTDCWTNFVNPKRPFWPWKGLEIISRRTQRRLRRIKESWGLQDLVNDFGNLQLGPPMS
(italic: nucleic acid editing domain)
Rat APOBEC-3
(SEQ ID NO: 275)
MGPFCLGCSHRKCYSPIRNLISQETFKFHFKNLRYAIDRKDTFLCYEVTRKDCDSPVSLHHGVFKNKDNIHA
EICFLYWFHDKVLKVLSPREEFKITWYMSWSPCFECAEQVLRFLATHHNLSLDIFSSRLYNIRDPENQQNLCRL
VQEGAQVAAMDLYEFKKCWKKFVDNGGRRFRPWKKLLTNFRYQDSKLQEILRPCYIPVPSSSSSTLSNICL
TKGLPETRFCVERRRVHLLSEEEFYSQFYNQRVKHLCYYHGVKPYLCYQLEQFNGQAPLKGCLLSEKGKQ
HAEILFLDKIRSMELSQVIITCYLTWSPCPNCAWQLAAFKRDRPDLILHIYTSRLYFHWKRPFQKGLCSLWQSG
ILVDVMDLPQFTDCWTNFVNPKRPFWPWKGLEIISRRTQRRLHRIKESWGLQDLVNDFGNLQLGPPMS
(italic: nucleic acid editing domain)
Rhesus macaque APOBEC-3G
(SEQ ID NO: 276)
MVEPMDPRTFVSNFNNRPILSGLNTVWLCCEVKTKDPSGPPLDAKIFQGKVYSKAKYHPEMRFLRWFHKW
RQLHHDQEYKVTWYVSWSPCTRCANSVATFLAKDPKVTLTIFVARLYYFWKPDYQQALRILCQKRGGPHAT
MKIMNYNEFQDCWNKFVDGRGKPFKPRNNLPKHYTLLQATLGELLRHLMDPGTFTSNFNNKPWVSGQHE
TYLCYKVERLHNDTWVPLNQHRGFLRNQAPNIHGFPKGRHAELCFLDLIPFWKLDGQQYRVTCFTSWSPCFS
CAQEMAKFISNNEHVSLCIFAARIYDDQGRYQEGLRALHRDGAKIAMMNYSEFEYCWDTFVDRQGRPFQP
WDGLDEHSQALSGRLRAI
(italic: nucleic acid editing domain; underline:
cytoplasmic localization signal)
Chimpanzee APOBEC-3G
(SEQ ID NO: 277)
MKPHFRNPVERMYQDTFSDNFYNRPILSHRNTVWLCYEVKTKGPSRPPLDAKIFRGQVYSKLKYHPEMRF
FHWFSKWRKLHRDQEYEVTWYISWSPCTKCTRDVATFLAEDPKVTLTIFVARLYYFWDPDYQEALRSLCQKR
DGPRATMKIMNYDEFQHCWSKFVYSQRELFEPWNNLPKYYILLHIMLGEILRHSMDPPTFTSNFNNELWVR
GRHETYLCYEVERLHNDTWVLLNQRRGFLCNQAPHKHGFLEGRHAELCFLDVIPFWKLDLHQDYRVTCFTS
WSPCFSCAQEMAKFISNNKHVSLCIFAARIYDDQGRCQEGLRTLAKAGAKISIMTYSEFKHCWDTFVDHQG
CPFQPWDGLEEHSQALSGRLRAILQNQGN
(italic: nucleic acid editing domain;
underline: cytoplasmic localization signal)
Green monkey APOBEC-3G
(SEQ ID NO: 278)
MNPQIRNMVEQMEPDIFVYYFNNRPILSGRNTVWLCYEVKTKDPSGPPLDANIFQGKLYPEAKDHPEMKFL
HWFRKWRQLHRDQEYEVTWYVSWSPCTRCANSVATFLAEDPKVTLTIFVARLYYFWKPDYQQALRILCQER
GGPHATMKIMNYNEFQHCWNEFVDGQGKPFKPRKNLPKHYTLLHATLGELLRHVMDPGTFTSNFNNKPW
VSGQRETYLCYKVERSHNDTWVLLNQHRGFLRNQAPDRHGFPKGRHAELCFLDLIPFWKLDDQQYRVTCFT
SWSPCFSCAQKMAKFISNNKHVSLCIFAARIYDDQGRCQEGLRTLHRDGAKIAVMNYSEFEYCWDTFVDR
QGRPFQPWDGLDEHSQALSGRLRAI
(italic: nucleic acid editing domain;
underline: cytoplasmic localization signal)
Human APOBEC-3G
(SEQ ID NO: 279)
MKPHFRNTVERMYRDTFSYNFYNRPILSRRNTVWLCYEVKTKGPSRPPLDAKIFRGQVYSELKYHPEMRFF
HWFSKWRKLHRDQEYEVTWYISWSPCTKCTRDMATFLAEDPKVTLTIFVARLYYFWDPDYQEALRSLCQKR
DGPRATMKIMNYDEFQHCWSKFVYSQRELFEPWNNLPKYYILLHIMLGEILRHSMDPPTFTFNFNNEPWVR
GRHETYLCYEVERMHNDTWVLLNQRRGFLCNQAPHKHGFLEGRHAELCFLDVIPFWKLDLDQDYRVTCFTS
WSPCFSCAQEMAKFISKNKHVSLCIFTARIYDDQGRCQEGLRTLAEAGAKISIMTYSEFKHCWDTFVDHQG
CPFQPWDGLDEHSQDLSGRLRAILQNQEN
(italic: nucleic acid editing domain;
underline: cytoplasmic localization signal)
Human APOBEC-3F
(SEQ ID NO: 280)
MKPHFRNTVERMYRDTFSYNFYNRPILSRRNTVWLCYEVKTKGPSRPRLDAKIFRGQVYSQPEHHAEMCFL
SWFCGNQLPAYKCFQITWFVSWTPCPDCVAKLAEFLAEHPNVTLTISAARLYYYWERDYRRALCRLSQAGA
RVKIMDDEEFAYCWENFVYSEGQPFMPWYKFDDNYAFLHRTLKEILRNPMEAMYPHIFYFHFKNLRKAY
GRNESWLCFTMEVVKHHSPVSWKRGVFRNQVDPETHCHAERCFLSWFCDDILSPNTNYEVTWYTSWSPCPE
CAGEVAEFLARHSNVNLTIFTARLYYFWDTDYQEGLRSLSQEGASVEIMGYKDFKYCWENFVYNDDEPFK
PWKGLKYNFLFLDSKLQEILE
(italic: nucleic acid editing domain)
Human APOBEC-3B
(SEQ ID NO: 281)
MNPQIRNPMERMYRDTFYDNFENEPILYGRSYTWLCYEVKIKRGRSNLLWDTGVFRGQVYFKPQYHAEM
CFLSWFCGNQLPAYKCFQITWFVSWTPCPDCVAKLAEFLSEHPNVTLTISAARLYYYWERDYRRALCRLSQA
GARVTIMDYEEFAYCWENFVYNEGQQFMPWYKFDENYAFLHRTLKEILRYLMDPDTFTFNFNNDPLVLRR
RQTYLCYEVERLDNGTWVLMDQHMGFLCNEAKNLLCGFYGRHAELRFLDLVPSLQLDPAQIYRVTWFISWS
PCFSWGCAGEVRAFLQENTHVRLRIFAARIYDYDPLYKEALQMLRDAGAQVSIMTYDEFEYCWDTFVYRQ
GCPFQPWDGLEEHSQALSGRLRAILQNQGN
(italic: nucleic acid editing domain)
Human APOBEC-3C:
(SEQ ID NO: 282)
MNPQIRNPMKAMYPGTFYFQFKNLWEANDRNETWLCFTVEGIKRRSVVSWKTGVFRNQVDSETHCHAER
CFLSWFCDDILSPNTKYQVTWYTSWSPCPDCAGEVAEFLARHSNVNLTIFTARLYYFQYPCYQEGLRSLSQEG
VAVEIMDYEDFKYCWENFVYNDNEPFKPWKGLKTNFRLLKRRLRESLQ
(italic: nucleic acid editing domain)
Human APOBEC-3A:
(SEQ ID NO: 283)
MEASPASGPRHLMDPHIFTSNFNNGIGRHKTYLCYEVERLDNGTSVKMDQHRGFLHNQAKNLLCGFYGRH
AELRFLDLVPSLQLDPAQIYRVTWFISWSPCFSWGCAGEVRAFLQENTHVRLRIFAARIYDYDPLYKEALQML
RDAGAQVSIMTYDEFKHCWDTFVDHQGCPFQPWDGLDEHSQALSGRLRAILQNQGN
(italic: nucleic acid editing domain)
Human APOBEC-3H:
(SEQ ID NO: 284)
MALLTAETFRLQFNNKRRLRRPYYPRKALLCYQLTPQNGSTPTRGYFENKKKCHAEICFINEIKSMGLDETQ
CYQVTCYLTWSPCSSCAWELVDFIKAHDHLNLGIFASRLYYHWCKPQQKGLRLLCGSQVPVEVMGFPKFAD
CWENFVDHEKPLSFNPYKMLEELDKNSRAIKRRLERIKIPGVRAQGRYMDILCDAEV
(italic: nucleic acid editing domain)
Human APOBEC-3D
(SEQ ID NO: 285)
MNPQIRNPMERMYRDTFYDNFENEPILYGRSYTWLCYEVKIKRGRSNLLWDTGVFRGPVLPKRQSNHRQE
VYFRFENHAEMCFLSWFCGNRLPANRRFQITWFVSWNPCLPCVVKVTKFLAEHPNVTLTISAARLYYYRDRD
WRWVLLRLHKAGARVKIMDYEDFAYCWENFVCNEGQPFMPWYKFDDNYASLHRTLKEILRNPMEAMYP
HIFYFHFKNLLKACGRNESWLCFTMEVTKHHSAVFRKRGVFRNQVDPETHCHAERCFLSWFCDDILSPNTN
YEVTWYTSWSPCPECAGEVAEFLARHSNVNLTIFTARLCYFWDTDYQEGLCSLSQEGASVKIMGYKDFVSC
WKNFVYSDDEPFKPWKGLQTNFRLLKRRLREILQ
(italic: nucleic acid editing domain)
Human APOBEC-1
(SEQ ID NO: 286)
MTSEKGPSTGDPTLRRRIEPWEFDVFYDPRELRKEACLLYEIKWGMSRKIWRSSGKNTTNHVEVNFIKKFTS
ERDFHPSMSCSITWFLSWSPCWECSQAIREFLSRHPGVTLVIYVARLFWHMDQQNRQGLRDLVNSGVTIQI
MRASEYYHCWRNFVNYPPGDEAHWPQYPPLWMMLYALELHCIILSLPPCLKISRRWQNHLTFFRLHLQNC
HYQTIPPHILLATGLIHPSVAWR
Mouse APOBEC-1
(SEQ ID NO: 287)
MSSETGPVAVDPTLRRRIEPHEFEVFFDPRELRKETCLLYEINWGGRHSVWRHTSQNTSNHVEVNFLEKFTT
ERYFRPNTRCSITWFLSWSPCGECSRAITEFLSRHPYVTLFIYIARLYHHTDQRNRQGLRDLISSGVTIQIMTE
QEYCYCWRNFVNYPPSNEAYWPRYPHLWVKLYVLELYCIILGLPPCLKILRRKQPQLTFFTITLQTCHYQRI
PPHLLWATGLK
Rat APOBEC-1
(SEQ ID NO: 288)
MSSETGPVAVDPTLRRRIEPHEFEVFFDPRELRKETCLLYEINWGGRHSIWRHTSQNTNKHVEVNFIEKFTTE
RYFCPNTRCSITWFLSWSPCGECSRAITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNILRRKQPQLTFFTIALQSCHYQRLP
PHILWATGLK
Petromyzon marinus CDA1 (pmCDA1)
(SEQ ID NO: 289)
MTDAEYVRIHEKLDIYTFKKQFFNNKKSVSHRCYVLFELKRRGERRACFWGYAVNKPQSGTERGIHAEIFSI
RKVEEYLRDNPGQFTINWYSSWSPCADCAEKILEWYNQELRGNGHTLKIWACKLYYEKNARNQIGLWNL
RDNGVGLNVMVSEHYQCCRKIFIQSSHNQLNENRWLEKTLKRAEKRRSELSIMIQVKILHTTKSPAV
Human APOBEC3G D316R_D317R
(SEQ ID NO: 290)
MKPHFRNTVERMYRDTFSYNFYNRPILSRRNTVWLCYEVKTKGPSRPPLDAKIFRGQVYSELKYHPEMRFF
HWFSKWRKLHRDQEYEVTWYISWSPCTKCTRDMATFLAEDPKVTLTIFVARLYYFWDPDYQEALRSLCQ
KRDGPRATMKIMNYDEFQHCWSKFVYSQRELFEPWNNLPKYYILLHIMLGEILRHSMDPPTFTFNFNNEPW
VRGRHETYLCYEVERMHNDTWVLLNQRRGFLCNQAPHKHGFLEGRHAELCFLDVIPFWKLDLDQDYRVT
CFTSWSPCFSCAQEMAKFISKNKHVSLCIFTARIYRRQGRCQEGLRTLAEAGAKISIMTYSEFKHCWDTFVD
HQGCPFQPWDGLDEHSQDLSGRLRAILQNQEN
Human APOBEC3G chain A
(SEQ ID NO: 291)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYDDQGRCQEGLRTLAEAGAKISI
MTYSEFKHCWDTFVDHQGCPFQPWDGLDEHSQDLSGRLRAILQ
Human APOBEC3G chain A D120R_D121R
(SEQ ID NO: 292)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYRRQGRCQEGLRTLAEAGAKISI
MTYSEFKHCWDTFVDHQGCPFQPWDGLDEHSQDLSGRLRAILQ

Non-limiting examples of fusion proteins/nucleobase editors are provided.

His₆-rAPOBEC1-XTEN-dCas9 for Escherichia coli expression
(SEQ ID NO: 293)
MGSSHHHHHHMSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHADPRNRQGLRDLI
SSGVTIQIMTEQESGYCWRNFVNYSPSNEAHWPRYPHLWVRLYVLELYCIILGLPPCLNILRRKQPQLTFFTI
ALQSCHYQRLPPHILWATGLKSGSETPGTSESATPESDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQNGRDMYVD
QELDINRLSDYDVDAIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLASHYEKLKGSPEDN
EQKQLFVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDSGGSPKKKRKV
rAPOBEC1-XTEN-dCas9-NLS for Mammalian expression (SEQ ID NO: 294)
MSSETGPVAVDPTLRRRIEPHEFEVFFDPRELRKETCLLYEINWGGRHSIWRHTSQNTNKHVEVNFIEKFTT
ERYFCPNTRCSITWFLSWSPCGECSRAITEFLSRYPHVTLFIYIARLYHHADPRNRQGLRDLISSGVTIQIMTE
QESGYCWRNFVNYSPSNEAHWPRYPHLWVRLYVLELYCIILGLPPCLNILRRKQPQLTFFTIALQSCHYQRL
PPHILWATGLKSGSETPGTSESATPESDKKYSIGLAIGTNSVGWAVITDEYKVPSKKFKVLGNTDRHSIKKN
LIGALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQT
YNQLFEENPINASGVDAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDINRLSDY
DVDAIVPQSFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYFFYSNIMNFFKT
EITLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDTTIDRKRY
TSTKEVLDATLIHQSITGLYETRIDLSQLGGDSGGSPKKKRKV
hAPOBEC1-XTEN-dCas9-NLS for Mammalian expression (SEQ ID NO: 295)
MTSEKGPSTGDPTLRRRIEPWEFDVFYDPRELRKEACLLYEIKWGMSRKIWRSSGKNTTNHVEVNFIKKFTS
ERDFHPSMSCSITWFLSWSPCWECSQAIREFLSRHPGVTLVIYVARLFWHMDQQNRQGLRDLVNSGVTIQI
MRASEYYHCWRNFVNYPPGDEAHWPQYPPLWMMLYALELHCIILSLPPCLKISRRWQNHLTFFRLHLQNC
HYQTIPPHILLATGLIHPSVAWRSGSETPGTSESATPESDKKYSIGLAIGTNSVGWAVITDEYKVPSKKFKVL
GNTDRHSIKKNLIGALLFDSGETALATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKAPLSASMIKR
YDEHHQDLTLLKALVRQQLPEKYKEIFFDQSKNGYAGYIDGGASQLEFYKFIKPILEKMDGTEELLVKLNR
EDLLRKQRTFDNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNASLGTYHDLLKIIKDKDFLDNEE
NEDILEDIVLTLTLFEDREMIEERLKTYAHLFDDKVMKQLKRRRYTGWGRLSRKLINGIRDKQSGKTILDFL
KSDGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRDMYV
DQELDINRLSDYDVDAIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIMERSSFEKNPI
DFLEAKGYKEVKKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIHLFTLTNLGAPAA
FKYFDTTIDRKRYTSTKEVLDATLIHQSITGLYETRIDLSQLGGDSGGSPKKKRKV
rAPOBEC1-XTEN-dCas9-UGI-NLS (SEQ ID NO: 296)
MSSETGPVAVDPTLRRRIEPHEFEVFFDPRELRKETCLLYEINWGGRHSIWRHTSQNTNKHVEVNFIEKFTT
ERYFCPNTRCSITWFLSWSPCGECSRAITEFLSRYPHVTLFIYIARLYHHADPRNRQGLRDLISSGVTIQIMTE
QESGYCWRNFVNYSPSNEAHWPRYPHLWVRLYVLELYCIILGLPPCLNILRRKQPQLTFFTIALQSCHYQRL
PPHILWATGLKSGSETPGTSESATPESDKKYSIGLAIGTNSVGWAVITDEYKVPSKKFKVLGNTDRHSIKKN
LIGALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQT
YNQLFEENPINASGVDAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIKRYDEHHQDLTLLK
ALVRQQLPEKYKEIFFDQSKNGYAGYIDGGASQLEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQELDINRLSDY
DVDAIVPQSFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYFFYSNIMNFFKT
EITLANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDTTIDRKRY
TSTKEVLDATLIHQSITGLYETRIDLSQLGGDSGGSTNLSDIIEKETGKQLVIQESILMLPEEVEEVIGNKPESD
ILVHTAYDESTDENVMLLTSDAPEYKPWALVIQDSNGENKIKMLSGGSPKKKRKV
rAPOBEC1-XTEN-Cas9 nickase-UGI-NLS (BE3, SEQ ID NO: 297)
MSSETGPVAVDPTLRRRIEPHEFEVFFDPRELRKETCLLYEINWGGRHSIWRHTSQNTNKHVEVNFIEKFTT
ERYFCPNTRCSITWFLSWSPCGECSRAITEFLSRYPHVTLFIYIARLYHHADPRNRQGLRDLISSGVTIQIMTE
QESGYCWRNFVNYSPSNEAHWPRYPHLWVRLYVLELYCIILGLPPCLNILRRKQPQLTFFTIALQSCHYQRL
PPHILWATGLKSGSETPGTSESATPESDKKYSIGLAIGTNSVGWAVITDEYKVPSKKFKVLGNTDRHSIKKN
LIGALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQT
YNQLFEENPINASGVDAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIKRYDEHHQDLTLLK
ALVRQQLPEKYKEIFFDQSKNGYAGYIDGGASQLEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNEENEDILEDIVLTITL
FEDREMIEERLKTYAHLFDDKVMKQLKRRRYTGWGRLSRKLINGIRDKQSGKTILDFLKSDGFANRNFMQ
LIHDDSLTFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDINRLSDYD
VDHIVPQSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFYKVREIN
NYHHAHDAYLNAVVGTALIKKYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPEDNEQKQLFVEQHK
HYLDEIIEQISEFSKRVILADANLDKVLSAYNKHRDKPIREQAENIIHLFTLTNLGAPAAFKYFDTTIDRKRYT
STKEVLDATLIHQSITGLYETRIDLSQLGGDSGGSTNLSDIIEKETGKQLVIQESILMLPEEVEEVIGNKPESDI
LVHTAYDESTDENVMLLTSDAPEYKPWALVIQDSNGENKIKMLSGGSPKKKRKV
pmCDA1-XTEN-dCas9-UGI (bacteria) (SEQ ID NO: 298)
MTDAEYVRIHEKLDIYTFKKQFFNNKKSVSHRCYVLFELKRRGERRACFWGYAVNKPQSGTERGIHAEIFSI
RKVEEYLRDNPGQFTINWYSSWSPCADCAEKILEWYNQELRGNGHTLKIWACKLYYEKNARNQIGLWNL
RDNGVGLNVMVSEHYQCCRKIFIQSSHNQLNENRWLEKTLKRAEKRRSELSIMIQVKILHTTKSPAVSGSET
PGTSESATPESDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDAIVPQSFLKDDSI
DNKVLTRSDKNRGKSDNVPSEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVLDATLIHQSI
TGLYETRIDLSQLGGDSGGSMTNLSDIIEKETGKQLVIQESILMLPEEVELVIGNKPESDILVHTAYDESTDEN
VMLLTSDAPEYKPWALVIQDSNGENKIKML
pmCDA1-XTEN-nCas9-UGI-NLS (mammalian construct) (SEQ ID NO: 299):
MTDAEYVRIHEKLDIYTFKKQFFNNKKSVSHRCYVLFELKRRGERRACFWGYAVNKPQSGTERGIHAEIFSI
RKVELYLRDNPGQFTINWYSSWSPCADCALKILEWYNQELRGNGHTLKIWACKLYYEKNARNQIGLWNL
RDNGVGLNVMVSEHYQCCRKIFIQSSHNQLNENRWLEKTLKRAEKRRSELSIMIQVKILHTTKSPAVSGSET
PGTSESATPESDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVLDATLIHQSI
TGLYETRIDLSQLGGDSGGSTNLSDIIEKETGKQLVIQESILMLPEEVEEVIGNKPESDILVHTAYDESTDENV
MLLTSDAPEYKPWALVIQDSNGENKIKMLSGGSPKKKRKV
huAPOBEC3G-XTEN-dCas9-UGI (bacteria) (SEQ ID NO: 300)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYDDQGRCQEGLRTLAEAGAKISI
MTYSEFKHCWDTFVDHQGCPFQPWDGLDEHSQDLSGRLRAILQSGSETPGTSESATPESDKKYSIGLAIGTN
SVGWAVITDEYKVPSKKFKVLGNTDRHSIKKNLIGALLFDSGETALATRLKRTARRRYTRRKNRICYLQEIF
SNEMAKVDDSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDGGASQLEFYK
FIKPILEKMDGTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKELGSQILKEHPVENTQ
LQNEKLYLYYLQNGRDMYVDQELDINRLSDYDVDAIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQLGGDSGGSMTN
LSDIIEKETGKQLVIQESILMLPEEVEEVIGNKPESDILVHTAYDESTDENVMLLTSDAPEYKPWALVIQDSN
GENKIKML
huAPOBEC3G-XTEN-nCas9-UGI-NLS (mammalian construct) (SEQ ID NO: 301)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYDDQGRCQEGLRTLAEAGAKISI
MTYSEFKHCWDTFVDHQGCPFQPWDGLDEHSQDLSGRLRAILQSGSETPGTSESATPESDKKYSIGLAIGTN
SVGWAVITDEYKVPSKKFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDGGASQLEFYK
FIKPILEKMDGTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKELGSQILKEHPVENTQ
LQNEKLYLYYLQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQLGGDSGGSTNLS
DIIEKETGKQLVIQESILMLPEEVEEVIGNKPESDILVHTAYDESTDENVMLLTSDAPEYKPWALVIQDSNGE
NKIKMLSGGSPKKKRKV
huAPOBEC3G (D316R_D317R)-XTEN-nCas9-UGI-NLS (mammalian construct)
(SEQ ID NO: 302)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYRRQGRCQEGLRTLAEAGAKISI
MTYSEFKHCWDTFVDHQGCPFQPWDGLDEHSQDLSGRLRAILQSGSETPGTSESATPESDKKYSIGLAIGTN
SVGWAVITDEYKVPSKKFKVLGNTDRHSIKKNLIGALLFDSGETALATRLKRTARRRYTRRKNRICYLQEIF
SNEMAKVDDSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKELGSQILKEHPVENTQ
LQNEKLYLYYLQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQLGGDSGGSTNLS
DIIEKETGKQLVIQESILMLPEEVEEVIGNKPESDILVHTAYDESTDENVMLLTSDAPEYKPWALVIQDSNGE
NKIKMLSGGSPKKKRKV

### Example 2: CRISPR/Cas9 Genome/Base-Editing Methods for Modifying PCSK9 and Other Liver Proteins to Improve Circulating Cholesterol and Lipid Levels

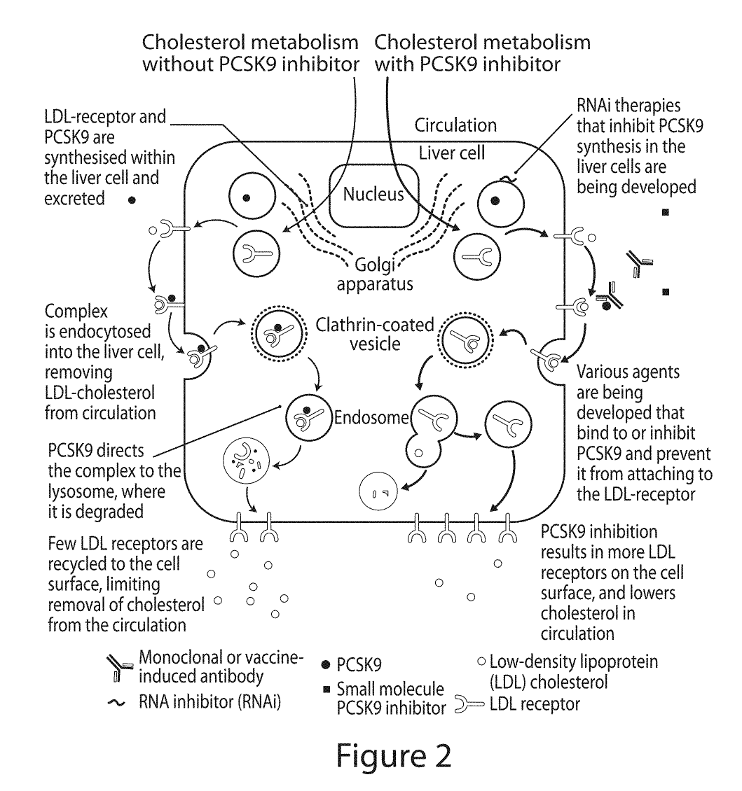

Approximately 70% of cholesterol in circulation is transported within low-density lipoproteins (LDL), which are cleared in the liver by LDL receptor (LDL-R)-mediated endocytosis, with the added consequence of downregulation of the endogenous cholesterol biosynthetic pathway. PCSK9 is a secreted, globular, serine protease capable of proteolytic auto-processing of its N-terminal pro-domain into a potent endogenous inhibitor, which permanently blocks its catalytic site (FIGS. 1A to 1C). A list of pharmaceutical agents used to block PCSK9 function can be found in Table 12. Mature PCSK9 exits through the secretory pathway and acts as a protein-binding adaptor in clathrin-coated vesicles to bridge a pH-dependent interaction with the LDL receptor during endocytosis of LDL particles, which prevents recycling of the LDL receptor to the cell surface (FIG. 2).¹ Knock-out mice models of PCSK9 display remarkably low circulating cholesterol levels,² due to enhanced presentation of LDLR on the cell surface and elevated uptake of LDL particles by hepatocytes. Human genome-wide association studies have identified deleterious gain-of-function variants of PCSK9 in hypercholesterolemic patients,³ as well as beneficial loss-of-function and unstable PCKS9 variants in hypo-cholesterolemic individuals (FIGS. 1A to 1C, Table 1).^{3b, c, 4} A list of known human PCSK9 variants can be found in Table 18.

Over the past decade there has been significant interest in the pharmaceutical industry to abrogate the interaction between PCSK9 and LDLR using various strategies including antibodies, small-molecules, peptidic ligands, RNA-interference, and antisense oligonucleotides (FIG. 2). Recently, the first generation of CRISPR/Cas9 tools have been used to ablate the PCSK9 gene in vivo in mouse models.⁵ However, due to the large number of cells that need to be modified in vivo to modulate cholesterol levels, there is a pressing concern about low-frequency off-target genomic instability and oncogenic modifications that could be caused by genome-editing treatments.⁶ Bridging the gap towards clinical applications will require safe and efficient strategies to modify PCSK9 in a way that maximizes the therapeutic benefits (Table 1). The precisely targeted methods for PCSK9 modifications disclosed here could be superior to previously proposed strategies that create random indels in the PCSK9 genomic site using engineered nucleases,⁶ including CRISPR/Cas9,⁷ as well as dCas9-Fok1 fusions,⁸ Cas9 nickase pairs,⁹ TALENs, zinc-finger nucleases, etc.¹⁰ Moreover, strategies that rely on “base-editors” such as BE2 or BE3,¹¹ may have a more favorable safety profile, due to the relatively low impact that off-target cytosine deamination has on genomic stability,¹² including oncogene activation or tumor suppressor inactivation.¹³

Importantly, PCSK9 is secreted by hepatocytes into the extracellular medium,¹⁴ where it acts in cis as a paracrine factor on neighboring hepatocytes' LDL receptors.¹⁴ Due to incomplete penetrance of gene/protein delivery into tissues in vivo, a significant fraction of the copies of PCSK9 genes remain as unmodified/wildtype.¹⁵ Therefore, loss-of-function variants of PCSK9 that are efficiently expressed, auto-activated, and exported to engage the clathrin-coated pits from unmodified cells in a paracrine mechanism should be prioritized for genome/base-editing therapeutics.

This carefully calibrated PCSK9 loss-of-function strategy could be accomplished by engineering variants of the key residues that make direct contacts with the LDL-R binding region, and specifically the EGF-A domain (FIGS. 1A to 1C), such as the PCSK9 residues R194, R237, F379, the beta-sheet 5372 to D374, the C375-378 disulfide, etc. (Table 3) as well as engineered and naturally-occurring variants that may affect global folding, such as residues R46 and R237, and A443 (Table 3). This therapeutic strategy would be beneficial to hypercholesterolemic patients that carry neutral PCSK9 variants, but even more so for carriers of deleterious gain-of-function mutations of PCSK9, LDLR, APOB, etc. (for example PCSK9-D374Y, FIGS. 1A to 1C).^{1b} Moreover, administration of multiple guide-RNAs in vivo could enable simultaneous introduction of other potentially synergistic genetic modifications, for example the rare cardio-protective alleles for APOC3 (A43T and R19X),16 the IDOL/MYLIP loss-of-function allele R266X,¹⁷ and the LDL-R non-coding variants that elevate gene expression (Table 9).¹⁸

Finally, new cardio-protective variants of PCSK9 could be identified by treating cells in vitro with guide-RNA libraries designed for all possible PAMs in the genomic site, coupled with FACS sorting using reporters/labeling methods and DNA-deep sequencing, to find the guide-RNAs that programmed base-editing reactions that change a reporter gene expression or display elevated LDL-R on the cell surface. These new PCSK9 variants, as well as other cardioprotective alleles identified by genome-wide association studies (and similarly for LDL-R, IDOL, APOC3/C5, etc.), could be recapitulated using the types of guide-RNA programmed base-editing reactions described herein (Tables 2 and 3).

Importantly, the introduction of STOP codons can be predicted to be most efficacious in generating truncations when targeting residues in flexible loops, or which can be edited processively in tandem using one guide-RNA BE complex (guide RNAs highlighted in blue).Examples of tandem introduction of premature stop codons into PCSK9 include: W10X-W11X, Q99X-Q101X, Q342X-Q344X, Q554X-Q555X. Similarly, a structurally destabilizing variants followed by a stop codon could also be efficacious, for example: P530S/L-Q531X, P581S/LR582X, P618S/L-Q619X (guide RNAs highlighted in red). Residues found in loop/linker regions are labeled + or ++.

TABLE 19
Examples of Pharmaceutical Agents for Blocking PCSK9 Function

| Mechanism of Action | Agent | Company/Sponsor | Phase |
|---|---|---|---|
| Monoclonal antibodies | SAR236553/REGN727 | Sanofi/Regeneron | Approved |
| | AMG 145 | Amgen | Approved |
| | RN316 | Pfizer | 3 |
| | RG7652 | Roche/Genentech | 2 |
| | LGT-209 | Novartis | 2 |
| | 1D05-IgG2 | Merck | Pre-clinical |
| | 1B20 | Merck | Pre-clinical |
| | J10, J16 | Pfizer | Pre-clinical |
| | J17 | Pfizer | Pre-clinical |
| Adnectins | BMS-962476 | Briston-Myers Squibb/Adnexus | 1 |
| Mimetic peptides | EGF-AB peptide | Schering-Plough | Pre-clinical |
| | fragment | | |
| | LDLR (H306Y) | U.S. National Institutes of | Pre-clinical |
| | subfragment | Health | |
| | LDLR DNA construct | U.S. National Institutes of | Pre-clinical |
| | | Health | |
| Small-molecule | SX-PCK9 | Serometrix | Pre-clinical |
| inhibitors | TBD | Shifa Biomedical | Pre-clinical |
| | ISIS 394814 | Isis | Pre-clinical |
| | SPC4061 | Santaris-Pharma | Pre-clinical |
| | SPC5011 | Santaris-Pharma | 1 (terminated) |
| RNA interference | ALN-PCS02 | Alnylam | 1 |

### REFERENCES

1. (a) Fisher, T. S.; Lo Surdo, P.; Pandit, S.; Mattu, M.; Santoro, J. C.; Wisniewski, D.; Cummings, R. T.; Calzetta, A.; Cubbon, R. M.; Fischer, P. A.; Tarachandani, A.; De Francesco, R.; Wright, S. D.; Sparrow, C. P.; Carfi, A.; Sitlani, A., Effects of pH and low density lipoprotein (LDL) on PCSK9-dependent LDL receptor regulation. The Journal of biological chemistry 2007, 282 (28), 20502-12; (b) Cunningham, D.; Danley, D. E.; Geoghegan, K. F.; Griffor, M. C.; Hawkins, J. L.; Subashi, T. A.; Varghese, A. H.; Ammirati, M. J.; Culp, J. S.; Hoth, L. R.; Mansour, M. N.; McGrath, K. M.; Seddon, A. P.; Shenolikar, S.; Stutzman-Engwall, K. J.; Warren, L. C.; Xia, D.; Qiu, X., Structural and biophysical studies of PCSK9 and its mutants linked to familial hypercholesterolemia. Nature structural & molecular biology 2007, 14 (5), 413-9.
2. Rashid, S.; Curtis, D. E.; Garuti, R.; Anderson, N. N.; Bashmakov, Y.; Ho, Y. K.; Hammer, R. E.; Moon, Y. A.; Horton, J. D., Decreased plasma cholesterol and hypersensitivity to statins in mice lacking Pcsk9. Proceedings of the National Academy of Sciences of the United States of America 2005, 102 (15), 5374-9.
3. (a) Abifadel, M.; Varret, M.; Rabes, J. P.; Allard, D.; Ouguerram, K.; Devillers, M.; Cruaud, C.; Benjannet, S.; Wickham, L.; Erlich, D.; Derre, A.; Villeger, L.; Farnier, M.; Beucler, I.; Bruckert, E.; Chambaz, J.; Chanu, B.; Lecerf, J. M.; Luc, G.; Moulin, P.; Weissenbach, J.; Prat, A.; Krempf, M.; Junien, C.; Seidah, N. G.; Boileau, C., Mutations in PCSK9 cause autosomal dominant hypercholesterolemia. Nature genetics 2003, 34 (2), 154-6; (b) Costet, P.; Krempf, M.; Cariou, B., PCSK9 and LDL cholesterol: unravelling the target to design the bullet. Trends in biochemical sciences 2008, 33 (9), 426-34; (c) Cohen, J. C.; Boerwinkle, E.; Mosley, T. H., Jr.; Hobbs, H. H., Sequence variations in PCSK9, low LDL, and protection against coronary heart disease. The New England journal of medicine 2006, 354 (12), 1264-72.
4. (a) Benjannet, S.; Rhainds, D.; Hamelin, J.; Nassoury, N.; Seidah, N. G., The proprotein convertase (PC) PCSK9 is inactivated by furin and/or PC5/6A: functional consequences of natural mutations and post-translational modifications. The Journal of biological chemistry 2006, 281 (41), 30561-72; (b) Cohen, J.; Pertsemlidis, A.; Kotowski, I. K.; Graham, R.; Garcia, C. K.; Hobbs, H. H., Low LDL cholesterol in individuals of African descent resulting from frequent nonsense mutations in PCSK9. Nature genetics 2005, 37 (2), 161-5.
5. (a) Ding, Q.; Strong, A.; Patel, K. M.; Ng, S. L.; Gosis, B. S.; Regan, S. N.; Cowan, C. A.; Rader, D. J.; Musunuru, K., Permanent alteration of PCSK9 with in vivo CRISPR-Cas9 genome editing. Circulation research 2014, 115 (5), 488-92; (b) Wang, X.; Raghavan, A.; Chen, T.; Qiao, L.; Zhang, Y.; Ding, Q.; Musunuru, K., CRISPR-Cas9 Targeting of PCSK9 in Human Hepatocytes In vivo-Brief Report. Arteriosclerosis, thrombosis, and vascular biology 2016, 36 (5), 783-6.
6. Cox, D. B.; Platt, R. J.; Zhang, F., Therapeutic genome editing: prospects and challenges. Nature medicine 2015, 21 (2), 121-31.
7. (a) Cong, L.; Ran, F. A.; Cox, D.; Lin, S.; Barretto, R.; Habib, N.; Hsu, P. D.; Wu, X.; Jiang, W.; Marraffini, L. A.; Zhang, F., Multiplex genome engineering using CRISPR/Cas systems. Science 2013, 339 (6121), 819-23; (b) Jinek, M.; Chylinski, K.; Fonfara, I.; Hauer, M.; Doudna, J. A.; Charpentier, E., A programmable dual-RNA-guided DNA endonuclease in adaptive bacterial immunity. Science 2012, 337 (6096), 816-21; (c) Mali, P.; Yang, L.; Esvelt, K. M.; Aach, J.; Guell, M.; DiCarlo, J. E.; Norville, J. E.; Church, G. M., RNA-guided human genome engineering via Cas9. Science 2013, 339 (6121), 823-6.
8. (a) Guilinger, J. P.; Thompson, D. B.; Liu, D. R., Fusion of catalytically inactive Cas9 to FokI nuclease improves the specificity of genome modification. Nature biotechnology 2014, 32 (6), 577-82; (b) Tsai, S. Q.; Wyvekens, N.; Khayter, C.; Foden, J. A.; Thapar, V.; Reyon, D.; Goodwin, M. J.; Aryee, M. J.; Joung, J. K., Dimeric CRISPR RNA-guided FokI nucleases for highly specific genome editing. Nature biotechnology 2014, 32 (6), 569-76.
9. Ran, F. A.; Hsu, P. D.; Lin, C. Y.; Gootenberg, J. S.; Konermann, S.; Trevino, A. E.; Scott, D. A.; Inoue, A.; Matoba, S.; Zhang, Y.; Zhang, F., Double nicking by RNAguided CRISPR Cas9 for enhanced genome editing specificity. Cell 2013, 154 (6), 1380-9.
10. (a) Cradick, T. J.; Fine, E. J.; Antico, C. J.; Bao, G., CRISPR/Cas9 systems targeting β-globin and CCR5 genes have substantial off-target activity. Nucleic acids research 2013; (b) Holt, N.; Wang, J.; Kim, K.; Friedman, G.; Wang, X.; Taupin, V.; Crooks, G. M.; Kohn, D. B.; Gregory, P. D.; Holmes, M. C.; Cannon, P. M., Human hematopoietic stem/progenitor cells modified by zinc-finger nucleases targeted to CCR5 control HIV-1 in vivo. Nature biotechnology 2010, 28 (8), 839-47.
11. Komor, A. C.; Kim, Y. B.; Packer, M. S.; Zuris, J. A.; Liu, D. R., Programmable editing of a target base in genomic DNA without double-stranded DNA cleavage. Nature 2016, advance online publication.
12. Koonin, E. V.; Novozhilov, A. S., Origin and evolution of the genetic code: the universal enigma. IUBMB life 2009, 61 (2), 99-111.
13. (a) Thomas, M. A.; Weston, B.; Joseph, M.; Wu, W.; Nekrutenko, A.; Tonellato, P. J., Evolutionary dynamics of oncogenes and tumor suppressor genes: higher intensities of purifying selection than other genes. Molecular biology and evolution 2003, 20 (6), 964-8; (b) Iengar, P., An analysis of substitution, deletion and insertion mutations in cancer genes. Nucleic acids research 2012, 40 (14), 6401-13.
14. (a) Lagace, T. A.; Curtis, D. E.; Garuti, R.; McNutt, M. C.; Park, S. W.; Prather, H. B.; Anderson, N. N.; Ho, Y. K.; Hammer, R. E.; Horton, J. D., Secreted PCSK9 decreases the number of LDL receptors in hepatocytes and in livers of parabiotic mice. The Journal of clinical investigation 2006, 116 (11), 2995-3005; (b) Ferri, N.; Tibolla, G.; Pirillo, A.; Cipollone, F.; Mezzetti, A.; Pacia, S.; Corsini, A.; Catapano, A. L., Proprotein convertase subtilisin kexin type 9 (PCSK9) secreted by cultured smooth muscle cells reduces macrophages LDLR levels. Atherosclerosis 2012, 220 (2), 381-6.
15. (a) Zuris, J. A.; Thompson, D. B.; Shu, Y.; Guilinger, J. P.; Bessen, J. L.; Hu, J. H.; Maeder, M. L.; Joung, J. K.; Chen, Z. Y.; Liu, D. R., Cationic lipid-mediated delivery of proteins enables efficient protein-based genome editing in vitro and in vivo. Nature biotechnology 2015, 33 (1), 73-80; (b) Yin, H.; Song, C. Q.; Dorkin, J. R.; Zhu, L. J.; Li, Y.; Wu, Q.; Park, A.; Yang, J.; Suresh, S.; Bizhanova, A.; Gupta, A.; Bolukbasi, M. F.; Walsh, S.; Bogorad, R. L.; Gao, G.; Weng, Z.; Dong, Y.; Koteliansky, V.; Wolfe, S. A.; Langer, R.; Xue, W.; Anderson, D. G., Therapeutic genome editing by combined viral and non-viral delivery of CRISPR system components in vivo. Nature biotechnology 2016, 34 (3), 328-33.
16. Jorgensen, A. B.; Frikke-Schmidt, R.; Nordestgaard, B. G.; Tybjaerg-Hansen, A., Loss-of-function mutations in APOC3 and risk of ischemic vascular disease. The New England journal of medicine 2014, 371 (1), 32-41.
17. Sorrentino, V.; Fouchier, S. W.; Motazacker, M. M.; Nelson, J. K.; Defesche, J. C.; Dallinga-Thie, G. M.; Kastelein, J. J.; Kees Hovingh, G.; Zelcer, N., Identification of a loss-of-function inducible degrader of the low-density lipoprotein receptor variant in individuals with low circulating low-density lipoprotein. European heart journal 2013, 34 (17), 1292-7.
18. (a) Scholtz, C. L.; Peeters, A. V.; Hoogendijk, C. F.; Thiart, R.; de Villiers, J. N.; Hillermann, R.; Liu, J.; Marais, A. D.; Kotze, M. J., Mutation-59c->t in repeat 2 of the LDL receptor promoter: reduction in transcriptional activity and possible allelic interaction in a South African family with familial hypercholesterolaemia. Human molecular genetics 1999, 8 (11), 2025-30; (b) Gretarsdottir, S.; Helgason, H.; Helgadottir, A.; Sigurdsson, A.; Thorleifsson, G.; Magnusdottir, A.; Oddsson, A.; Steinthorsdottir, V.; Rafnar, T.; de Graaf, J.; Daneshpour, M. S.; Hedayati, M.; Azizi, F.; Grarup, N.; Jorgensen, T.; Vestergaard, H.; Hansen, T.; Eyjolfsson, G.; Sigurdardottir, O.; Olafsson, I.; Kiemeney, L. A.; Pedersen, O.; Sulem, P.; Thorgeirsson, G.; Gudbjartsson, D. F.; Holm, H.; Thorsteinsdottir, U.; Stefansson, K., A Splice Region Variant in LDLR Lowers Non-high Density Lipoprotein Cholesterol and Protects against Coronary Artery Disease. PLoS genetics 2015, 11 (9), e1005379; (c) van Zyl, T.; Jerling, J. C.; Conradie, K. R.; Feskens, E. J., Common and rare single nucleotide polymorphisms in the LDLR gene are present in a black South African population and associate with low-density lipoprotein cholesterol levels. Journal of human genetics 2014, 59 (2), 88-94; (d) De Castro-Oros, I.; Perez-Lopez, J.; Mateo-Gallego, R.; Rebollar, S.; Ledesma, M.; Leon, M.; Cofan, M.; Casasnovas, J. A.; Ros, E.; Rodriguez-Rey, J. C.; Civeira, F.; Pocovi, M., A genetic variant in the LDLR promoter is responsible for part of the LDL-cholesterol variability in primary hypercholesterolemia. BMC medical genomics 2014, 7, 17.
19. Kwon, H. J.; Lagace, T. A.; McNutt, M. C.; Horton, J. D.; Deisenhofer, J., Molecular basis for LDL receptor recognition by PCSK9. Proceedings of the National Academy of Sciences of the U.S. Pat. No. 2,008,105 (6), 1820-5.
20. Dewpura, T.; Raymond, A.; Hamelin, J.; Seidah, N. G.; Mbikay, M.; Chretien, M.; Mayne, J., PCSK9 is phosphorylated by a Golgi casein kinase-like kinase ex vivo and circulates as a phosphoprotein in humans. The FEBS journal 2008, 275 (13), 3480-93.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of editing a polynucleotide encoding a Proprotein Convertase Subtilisin/Kexin Type 9 (PCSK9) protein, the method comprising contacting the PCSK9-encoding polynucleotide with:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a target cytosine (C) base in the PCSK9-encoding polynucleotide,
wherein the contacting results in deamination of the target C base by the fusion protein, resulting in a cytosine (C) to thymine (T) change in the PCSK9-encoding polynucleotide.

2. The method of claim 1, wherein the guide nucleotide sequence-programmable DNA binding protein is a nickase.

3. The method of claim 1, wherein the guide nucleotide sequence-programmable DNA binding protein domain is a Cas9 nickase.

4. The method of claim 3, wherein the Cas9 nickase comprises a mutation corresponding to a D10A mutation or an H840A mutation in SEQ ID NO: 1.

5. (canceled)

6. The method of claim 1, wherein the guide nucleotide sequence-programmable DNA binding protein domain is selected from the group consisting of: nuclease inactive Cas9 (dCas9) domains, nuclease inactive Cpf1 domains, nuclease inactive Argonaute domains, and variants thereof.

7-13. (canceled)

14. The method of claim 1, wherein the cytosine deaminase domain comprises an apolipoprotein B mRNA-editing complex (APOBEC) family deaminase.

15-16. (canceled)

17. The method of claim 1, wherein the fusion protein of (i) further comprises a Gam protein.

18. (canceled)

19. The method of claim 1, wherein the fusion protein of (i) further comprises a uracil glycosylase inhibitor (UGI) domain.

20-29. (canceled)

30. The method of claim 1, wherein the polynucleotide encoding the PCSK9 protein comprises a coding strand and a complementary strand.

31. (canceled)

32. The method of claim 1, wherein the C to T change in the PCSK9-encoding polynucleotide leads to a mutation in the PCSK9 protein.

33. (canceled)

34. The method of claim 32, wherein the mutation in the PCSK9 protein is a loss-of-function mutation.

35. The method of claim 34, wherein the mutation is selected from the mutations listed in Table 3.

36. The method of claim 35, wherein the guide nucleotide sequence comprises a guide sequence listed in Table 3.

37. The method of claim 34, wherein the loss-of-function mutation is a premature stop codon that leads to a truncated or non-functional PCSK9 protein.

38-44. (canceled)

45. The method of claim 37, wherein the guide nucleotide sequence comprises a guide sequence listed in Table 6.

46-47. (canceled)

48. The method of claim 37, wherein the premature stop codon is introduced after a structurally destabilizing mutation, wherein the destabilizing mutation is selected from the group consisting of P530S/L, P581S/L, and P618S/L, and wherein the premature stop codon is selected from the group consisting of Q531X, R582X, and Q619X, wherein X is a stop codon.

49-51. (canceled)

52. The method of claim 34, wherein the mutation destabilizes PCSK9 protein folding.

53. The method of claim 52, wherein the mutation is selected from the mutations listed in Table 4.

54. The method of claim 53, wherein the guide nucleotide sequence comprises a guide sequence listed in Table 4.

55. The method of claim 1, wherein the C to T change occurs at a splicing site of the PCSK9-encoding polynucleotide.

56-59. (canceled)

60. The method of claim 55, wherein the C to T change prevents PCSK9 mRNA maturation or abrogates PCSK9 expression.

61. The method of claim 60, wherein the guide nucleotide sequence comprises a guide sequence listed in Table 8.

62-69. (canceled)

70. The method of claim 1, wherein the guide nucleotide sequence is RNA (gRNA).

71. (canceled)

72. A method of editing a polynucleotide encoding an Apolipoprotein C3 (APOC3) protein, the method comprising contacting the APOC3-encoding polynucleotide with:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a target cytosine (C) base in the APOC3-encoding polynucleotide,
wherein the contacting results in deamination of the target C base by the fusion protein, resulting in a cytosine (C) to thymine (T) change in the APOC3-encoding polynucleotide.

73-91. (canceled)

92. A method of editing a polynucleotide encoding a Low-Density Lipoprotein Receptor (LDL-R) protein, the method comprising contacting the LDL-R-encoding polynucleotide with:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a target cytosine (C) base in the LDL-R-encoding polynucleotide,
wherein the contacting results in deamination of the target C base by the fusion protein, resulting in a cytosine (C) to thymine (T) change in the LDLR-encoding polynucleotide.

93. (canceled)

94. A method of editing a polynucleotide encoding an Inducible Degrader of the LDL receptor (IDOL) protein, the method comprising contacting the IDOL-encoding polynucleotide with:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a target C base in the IDOL-encoding polynucleotide,
wherein the contacting results in deamination of the target C base by the fusion protein, resulting in a cytosine (C) to thymine (T) change in the IDOL-encoding polynucleotide.

95-101. (canceled)

102. A method of editing a polynucleotide encoding a Proprotein Convertase Subtilisin/Kexin Type 9 (PCSK9) protein, the method comprising contacting the PCSK9-encoding polynucleotide with a fusion protein comprising: (a) a programmable DNA binding protein domain; and (b) a deaminase domain,
wherein the contacting results in deamination of the target base by the fusion protein, resulting in base change in the PCSK9-encoding polynucleotide.

103-109. (canceled)

110. A composition comprising:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a polynucleotide encoding a Proprotein Convertase subtilisin/Kexin Type 9 (PCSK9) protein.

111-123. (canceled)

124. A method of boosting LDL receptor-mediated clearance of LDL cholesterol, the method comprising administering to a subject in need thereof an therapeutically effective amount of the composition of claim 110.

125. A method of reducing circulating cholesterol level in a subject, the method comprising administering to a subject in need thereof an therapeutically effective amount of the composition of claim 110.

126-128. (canceled)
